(19) United States
(12) Patent Application Publication
(10) Pub. No.: US 2011/0230350 A1
(43) Pub. Date: Sep. 22, 2011

(54) ARYL-AND HETARYLSULFONAMIDES AS ACTIVE INGREDIENTS AGAINST ABIOTIC PLANT STRESS

(75) Inventors: Jens Frackenpohl, Frankfurt (DE); Ines Heinemann, Hofheim (DE); Thomas Müller, Frankfurt (DE); Pascal Von Koskull-Döring, Frankfurt am Main (DE); Jan Dittgen, Frankfurt (DE); Dirk Schmutzler, Hattersheim (DE); Christopher Hugh Rosinger, Hofheim (DE); Isolde Häuser-Hahn, Leverkusen (DE); Martin Jeffrey Hills, Idstein (DE)

(73) Assignee: BAYER CROPSCIENCE AG, Monheim (DE)

(21) Appl. No.: 13/050,273

(22) Filed: Mar. 17, 2011

Related U.S. Application Data
(60) Provisional application No. 61/315,583, filed on Mar. 19, 2010.

(30) Foreign Application Priority Data
Mar. 18, 2010 (EP) 10156922.6

(51) Int. Cl. A01N 43/40; C07D 231/16; C07D 213/46; C07D 401/12; C07D 209/14; C07D 307/81; C07D 405/12; A01N 43/56; A01N 43/42; A01N 43/38; A01N 43/08; A01P 3/00; A01P 1/00; A01P 7/04; A01P 21/00

(52) U.S. Cl. 504/247; 548/375.1; 546/338; 546/172; 548/503; 548/467; 549/469; 546/284.1; 504/280; 504/244; 504/284; 504/252; 504/298; 504/251

(57) ABSTRACT

Use of substituted sulfonamides of the formula (I) or salts thereof

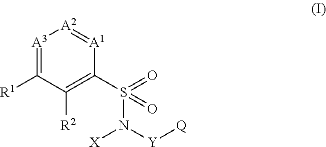

for enhancing stress tolerance in plants to abiotic stress, especially for enhancing plant growth and/or for increasing plant yield.

### CROSS REFERENCE TO RELATED APPLICATIONS

This application claims priority to EP 10156922.6 filed on Mar. 18, 2010 and U.S. Application No. 61/315,583 filed Mar. 19, 2010, the entire contents are incorporated by reference in their entireties.

### BACKGROUND OF THE INVENTION

1. Field of Invention

The invention relates to aryl- and hetarylsulfonamides and the analogs thereof, to processes for preparation thereof and to the use thereof for enhancing stress tolerance in plants to abiotic stress, especially for enhancing plant growth and/or for increasing plant yield.

2. Description of Related Art

It is known that particular arylsulfonamides, for example 2-cyanobenzenesulfonamides, have insecticidal properties (cf., for example, EP0033984 and WO2005/035486, WO2006/056433, WO2007/060220). 2-Cyanobenzenesulfonamides with particular heterocyclic substituents are described in EP2065370. It is also known that particular substituted 2-cyanobenzenesulfonamides can be used for soil and seed applications (cf. WO2006/100271 and WO2006/100288). The use of arylsulfonamides with 2-aminomethyl-3-chloro-5-trifluoromethylpyridyl substituents as fungicides is described in WO9942447. It is also known that particular bisarylsulfonamides can be used as fungicides (cf. WO2009101082).

It is also known that substituted arylsulfonamides (cf., for example, WO2009105774) and substituted hetarylsulfonamides (cf. WO2009113600) can be used as active pharmaceutical ingredients. WO2003007931 likewise describes the pharmaceutical use of substituted naphthylsulfonamides, while Bioorg. Med. Chem. 2007, 15, 7553 describes N-quinolin-8-ylarylsulfonamides and the antiparasitic action thereof. The use of arylsulfonamides with aminomethylisoxazolyl, -pyrazolyl and -isothiazolyl substituents as active pharmaceutical ingredients is described in WO2007129019.

It is known that plants react to natural stress conditions, for example cold, heat, drought, injury, pathogenic attack (viruses, bacteria, fungi, insects), etc., but also to herbicides, with specific or unspecific defense mechanisms [Pflanzenbiochemie, p. 393-462, Spektrum Akademischer Verlag, Heidelberg, Berlin, Oxford, Hans W. Heldt, 1996; Biochemistry and Molecular Biology of Plants, p. 1102-1203, American Society of Plant Physiologists, Rockville, Md., eds. Buchanan, Gruissem, Jones, 2000].

In plants, there is knowledge of numerous proteins, and the genes which code for them, which are involved in defense reactions to abiotic stress (for example cold, heat, drought, salt, flooding). Some of these form part of signal transduction chains (for example transcription factors, kinases, phosphatases) or cause a physiological response of the plant cell (for example ion transport, deactivation of reactive oxygen species). The signaling chain genes of the abiotic stress reaction include transcription factors of the DREB and CBF classes (Jaglo-Ottosen et al., 1998, Science 280: 104-106). The reaction to salinity stress involves phosphatases of the ATPK and MP2C types. In addition, in the event of salinity stress, the biosynthesis of osmolytes such as proline or sucrose is often activated. This involves, for example, sucrose synthase and proline transporter (Hasegawa et al., 2000, Annu Rev Plant Physiol Plant Mol Biol 51: 463-499). The stress defense of the plants to cold and drought uses some of the same molecular mechanisms. There is a known accumulation of what are called late embryogenesis abundant proteins (LEA proteins), which include the dehydrins as an important class (Ingram and Bartels, 1996, Annu Rev Plant Physiol Plant Mol Biol 47: 277-403, Close, 1997, Physiol Plant 100: 291-296). These are chaperones which stabilize vesicles, proteins and membrane structures in stressed plants (Bray, 1993, Plant Physiol 103: 1035-1040). In addition, there is frequently induction of aldehyde dehydrogenases, which deactivate the reactive oxygen species (ROS) which form in the event of oxidative stress (Kirch et al., 2005, Plant Mol Biol 57: 315-332). Heat shock factors (HSF) and heat shock proteins (HSP) are activated in the event of heat stress and play a similar role here as chaperones to that of dehydrins in the event of cold and drought stress (Yu et al., 2005, Mol Cells 19: 328-333).

A number of signaling substances which are endogenous to plants and are involved in stress tolerance or pathogenic defense are already known. Examples here include salicylic acid, benzoic acid, jasmonic acid or ethylene [Biochemistry and Molecular Biology of Plants, p. 850-929, American Society of Plant Physiologists, Rockville, Md., eds. Buchanan, Gruissem, Jones, 2000]. Some of these substances or the stable synthetic derivatives and derived structures thereof are also effective on external application to plants or in seed dressing, and activate defense reactions which cause elevated stress tolerance or pathogen tolerance of the plant [Sembdner, and Parthier, 1993, Ann. Rev. Plant Physiol. Plant Mol. Biol. 44: 569-589].

It is known that the tolerance of plants to abiotic stress can be increased by a modification of the activity of endogenous poly-ADP-ribose polymerases (PARP) or poly-(ADP-ribose) glycohydrolases (PARG) (de. Block et al., The Plant Journal, 2004, 41, 95; Levine et al., FEBS Lett. 1998, 440, 1; WO0004173; WO04090140).

It is additionally known that chemical substances can increase the tolerance of plants to abiotic stress. Such substances are applied by seed dressing, by leaf spraying or by soil treatment. For instance, an increase in the abiotic stress tolerance of crop plants by treatment with elicitors of systemic acquired resistance (SAR) or abscisic acid derivatives is described (Schading and Wei, WO200028055; Abrams and Gusta, U.S. Pat. No. 5,201,931; Abrams et al, WO9723441, Churchill et al., 1998, Plant Growth Regul 25: 35-45). In addition, effects of growth regulators on the stress tolerance of crop plants have been described (Morrison and Andrews, 1992, J Plant Growth Regul 11: 113-117, RD-259027). In this context, it is likewise known that a growth-regulating naphthylsulfonamide (4-bromo-N-(pyridin-2-ylmethyl)naphthalene-1-sulfonamide) influences the germination of plant seeds in the same way as abscisic acid (Park et al. Science 2009, 324, 1068-1071). It is also known that a further naphthylsulfonamide, N-(6-aminohexyl)-5-chloronaphthalene-1-sulfonamide, influences the calcium level in plants which have been exposed to cold shock (Cholewa et al. Can. J. Botany 1997, 75, 375-382).

Similar effects are also observed on application of fungicides, especially from the group of the strobilurins or of the succinate dehydrogenase inhibitors, and are frequently also accompanied by an increase in yield (Draber et al., DE-3534948, Bartlett et al., 2002, Pest Manag Sci 60: 309). It is likewise known that the herbicide glyphosate in low dosage stimulates the growth of some plant species (Cedergreen, Env. Pollution 2008, 156, 1099).

In the event of osmotic stress, a protective effect has been observed as a result of application of osmolytes, for example glycine betaine or the biochemical precursors thereof, e.g. choline derivatives (Chen et al., 2000, Plant Cell Environ 23: 609-618, Bergmann et al., DE-4103253). The effect of antioxidants, for example naphthols and xanthines, for increasing abiotic stress, tolerance in plants has likewise already been described (Bergmann et al., DD-277832, Bergmann et al., DD-277835). However, the molecular causes of the antistress action of these substances are substantially unknown.

It is thus known that plants possess several endogenous reaction mechanisms which can bring about effective defense against a wide variety of different harmful organisms and/or natural abiotic stress.

However, since the environmental and economic demands made on modern crop treatment compositions are increasing constantly, for example in relation to toxicity, selectivity, application rate, formation of residues and favorable manufacture, and there can also be problems, for example, with resistances, it is a constant objective to develop novel crop treatment compositions, which have advantages over the known compositions at least in some areas.

### SUMMARY

The object of the present invention was therefore to provide further improved compounds which increase tolerance to abiotic stress in plants.

The present invention accordingly provides for the use of substituted sulfonamides of the general formula (I) or salts thereof

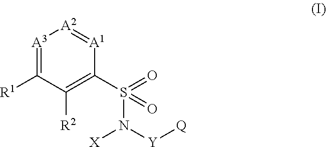

in which
A¹, A², A³ are the same or different and are each independently N (nitrogen) or the C—W moiety, but there are never more than two adjacent nitrogen atoms, and where W in the C—W moiety in each case has identical or different definitions as defined below,
W in each case is hydrogen, nitro, amino, cyano, thiocyanato, isothiocyanato, halogen, alkyl, cycloalkyl, alkenyl, alkynyl, aryl, arylalkyl, arylalkoxy, heteroaryl, alkoxyalkyl, haloalkyl, halocycloalkyl, alkoxy, haloalkoxy, alkoxycarbonyl, hydroxycarbonyl, aminocarbonyl, alkylaminocarbonyl, cycloalkylaminocarbonyl, alkylamino, alkylthio, haloalkylthio, bisalkylamino, cycloalkylamino, alkylcarbonylamino, cycloalkylcarbonylamino, formylamino, haloalkylcarbonylamino, alkoxycarbonylamino, (alkyl)aminocarbonylamino, alkylsulfonylamino, cycloalkylsulfonylamino, arylsulfonylamino, hetarylsulfonylamino, sulfonylhaloalkylamino, aminoalkylsulfonyl, aminohaloalkylsulfonyl, alkylsulfonyl, cycloalkylsulfonyl, arylsulfonyl, alkylsulfinyl, cycloalkylsulfinyl, arylsulfinyl, N,S-dialkylsulfonimidoyl, S-alkylsulfonimidoyl, alkylsulfonylaminocarbonyl, cycloalkylsulfonylaminocarbonyl, cycloalkylaminosulfonyl,
R¹ is H, nitro, amino, cyano, halogen, alkyl, cycloalkyl, alkenyl, alkynyl, aryl, arylalkyl, heteroaryl, alkoxyalkyl, haloalkyl, halocycloalkyl, alkoxy, haloalkoxy, alkylthio, haloalkylthio, alkoxycarbonyl, hydroxycarbonyl, aminocarbonyl, alkylaminocarbonyl, alkylamino, bisalkylamino, cycloalkylamino, alkylcarbonylamino, cycloalkylcarbonylamino, formylamino, haloalkylcarbonylamino, alkoxycarbonylamino, (alkyl)aminocarbonylamino, sulfonylamino, alkylsulfonylamino, haloalkylsulfonylamino, cycloalkylsulfonylamino, aminoalkylsulfonyl, aminohaloalkylsulfonyl, alkylsulfonyl, cycloalkylsulfonyl, arylsulfonyl, alkylsulfinyl, cycloalkylsulfinyl, arylsulfinyl, N,S-dialkylsulfonimidoyl, S-alkylsulfonimidoyl, alkylsulfonylaminocarbonyl, cycloalkylsulfonylaminocarbonyl, cycloalkylaminosulfonyl,
R² is H, nitro, amino, cyano, halogen, alkyl, cycloalkyl, alkenyl, alkynyl, aryl, arylalkyl, heteroaryl, alkoxyalkyl, haloalkyl, halocycloalkyl, alkoxy, haloalkoxy, alkylthio, haloalkylthio, alkoxycarbonyl, hydroxycarbonyl, aminocarbonyl, alkylaminocarbonyl, alkylamino, bisalkylamino, cycloalkylamino, alkylcarbonylamino, cycloalkylcarbonylamino, formylamino, haloalkylcarbonylamino, alkoxycarbonylamino, (alkyl)aminocarbonylamino, sulfonylamino, alkylsulfonylamino, haloalkylsulfonylamino, cycloalkylsulfonylamino, aminoalkylsulfonyl, aminohaloalkylsulfonyl, alkylsulfonyl, cycloalkylsulfonyl, arylsulfonyl, alkylsulfinyl, cycloalkylsulfinyl, arylsulfinyl, N,S-dialkylsulfonimidoyl, S-alkylsulfonimidoyl, alkylsulfonylaminocarbonyl, cycloalkylsulfonylaminocarbonyl, cycloalkylaminosulfonyl,
R¹ and R² with the atoms to which they are bonded form a fully saturated, partly saturated or fully unsaturated, 5 to 7-membered ring optionally interrupted by heteroatoms and optionally with further substitution,
R¹ and A³, when A³ is a C—W group, with the atoms to which they are bonded form a fully saturated, partly saturated or fully unsaturated, 5 to 7-membered ring optionally interrupted by heteroatoms and optionally with further substitution,
X is H, alkyl, alkylalkenyl, alkynyl, alkylcarbonyl, cycloalkylcarbonyl, arylcarbonyl, heteroarylcarbonyl, alkoxycarbonyl, alkylsulfonyl, cycloalkylsulfonyl, arylsulfonyl, alkoxycarbonylcarbonyl, arylalkoxycarbonylcarbonyl, alkylaminothiocarbonyl, alkylaminocarbonyl, arylalkyl, hetaroarylalkyl, cyanoalkyl, haloalkyl, cycloalkyl, cycloalkylalkyl,
Y is alkanediyl, cycloalkanediyl, alkenediyl, alkynediyl, alkylalkenediyl, arylalkanediyl, alkoxydiyl, alkoxyalkyldiyl, alkylthioalkyldiyl, alkylsulfinylalkyldiyl, alkylsulfonylalkyldiyl or a direct bond between N and Q,
Q is a phenyl ring or a 5-, 6- or 7-membered saturated or unsaturated heterocyclic ring or a 9- or 10-membered fused bicyclic or tricyclic ring system which optionally contains further heteroatoms, where the particular aforementioned ring or the ring system is optionally mono- or polysubstituted, identically or differently, and where the substituents are each independently selected from H, halogen, cyano, nitro, branched or unbranched alkyl, cycloalkyl, alkoxycycloalkyl, arylcycloalkyl, cycloalkylcycloalkyl, cycloalkylalkyl, alkenyl, alkynyl, arylalkyl, alkoxyalkyl, haloalkyl, halocycloalkyl, alkoxy, haloalkoxy, alkoxycarbonyl, hydroxycarbonyl, aminocarbonyl, alkylaminocarbonyl, amino, alkylamino, bisalkylamino, cycloalkylamino, alkylcarbonylamino, alkylcarbonyl, alkylthio, haloalkylthio, haloalkylsulfinyl, haloalkylsulfonyl or where the substituents are each independently selected from phenyl or a 5- or 6-membered heteroaromatic ring, where phenyl or the heteroaromatic ring is optionally mono- or polysubstituted, identically or differently, by alkyl, alkenyl, alkynyl, cycloalkyl, haloalkyl, haloalkenyl, haloalkynyl, halocycloalkyl, halogen, cyano, nitro, alkoxy, haloalkoxy, alkylthio, haloalkylthio,
   for increasing tolerance to abiotic stress in plants.

### DETAILED DESCRIPTION OF A PREFERRED EMBODIMENT

The compounds of the formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. These salts then contain the conjugate base of the acid as the anion.

Suitable substituents present in deprotonated form, for example sulfonic acids or carboxylic acids, can form internal salts with groups which are themselves protonatable, such as amino groups.

The compounds of the formula (I) used in accordance with the invention and salts thereof are referred to hereinafter as “compounds of the general formula (I)”.

Preference is given to the inventive use of compounds of the formula (I) in which
A¹, A², A³ are the same or different and are each independently N (nitrogen) or the C—W moiety, but there are never more than two adjacent nitrogen atoms, and where W in the C—W moiety in each case may have identical or different definitions as defined below,
W in each case is hydrogen, nitro, amino, cyano, halogen, (C₁-C₆)-alkyl, (C₃-C₈)-cycloalkyl, (C₂-C₈)-alkenyl, (C₂-C₈)-alkynyl, aryl, aryl-(C₁-C₆)-alkyl, heteroaryl, (C₁-C₆)-alkoxy-(C₁-C₆)-alkyl, (C₁-C₆)-haloalkyl, (C₃-C₈)-halocycloalkyl, (C₁-C₆)-alkoxy, aryl-(C₁-C₆)-alkoxy, (C₁-C₆)-haloalkoxy, (C₁-C₆)-alkoxycarbonyl, hydroxycarbonyl, aminocarbonyl, (C₁-C₆)-alkylaminocarbonyl, (C₃-C₈)-cycloalkylaminocarbonyl, (C₁-C₆)-alkylamino, (C₁-C₆)-alkylthio, (C₁-C₆)-haloalkylthio, bis(C₁-C₆)-alkylamino, (C₃-C₈)-cycloalkylamino, (C₁-C₆)-alkylcarbonylamino, (C₃-C₈)-cycloalkylcarbonylamino, formylamino, (C₁-C₆)-haloalkylcarbonylamino, (C₁-C₆)-alkoxycarbonylamino, (C₁-C₆)-(alkyl)aminocarbonylamino, (C₁-C₆)-alkylsulfonylamino, (C₃-C₈)-cycloalkylsulfonylamino, arylsulfonylamino, hetarylsulfonylamino, sulfonyl-(C₁-C₆)-haloalkylamino, amino-(C₁-C₆)-alkylsulfonyl, amino-(C₁-C₆)-haloalkylsulfonyl, (C₁-C₆)-alkylsulfonyl, (C₃-C₈)-cycloalkylsulfonyl, arylsulfonyl, (C₁-C₆)-alkylsulfinyl, (C₃-C₈)-cycloalkylsulfinyl, arylsulfinyl, N,S-di-(C₁-C₆)-alkylsulfonimidoyl, S—(C₁-C₆)-alkylsulfonimidoyl, (C₁-C₆)-alkylsulfonylaminocarbonyl, (C₃-C₈)-cycloalkylsulfonylaminocarbonyl, (C₁-C₆)-cycloalkylaminosulfonyl,
R¹ is H, nitro, amino, cyano, halogen, (C₁-C₆)-alkyl, (C₃-C₈)-cycloalkyl, (C₂-C₈)-alkenyl, (C₂-C₈)-alkynyl, aryl, aryl-(C₁-C₆)-alkyl, heteroaryl, (C₁-C₆)-alkoxy-(C₁-C₆)-alkyl, (C₁-C₆)-haloalkyl, (C₃-C₈)-halocycloalkyl, (C₁-C₆)-alkoxy, (C₁-C₆)-haloalkoxy, (C₁-C₆)-alkylthio, (C₁-C₆)-haloalkylthio, (C₁-C₆)-alkoxycarbonyl, hydroxycarbonyl, aminocarbonyl, (C₁-C₆)-alkylaminocarbonyl, (C₃-C₈)-cycloalkylaminocarbonyl, (C₁-C₆)-alkylamino, bis(C₁-C₆)-alkylamino, (C₃-C₈)-cycloalkylamino, (C₁-C₆)-alkylcarbonylamino, (C₃-C₈)-cycloalkylcarbonylamino, formylamino, (C₁-C₆)-haloalkylcarbonylamino, (C₁-C₆)-alkoxycarbonylamino, (C₁-C₆)-(alkyl)aminocarbonylamino, (C₁-C₆)-alkylsulfonylamino, (C₃-C₈)-cycloalkylsulfonylamino, arylsulfonylamino, hetarylsulfonylamino, sulfonyl(C₁-C₆)-haloalkylamino, amino-(C₁-C₆)-alkylsulfonyl, amino-(C₁-C₆)-haloalkylsulfonyl, (C₁-C₆)-alkylsulfonyl, (C₃-C₈)-cycloalkylsulfonyl, arylsulfonyl, (C₁-C₆)-alkylsulfinyl, (C₃-C₈)-cycloalkylsulfinyl, arylsulfinyl, N,S-di-(C₁-C₆)-alkylsulfonimidoyl, S—(C₁-C₆)-alkylsulfonimidoyl, (C₁-C₆)-alkylsulfonylaminocarbonyl, (C₃-C₈)-cycloalkylsulfonylaminocarbonyl, (C₁-C₆)-cycloalkylaminosulfonyl,
R² is H, nitro, amino, cyano, halogen, (C₁-C₆)-alkyl, (C₃-C₈)-cycloalkyl, (C₂-C₅)-alkenyl, (C₂-C₈)-alkynyl, aryl, aryl-(C₁-C₆)-alkyl, heteroaryl, (C₁-C₆)-alkoxy-(C₁-C₆)-alkyl, (C₁-C₆)-haloalkyl, (C₃-C₈)-halocycloalkyl, (C₁-C₆)-alkoxy, (C₁-C₆)-haloalkoxy, (C₁-C₆)-alkylthio, (C₁-C₆)-haloalkylthio, (C₁-C₆)-alkoxycarbonyl, hydroxycarbonyl, aminocarbonyl, (C₁-C₆)-alkylaminocarbonyl, (C₃-C₈)-cycloalkylaminocarbonyl, (C₁-C₆)-alkylamino, bis(C₁-C₆)-alkylamino, (C₃-C₈)-cycloalkylamino, (C₁-C₆)-alkylcarbonylamino, (C₃-C₈)-cycloalkylcarbonylamino, formylamino, (C₁-C₆)-haloalkylcarbonylamino, (C₁-C₆)-alkoxycarbonylamino, (C₁-C₆)-(alkyl)aminocarbonylamino, (C₁-C₆)-alkylsulfonylamino, (C₃-C₈)-cycloalkylsulfonylamino, arylsulfonylamino, hetarylsulfonylamino, sulfonyl(C₁-C₆)-haloalkylamino, amino-(C₁-C₆)-alkylsulfonyl, amino-(C₁-C₆)-haloalkylsulfonyl, (C₁-C₆)-alkylsulfonyl, (C₃-C₈)-cycloalkylsulfonyl, arylsulfonyl, (C₁-C₆)-alkylsulfinyl, (C₃-C₈)-cycloalkylsulfinyl, arylsulfinyl, N,S-di-(C₁-C₆)-alkylsulfonimidoyl, S—(C₁-C₆)-alkylsulfonimidoyl, (C₁-C₆)-alkylsulfonylaminocarbonyl, (C₃-C₈)-cycloalkylsulfonylaminocarbonyl, (C₁-C₆)-cycloalkylaminosulfonyl,
R¹ and R² with the atoms to which they are bonded form a fully saturated, partly saturated or fully unsaturated, 5 to 7-membered ring optionally interrupted by heteroatoms and optionally with further substitution,
R¹ and A³, when A³ is a C—W group, with the atoms to which they are bonded form a fully saturated, partly saturated or fully unsaturated, 5 to 7-membered ring optionally interrupted by heteroatoms and optionally with further substitution,
X is H, (C₁-C₆)-alkyl, (C₁-C₆)-alkyl-(C₂-C₈)-alkenyl, (C₁-C₆)-alkylcarbonyl, (C₃-C₈)-cycloalkylcarbonyl, arylcarbonyl, heteroarylcarbonyl, (C₁-C₆)-alkoxycarbonyl, (C₁-C₆)-alkylsulfonyl, (C₃-C₈)-cycloalkylsulfonyl, arylsulfonyl, (C₁-C₆)-alkoxycarbonylcarbonyl, aryl-(C₁-C₆)-alkoxycarbonylcarbonyl, (C₁-C₆)-alkylaminothiocarbonyl, (C₁-C₆)-alkylaminocarbonyl, aryl-(C₁-C₆)-alkyl, heteroaryl-(C₁-C₆)-alkyl, (C₂-C₈)-alkynyl, (C₁-C₆)-cyanoalkyl, (C₁-C₆)-haloalkyl, (C₃-C₈)-cycloalkyl, (C₃-C₇)-cycloalkyl-(C₁-C₆)-alkyl,
Y is (C₁-C₆)-alkanediyl, (C₂-C₈)-alkynyl, (C₃-C₇)-cycloalkanediyl, (C₂-C₇)-alkenediyl, (C₁-C₆)-alkyl-(C₂-C₇)-alkenediyl, aryl-(C₁-C₆)-alkanediyl, (C₁-C₆)-alkoxydiyl, (C₁-C₆)-alkoxy-(C₁-C₆)-alkyldiyl, (C₁-C₆)-alkoxycarbonyl-(C₁-C₆)-alkyldiyl, (C₁-C₆)-alkylthio-(C₁-C₆)-alkyldiyl, (C₁-C₆)-alkylsulfinyl-(C₁-C₆)-alkyldiyl, (C₁-C₆)-alkylsulfonyl-(C₁-C₆)-alkyldiyl or a direct bond between N and Q,
Q is a phenyl ring or a 5-, 6- or 7-membered saturated or unsaturated heterocyclic ring or a 9- or 10-membered fused bicyclic or tricyclic ring system which may optionally contain further heteroatoms, where the particular aforementioned ring or the ring system is optionally mono- or polysubstituted, identically or differently, and where the substituents are each independently selected from H, halogen, cyano, nitro, branched or unbranched (C₁-C₆)-alkyl, (C₃-C₈)-cycloalkyl, (C₂-C₈)-alkenyl, (C₂-C₈)-alkynyl, aryl-(C₁-C₆)-alkyl, (C₁-C₆)-alkoxy-(C₁-C₆)-alkyl, (C₁-C₆)-haloalkyl, (C₃-C₈)-halocycloalkyl, (C₃-C₈)-alkoxycycloalkyl, (C₃-C₈)-arylcycloalkyl, (C₃-C₈)-cycloalkyl-(C₃-C₈)-cycloalkyl, (C₃-C₈)-cycloalkyl-(C₁-C₆)-alkyl, (C₁-C₆)-alkoxy, (C₁-C₆)-haloalkoxy, (C₁-C₆)-alkoxycarbonyl, hydroxycarbonyl, aminocarbonyl, (C₁-C₆)-alkylaminocarbonyl, amino, (C₁-C₆)-alkylamino, bis(C₁-C₆)-alkylamino, (C₃-C₈)-cycloalkylamino, (C₁-C₆)-alkylcarbonylamino, (C₁-C₆)-alkylcarbonyl, (C₁-C₆)-alkylthio, (C₁-C₆)-haloalkylthio, (C₁-C₆)-haloalkylsulfinyl, (C₁-C₆)-haloalkylsulfonyl or where the substituents are each independently selected from phenyl or a 5- or 6-membered heteroaromatic ring, where phenyl or the heteroaromatic ring is optionally mono- or polysubstituted, identically or differently, by (C₁-C₆)-alkyl, (C₂-C₈)-alkenyl, (C₂-C₈)-alkynyl, (C₃-C₈)-cycloalkyl, (C₁-C₆)-haloalkyl, (C₂-C₈)-haloalkenyl, (C₂-C₈)-haloalkynyl, (C₃-C₈)-halocycloalkyl, halogen, cyano, nitro, (C₁-C₆)-alkoxy, (C₁-C₆)-haloalkoxy, (C₁-C₆)-alkylthio, (C₁-C₆)-haloalkylthio, and Q is additionally one of the following radicals Q-1 to Q-192:

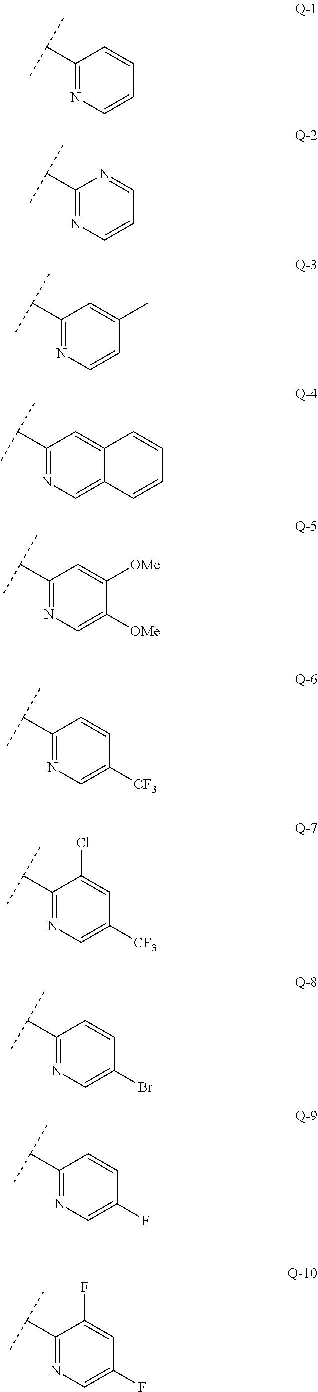

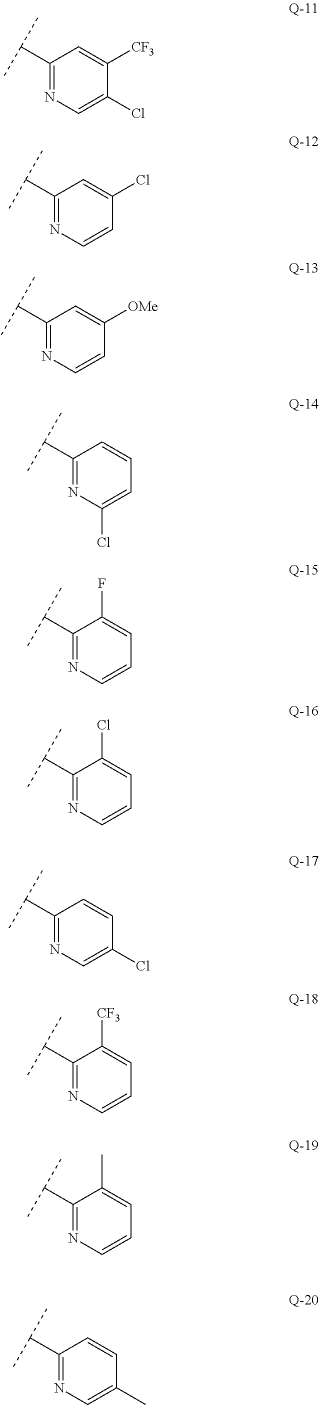

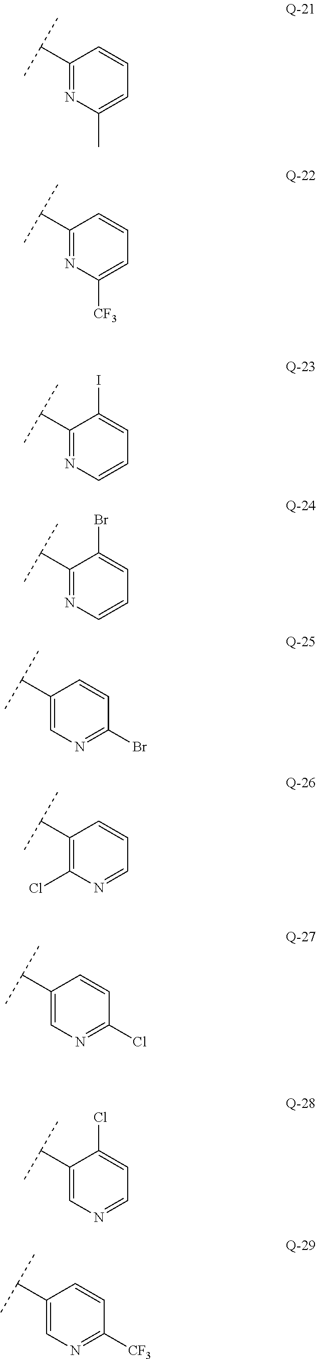

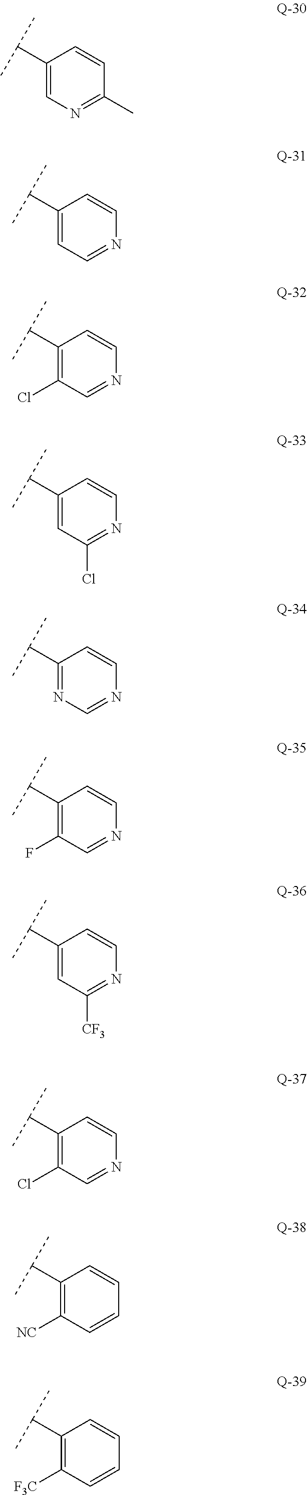

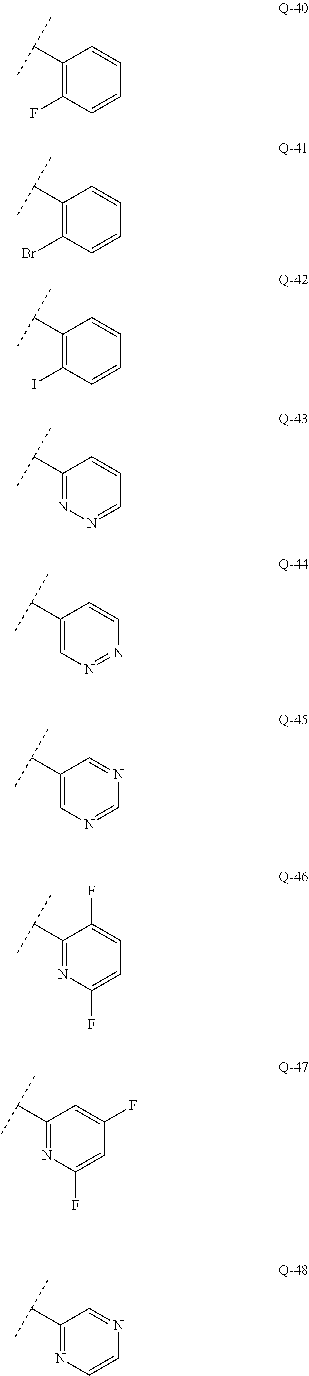

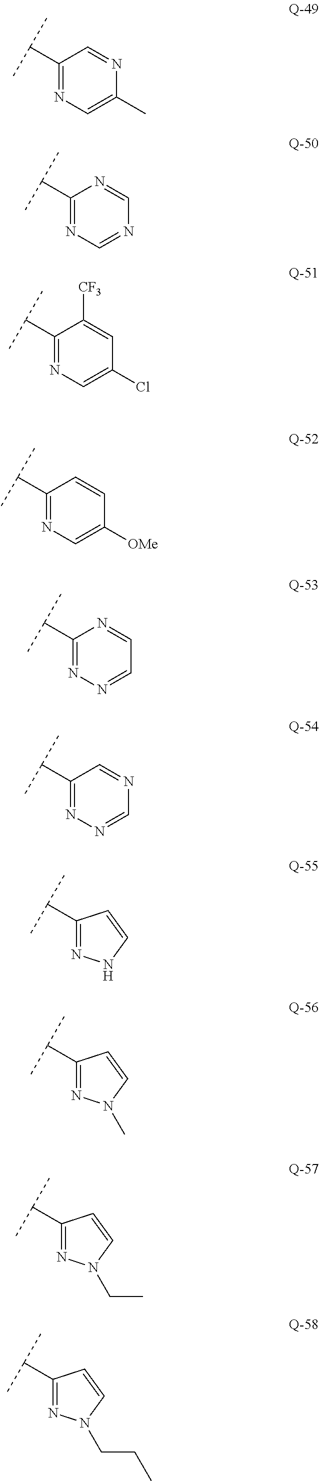

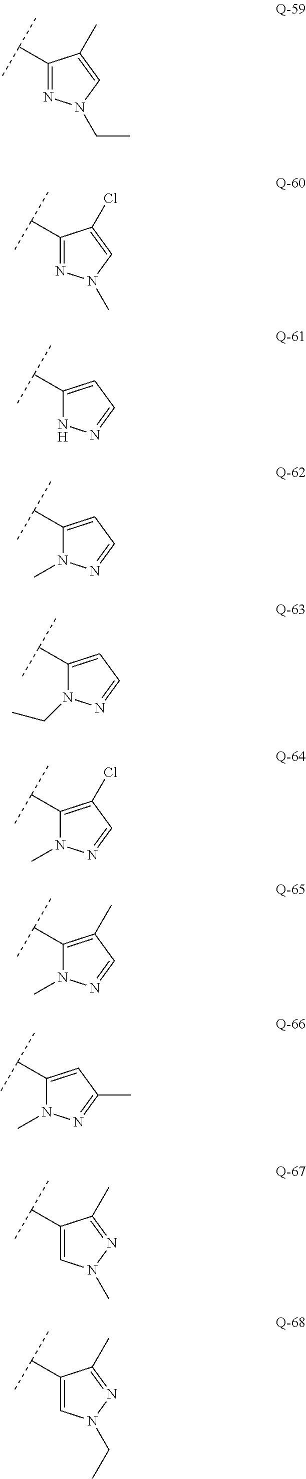

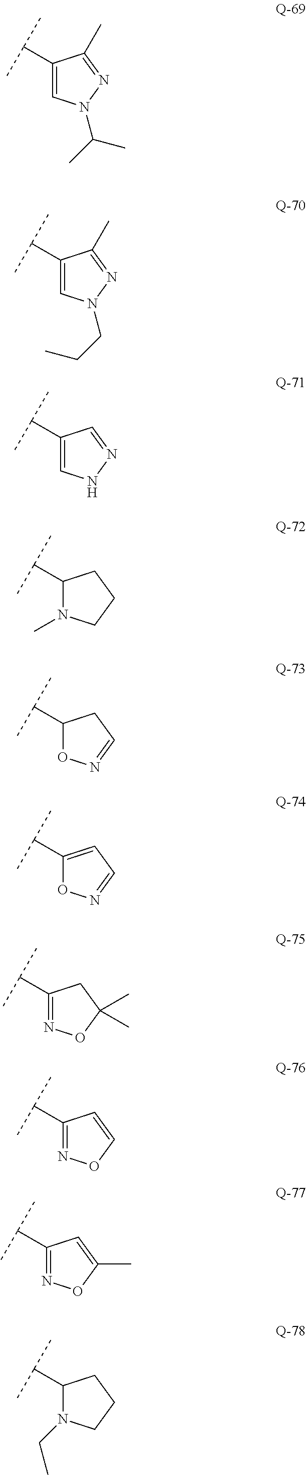

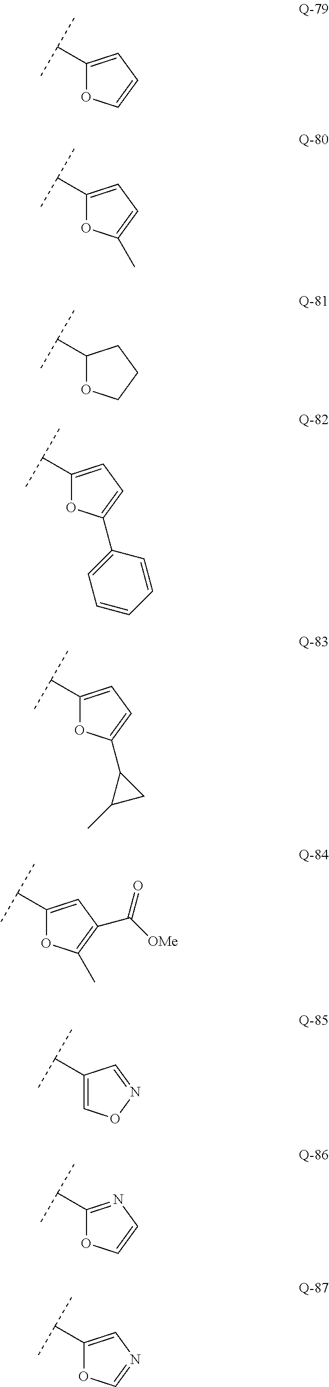

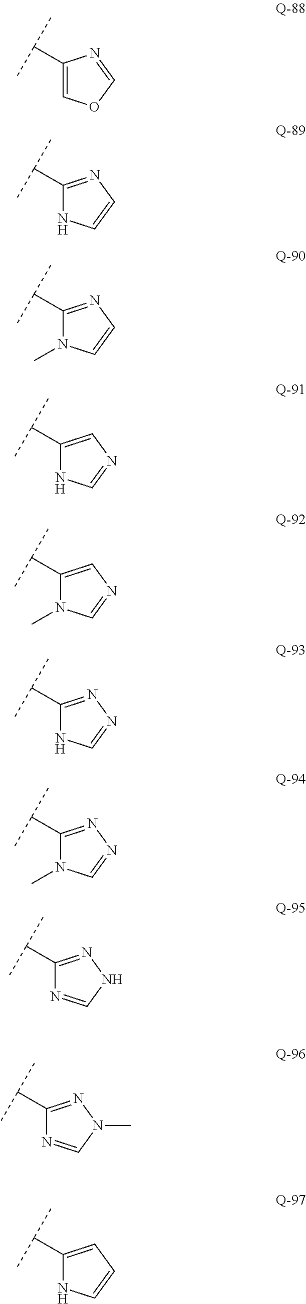

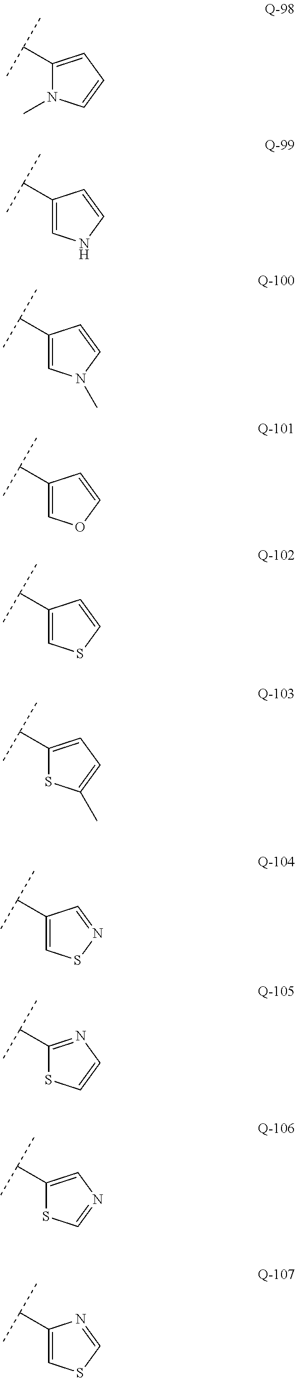

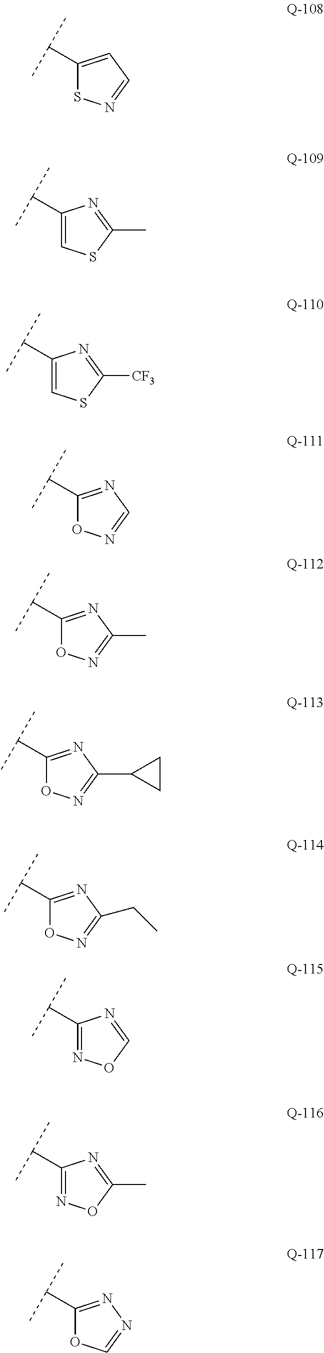

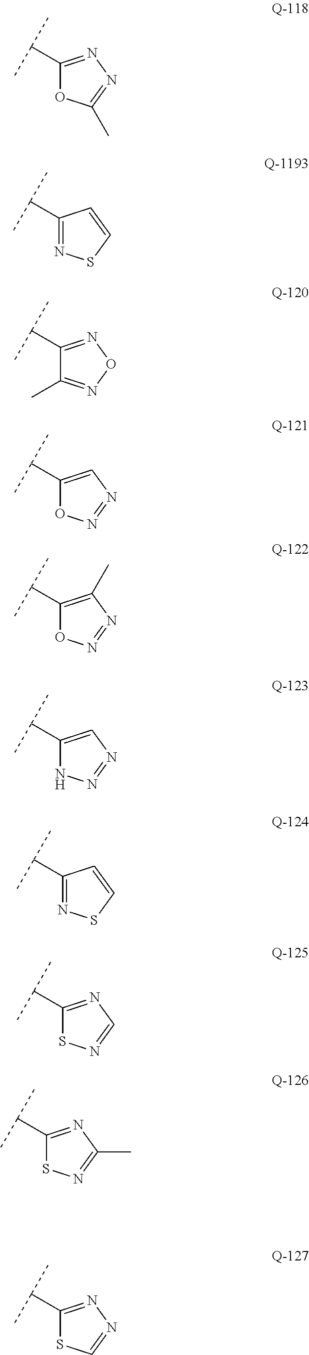

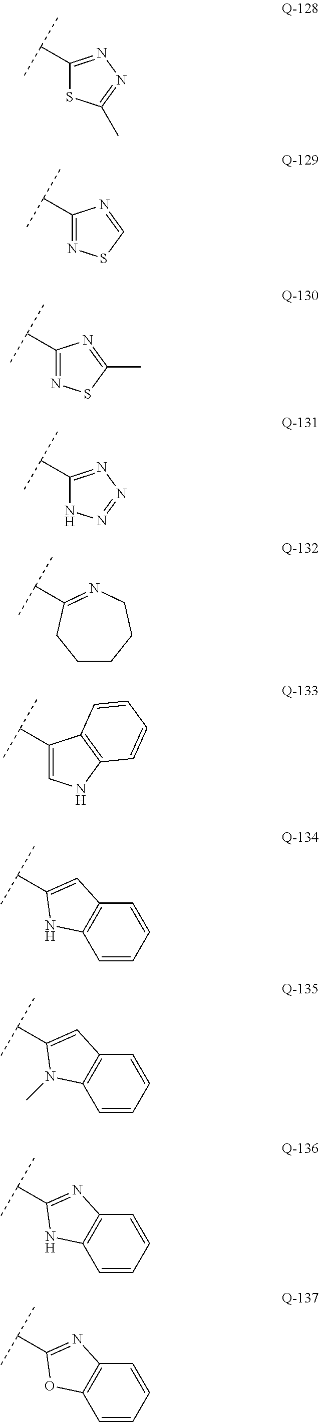

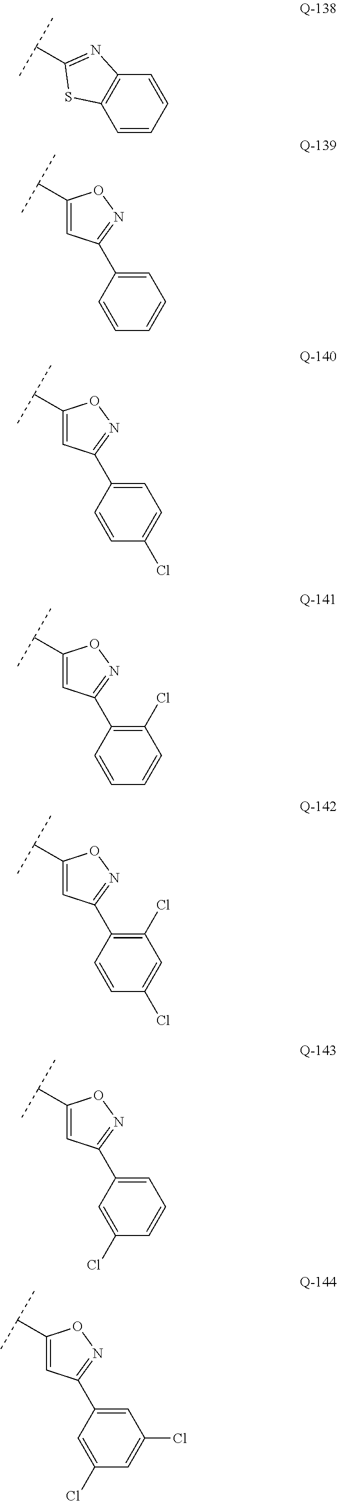

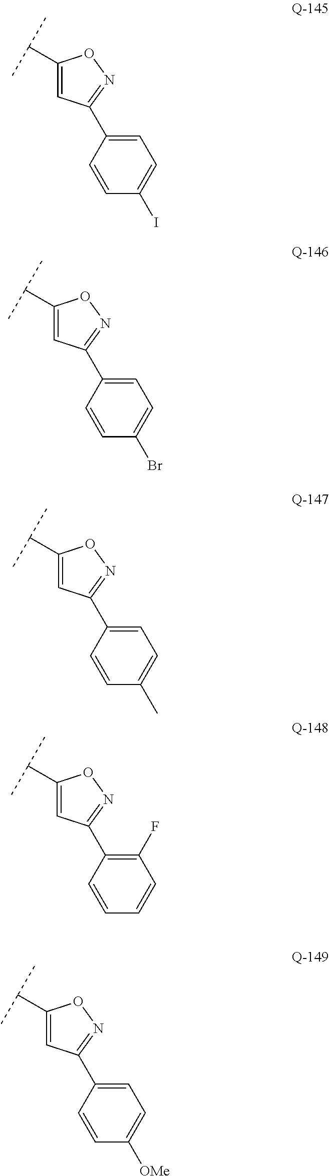

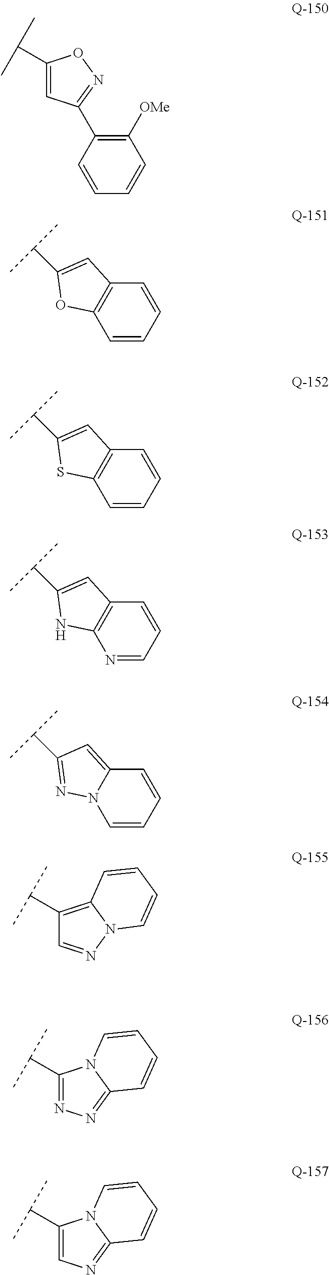

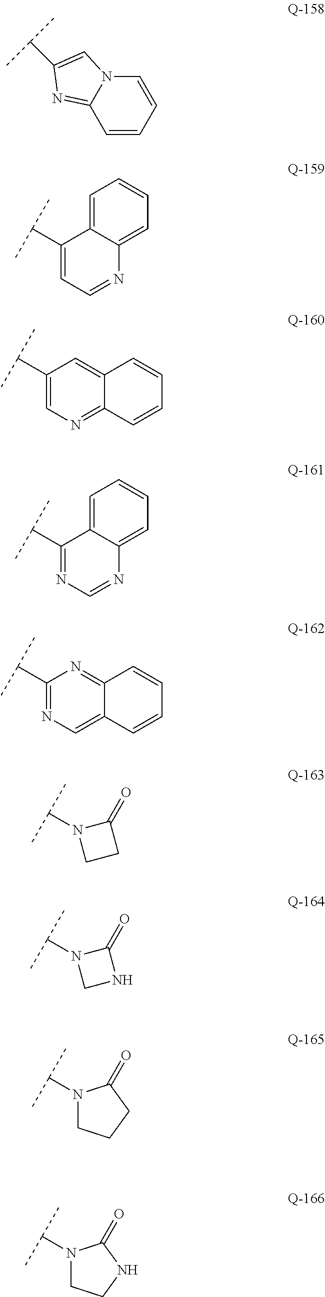

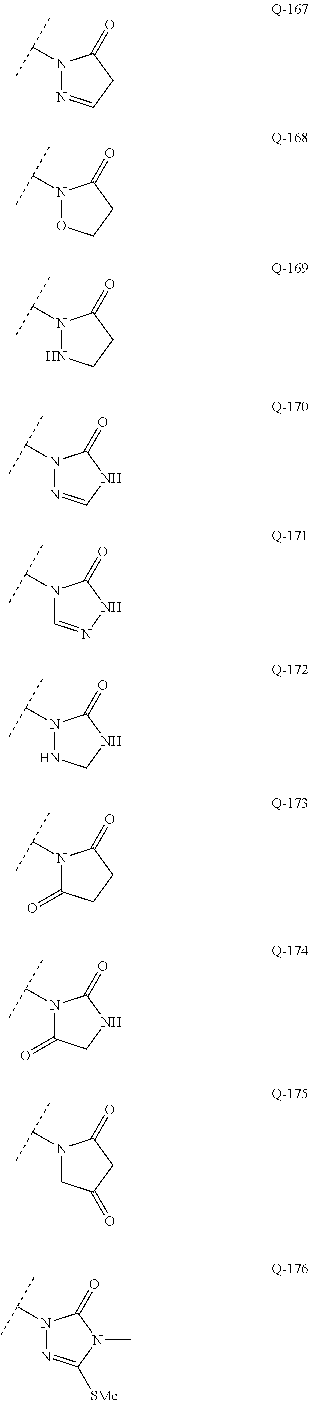

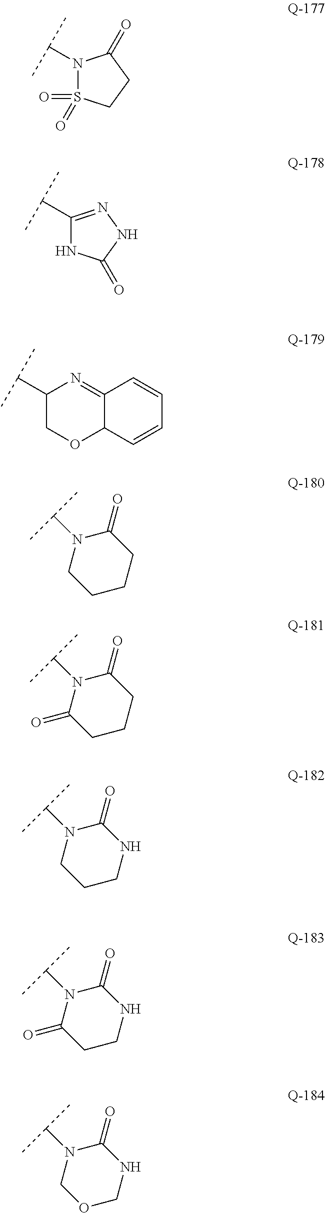

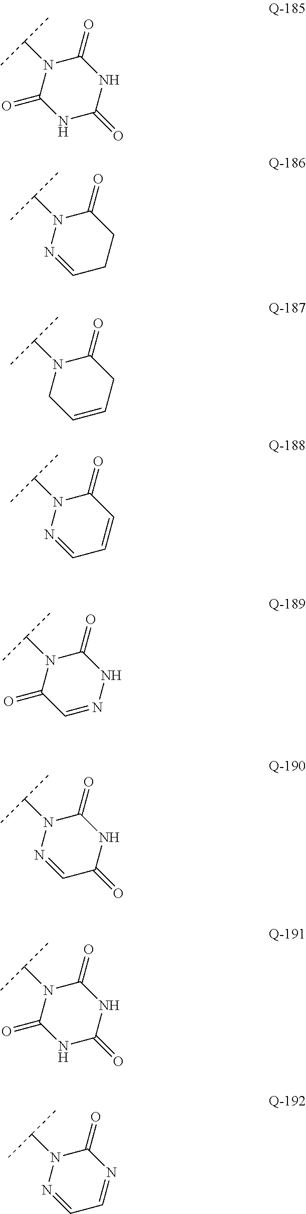

Particular preference is given to the inventive use of compounds of the formula (I) in which
A¹, A², A³ are the same or different and are each independently N (nitrogen) or the C—W moiety, but there are never more than two adjacent nitrogen atoms, and where W in the C—W moiety in each case has identical or different definitions as defined below;
W in each case is hydrogen, nitro, amino, cyano, halogen, (C₁-C₄)-alkyl, (C₃-C₆)-cycloalkyl, (C₂-C₆)-alkenyl, (C₂-C₆)-alkynyl, aryl, aryl-(C₁-C₄)-alkyl, heteroaryl, (C₁-C₄)-alkoxy-(C₁-C₄)-alkyl, (C₁-C₄)-haloalkyl, (C₃-C₆)-halocycloalkyl, (C₁-C₄)-alkoxy, aryl-(C₁-C₄)-alkoxy, (C₁-C₄)-haloalkoxy, (C₁-C₄)-alkoxycarbonyl, hydroxycarbonyl, aminocarbonyl, (C₁-C₄)-alkylaminocarbonyl, (C₃-C₇)-cycloalkylaminocarbonyl, (C₁-C₄)-alkylamino, (C₁-C₄)-alkylthio, (C₁-C₄)-haloalkylthio, bis(C₁-C₄)-alkylamino, (C₃-C₆)-cycloalkylamino, (C₁-C₄)-alkylcarbonylamino, (C₃-C₇)-cycloalkylcarbonylamino, formylamino, (C₁-C₄)-haloalkylcarbonylamino, (C₁-C₄)-alkoxycarbonylamino, (C₁-C₄)-(alkyl)aminocarbonylamino, (C₁-C₄)-alkylsulfonylamino, (C₃-C₆)-cycloalkylsulfonylamino, arylsulfonylamino, hetarylsulfonylamino, sulfonyl-(C₁-C₄)-haloalkylamino, amino-(C₁-C₄)-alkylsulfonyl, amino-(C₁-C₄)-haloalkylsulfonyl, (C₁-C₄)-alkylsulfonyl, (C₃-C₇)-cycloalkylsulfonyl, arylsulfonyl, (C₁-C₄)-alkylsulfinyl, (C₃-C₇)-cycloalkylsulfinyl, arylsulfinyl, N,S-di-(C₁-C₄)-alkylsulfonimidoyl, S—(C₁-C₄)-alkylsulfonimidoyl, (C₁-C₄)-alkylsulfonylaminocarbonyl, (C₃-C₇)-cycloalkylsulfonylaminocarbonyl, (C₁-C₄)-cycloalkylaminosulfonyl,
R¹ is H, nitro, amino, cyano, halogen, (C₁-C₄)-alkyl, (C₃-C₆)-cycloalkyl, (C₂-C₆)-alkenyl, (C₂-C₆)-alkynyl, aryl, aryl-(C₁-C₄)-alkyl, heteroaryl, (C₁-C₄)-alkoxy-(C₁-C₄)-alkyl, (C₁-C₄)-haloalkyl, (C₃-C₆)-halocycloalkyl, (C₁-C₄)-alkoxy, (C₁-C₄)-haloalkoxy, (C₁-C₄)-alkylthio, (C₁-C₄)-haloalkylthio, (C₁-C₄)-alkoxycarbonyl, hydroxycarbonyl, aminocarbonyl, (C₁-C₄)-alkylaminocarbonyl, (C₃-C₇)-cycloalkylaminocarbonyl, (C₁-C₄)-alkylamino, bis(C₁-C₄)-alkylamino, (C₃-C₆)-cycloalkylamino, (C₁-C₄)-alkylcarbonylamino, (C₃-C₇)-cycloalkylcarbonylamino, formylamino, (C₁-C₄)-haloalkylcarbonylamino, (C₁-C₄)-alkoxycarbonylamino, (C₁-C₄)-(alkyl)aminocarbonylamino, (C₁-C₄)-alkylsulfonylamino, (C₃-C₆)-cycloalkylsulfonylamino, arylsulfonylamino, hetarylsulfonylamino, sulfonyl(C₁-C₄)-haloalkylamino, amino-(C₁-C₄)-alkylsulfonyl, amino-(C₁-C₄)-haloalkylsulfonyl, (C₁-C₄)-alkylsulfonyl, (C₃-C₇)-cycloalkylsulfonyl, arylsulfonyl, (C₁-C₄)-alkylsulfinyl, (C₃-C₇)-cycloalkylsulfinyl, arylsulfinyl, N,S-di-(C₁-C₄)-alkylsulfonimidoyl, S—(C₁-C₄)-alkylsulfonimidoyl, (C₁-C₄)-alkylsulfonylaminocarbonyl, (C₃-C₇)-cycloalkylsulfonylaminocarbonyl, (C₁-C₄)-cycloalkylaminosulfonyl,
R² is H, nitro, amino, cyano, halogen, (C₁-C₄)-alkyl, (C₃-C₆)-cycloalkyl, (C₂-C₆)-alkenyl, (C₂-C₆)-alkynyl, aryl, aryl-(C₁-C₄)-alkyl, heteroaryl, (C₁-C₄)-alkoxy-(C₁-C₄)-alkyl, (C₁-C₄)-haloalkyl, (C₃-C₆)-halocycloalkyl, (C₁-C₄)-alkoxy, (C₁-C₄)-haloalkoxy, (C₁-C₄)-alkylthio, (C₁-C₄)-haloalkylthio, (C₁-C₄)-alkoxycarbonyl, hydroxycarbonyl, aminocarbonyl, (C₁-C₄)-alkylaminocarbonyl, (C₃-C₇)-cycloalkylaminocarbonyl, (C₁-C₄)-alkylamino, bis(C₁-C₄)-alkylamino, (C₃-C₆)-cycloalkylamino, (C₁-C₄)-alkylcarbonylamino, (C₃-C₇)-cycloalkylcarbonylamino, formylamino, (C₁-C₄)-haloalkylcarbonylamino, (C₁-C₄)-alkoxycarbonylamino, (C₁-C₄)-(alkyl)aminocarbonylamino, (C₁-C₄)-alkylsulfonylamino, (C₃-C₆)-cycloalkylsulfonylamino, arylsulfonylamino, hetarylsulfonylamino, sulfonyl(C₁-C₄)-haloalkylamino, amino-(C₁-C₄)-alkylsulfonyl, amino-(C₁-C₄)-haloalkylsulfonyl, (C₁-C₄)-alkylsulfonyl, (C₃-C₇)-cycloalkylsulfonyl, arylsulfonyl, (C₁-C₄)-alkylsulfinyl, (C₃-C₇)-cycloalkylsulfinyl, arylsulfinyl, N,S-di-(C₁-C₄)-alkylsulfonimidoyl, S—(C₁-C₄)-alkylsulfonimidoyl, (C₁-C₄)-alkylsulfonylaminocarbonyl, (C₃-C₇)-cycloalkylsulfonylaminocarbonyl, (C₁-C₄)-cycloalkylaminosulfonyl,
R¹ and R² with the atoms to which they are bonded form a fully saturated, partly saturated or fully unsaturated, 5 to 6-membered ring optionally interrupted by heteroatoms and optionally with further substitution,
R¹ and A³, when A³ is a C—W group, with the atoms to which they are bonded form a fully saturated, partly saturated or fully unsaturated, 5 to 6-membered ring optionally interrupted by heteroatoms and optionally with further substitution,
X is H, (C₁-C₄)-alkyl, (C₁-C₄)-alkyl-(C₂-C₆)-alkenyl, (C₁-C₄)-alkylcarbonyl, (C₃-C₆)-cycloalkylcarbonyl, arylcarbonyl, heteroarylcarbonyl, (C₁-C₄)-alkoxycarbonyl, (C₁-C₄)-alkylsulfonyl, (C₃-C₆)-cycloalkylsulfonyl, arylsulfonyl, (C₁-C₄)-alkoxycarbonylcarbonyl, aryl-(C₁-C₄)-alkoxycarbonylcarbonyl, (C₁-C₄)-alkylaminothiocarbonyl, (C₁-C₄)-alkylaminocarbonyl, aryl-(C₁-C₄)-alkyl; hetaroaryl-(C₁-C₄)-alkyl, (C₂-C₆)-alkynyl, (C₁-C₄)-cyanoalkyl, (C₁-C₄)-haloalkyl, (C₃-C₇)-cycloalkyl, (C₃-C₇)-cycloalkyl-(C₁-C₆)-alkyl,
Y is (C₁-C₅)-alkanediyl, (C₃-C₆)-cycloalkanediyl, (C₂-C₆)-alkenediyl, (C₂-C₆)-alkynediyl, (C₁-C₄)-alkyl-(C₂-C₆)-alkenediyl, aryl-(C₁-C₄)-alkanediyl, (C₁-C₄)-alkoxydiyl, (C₁-C₄)-alkoxy-(C₁-C₄)-alkyldiyl, (C₁-C₄)-alkoxycarbonyl-(C₁-C₄)-alkyldiyl, (C₁-C₄)-alkylthio-(C₁-C₄)-alkyldiyl, (C₁-C₄)-alkylsulfinyl-(C₁-C₄)-alkyldiyl, (C₁-C₄)-alkylsulfonyl-(C₁-C₄)-alkyldiyl or a direct bond between N and Q,
Q is a phenyl ring or a 5-, 6- or 7-membered saturated or unsaturated
   heterocyclic ring or a 9- or 10-membered fused bicyclic or tricyclic ring system which optionally contains further heteroatoms, where the particular aforementioned ring or the ring system is optionally mono- or polysubstituted, identically or differently, and where the substituents are each independently selected from H, halogen, cyano, nitro, branched or unbranched (C₁-C₄)-alkyl, (C₃-C₆)-cycloalkyl, (C₃-C₇)-alkoxycycloalkyl, (C₃-C₇)-arylcycloalkyl, (C₃-C₇)-cycloalkyl-(C₃-C₇)-cycloalkyl, (C₃-C₇)-cycloalkyl-(C₁-C₄)-alkyl, (C₂-C₆)-alkenyl, (C₂-C₆)-alkynyl, aryl-(C₁-C₄)-alkyl, (C₁-C₄)-alkoxy-(C₁-C₄)-alkyl, (C₁-C₄)-haloalkyl, (C₃-C₆)-halocycloalkyl, (C₁-C₄)-alkoxy, (C₁-C₄)-haloalkoxy, (C₁-C₄)-alkoxycarbonyl, hydroxycarbonyl, aminocarbonyl, (C₁-C₄)-alkylaminocarbonyl, amino, (C₁-C₄)-alkylamino, bis(C₁-C₄)-alkylamino, (C₃-C₆)-cycloalkylamino, (C₁-C₄)-alkylcarbonylamino, (C₁-C₄)-alkylcarbonyl, (C₁-C₄)-alkylthio, (C₁-C₄)-haloalkylthio, (C₁-C₄)-haloalkylsulfinyl, (C₁-C₄)-haloalkylsulfonyl, or where the substituents are each independently selected from phenyl or a 5- or 6-membered heteroaromatic ring, where phenyl or the heteroaromatic ring is optionally mono- or polysubstituted, identically or differently, by (C₁-C₄)-alkyl, (C₂-C₆)-alkenyl, (C₂-C₆)-alkynyl, (C₃-C₇)-cycloalkyl, (C₁-C₄)-haloalkyl, (C₂-C₆)-haloalkenyl, (C₂-C₆)-haloalkynyl, (C₃-C₆)-halocycloalkyl, halogen, cyano, nitro, (C₁-C₄)-alkoxy, (C₁-C₄)-haloalkoxy, (C₁-C₄)-alkylthio, (C₁-C₄)-haloalkylthio, and Q is additionally one of the above-described Q-1 to Q-192 radicals.

Very particular preference is given to the inventive use of compounds of the formula (I) in which
A¹, A², A³ are the same or different and are each independently N (nitrogen) or the C—W moiety, but there are never more than two adjacent nitrogen atoms, and where W in the C—W moiety in each case has identical or different definitions as defined below;
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene,
Q is an optionally mono- or polysubstituted phenyl ring or an optionally mono- or polysubstituted 5-, 6- or 7-membered saturated or unsaturated heterocyclic ring or a 9- or 10-membered fused heterobicyclic ring system from the above-described group of Q-1 to Q-192, where the substituents are each independently selected from hydrogen, methyl, ethyl, n-propyl, isopropyl, cyclopropyl, methylcyclopropyl, methoxy, ethoxy, trifluoromethyl, fluorine, chlorine, bromine, iodine, cyano, nitro, trifluoromethoxy, methylthio, trifluoromethylthio, OCHF₂, OCCIF₂, methoxycarbonyl, phenyl, p-chlorophenyl, m-chlorophenyl, o-chlorophenyl, o,p-dichlorophenyl, m,m-dichlorophenyl, o-iodophenyl, m-iodophenyl, p-iodophenyl, o-bromophenyl, m-bromophenyl, p-bromophenyl, o-methylphenyl, m-methylphenyl, p-methylphenyl, o-fluorophenyl, m-fluorophenyl, p-fluorophenyl, o-methoxyphenyl, m-methoxyphenyl, p-methoxyphenyl, o-trifluoromethylphenyl, m-trifluoromethylphenyl, p-trifluoromethylphenyl, p-trifluoromethoxyphenyl, p-trifluoromethylthiophenyl, o-nitrophenyl, m-nitrophenyl, p-nitrophenyl, o-cyanophenyl, m-cyanophenyl, p-cyanophenyl.

Particular preference is additionally given to the inventive use of compounds of the formula (I) in which
A¹, A², A³ are the same or different and are each independently N (nitrogen) or the C—W moiety, but there are never more than two adjacent nitrogen atoms, and where W in the C—W moiety in each case has identical or different definitions as defined below,
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, -methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene,
Q is a moiety from the above-described group of Q-2 to Q-192.

Particular preference is further given to the inventive use of compounds of the formula (I) in which
A¹, A², A³ are the same or different and are each independently N (nitrogen) or the C—W moiety, but there are never more than two adjacent nitrogen atoms, and where W in the C—W moiety in each case has identical or different definitions as defined below,
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene,
Q is the moieties Q-2, Q-34, Q-43 to Q-45, Q-48 to Q-50 and Q-53 to Q-192 from the above-described groups.

Especial preference is given to the inventive use of compounds of the formula (I) in which
A¹, A², A³ are the same or different and are each independently N (nitrogen) or the C—W moiety, but there are never more than two adjacent nitrogen atoms, and where W in the C—W moiety in each case has identical or different definitions as defined below;
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino; cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene,
Q is a moiety Q-55 to Q-192 from the above-described group.

More especial preference is given to the inventive use of compounds of the formula (I) in which
A¹, A², A³ are the same or different and are each independently N (nitrogen) or the C—W moiety, but there are never more than two adjacent nitrogen atoms, and where W in the C—W moiety in each case has identical or different definitions as defined below;
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene and
Q is a moiety Q-72, Q-73, Q-75, Q-78, Q-81, Q-132 and Q-163 to Q-192 from the above-described group.

The definitions of radicals stated above in general terms or in areas of preference apply both to the end products of the formula (I) and correspondingly to the starting materials or intermediates required in each case for preparation thereof.

These radical definitions can be combined with one another as desired, i.e. including combinations between the given preferred ranges.

Essentially, some of the aforementioned sulfonamides of the formula (I) are likewise as yet unknown in the prior art. Thus, a further part of the invention is aryl- and hetarylsulfonamides of the formula (I), or salts thereof,

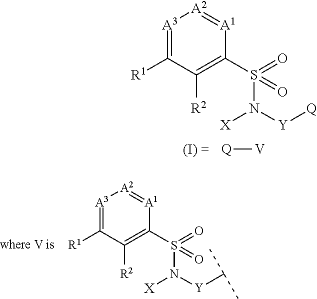

in which Q
is Q-1 and V is defined as 5-iodonaphthalene-1-sulfonylaminomethyl, 4-fluoronaphthalene-1-sulfonylaminomethyl, 4-methylnaphthalene-1-sulfonylaminomethyl, 4-isopropoxynaphthalene-1-sulfonylaminomethyl, 4-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethyl-benzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 2-chloro-6-methylbenzenesulfonylaminomethyl, 2-chloro-6-methoxybenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 4-trifluoromethylthio-benzenesulfonylaminomethyl, 4-difluoromethoxybenzenesulfonylaminomethyl, 1-(4-bromobenzenesulfonylamino)cyclopropan-1-yl, 1-(4-chlorobenzenesulfonylamino)cyclopropan-1-yl, 1-(4-iodobenzenesulfonylamino)cyclopropan-1-yl, 1-(4-methoxybenzenesulfonylamino)cyclopropan-1-yl, 1-(4-bromonaphthalene-1-sulfonylamino)cyclopropan-1-yl, 1-(4-methoxynaphthalene-1-sulfonylamino)cyclopropan-1-yl, 1-(naphthalene-1-sulfonylamino)cyclopropan-1-yl, 1-(5-iodonaphthalene-1-sulfonylamino)cyclopropan-1-yl, 1-(4-fluoronaphthalene-1-sulfonylamino)cyclopropan-1-yl, 1-(4-methylnaphthalene-1-sulfonylamino)cyclopropan-1-yl, 1-(4-isopropoxynaphthalene-1-sulfonylamino)cyclopropan-1-yl, 1-(5-bromonaphthalene-1-sulfonylamino)cyclopropan-1-yl, 1-(4-bromo-2-fluorobenzenesulfonylamino)cyclopropan-1-yl, 1-(4-bromo-2-trifluoromethylbenzenesulfonylamino)cyclopropan-1-yl, 1-(4-bromo-2-methylbenzenesulfonylamino)cyclopropan-1-yl, 1-(4-trifluoromethoxybenzenesulfonylamino)cyclopropan-1-yl, 1-(4-difluoromethoxybenzenesulfonylamino)cyclopropan-1-yl, 1-(4-trifluoromethylthiobenzenesulfonylamino)cyclopropan-1-yl, 1-(4-methylbenzenesulfonylamino)cyclopropan-1-yl, 1-(4-fluorobenzenesulfonylamino)cyclopropan-1-yl, 1-(4-trifluoromethylbenzenesulfonylamino)cyclopropan-1-yl, 1-(4-bromo-3-methylbenzenesulfonylamino)cyclopropan-1-yl, 1-(2,4-dichlorobenzenesulfonylamino)cyclopropan-1-yl, where the Q-1 radical is as defined above, or
is Q-2 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-2 radical is as defined above, or
is Q-6 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-6 radical is as defined above, or
is Q-7 and V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, where the Q-7 radical is as defined above, or
is Q-9 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-9 radical is as defined above, or
is Q-10 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-10 radical is as defined above, or
is Q-11 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-11 radical is as defined above, or
is Q-12 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-12 radical is as defined above, or
is Q-13 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 3,4-dichlorobenzenesulfonylaminomethyl, 2,4-dichlorobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-13 radical is as defined above, or
is Q-14 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-14 radical is as defined above, or
is Q-17 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-17 radical is as defined above, or
is Q-26 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-26 radical is as defined above, or
is Q-27 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-27 radical is as defined above, or
is Q-29 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenienesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-29 radical is as defined above, or
is Q-31 and V is defined as 5-iodonaphthalene-1-sulfonylaminomethyl, 4-fluoronaphthalene-1-sulfonylaminomethyl, 4-N-acetylaminonaphthalene-1-sulfonylaminomethyl, 4-methylnaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, 5-bromonaphthalene-1-sulfonylaminomethyl, 4-bromo-2-fluorobenzenesulfonylaminomethyl, 4-trifluoromethylthiobenzenesulfonylaminomethyl, 4-difluoromethoxybenzenesulfonylaminomethyl, where the Q-31 radical is as defined above, or
is Q-32 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-32 radical is as defined above, or
is Q-51 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-51 radical is as defined above, or
is Q-52 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-52 radical is as defined above, or
is Q-57 and V is defined as 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-iodobenzenesulfonylaminomethyl, 4-bromo-3-methylbenzenesulfonylaminomethyl, 4-trifluoromethoxybenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-57 radical is as defined above, or
is Q-58 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-58 radical is as defined above, or
is Q-60 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-60 radical is as defined above, or
is Q-64 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-64 radical is as defined above, or
is Q-77 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 3,4-dichlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 2,4-difluorobenzenesulfonylaminomethyl, 2,5-dichlorobenzenesulfonylaminomethyl, 2-chloro-6-fluorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-77 radical is as defined above, or
is Q-78 and V is defined as 4-bromonaphthalene-1-sulfonylaminomethyl, 5-iodonaphthalene-1-sulfonylaminomethyl, 4-fluoronaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-trifluoromethoxybenzenesulfonylaminomethyl, 2-chloro-6-methoxybenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-difluoromethoxybenzenesulfonylaminomethyl, where the Q-78 radical is as defined above, or
is Q-84 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-84 radical is as defined above, or
is Q-109 and V is defined as 4-bromonaphthalene-1-sulfonylaminomethyl, 5-iodonaphthalene-1-sulfonylaminomethyl, 4-fluoronaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-trifluoromethoxybenzenesulfonylaminomethyl, 2-chloro-6-methoxybenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, where the Q-109 radical is as defined above, or
is Q-110 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-110 radical is as defined above, or
is Q-113 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-113 radical is as defined above, or
is Q-114 and V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, where the Q-114 radical is as defined above, or
is Q-139 and V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 3-bromobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, where the Q-139 radical is as defined above, or
is Q-140 and V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 3-bromobenzenesulfonylaminomethyl, 2-fluoropyridine-3-sulfonylaminomethyl, 2-thfluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, where the Q-140 radical is as defined above, or
is Q-141 and V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 3-bromobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, where the Q-141 radical is as defined above, or
is Q-149 and V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 3-bromobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, where the Q-149 radical is as defined above.

With regard to the inventive compounds, the designations used above and below are explained. These are familiar to the person skilled in the art and especially have the definitions elucidated hereinafter:

According to the invention, “arylsulfonyl” represents optionally substituted phenylsulfonyl or optionally substituted polycyclic arylsulfonyl, here especially optionally substituted naphthylsulfonyl, for example substituted by halogen, cyano, nitro, alkyl, haloalkyl, haloalkoxy, amino, alkylamino, alkylcarbonylamino, dialkylamino or alkoxy groups.

According to the invention, “cycloalkylsulfonyl”—alone or as a constituent of a chemical group—represents optionally substituted cycloalkylsulfonyl, preferably having 3 to 6 carbon atoms, for example cyclopropylsulfonyl, cyclobutylsulfonyl, cyclopentylsulfonyl or cyclohexylsulfonyl.

According to the invention, “alkylsulfonyl”—alone or as a constituent of a chemical group—represents straight-chain or branched alkylsulfonyl, preferably having 1 to 8 or having 1 to 6 carbon atoms, for example methylsulfonyl, ethylsulfonyl, n-propylsulfonyl, isopropylsulfonyl, n-butylsulfonyl, isobutylsulfonyl, sec-butylsulfonyl and tert-butylsulfonyl.

According to the invention, “alkylthio”—alone or as a constituent of a chemical group—represents straight-chain or branched S-alkyl, preferably having 1 to 8 or having 1 to 6 carbon atoms, for example methylthio, ethylthio, n-propylthio, isopropylthio, n-butylthio, isobutylthio, sec-butylthio and tert-butylthio. Alkenylthio is an alkenyl radical bonded via a sulfur atom, alkynylthio is an alkynyl radical bonded via a sulfur atom, cycloalkylthio is a cycloalkyl radical bonded via a sulfur atom, and cycloalkenylthio is a cycloalkenyl radical bonded via a sulfur atom.

“Alkoxy” is an alkyl radical bonded via an oxygen atom, alkenyloxy is an alkenyl radical bonded via an oxygen atom, alkynyloxy is an alkynyl radical bonded via an oxygen atom, cycloalkyloxy is a cycloalkyl radical bonded via an oxygen atom, and cycloalkenyloxy is a cycloalkenyl radical bonded via an oxygen atom.

The term “aryl” is an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphtyl, indenyl, indanyl, fluorenyl, biphenylyl, where the bonding site is on the aromatic system.

In systematic terms, “aryl” is generally also encompassed by the term “optionally substituted phenyl”.

According to the invention, the expression “heteroaryl” represents heteroaromatic compounds, i.e. fully unsaturated aromatic heterocyclic compounds, preferably 5- to 7-membered rings having 1 to 3, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The inventive heteroaryl groups may also be substituted by one or more identical or different radicals. When two adjacent carbon atoms are part of a further aromatic ring, the systems are fused heteroaromatic systems, such as benzofused or polyannulated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partly saturated or heteroaromatic and may be unsubstituted or substituted, where the bonding site is localized on a ring atom. When the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. In the case of optionally substituted heterocyclyl, spirocyclic systems are also included, for example 1-oxa-5-azaspiro[2.3]hexyl. Unless defined differently, the heterocyclic ring contains preferably 3 to 9 ring atoms and especially 3 to 6 ring atoms, and one or more, preferably 1 to 4 and especially 1, 2 or 3 heteroatoms in the heterocyclic ring, preferably from the group of N, O and S, although no two oxygen atoms should be directly adjacent, for example, with one heteroatom from the group of N, O and S, 1- or 2- or 3-pyrrolidinyl, 3,4-dihydro-2H-pyrrol-2- or 3-yl, 2,3-dihydro-1H-pyrrol-1- or 2- or 3- or 4- or 5-yl; 2,5-dihydro-1H-pyrrol-1- or 2- or 3-yl, 1- or 2- or 3- or 4-piperidinyl; 2,3,4,5-tetrahydropyridin-2- or 3- or 4- or 5-yl or 6-yl; 1,2,3,6-tetrahydropyridin-1- or 2- or 3- or 4- or 5- or 6-yl; 1,2,3,4-tetrahydropyridin-1- or 2- or 3- or 4- or 5- or 6-yl; 1,4-dihydropyridin-1- or 2- or 3- or 4-yl; 2,3-dihydropyridin-2- or 3- or 4- or 5- or 6-yl; 2,5-dihydropyridin-2- or 3- or 4- or 5- or 6-yl, 1- or 2- or 3- or 4-azepanyl; 2,3,4,5-tetrahydro-1H-azepin-1- or 2- or 3- or 4- or 5- or 6- or 7-yl; 2,3,4,7-tetrahydro-1H-azepin-1- or 2- or 3- or 4- or 5- or 6- or 7-yl; 2,3,6,7-tetrahydro-1H-azepin-1- or 2- or 3- or 4-yl; 3,4,5,6-tetrahydro-2H-azepin-2- or 3- or 4- or 5- or 6- or 7-yl; 4,5-dihydro-1H-azepin-1- or 2- or 3- or 4-yl; 2,5-dihydro-1H-azepin-1- or -2- or 3- or 4- or 5- or 6- or 7-yl; 2,7-dihydro-1H-azepin-1- or -2- or 3- or 4-yl; 2,3-dihydro-1H-azepin-1- or -2- or 3- or 4- or 5- or 6- or 7-yl; 3,4-dihydro-2H-azepin-2- or 3- or 4- or 5- or 6- or 7-yl; 3,6-dihydro-2H-azepin-2- or 3- or 4- or 5- or 6- or 7-yl; 5,6-dihydro-2H-azepin-2- or 3- or 4- or 5- or 6- or 7-yl; 4,5-dihydro-3H-azepin-2- or 3- or 4- or 5- or 6- or 7-yl; 1H-azepin-1- or -2- or 3- or 4- or 5- or 6- or 7-yl; 2H-azepin-2- or 3- or 4- or 5- or 6- or 7-yl; 3H-azepin-2- or 3- or 4- or 5- or 6- or 7-yl; 4H-azepin-2- or 3- or 4- or 5- or 6- or 7-yl, 2- or 3-oxolanyl (=2- or 3-tetrahydrofuranyl); 2,3-dihydrofuran-2- or 3- or 4- or 5-yl; 2,5-dihydrofuran-2- or 3-yl, 2- or 3- or 4-oxanyl (=2- or 3- or 4-tetrahydropyranyl); 3,4-dihydro-2H-pyran-2- or 3- or 4- or 5- or 6-yl; 3,6-dihydro-2H-pyran-2- or 3- or 4- or 5- or 6-yl; 2H-pyran-2- or 3- or 4- or 5- or 6-yl; 4H-pyran-2- or 3- or 4-yl, 2- or 3- or 4-oxepanyl; 2,3,4,5-tetrahydrooxepin-2- or 3- or 4- or 5- or 6- or 7-yl; 2,3,4,7-tetrahydrooxepin-2- or 3- or 4- or 5- or 6- or 7-yl; 2,3,6,7-tetrahydrooxepin-2- or 3- or 4-yl; 2,3-dihydrooxepin-2- or 3- or 4- or 5- or 6- or 7-yl; 4,5-dihydrooxepin-2- or 3- or 4-yl; 2,5-dihydrooxepin-2- or 3- or 4- or 5- or 6- or 7-yl; oxepin-2- or 3- or 4- or 5- or 6- or 7-yl; 2- or 3-tetrahydrothiophenyl; 2,3-dihydrothiophen-2- or 3- or 4- or 5-yl; 2,5-dihydrothiophen-2- or 3-yl; tetrahydro-2H-thiopyran-2- or 3- or 4-yl; 3,4-dihydro-2H-thiopyran-2- or 3- or 4- or 5- or 6-yl; 3,6-dihydro-2H-thiopyran-2- or 3- or 4- or 5- or 6-yl; 2H-thiopyran-2- or 3- or 4- or 5- or 6-yl; 4H-thiopyran-2- or 3- or 4-yl. Preferred 3-membered and 4-membered heterocyclic rings are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partly or fully hydrogenated heterocyclic radical having two heteroatoms from the group of N, O and S, for example 1- or 2- or 3- or 4-pyrazolidinyl; 4,5-dihydro-3H-pyrazol-3- or 4- or 5-yl; 4,5-dihydro-1H-pyrazol-1- or 3- or 4- or 5-yl; 2,3-dihydro-1H-pyrazol-1- or 2- or 3- or 4- or 5-yl; 1- or 2- or 3- or 4-imidazolidinyl; 2,3-dihydro-1H-imidazol-1- or 2- or 3- or 4-yl; 2,5-dihydro-1H-imidazol-1- or 2- or 4- or 5-yl; 4,5-dihydro-1H-imidazol-1- or 2- or 4- or 5-yl; hexahydropyridazin-1- or 2- or 3- or 4-yl; 1,2,3,4-tetrahydropyridazin-1- or 2- or 3- or 4- or 5- or 6-yl; 1,2,3,6-tetrahydropyridazin-1- or 2- or 3- or 4- or 5- or 6-yl; 1,4,5,6-tetrahydropyridazin-1- or 3- or 4- or 5- or 6-yl; 3,4,5,6-tetrahydropyridazin-3- or 4- or 5-yl; 4,5-dihydropyridazin-3- or 4-yl; 3,4-dihydropyridazin-3- or 4- or 5- or 6-yl; 3,6-dihydropyridazin-3- or 4-yl; 1,6-dihydropyriazin-1- or 3- or 4- or 5- or 6-yl; hexahydropyrimidin-1- or 2- or 3- or 4-yl; 1,4,5,6-tetrahydropyrimidin-1- or 2- or 4- or 5- or 6-yl; 1,2,5,6-tetrahydropyrimidin-1- or 2- or 4- or 5- or 6-yl; 1,2,3,4-tetrahydropyrimidin-1- or 2- or 3- or 4- or 5- or 6-yl; 1,6-dihydropyrimidin-1- or 2- or 4- or 5- or 6-yl; 1,2-dihydropyrimidin-1- or 2- or 4- or 5- or 6-yl; 2,5-dihydropyrimidin-2- or 4- or 5-yl; 4,5-dihydropyrimidin-4- or 5- or 6-yl; 1,4-dihydropyrimidin-1- or 2- or 4- or 5- or 6-yl; 1- or 2- or 3-piperazinyl; 1,2,3,6-tetrahydropyrazin-1- or 2- or 3- or 5- or 6-yl; 1,2,3,4-tetrahydropyrazin-1- or 2- or 3- or 4- or 5- or 6-yl; 1,2-dihydropyrazin-1- or 2- or 3- or 5- or 6-yl; 1,4-dihydropyrazin-1- or 2- or 3-yl; 2,3-dihydropyrazin-2- or 3- or 5- or 6-yl; 2,5-dihydropyrazin-2- or 3-yl; 1,3-dioxolan-2- or 4- or 5-yl; 1,3-dioxol-2- or 4-yl; 1,3-dioxan-2- or 4- or 5-yl; 4H-1,3-dioxin-2- or 4- or 5- or 6-yl; 1,4-dioxan-2- or 3- or 5- or 6-yl; 2,3-dihydro-1,4-dioxin-2- or 3- or 5- or 6-yl; 1,4-dioxin-2- or 3-yl; 1,2-dithiolan-3- or 4-yl; 3H-1,2-dithiol-3- or 4- or 5-yl; 1,3-dithiolan-2- or 4-yl; 1,3-dithiol-2- or 4-yl; 1,2-dithian-3- or 4-yl; 3,4-dihydro-1,2-dithiin-3- or 4- or 5- or 6-yl; 3,6-dihydro-1,2-dithiin-3- or 4-yl; 1,2-dithiin-3- or 4-yl; 1,3-dithian-2- or 4- or 5-yl; 4H-1,3-dithiin-2- or 4- or 5- or 6-yl; isoxazolidin-2- or 3- or 4- or 5-yl; 2,3-dihydroisoxazol-2- or 3- or 4- or 5-yl; 2,5-dihydroisoxazol-2- or 3- or 4- or 5-yl; 4,5-dihydroisoxazol-3- or 4- or 5-yl; 1,3-oxazolidin-2- or 3- or 4- or 5-yl; 2,3-dihydro-1,3-oxazol-2- or 3- or 4- or 5-yl; 2,5-dihydro-1,3-oxazol-2- or 4- or 5-yl; 4,5-dihydro-1,3-oxazol-2- or 4- or 5-yl; 1,2-oxazinan-2- or 3- or 4- or 5- or 6-yl; 3,4-dihydro-2H-1,2-oxazin-2- or 3- or 4- or 5- or 6-yl; 3,6-dihydro-2H-1,2-oxazin-2- or 3- or 4- or 5- or 6-yl; 5,6-dihydro-2H-1,2-oxazin-2- or 3- or 4- or 5- or 6-yl; 5,6-dihydro-4H-1,2-oxazin-3- or 4- or 5- or 6-yl; 2H-1,2-oxazin-2- or 3- or 4- or 5- or 6-yl; 6H-1,2-oxazin-3- or 4- or 5- or 6-yl; 4H-1,2-oxazin-3- or 4- or 5- or 6-yl; 1,3-oxazinan-2- or 3- or 4- or 5- or 6-yl; 3,4-dihydro-2H-1,3-oxazin-2- or 3- or 4- or 5- or 6-yl; 3,6-dihydro-2H-1,3-oxazin-2- or 3- or 4- or 5- or 6-yl; 5,6-dihydro-2H-1,3-oxazin-2- or 4- or 5- or 6-yl; 5,6-dihydro-4H-1,3-oxazin-2- or 4- or 5- or 6-yl; 2H-1,3-oxazin-2- or 4- or 5- or 6-yl; 6H-1,3-oxazin-2- or 4- or 5- or 6-yl; 4H-1,3-oxazin-2- or 4- or 5- or 6-yl; morpholin-2- or 3- or 4-yl; 3,4-dihydro-2H-1,4-oxazin-2- or 3- or 4- or 5- or 6-yl; 3,6-dihydro-2H-1,4-oxazin-2- or 3- or 5- or 6-yl; 2H-1,4-oxazin-2- or 3- or 5- or 6-yl; 4H-1,4-oxazin-2- or 3-yl; 1,2-oxazepan-2- or 3- or 4- or 5- or 6- or 7-yl; 2,3,4,5-tetrahydro-1,2-oxazepin-2- or 3- or 4- or 5- or 6- or 7-yl; 2,3,4,7-tetrahydro-1,2-oxazepin-2- or 3- or 4- or 5- or 6- or 7-yl; 2,3,6,7-tetrahydro-1,2-oxazepin-2- or 3- or 4- or 5- or 6- or 7-yl; 2,5,6,7-tetrahydro-1,2-oxazepin-2- or 3- or 4- or 5- or 6- or 7-yl; 4,5,6,7-tetrahydro-1,2-oxazepin-3- or 4- or 5- or 6- or 7-yl; 2,3-dihydro-1,2-oxazepin-2- or 3- or 4- or 5- or 6- or 7-yl; 2,5-dihydro-1,2-oxazepin-2- or 3- or 4- or 5- or 6- or 7-yl; 2,7-dihydro-1,2-oxazepin-2- or 3- or 4- or 5- or 6- or 7-yl; 4,5-dihydro-1,2-oxazepin-3- or 4- or 5- or 6- or 7-yl; 4,7-dihydro-1,2-oxazepin-3- or 4- or 5- or 6- or 7-yl; 6,7-dihydro-1,2-oxazepin-3- or 4- or 5- or 6- or 7-yl; 1,2-oxazepin-3- or 4- or 5- or 6- or 7-yl; 1,3-oxazepan-2- or 3- or 4- or 5- or 6- or 7-yl; 2,3,4,5-tetrahydro-1,3-oxazepin-2- or 3- or 4- or 5- or 6- or 7-yl; 2,3,4,7-tetrahydro-1,3-oxazepin-2- or 3- or 4- or 5- or 6- or 7-yl; 2,3,6,7-tetrahydro-1,3-oxazepin-2- or 3- or 4- or 5- or 6- or 7-yl; 2,5,6,7-tetrahydro-1,3-oxazepin-2- or 4- or 5- or 6- or 7-yl; 4,5,6,7-tetrahydro-1,3-oxazepin-2- or 4- or 5- or 6- or 7-yl; 2,3-dihydro-1,3-oxazepin-2- or 3- or 4- or 5- or 6- or 7-yl; 2,5-dihydro-1,3-oxazepin-2- or 4- or 5- or 6- or 7-yl; 2,7-dihydro-1,3-oxazepin-2- or 4- or 5- or 6- or 7-yl; 4,5-dihydro-1,3-oxazepin-2- or 4- or 5- or 6- or 7-yl; 4,7-dihydro-1,3-oxazepin-2- or 4- or 5- or 6- or 7-yl; 6,7-dihydro-1,3-oxazepin-2- or 4- or 5- or 6- or 7-yl; 1,3-oxazepin-2- or 4- or 5- or 6- or 7-yl; 1,4-oxazepan-2- or 3- or 5- or 6- or 7-yl; 2,3,4,5-tetrahydro-1,4-oxazepin-2- or 3- or 4- or 5- or 6- or 7-yl; 2,3,4,7-tetrahydro-1,4-oxazepin-2- or 3- or 4- or 5- or 6- or 7-yl; 2,3,6,7-tetrahydro-1,4-oxazepin-2- or 3- or 5- or 6- or 7-yl; 2,5,6,7-tetrahydro-1,4-oxazepin-2- or 3- or 5- or 6- or 7-yl; 4,5,6,7-tetrahydro-1,4-oxazepin-2- or 3- or 4- or 5- or 6- or 7-yl; 2,3-dihydro-1,4-oxazepin-2- or 3- or 5- or 6- or 7-yl; 2,5-dihydro-1,4-oxazepin-2- or 3- or 5- or 6- or 7-yl; 2,7-dihydro-1,4-oxazepin-2- or 3- or 5- or 6- or 7-yl; 4,5-dihydro-1,4-oxazepin-2- or 3- or 4- or 5- or 6- or 7-yl; 4,7-dihydro-1,4-oxazepin-2- or 3- or 4- or 5- or 6- or 7-yl; 6,7-dihydro-1,4-oxazepin-2- or 3- or 5- or 6- or 7-yl; 1,4-oxazepin-2- or 3- or 5- or 6- or 7-yl; isothiazolidin-2- or 3- or 4- or 5-yl; 2,3-dihydroisothiazol-2- or 3- or 4- or 5-yl; 2,5-dihydroisothiazol-2- or 3- or 4- or 5-yl; 4,5-dihydroisothiazol-3- or 4- or 5-yl; 1,3-thiazolidin-2- or 3- or 4- or 5-yl; 2,3-dihydro-1,3-thiazol-2- or 3- or 4- or 5-yl; 2,5-dihydro-1,3-thiazol-2- or 4- or 5-yl; 4,5-dihydro-1,3-thiazol-2- or 4- or 5-yl; 1,3-thiazinan-2- or 3- or 4- or 5- or 6-yl; 3,4-dihydro-2H-1,3-thiazin-2- or 3- or 4- or 5- or 6-yl; 3,6-dihydro-2H-1,3-thiazin-2- or 3- or 4- or 5- or 6-yl; 5,6-dihydro-2H-1,3-thiazin-2- or 4- or 5- or 6-yl; 5,6-dihydro-4H-1,3-thiazin-2- or 4- or 5- or 6-yl; 2H-1,3-thiazin-2- or 4- or 5- or 6-yl; 6H-1,3-thiazin-2- or 4- or 5- or 6-yl; 4H-1,3-thiazin-2- or 4- or 5- or 6-yl. Further examples of “heterocyclyl” are a partly or fully hydrogenated heterocyclic radical having 3 heteroatoms from the group of N, O and S, for example 1, 4,2-dioxazolidin-2- or 3- or 5-yl; 1,4,2-dioxazol-3- or 5-yl; 1,4,2-dioxazinan-2- or -3- or 5- or 6-yl; 5,6-dihydro-1,4,2-dioxazin-3- or 5- or 6-yl; 1,4,2-dioxazin-3- or 5- or 6-yl; 1,4,2-dioxazepan-2- or 3- or 5- or 6- or 7-yl; 6,7-dihydro-5H-1,4,2-dioxazepin-3- or 5- or 6- or 7-yl; 2,3-dihydro-7H-1,4,2-dioxazepin-2- or 3- or 5- or 6- or 7-yl; 2,3-dihydro-5H-1,4,2-dioxazepin-2- or 3- or 5- or 6- or 7-yl; 5H-1,4,2-dioxazepin-3- or 5- or 6- or 7-yl; 7H-1,4,2-dioxazepin-3- or 5- or 6- or 7-yl.

When a base structure is substituted “by one or more radicals” from a list of radicals (=group) or a generically defined group of radicals, this in each case includes simultaneous substitution by a plurality of identical and/or structurally different radicals.

In the case of a partly or fully saturated nitrogen heterocycle, this may be joined to the rest of the molecule either via carbon or via the nitrogen.

The term “halogen” means, for example, fluorine, chlorine, bromine or iodine. When the term is used for a radical, “halogen” means, for example, a fluorine, chlorine, bromine or iodine atom.

According to the invention, “alkyl” means a straight-chain or branched open-chain, saturated hydrocarbyl radical which is optionally mono- or polysubstituted. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine.

“Fluoroalkyl” means a straight-chain or branched open-chain, saturated and fluorine-substituted hydrocarbyl radical, where at least one fluorine atom is at one of the possible positions.

“Perfluoroalkyl” means a straight-chain or branched open-chain, saturated and fully fluorine-substituted hydrocarbyl radical, for example CF₃, CF₂CF₃, CF₂CF₂CF₃.

“Partly fluorinated alkyl” means a straight-chain or branched, saturated hydrocarbon which is mono- or polysubstituted by fluorine, where the fluorine atoms in question may be present as substituents on one or more different carbon atoms of the straight-chain or branched hydrocarbyl chain, for example CHFCH₃, CH₂CH₂F, CH₂CH₂CF₃, CHF₂, CH₂F, CHFCF₂CF₃.

“Partly fluorinated haloalkyl” means a straight-chain or branched, saturated hydrocarbon which is substituted by different halogen atoms with at least one fluorine atom, where any other halogen atoms are all selected from the group of fluorine, chlorine or bromine, iodine. The halogen atoms in question may be present as substituents on one or more different carbon atoms of the straight-chain or branched hydrocarbyl chain. Partly fluorinated haloalkyl also includes full substitution of the straight or branched chain by halogen including at least one fluorine atom.

“Haloalkyl”, “-alkenyl” and “-alkynyl” mean, respectively, alkyl, alkenyl and alkynyl partly or fully substituted by identical or different halogen atoms, e.g. monohaloalkyl, for example CH₂CH₂Cl, CH₂CH₂Br, CHClCH₃, CH₂Cl, CH₂F; perhaloalkyl, for example CCl₃, CClF₂, CFCl₂; CF₂CClF₂, CF₂CClFCF₃; polyhaloalkyl, for example CH₂CHFCl, CF₂CClFH, CF₂CBrFH, CH₂CF₃; the term “perhaloalkyl” also includes the term “perfluoroalkyl”.

Haloalkoxy is, for example, OCF₃, OCHF₂, OCH₂F, OCF₂CF₃, OCH₂CF₃ and OCH₂CH₂Cl; the situation is equivalent for haloalkenyl and other halogen-substituted radicals.

The expression “(C₁-C₄)-alkyl” is a brief notation for alkyl having one to 4 carbon atoms according to the range stated for carbon atoms, i.e. comprises the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, for the hydrocarbon radicals such as alkyl, alkenyl and alkynyl radicals, including in composite radicals, preference is given to the lower carbon skeletons, for example having 1 to 6 carbon atoms, or having 2 to 6 carbon atoms in the case of unsaturated groups. Alkyl radicals, including in composite radicals such as alkoxy, haloalkyl, etc., are, for example, methyl, ethyl, n-propyl or i-propyl, n-, i- or t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

Alkenyl especially also includes straight-chain or branched hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl is, for example, vinyl which may optionally be substituted by further alkyl radicals, for example prop-1-en-1-yl, but-1-en-1-yl, allyl, 1-methylprop-2-en-1-yl, 2-methylprop-2-en-1-yl, but-2-en-1-yl, 1-methylbut-3-en-1-yl and 1-methylbut-2-en-1-yl, 2-methylprop-1-en-1-yl, 1-methylprop-1-en-1-yl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl, pentenyl, 2-methylpentenyl or hexenyl.

Alkynyl especially also includes straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-Alkynyl is, for example, ethynyl, propargyl, 1-methylprop-2-yn-1-yl, 2-butynyl, 2-pentynyl or 2-hexynyl, preferably propargyl, but-2-yn-1-yl, but-3-yn-1-yl or 1-methylbut-3-yn-1-yl.

The term “cycloalkyl” means a carbocyclic saturated ring system having preferably 3-8 ring carbon atoms, for example cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, bicyclo[2.2.1]hept-2-yl(norbornyl), bicyclo[2.2.2]octan-2-yl, adamantan-1-yl and adamantan-2-yl. The expression “(C₃-C₇)-cycloalkyl” means a brief notation for cycloalkyl having three to 7 carbon atoms corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl.

“Cycloalkenyl” means a carbocyclic, nonaromatic, partly unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

According to the nature and the bonding of the substituents, the compounds of the formula (I) may be present as stereoisomers. The formula (I) embraces all possible stereoisomers defined by their specific three-dimensional form, such as enantiomers, diastereomers, Z and E isomers. When, for example, one or more alkenyl groups are present, diastereomers (Z and E isomers) may occur. When, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or on the preparative scale to prepare test specimens for biological testing. It is equally possible to selectively prepare stereoisomers by using stereoselective reactions using optically active starting materials and/or auxiliaries. The invention thus also relates to all stereoisomers which are embraced by the formula (I) but are not shown in their specific stereomeric form, and to mixtures thereof.

### Synthesis of Aryl- and Hetarylsulfonamides:

Aryl- and hetarylsulfonamides can be prepared by known processes (cf. Comb. Chem. & High Throughput Scr. 2009, 12, 275; Bioorg. Med. Chem. 2007, 15, 7553; EP2065370). The synthesis routes used and examined proceed from commercially available or easily preparable amines and sulfonyl chlorides. The synthesis of substituted azinylalkylamines is described, for example, in WO 2005113553, J. Heterocycl. Chem. 1980, 17, 1061; J. Med. Chem. 2003, 46, 461; WO2003048133. Thus, synthesis routes described in the literature were employed, and some were optimized (scheme 1). Selected detailed synthesis examples are detailed hereinafter.

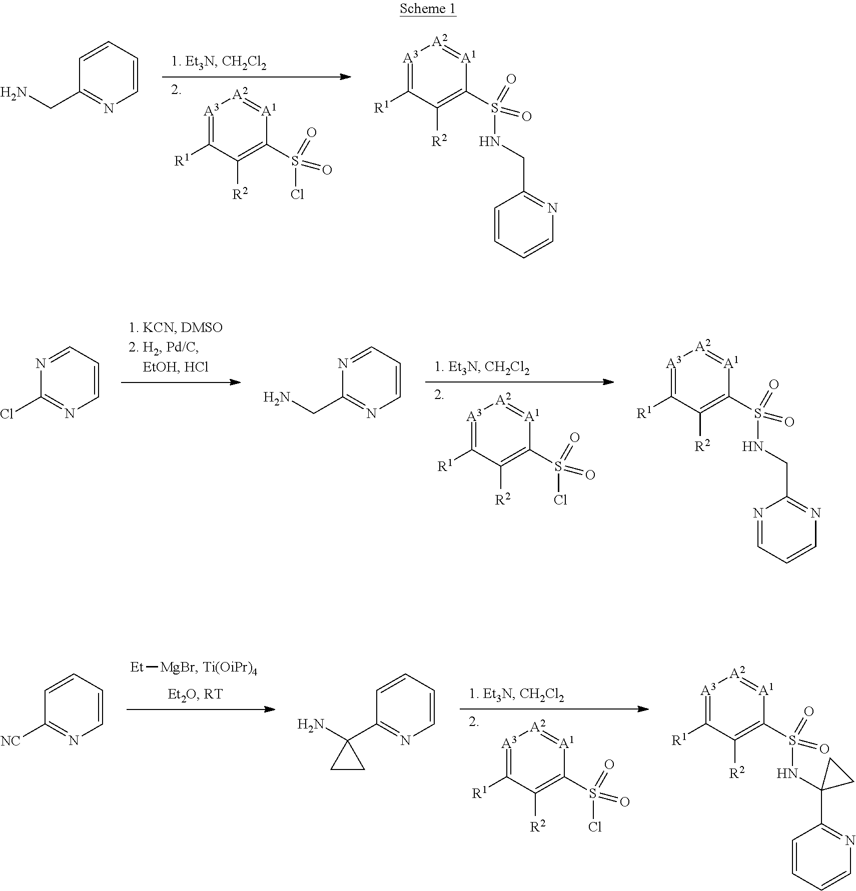

### Example No. 2-50

### N-[(4-Chloro-1-methyl-1H-pyrazol-5-yl)methyl]naphthalene-1-sulfonamide

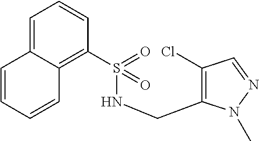

5-Aminomethyl-4-chloro-1-methyl-1H-pyrazole (62 mg, 0.34 mmol) was dissolved in dichloromethane (4 ml) and triethylamine (0.13 ml, 0.93 mmol) under argon. After stirring at room temperature for 10 min, naphthalene-1-sulfonyl chloride (70 mg, 0.31 mmol) was added. The resulting reaction mixture was stirred at room temperature for 3 h, then water and sat. sodium hydrogencarbonate solution were added, and the aqueous phases were subsequently extracted repeatedly with dichloromethane. The combined organic phases were dried over magnesium sulfate, filtered and concentrated under reduced pressure. By column chromatography purification of the crude product (ethyl acetate/heptane gradient), N-[(4-chloro-1-methyl-1H-pyrazol-5-yl)methyl]naphthalene-1-sulfonamide (100 mg, 91%) was obtained as a colorless solid.

¹H NMR (400 MHz, CDCl₃ δ, ppm) 8.58 (d, 1H), 8.23 (d, 1H), 8.07 (d, 1H), 7.96 (d, 1H), 7.67 (dd, 1H), 7.60 (dd, 1H), 7.52 (dd, 1H), 7.14 (s, 1H), 4.94 (br. t, 1H, NH), 4.13 (d, 2H), 3.63 (s, 3H).

### Example No. 3-22

### 4-Bromo-N-[1-(pyridin-2-yl)cyclopropyl]naphthalene-1-sulfonamide

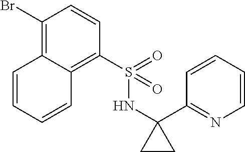

2-Cyanopyridine (1.00 g, 9.52 mmol) was dissolved under argon in diethyl ether (30 ml), titanium(IV) isopropoxide (3.12 ml, 10.47 mmol) and ethylmagnesium bromide in diethyl ether (6.34 ml, 19.03 mmol) were added, and the mixture was stirred at room temperature for 7 h. Subsequently, the reaction mixture was concentrated under reduced pressure and a portion (53 mg, 0.39 mmol) was used unpurified in the next reaction step, by adding dichloromethane (5 ml) and triethylamine (0.09 ml, 0.65 mmol) under argon. After stirring at room temperature for 10 min, 4-bromonaphthalene-1-sulfonyl chloride (100 mg, 0.33 mmol) was added. The resulting reaction mixture was stirred at room temperature for 3 h, then water and sat. sodium hydrogencarbonate solution were added, and the aqueous phases were extracted repeatedly with dichloromethane. The combined organic phases were dried over magnesium sulfate, filtered and concentrated under reduced pressure. By column chromatography purification of the crude product (ethyl acetate/heptane gradient), 4-bromo-N-[1-(pyridin-2-yl)cyclopropyl]naphthalene-1-sulfonamide (130 mg, 94%) was obtained as a colorless solid. ¹H NMR (400 MHz, CDCl₃ δ, ppm) 8.65 (d, 1H), 8.22 (d, 1H), 7.99 (d, 1H), 7.76 (d, 1H), 7.72 (d, 1H), 7.66 (dd, 1H), 7.62 (dd, 1H), 7.21 (dd, 1H), 6.81 (d, 1H), 6.73 (dd, 1H), 6.29 (br. s, 1H, NH), 1.38 (m, 2H), 1.17 (m, 2H).

In analogy to the preparation examples detailed above, and taking account of the general information regarding the preparation of the compounds of the formula (I), the following compounds were obtained with the base structures I.1-I.83 specified in the table below:

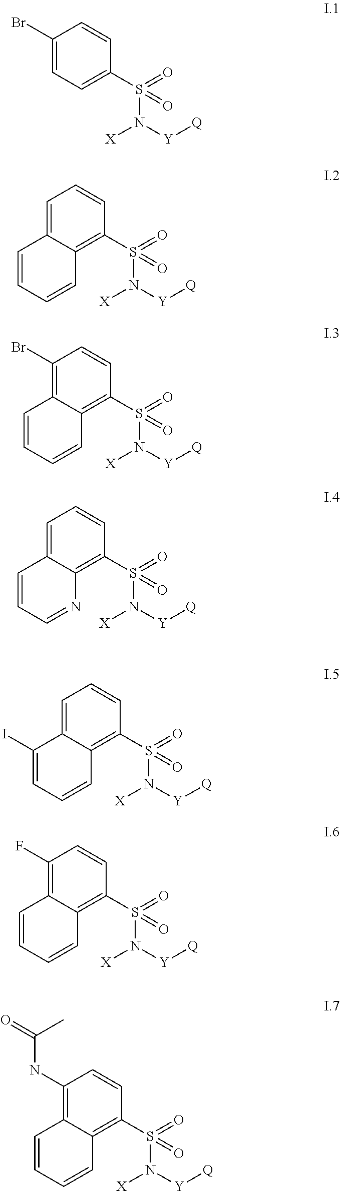

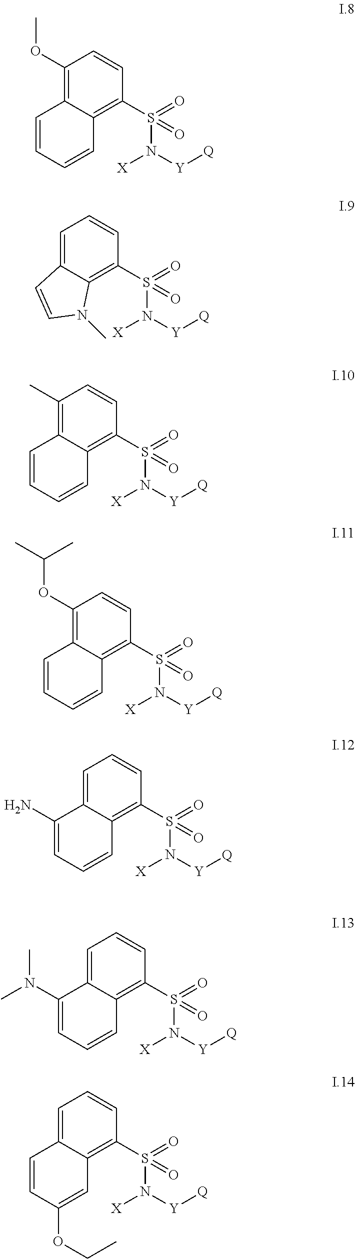

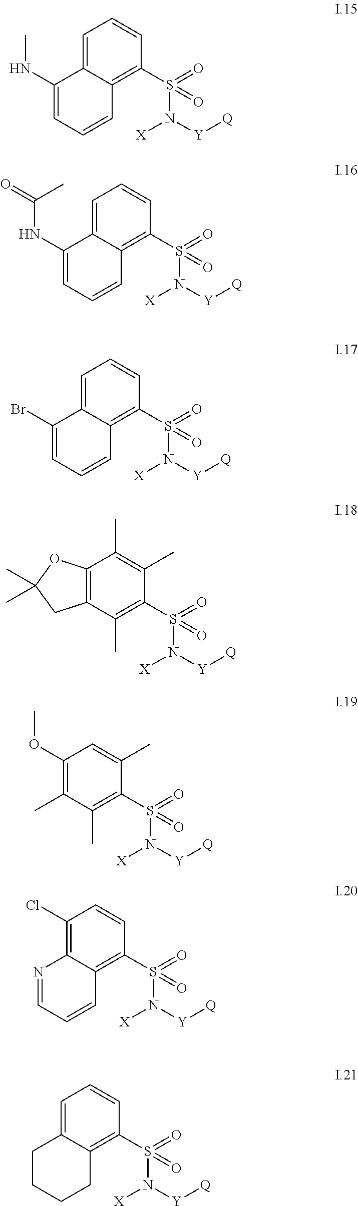

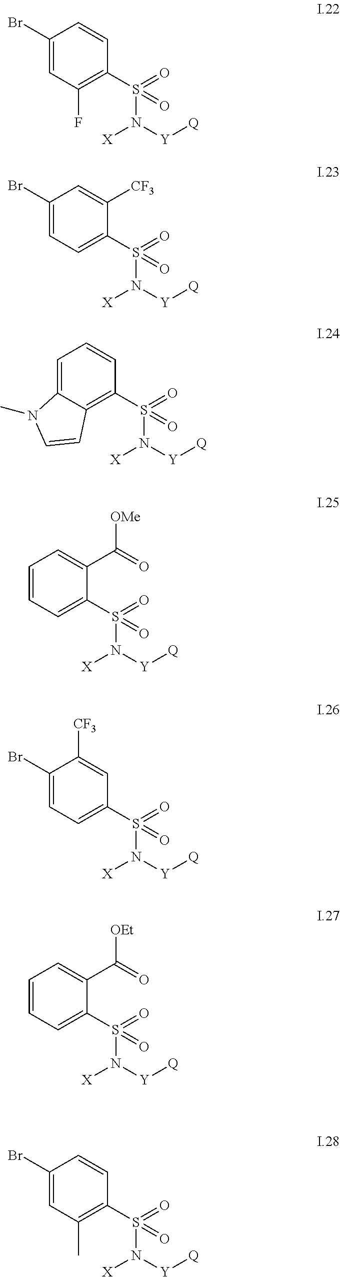

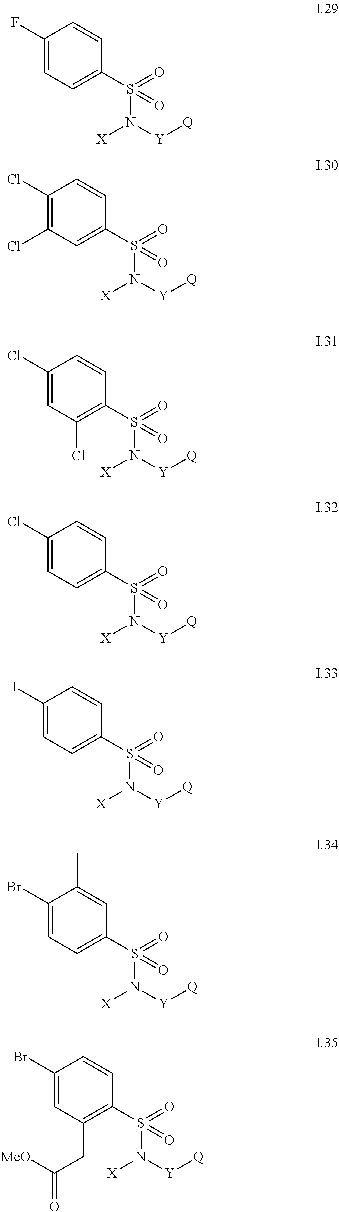

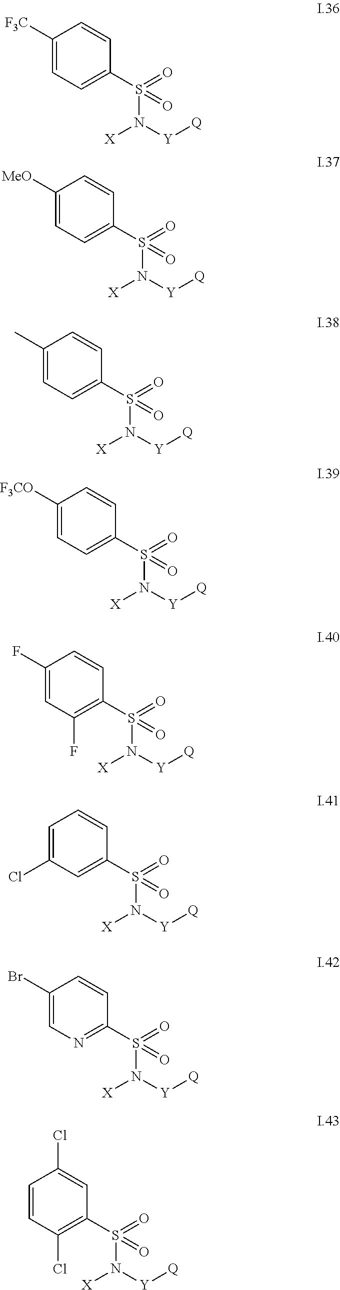

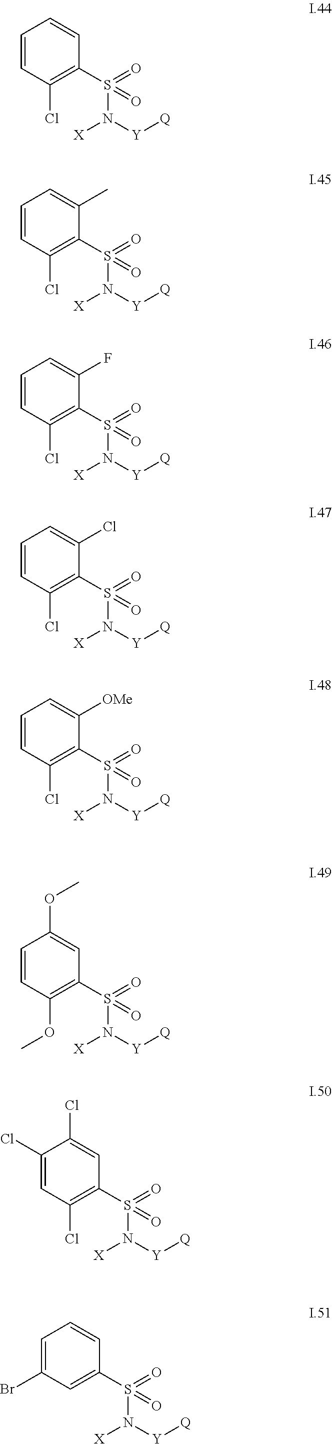

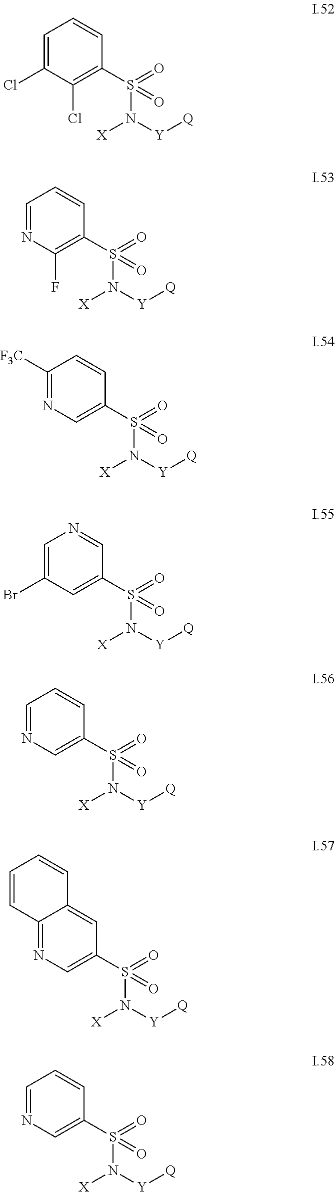

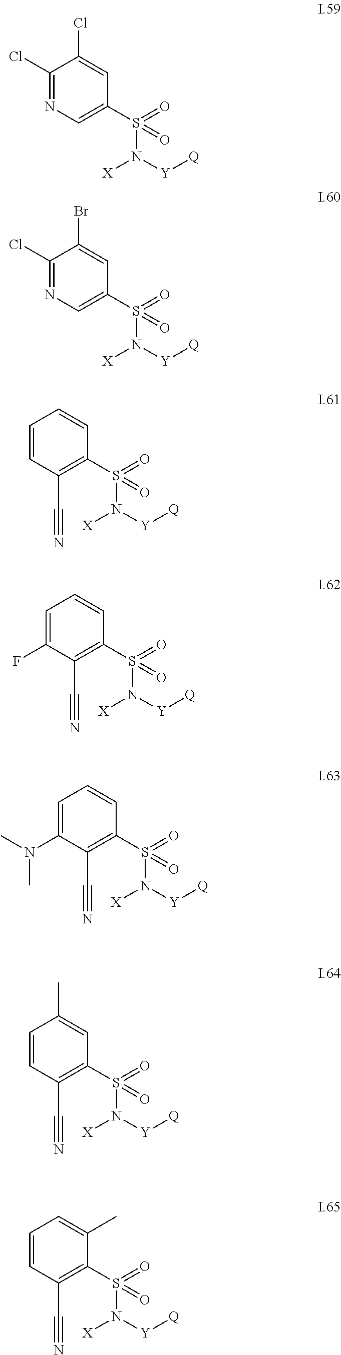

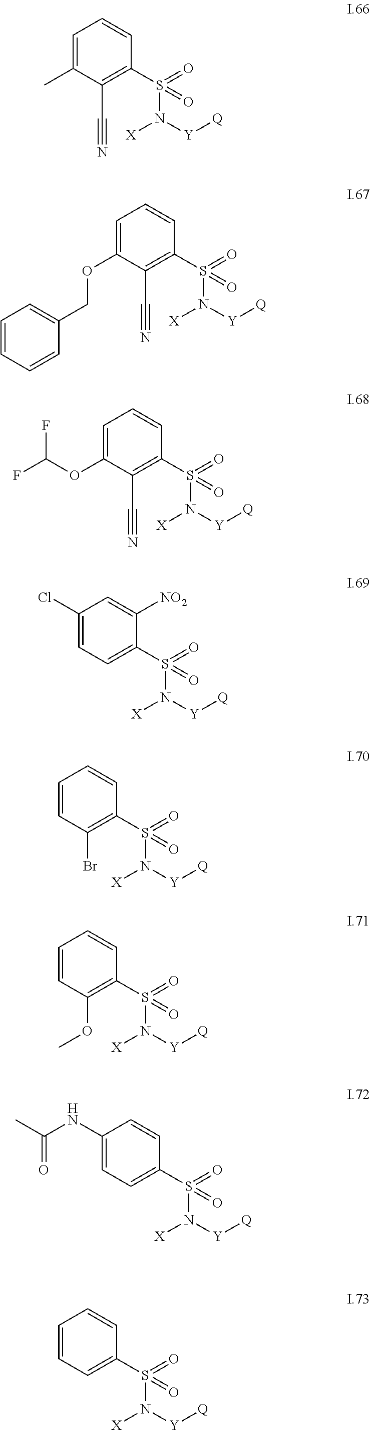

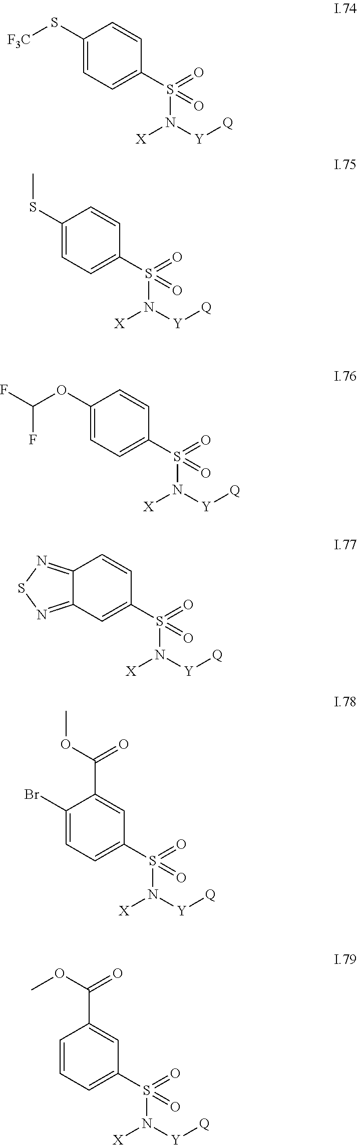

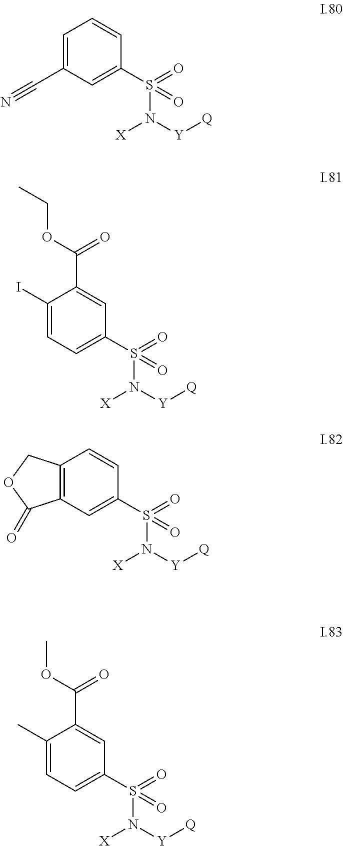

TABLE 1
with base structure I.1 and the radical definitions specified hereinafter:

| No. | I.1-1 | I.1-2 | I.1-3 | I.1-4 | I.1-5 | I.1-6 | I.1-7 | I.1-8 | I.1-9 | I.1-10 | I.1-11 | I.1-12 | I.1-13 | I.1-14 |
|---|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 | Q-14 |
| X | H | H | H | H | H | H | H | H | H | H | CH₃ | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.1-15 | I.1-16 | I.1-17 | I.1-18 | I.1-19 | I.1-20 | I.1-21 | I.1-22 | I.1-23 |
|---|---|---|---|---|---|---|---|---|---|
| Q | Q-1 | Q-6 | Q-1 | Q-1 | Q-12 | Q-1 | Q-1 | Q-1 | Q-17 |
| X | H | H | c-Pr | 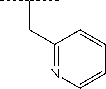 | CH₃ | CH₃ | H | H | H |
| Y | 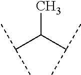 | 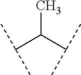 | CH₂ | CH₂ | CH₂ | 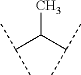 | 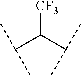 | 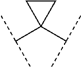 | CH₂ |

| No. | I.1-24 | I.1-25 | I.1-26 | I.1-27 | I.1-28 | I.1-29 | I.1-30 | I.1-31 | I.1-32 | I.1-33 | I.1-34 | I.1-35 | I.1-36 | I.1-37 |
|---|---|---|---|---|---|---|---|---|---|---|---|---|---|---|
| Q | Q-8 | Q-37 | Q-38 | Q-1 | Q-25 | Q-81 | Q-136 | Q-26 | Q-27 | Q-29 | Q-31 | Q-32 | Q-33 | Q-34 |
| X | H | H | H | i-Pr | H | CH₃ | H | H | H | H | H | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.1-38 | I.1-39 | I.1-40 | I.1-41 | I.1-42 | I.1-43 | I.1-44 | I.1-45 | I.1-46 | I.1-47 | I.1-48 | I.1-49 | I.1-50 |
|---|---|---|---|---|---|---|---|---|---|---|---|---|---|
| Q | Q-31 | Q-52 | Q-79 | Q-80 | Q-81 | Q-82 | Q-83 | Q-84 | Q-79 | Q-57 | Q-58 | Q-60 | Q-64 |
| X | H | H | H | H | H | H | H | H | H | H | H | H | H |
| Y | 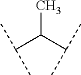 | CH₂ | CH₂ | CH₂ | CH₂ | CH₂ | CH₂ | CH₂ | 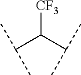 | CH₂ | CH₂ | CH₂ | CH₂ |

| No. | I.1-51 | I.1-52 | I.1-53 | I.1-54 | I.1-55 | I.1-56 | I.1-57 | I.1-58 | I.1-59 | I.1-60 | I.1-61 | I.1-62 | I.1-63 |
|---|---|---|---|---|---|---|---|---|---|---|---|---|---|
| Q | Q-78 | Q-77 | Q-113 | Q-114 | Q-109 | Q-110 | Q-51 | Q-134 | Q-109 | Q-139 | Q-140 | Q-149 | Q-141 |
| X | H | H | H | H | H | H | H | H | CH₃ | H | H | H | H |
| Y | CH₂ | CH₂ | CH₂ | CH₂ | CH₂ | CH₂ | CH₂ | 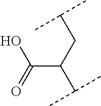 | CH₂ | CH₂ | CH₂ | CH₂ | CH₂ |

TABLE 2
with base structure I.2 and the radical definitions specified hereinafter:

| No. | I.2-1 | I.2-2 | I.2-3 | I.2-4 | I.2-5 | I.2-6 | I.2-7 | I.2-8 | I.2-9 | I.2-10 | I.2-11 | I.2-12 | I.2-13 | I.2-14 |
|---|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 | Q-14 |
| X | H | H | H | H | H | H | H | H | H | H | CH₃ | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.2-15 | I.2-16 | I.2-17 | I.2-18 | I.2-19 | I.2-20 | I.2-21 | I.2-22 | I.2-23 |
|---|---|---|---|---|---|---|---|---|---|
| Q | Q-1 | Q-6 | Q-1 | Q-1 | Q-12 | Q-1 | Q-1 | Q-1 | Q-17 |
| X | H | H | c-Pr | 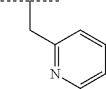 | CH₃ | CH₃ | H | H | H |
| Y | 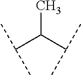 | 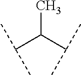 | CH₂ | CH₂ | CH₂ | 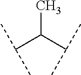 | 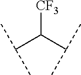 | 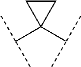 | CH₂ |

| No. | I.2-24 | I.2-25 | I.2-26 | I.2-27 | I.2-28 | I.2-29 | I.2-30 | I.2-31 | I.2-32 | I.2-33 | I.2-34 | I.2-35 | I.2-36 | I.2-37 |
|---|---|---|---|---|---|---|---|---|---|---|---|---|---|---|
| Q | Q-8 | Q-37 | Q-38 | Q-1 | Q-25 | Q-81 | Q-136 | Q-26 | Q-27 | Q-29 | Q-31 | Q-32 | Q-33 | Q-34 |
| X | H | H | H | i-Pr | H | CH₃ | H | H | H | H | H | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.2-38 | I.2-39 | I.2-40 | I.2-41 | I.2-42 | I.2-43 | I.2-44 | I.2-45 | I.2-46 | I.2-47 | I.2-48 | I.2-49 | I.2-50 |
|---|---|---|---|---|---|---|---|---|---|---|---|---|---|
| Q | Q-31 | Q-52 | Q-79 | Q-80 | Q-81 | Q-82 | Q-83 | Q-84 | Q-79 | Q-57 | Q-58 | Q-60 | Q-64 |
| X | H | H | H | H | H | H | H | H | H | H | H | H | H |
| Y | 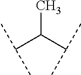 | CH₂ | CH₂ | CH₂ | CH₂ | CH₂ | CH₂ | CH₂ | 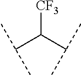 | CH₂ | CH₂ | CH₂ | CH₂ |

| No. | I.2-51 | I.2-52 | I.2-53 | I.2-54 | I.2-55 | I.2-56 | I.2-57 | I.2-58 | I.2-59 | I.2-60 | I.2-61 | I.2-62 | I.2-63 |
|---|---|---|---|---|---|---|---|---|---|---|---|---|---|
| Q | Q-78 | Q-77 | Q-113 | Q-114 | Q-109 | Q-110 | Q-51 | Q-134 | Q-109 | Q-139 | Q-140 | Q-149 | Q-141 |
| X | H | H | H | H | H | H | H | H | CH₃ | H | H | H | H |
| Y | CH₂ | CH₂ | CH₂ | CH₂ | CH₂ | CH₂ | CH₂ | 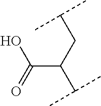 | CH₂ | CH₂ | CH₂ | CH₂ | CH₂ |

TABLE 3
with base structure I.3 and the radical definitions specified hereinafter:

| No. | I.3-1 | I.3-2 | I.3-3 | I.3-4 | I.3-5 | I.3-6 | I.3-7 | I.3-8 | I.3-9 | I.3-10 | I.3-11 | I.3-12 | I.3-13 | I.3-14 |
|---|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 | Q-14 |
| X | H | H | H | H | H | H | H | H | H | H | CH₃ | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.3-15 | I.3-16 | I.3-17 | I.3-18 | I.3-19 | I.3-20 | I.3-21 | I.3-22 | I.3-23 |
|---|---|---|---|---|---|---|---|---|---|
| Q | Q-1 | Q-6 | Q-1 | Q-1 | Q-12 | Q-1 | Q-1 | Q-1 | Q-17 |
| X | H | H | c-Pr | 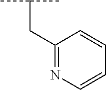 | CH₃ | CH₃ | H | H | H |
| Y | 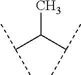 | 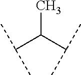 | CH₂ | CH₂ | CH₂ | 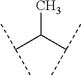 | 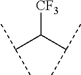 | 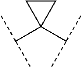 | CH₂ |

| No. | I.3-24 | I.3-25 | I.3-26 | I.3-27 | I.3-28 | I.3-29 | I.3-30 | I.3-31 | I.3-32 | I.3-33 | I.3-34 | I.3-35 | I.3-36 | I.3-37 |
|---|---|---|---|---|---|---|---|---|---|---|---|---|---|---|
| Q | Q-8 | Q-37 | Q-38 | Q-1 | Q-25 | Q-81 | Q-136 | Q-26 | Q-27 | Q-29 | Q-31 | Q-32 | Q-33 | Q-34 |
| X | H | H | H | i-Pr | H | CH₃ | H | H | H | H | H | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.3-38 | I.3-39 | I.3-40 | I.3-41 | I.3-42 | I.3-43 | I.3-44 | I.3-45 | I.3-46 | I.3-47 | I.3-48 | I.3-49 | I.3-50 |
|---|---|---|---|---|---|---|---|---|---|---|---|---|---|
| Q | Q-31 | Q-52 | Q-79 | Q-80 | Q-81 | Q-82 | Q-83 | Q-84 | Q-79 | Q-57 | Q-58 | Q-60 | Q-64 |
| X | H | H | H | H | H | H | H | H | H | H | H | H | H |
| Y | 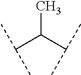 | CH₂ | CH₂ | CH₂ | CH₂ | CH₂ | CH₂ | CH₂ | 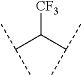 | CH₂ | CH₂ | CH₂ | CH₂ |

| No. | I.3-51 | I.3-52 | I.3-53 | I.3-54 | I.3-55 | I.3-56 | I.3-57 | I.3-58 | I.3-59 | I.3-60 | I.3-61 | I.3-62 | I.3-63 |
|---|---|---|---|---|---|---|---|---|---|---|---|---|---|
| Q | Q-78 | Q-77 | Q-113 | Q-114 | Q-109 | Q-110 | Q-51 | Q-134 | Q-109 | Q-139 | Q-140 | Q-149 | Q-141 |
| X | H | H | H | H | H | H | H | H | CH₃ | H | H | H | H |
| Y | CH₂ | CH₂ | CH₂ | CH₂ | CH₂ | CH₂ | CH₂ | 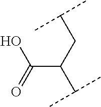 | CH₂ | CH₂ | CH₂ | CH₂ | CH₂ |

TABLE 4
with base structure I.4 and the radical definitions specified hereinafter:

| No. | I.4-1 | I.4-2 | I.4-3 | I.4-4 | I.4-5 | I.4-6 | I.4-7 | I.4-8 | I.4-9 | I.4-10 | I.4-11 | I.4-12 | I.4-13 | I.4-14 |
|---|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 | Q-14 |
| X | H | H | H | H | H | H | H | H | H | H | CH₃ | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.4-15 | I.4-16 | I.4-17 | I.4-18 | I.4-19 | I.4-20 | I.4-21 | I.4-22 | I.4-23 |
|---|---|---|---|---|---|---|---|---|---|
| Q | Q-1 | Q-6 | Q-1 | Q-1 | Q-12 | Q-1 | Q-1 | Q-1 | Q-17 |
| X | H | H | c-Pr | 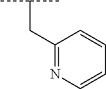 | CH₃ | CH₃ | H | H | H |
| Y | 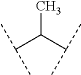 | 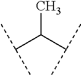 | CH₂ | CH₂ | CH₂ | 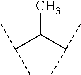 | 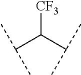 | 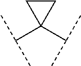 | CH₂ |

| No. | I.4-24 | I.4-25 | I.4-26 | I.4-27 | I.4-28 | I.4-29 | I.4-30 | I.4-31 | I.4-32 | I.4-33 | I.4-34 | I.4-35 | I.4-36 | I.4-37 |
|---|---|---|---|---|---|---|---|---|---|---|---|---|---|---|
| Q | Q-8 | Q-37 | Q-38 | Q-1 | Q-25 | Q-81 | Q-136 | Q-26 | Q-27 | Q-29 | Q-31 | Q-32 | Q-33 | Q-34 |
| X | H | H | H | i-Pr | H | CH₃ | H | H | H | H | H | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.4-38 | I.4-39 | I.4-40 | I.4-41 | I.4-42 | I.4-43 | I.4-44 | I.4-45 | I.4-46 | I.4-47 | I.4-48 | I.4-49 | I.4-50 |
|---|---|---|---|---|---|---|---|---|---|---|---|---|---|
| Q | Q-31 | Q-52 | Q-79 | Q-80 | Q-81 | Q-82 | Q-83 | Q-84 | Q-79 | Q-57 | Q-58 | Q-60 | Q-64 |
| X | H | H | H | H | H | H | H | H | H | H | H | H | H |
| Y | 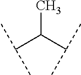 | CH₂ | CH₂ | CH₂ | CH₂ | CH₂ | CH₂ | CH₂ | 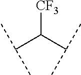 | CH₂ | CH₂ | CH₂ | CH₂ |

| No. | I.4-51 | I.4-52 | I.4-53 | I.4-54 | I.4-55 | I.4-56 | I.4-57 | I.4-58 | I.4-59 | I.4-60 | I.4-61 | I.4-62 | I.4-63 |
|---|---|---|---|---|---|---|---|---|---|---|---|---|---|
| Q | Q-78 | Q-77 | Q-113 | Q-114 | Q-109 | Q-110 | Q-51 | Q-134 | Q-109 | Q-139 | Q-140 | Q-149 | Q-141 |
| X | H | H | H | H | H | H | H | H | CH₃ | H | H | H | H |
| Y | CH₂ | CH₂ | CH₂ | CH₂ | CH₂ | CH₂ | CH₂ | 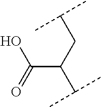 | CH₂ | CH₂ | CH₂ | CH₂ | CH₂ |

TABLE 5
with base structure I.5 and the radical definitions specified hereinafter:

| No. | I.5-1 | I.5-2 | I.5-3 | I.5-4 | I.5-5 | I.5-6 | I.5-7 | I.5-8 | I.5-9 | I.5-10 | I.5-11 | I.5-12 | I.5-13 | I.5-14 |
|---|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 | Q-14 |
| X | H | H | H | H | H | H | H | H | H | H | CH₃ | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.5-15 | I.5-16 | I.5-17 | I.5-18 | I.5-19 | I.5-20 | I.5-21 | I.5-22 | I.5-23 |
|---|---|---|---|---|---|---|---|---|---|
| Q | Q-1 | Q-6 | Q-1 | Q-1 | Q-12 | Q-1 | Q-1 | Q-1 | Q-17 |
| X | H | H | c-Pr | 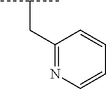 | CH₃ | CH₃ | H | H | H |
| Y | 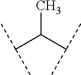 | 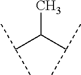 | CH₂ | CH₂ | CH₂ | 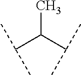 | 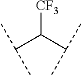 | 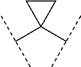 | CH₂ |

| No. | I.5-24 | I.5-25 | I.5-26 | I.5-27 | I.5-28 | I.5-29 | I.5-30 | I.5-31 | I.5-32 | I.5-33 | I.5-34 | I.5-35 | I.5-36 | I.5-37 |
|---|---|---|---|---|---|---|---|---|---|---|---|---|---|---|
| Q | Q-8 | Q-37 | Q-38 | Q-1 | Q-25 | Q-81 | Q-136 | Q-26 | Q-27 | Q-29 | Q-31 | Q-32 | Q-33 | Q-34 |
| X | H | H | H | i-Pr | H | CH₃ | H | H | H | H | H | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.5-38 | I.5-39 | I.5-40 | I.5-41 | I.5-42 | I.5-43 | I.5-44 | I.5-45 | I.5-46 | I.5-47 | I.5-48 | I.5-49 | I.5-50 |
|---|---|---|---|---|---|---|---|---|---|---|---|---|---|
| Q | Q-31 | Q-52 | Q-79 | Q-80 | Q-81 | Q-82 | Q-83 | Q-84 | Q-79 | Q-57 | Q-58 | Q-60 | Q-64 |
| X | H | H | H | H | H | H | H | H | H | H | H | H | H |
| Y | 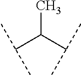 | CH₂ | CH₂ | CH₂ | CH₂ | CH₂ | CH₂ | CH₂ | 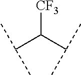 | CH₂ | CH₂ | CH₂ | CH₂ |

| No. | I.5-51 | I.5-52 | I.5-53 | I.5-54 | I.5-55 | I.5-56 | I.5-57 | I.5-58 | I.5-59 | I.5-60 | I.5-61 | I.5-62 | I.5-63 |
|---|---|---|---|---|---|---|---|---|---|---|---|---|---|
| Q | Q-78 | Q-77 | Q-113 | Q-114 | Q-109 | Q-110 | Q-51 | Q-134 | Q-109 | Q-139 | Q-140 | Q-149 | Q-141 |
| X | H | H | H | H | H | H | H | H | CH₃ | H | H | H | H |
| Y | CH₂ | CH₂ | CH₂ | CH₂ | CH₂ | CH₂ | CH₂ | 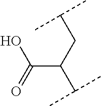 | CH₂ | CH₂ | CH₂ | CH₂ | CH₂ |

TABLE 6
with base structure I.6 and the radical definitions specified hereinafter:

| No. | I.6-1 | I.6-2 | I.6-3 | I.6-4 | I.6-5 | I.6-6 | I.6-7 | I.6-8 | I.6-9 | I.6-10 | I.6-11 | I.6-12 | I.6-13 | I.6-14 |
|---|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 | Q-14 |
| X | H | H | H | H | H | H | H | H | H | H | CH₃ | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.6-15 | I.6-16 | I.6-17 | I.6-18 | I.6-19 | I.6-20 | I.6-21 | I.6-22 | I.6-23 |
|---|---|---|---|---|---|---|---|---|---|
| Q | Q-1 | Q-6 | Q-1 | Q-1 | Q-12 | Q-1 | Q-1 | Q-1 | Q-17 |
| X | H | H | c-Pr | 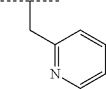 | CH₃ | CH₃ | H | H | H |
| Y | 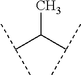 | 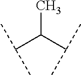 | CH₂ | CH₂ | CH₂ | 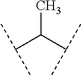 | 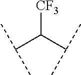 | 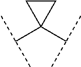 | CH₂ |

| No. | I.6-24 | I.6-25 | I.6-26 | I.6-27 | I.6-28 | I.6-29 | I.6-30 | I.6-31 | I.6-32 | I.6-33 | I.6-34 | I.6-35 | I.6-36 | I.6-37 |
|---|---|---|---|---|---|---|---|---|---|---|---|---|---|---|
| Q | Q-8 | Q-37 | Q-38 | Q-1 | Q-25 | Q-81 | Q-136 | Q-26 | Q-27 | Q-29 | Q-31 | Q-32 | Q-33 | Q-34 |
| X | H | H | H | i-Pr | H | CH₃ | H | H | H | H | H | H | H | H |
| Y | CH₂ | CH₂ | CH₂ | CH₂ | CH₂ | CH₂ | CH₂ | CH₂ | CH₂ | CH₂ | CH₂ | CH₂ | CH₂ | CH₂ |

| No. | I.6-38 | I.6-39 | I.6-40 | I.6-41 | I.6-42 | I.6-43 | I.6-44 | I.6-45 | I.6-46 | I.6-47 | I.6-48 | I.6-49 | I.6-50 |
|---|---|---|---|---|---|---|---|---|---|---|---|---|---|
| Q | Q-31 | Q-52 | Q-79 | Q-80 | Q-81 | Q-82 | Q-83 | Q-84 | Q-79 | Q-57 | Q-58 | Q-60 | Q-64 |
| X | H | H | H | H | H | H | H | H | H | H | H | H | H |
| Y | 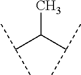 | CH₂ | CH₂ | CH₂ | CH₂ | CH₂ | CH₂ | CH₂ | 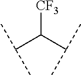 | CH₂ | CH₂ | CH₂ | CH₂ |

| No. | I.6-51 | I.6-52 | I.6-53 | I.6-54 | I.6-55 | I.6-56 | I.6-57 | I.6-58 | I.6-59 | I.6-60 | I.6-61 | I.6-62 | I.6-63 |
|---|---|---|---|---|---|---|---|---|---|---|---|---|---|
| Q | Q-78 | Q-77 | Q-113 | Q-114 | Q-109 | Q-110 | Q-51 | Q-134 | Q-109 | Q-139 | Q-140 | Q-149 | Q-141 |
| X | H | H | H | H | H | H | H | H | CH₃ | H | H | H | H |
| Y | CH₂ | CH₂ | CH₂ | CH₂ | CH₂ | CH₂ | CH₂ | 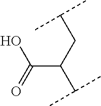 | CH₂ | CH₂ | CH₂ | CH₂ | CH₂ |

TABLE 7
with base structure I.7 and the radical definitions specified hereinafter:

| No. | I.7-1 | I.7-2 | I.7-3 | I.7-4 | I.7-5 | I.7-6 | I.7-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.7-8 | I.7-9 | I.7-10 | I.7-11 | I.7-12 | I.7-13 | I.7-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.7-15 | I.7-16 | I.7-17 | I.7-18 | I.7-19 | I.7-20 | I.7-21 |
| Q | Q-1 | Q-6 | Q-1 | Q-1 | Q12 | Q-1 | Q-1 |
| X | H | H | c-Pr | 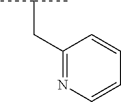 | CH₃ | CH₃ | H |
| Y | 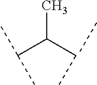 | 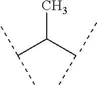 | CH₂ | CH₂ | CH₂ | 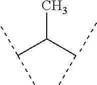 | 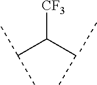 |
| No. | I.7-22 | I.7-23 | I.7-24 | I.7-25 | I.7-26 | I.7-27 | I.7-28 |
| Q | Q-1 | Q-17 | Q-8 | Q-37 | Q-38 | Q-1 | Q-25 |
| X | H | H | H | H | H | i-Pr | H |
| Y | 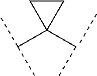 | CH₂ | CH₂ | CH₂ | CH₂ | CH₂ | CH₂ |
| No. | I.7-29 | I.7-30 | I.7-31 | I.7-32 | I.7-33 | I.7-34 | I.7-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.7-36 | I.7-37 | I.7-38 | I.7-39 | I.7-40 | I.7-41 | I.7-42 |
| Q | Q-33 | Q-34 | Q-31 | Q-52 | Q-79 | Q-80 | Q-81 |
| X | H | H | H | H | H | H | H |
| Y | CH₂ | CH₂ | 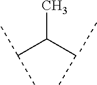 | CH₂ | CH₂ | CH₂ | CH₂ |
| No. | I.7-43 | I.7-44 | I.7-45 | I.7-46 | I.7-47 | I.7-48 | I.7-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 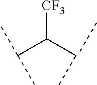 | CH₂ | CH₂ | CH₂ |
| No. | I.7-50 | I.7-51 | I.7-52 | I.7-53 | I.7-54 | I.7-55 | I.7-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.7-57 | I.7-58 | I.7-59 | I.7-60 | I.7-61 | I.7-62 | I.7-63 |
| Q | Q-51 | Q-134 | Q-109 | Q-139 | Q-140 | Q-149 | Q-141 |
| X | H | H | CH₃ | H | H | H | H |
| Y | CH₂ | 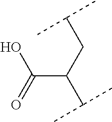 | CH₂ | CH₂ | CH₂ | CH₂ | CH₂ |

TABLE 8
with base structure I.8 and the radical definitions specified hereinafter:

| No. | I.8-1 | I.8-2 | I.8-3 | I.8-4 | I.8-5 | I.8-6 | I.8-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.8-8 | I.8-9 | I.8-10 | I.8-11 | I.8-12 | I.8-13 | I.8-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.8-15 | I.8-16 | I.8-17 | I.8-18 | I.8-19 | I.8-20 | I.8-21 |
| Q | Q-1 | Q-6 | Q-1 | Q-1 | Q12 | Q-1 | Q-1 |
| X | H | H | c-Pr | 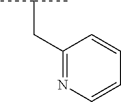 | CH₃ | CH₃ | H |
| Y | 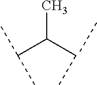 | 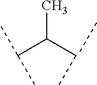 | CH₂ | CH₂ | CH₂ | 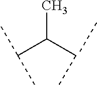 | 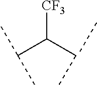 |
| No. | I.8-22 | I.8-23 | I.8-24 | I.8-25 | I.8-26 | I.8-27 | I.8-28 |
| Q | Q-1 | Q-17 | Q-8 | Q-37 | Q-38 | Q-1 | Q-25 |
| X | H | H | H | H | H | i-Pr | H |
| Y | 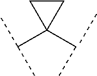 | CH₂ | CH₂ | CH₂ | CH₂ | CH₂ | CH₂ |
| No. | I.8-29 | I.8-30 | I.8-31 | I.8-32 | I.8-33 | I.8-34 | I.8-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.8-36 | I.8-37 | I.8-38 | I.8-39 | I.8-40 | I.8-41 | I.8-42 |
| Q | Q-33 | Q-34 | Q-31 | Q-52 | Q-79 | Q-80 | Q-81 |
| X | H | H | H | H | H | H | H |
| Y | CH₂ | CH₂ | 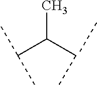 | CH₂ | CH₂ | CH₂ | CH₂ |
| No. | I.8-43 | I.8-44 | I.8-45 | I.8-46 | I.8-47 | I.8-48 | I.8-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 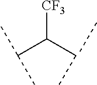 | CH₂ | CH₂ | CH₂ |
| No. | I.8-50 | I.8-51 | I.8-52 | I.8-53 | I.8-54 | I.8-55 | I.8-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.8-57 | I.8-58 | I.8-59 | I.8-60 | I.8-61 | I.8-62 | I.8-63 |
| Q | Q-51 | Q-134 | Q-109 | Q-139 | Q-140 | Q-149 | Q-141 |
| X | H | H | CH₃ | H | H | H | H |
| Y | HO | 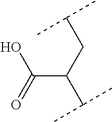 | CH₂ | CH₂ | CH₂ | CH₂ | CH₂ |

TABLE 9
with base structure I.9 and the radical definitions specified hereinafter:

| No. | I.9-1 | I.9-2 | I.9-3 | I.9-4 | I.9-5 | I.9-6 | I.9-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.9-8 | I.9-9 | I.9-10 | I.9-11 | I.9-12 | I.9-13 | I.9-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.9-15 | I.9-16 | I.9-17 | I.9-18 | I.9-19 | I.9-20 | I.9-21 |
| Q | Q-1 | Q-6 | Q-1 | Q-1 | Q12 | Q-1 | Q-1 |
| X | H | H | c-Pr | 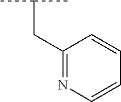 | CH₃ | CH₃ | H |
| Y | 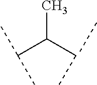 | 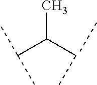 | CH₂ | CH₂ | CH₂ | 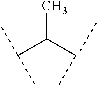 | 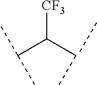 |
| No. | I.9-22 | I.9-23 | I.9-24 | I.9-25 | I.9-26 | I.9-27 | I.9-28 |
| Q | Q-1 | Q-17 | Q-8 | Q-37 | Q-38 | Q-1 | Q-25 |
| X | H | H | H | H | H | i-Pr | H |
| Y | 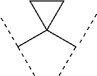 | CH₂ | CH₂ | CH₂ | CH₂ | CH₂ | CH₂ |
| No. | I.9-29 | I.9-30 | I.9-31 | I.9-32 | I.9-33 | I.9-34 | I.9-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.9-36 | I.9-37 | I.9-38 | I.9-39 | I.9-40 | I.9-41 | I.9-42 |
| Q | Q-33 | Q-34 | Q-31 | Q-52 | Q-79 | Q-80 | Q-81 |
| X | H | H | H | H | H | H | H |
| Y | CH₂ | CH₂ | 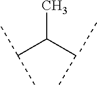 | CH₂ | CH₂ | CH₂ | CH₂ |
| No. | I.9-43 | I.9-44 | I.9-45 | I.9-46 | I.9-47 | I.9-48 | I.9-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 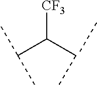 | CH₂ | CH₂ | CH₂ |
| No. | I.9-50 | I.9-51 | I.9-52 | I.9-53 | I.9-54 | I.9-55 | I.9-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.9-57 | I.9-58 | I.9-59 | I.9-60 | I.9-61 | I.9-62 | I.9-63 |
| Q | Q-51 | Q-134 | Q-109 | Q-139 | Q-140 | Q-149 | Q-141 |
| X | H | H | CH₃ | H | H | H | H |
| Y | CH₂ | 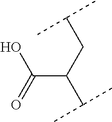 | CH₂ | CH₂ | CH₂ | CH₂ | CH₂ |

TABLE 10
with base structure I.10 and the radical definitions specified hereinafter:

| No. | I.10-1 | I.10-2 | I.10-3 | I.10-4 | I.10-5 | I.10-6 | I.10-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.10-8 | I.10-9 | I.10-10 | I.10-11 | I.10-12 | I.10-13 | I.10-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.10-15 | I.10-16 | I.10-17 | I.10-18 | I.10-19 | I.10-20 | I.10-21 |
| Q | Q-1 | Q-6 | Q-1 | Q-1 | Q12 | Q-1 | Q-1 |
| X | H | H | c-Pr | 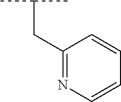 | CH₃ | CH₃ | H |
| Y | 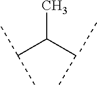 | 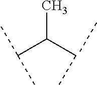 | CH₂ | CH₂ | CH₂ | 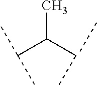 | 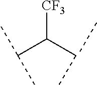 |
| No. | I.10-22 | I.10-23 | I.10-24 | I.10-25 | I.10-26 | I.10-27 | I.10-28 |
| Q | Q-1 | Q-17 | Q-8 | Q-37 | Q-38 | Q-1 | Q-25 |
| X | H | H | H | H | H | i-Pr | H |
| Y | 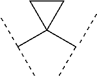 | CH₂ | CH₂ | CH₂ | CH₂ | CH₂ | CH₂ |
| No. | I.10-29 | I.10-30 | I.10-31 | I.10-32 | I.10-33 | I.10-34 | I.10-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.10-36 | I.10-37 | I.10-38 | I.10-39 | I.10-40 | I.10-41 | I.10-42 |
| Q | Q-33 | Q-34 | Q-31 | Q-52 | Q-79 | Q-80 | Q-81 |
| X | H | H | H | H | H | H | H |
| Y | CH₂ | CH₂ | 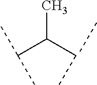 | CH₂ | CH₂ | CH₂ | CH₂ |
| No. | I.10-43 | I.10-44 | I.10-45 | I.10-46 | I.10-47 | I.10-48 | I.10-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 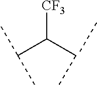 | CH₂ | CH₂ | CH₂ |
| No. | I.10-50 | I.10-51 | I.10-52 | I.10-53 | I.10-54 | I.10-55 | I.10-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.10-57 | I.10-58 | I.10-59 | I.10-60 | I.10-61 | I.10-62 | I.10-63 |
| Q | Q-51 | Q-134 | Q-109 | Q-139 | Q-140 | Q-149 | Q-141 |
| X | H | H | CH₃ | H | H | H | H |
| Y | CH₂ | 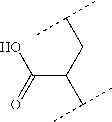 | CH₂ | CH₂ | CH₂ | CH₂ | CH₂ |

TABLE 11
with base structure I.11 and the radical definitions specified hereinafter:

| No. | I.11-1 | I.11-2 | I.11-3 | I.11-4 | I.11-5 | I.11-6 | I.11-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.11-8 | I.11-9 | I.11-10 | I.11-11 | I.11-12 | I.11-13 | I.11-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.11-15 | I.11-16 | I.11-17 | I.11-18 | I.11-19 | I.11-20 | I.11-21 |
| Q | Q-1 | Q-6 | Q-1 | Q-1 | Q12 | Q-1 | Q-1 |
| X | H | H | c-Pr | 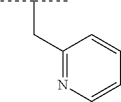 | CH₃ | CH₃ | H |
| Y | 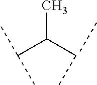 | 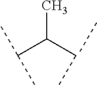 | CH₂ | CH₂ | CH₂ | 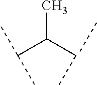 | 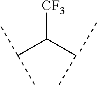 |
| No. | I.11-22 | I.11-23 | I.11-24 | I.11-25 | I.11-26 | I.11-27 | I.11-28 |
| Q | Q-1 | Q-17 | Q-8 | Q-37 | Q-38 | Q-1 | Q-25 |
| X | H | H | H | H | H | i-Pr | H |
| Y | 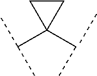 | CH₂ | CH₂ | CH₂ | CH₂ | CH₂ | CH₂ |
| No. | I.11-29 | I.11-30 | I.11-31 | I.11-32 | I.11-33 | I.11-34 | I.11-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.11-36 | I.11-37 | I.11-38 | I.11-39 | I.11-40 | I.11-41 | I.11-42 |
| Q | Q-33 | Q-34 | Q-31 | Q-52 | Q-79 | Q-80 | Q-81 |
| X | H | H | H | H | H | H | H |
| Y | CH₂ | CH₂ | 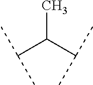 | CH₂ | CH₂ | CH₂ | CH₂ |
| No. | I.11-43 | I.11-44 | I.11-45 | I.11-46 | I.11-47 | I.11-48 | I.11-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 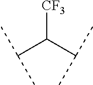 | CH₂ | CH₂ | CH₂ |
| No. | I.11-50 | I.11-51 | I.11-52 | I.11-53 | I.11-54 | I.11-55 | I.11-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.11-57 | I.11-58 | I.11-59 | I.11-60 | I.11-61 | I.11-62 | I.11-63 |
| Q | Q-51 | Q-134 | Q-109 | Q-139 | Q-140 | Q-149 | Q-141 |
| X | H | H | CH₃ | H | H | H | H |
| Y | CH₂ | 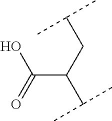 | CH₂ | CH₂ | CH₂ | CH₂ | CH₂ |

TABLE 12
with base structure I.12 and the radical definitions specified hereinafter:

| No. | I.12-1 | I.12-2 | I.12-3 | I.12-4 | I.12-5 | I.12-6 | I.12-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.12-8 | I.12-9 | I.12-10 | I.12-11 | I.12-12 | I.12-13 | I.12-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.12-15 | I.12-16 | I.12-17 | I.12-18 | I.12-19 | I.12-20 | I.12-21 |
| Q | Q-1 | Q-6 | Q-1 | Q-1 | Q12 | Q-1 | Q-1 |
| X | H | H | c-Pr | 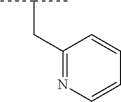 | CH₃ | CH₃ | H |
| Y | 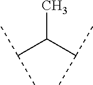 | 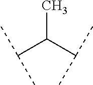 | CH₂ | CH₂ | CH₂ | 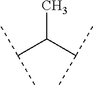 | 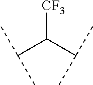 |
| No. | I.12-22 | I.12-23 | I.12-24 | I.12-25 | I.12-26 | I.12-27 | I.12-28 |
| Q | Q-1 | Q-17 | Q-8 | Q-37 | Q-38 | Q-1 | Q-25 |
| X | H | H | H | H | H | i-Pr | H |
| Y | 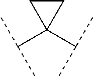 | CH₂ | CH₂ | CH₂ | CH₂ | CH₂ | CH₂ |
| No. | I.12-29 | I.12-30 | I.12-31 | I.12-32 | I.12-33 | I.12-34 | I.12-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.12-36 | I.12-37 | I.12-38 | I.12-39 | I.12-40 | I.12-41 | I.12-42 |
| Q | Q-33 | Q-34 | Q-31 | Q-52 | Q-79 | Q-80 | Q-81 |
| X | H | H | H | H | H | H | H |
| Y | CH₂ | CH₂ | 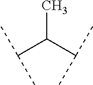 | CH₂ | CH₂ | CH₂ | CH₂ |
| No. | I.12-43 | I.12-44 | I.12-45 | I.12-46 | I.12-47 | I.12-48 | I.12-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 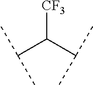 | CH₂ | CH₂ | CH₂ |
| No. | I.12-50 | I.12-51 | I.12-52 | I.12-53 | I.12-54 | I.12-55 | I.12-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.12-57 | I.12-58 | I.12-59 | I.12-60 | I.12-61 | I.12-62 | I.12-63 |
| Q | Q-51 | Q-134 | Q-109 | Q-139 | Q-140 | Q-149 | Q-141 |
| X | H | H | CH₃ | H | H | H | H |
| Y | CH₂ | 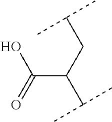 | CH₂ | CH₂ | CH₂ | CH₂ | CH₂ |

TABLE 13
with base structure I.13 and the radical definitions specified hereinafter:

| No. | I.13-1 | I.13-2 | I.13-3 | I.13-4 | I.13-5 | I.13-6 | I.13-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.13-8 | I.13-9 | I.13-10 | I.13-11 | I.13-12 | I.13-13 | I.13-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.13-15 | I.13-16 | I.13-17 | I.13-18 | I.13-19 | I.13-20 | I.13-21 |
| Q | Q-1 | Q-6 | Q-1 | Q-1 | Q12 | Q-1 | Q-1 |
| X | H | H | c-Pr | 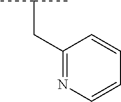 | CH₃ | CH₃ | H |
| Y | 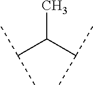 | 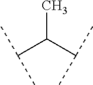 | CH₂ | CH₂ | CH₂ | 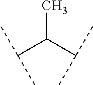 | 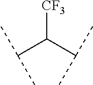 |
| No. | I.13-22 | I.13-23 | I.13-24 | I.13-25 | I.13-26 | I.13-27 | I.13-28 |
| Q | Q-1 | Q-17 | Q-8 | Q-37 | Q-38 | Q-1 | Q-25 |
| X | H | H | H | H | H | i-Pr | H |
| Y | 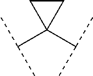 | CH₂ | CH₂ | CH₂ | CH₂ | CH₂ | CH₂ |
| No. | I.13-29 | I.13-30 | I.13-31 | I.13-32 | I.13-33 | I.13-34 | I.13-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.13-36 | I.13-37 | I.13-38 | I.13-39 | I.13-40 | I.13-41 | I.13-42 |
| Q | Q-33 | Q-34 | Q-31 | Q-52 | Q-79 | Q-80 | Q-81 |
| X | H | H | H | H | H | H | H |
| Y | CH₂ | CH₂ | 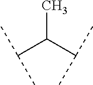 | CH₂ | CH₂ | CH₂ | CH₂ |
| No. | I.13-43 | I.13-44 | I.13-45 | I.13-46 | I.13-47 | I.13-48 | I.13-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 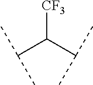 | CH₂ | CH₂ | CH₂ |
| No. | I.13-50 | I.13-51 | I.13-52 | I.13-53 | I.13-54 | I.13-55 | I.13-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.13-57 | I.13-58 | I.13-59 | I.13-60 | I.13-61 | I.13-62 | I.13-63 |
| Q | Q-51 | Q-134 | Q-109 | Q-139 | Q-140 | Q-149 | Q-141 |
| X | H | H | CH₃ | H | H | H | H |
| Y | CH₂ | 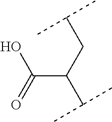 | CH₂ | CH₂ | CH₂ | CH₂ | CH₂ |

TABLE 14
with base structure I.14 and the radical definitions specified hereinafter:

| No. | I.14-1 | I.14-2 | I.14-3 | I.14-4 | I.14-5 | I.14-6 | I.14-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.14-8 | I.14-9 | I.14-10 | I.14-11 | I.14-12 | I.14-13 | I.14-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.14-15 | I.14-16 | I.14-17 | I.14-18 | I.14-19 | I.14-20 | I.14-21 |
| Q | Q-1 | Q-6 | Q-1 | Q-1 | Q12 | Q-1 | Q-1 |
| X | H | H | c-Pr | 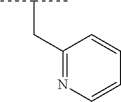 | CH₃ | CH₃ | H |
| Y | 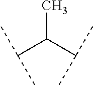 | 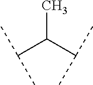 | CH₂ | CH₂ | CH₂ | 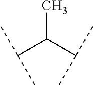 | 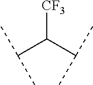 |
| No. | I.14-22 | I.14-23 | I.14-24 | I.14-25 | I.14-26 | I.14-27 | I.14-28 |
| Q | Q-1 | Q-17 | Q-8 | Q-37 | Q-38 | Q-1 | Q-25 |
| X | H | H | H | H | H | i-Pr | H |
| Y | 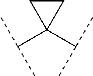 | CH₂ | CH₂ | CH₂ | CH₂ | CH₂ | CH₂ |
| No. | I.14-29 | I.14-30 | I.14-31 | I.14-32 | I.14-33 | I.14-34 | I.14-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.14-36 | I.14-37 | I.14-38 | I.14-39 | I.14-40 | I.14-41 | I.14-42 |
| Q | Q-33 | Q-34 | Q-31 | Q-52 | Q-79 | Q-80 | Q-81 |
| X | H | H | H | H | H | H | H |
| Y | CH₂ | CH₂ | 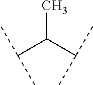 | CH₂ | CH₂ | CH₂ | CH₂ |
| No. | I.14-43 | I.14-44 | I.14-45 | I.14-46 | I.14-47 | I.14-48 | I.14-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 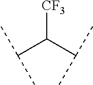 | CH₂ | CH₂ | CH₂ |
| No. | I.14-50 | I.14-51 | I.14-52 | I.14-53 | I.14-54 | I.14-55 | I.14-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.14-57 | I.14-58 | I.14-59 | I.14-60 | I.14-61 | I.14-62 | I.14-63 |
| Q | Q-51 | Q-134 | Q-109 | Q-139 | Q-140 | Q-149 | Q-141 |
| X | H | H | CH₃ | H | H | H | H |
| Y | CH₂ | 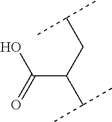 | CH₂ | CH₂ | CH₂ | CH₂ | CH₂ |

TABLE 15
with base structure I.15 and the radical definitions specified hereinafter:

| No. | I.15-1 | I.15-2 | I.15-3 | I.15-4 | I.15-5 | I.15-6 | I.15-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.15-8 | I.15-9 | I.15-10 | I.15-11 | I.15-12 | I.15-13 | I.15-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.15-15 | I.15-16 | I.15-17 | I.15-18 | I.15-19 | I.15-20 | I.15-21 |
| Q | Q-1 | Q-6 | Q-1 | Q-1 | Q12 | Q-1 | Q-1 |
| X | H | H | c-Pr | 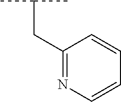 | CH₃ | CH₃ | H |
| Y | 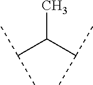 | 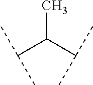 | CH₂ | CH₂ | CH₂ | 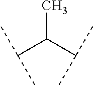 | 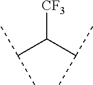 |
| No. | I.15-22 | I.15-23 | I.15-24 | I.15-25 | I.15-26 | I.15-27 | I.15-28 |
| Q | Q-1 | Q-17 | Q-8 | Q-37 | Q-38 | Q-1 | Q-25 |
| X | H | H | H | H | H | i-Pr | H |
| Y | 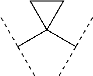 | CH₂ | CH₂ | CH₂ | CH₂ | CH₂ | CH₂ |
| No. | I.15-29 | I.15-30 | I.15-31 | I.15-32 | I.15-33 | I.15-34 | I.15-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.15-36 | I.15-37 | I.15-38 | I.15-39 | I.15-40 | I.15-41 | I.15-42 |
| Q | Q-33 | Q-34 | Q-31 | Q-52 | Q-79 | Q-80 | Q-81 |
| X | H | H | H | H | H | H | H |
| Y | CH₂ | CH₂ | 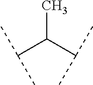 | CH₂ | CH₂ | CH₂ | CH₂ |
| No. | I.15-43 | I.15-44 | I.15-45 | I.15-46 | I.15-47 | I.15-48 | I.15-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 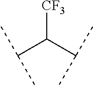 | CH₂ | CH₂ | CH₂ |
| No. | I.15-50 | I.15-51 | I.15-52 | I.15-53 | I.15-54 | I.15-55 | I.15-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.15-57 | I.15-58 | I.15-59 | I.15-60 | I.15-61 | I.15-62 | I.15-63 |
| Q | Q-51 | Q-134 | Q-109 | Q-139 | Q-140 | Q-149 | Q-141 |
| X | H | H | CH₃ | H | H | H | H |
| Y | CH₂ | 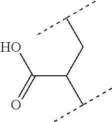 | CH₂ | CH₂ | CH₂ | CH₂ | CH₂ |

TABLE 16
with base structure I.16 and the radical definitions specified hereinafter:

| No. | I.16-1 | I.16-2 | I.16-3 | I.16-4 | I.16-5 | I.16-6 | I.16-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.16-8 | I.16-9 | I.16-10 | I.16-11 | I.16-12 | I.16-13 | I.16-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.16-15 | I.16-16 | I.16-17 | I.16-18 | I.16-19 | I.16-20 | I.16-21 |
| Q | Q-1 | Q-6 | Q-1 | Q-1 | Q12 | Q-1 | Q-1 |
| X | H | H | c-Pr | 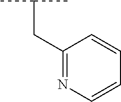 | CH₃ | CH₃ | H |
| Y | 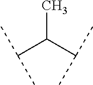 | 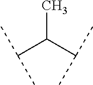 | CH₂ | CH₂ | CH₂ | 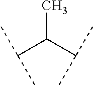 | 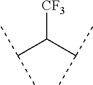 |
| No. | I.16-22 | I.16-23 | I.16-24 | I.16-25 | I.16-26 | I.16-27 | I.16-28 |
| Q | Q-1 | Q-17 | Q-8 | Q-37 | Q-38 | Q-1 | Q-25 |
| X | H | H | H | H | H | i-Pr | H |
| Y | 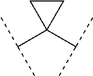 | CH₂ | CH₂ | CH₂ | CH₂ | CH₂ | CH₂ |
| No. | I.16-29 | I.16-30 | I.16-31 | I.16-32 | I.16-33 | I.16-34 | I.16-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.16-36 | I.16-37 | I.16-38 | I.16-39 | I.16-40 | I.16-41 | I.16-42 |
| Q | Q-33 | Q-34 | Q-31 | Q-52 | Q-79 | Q-80 | Q-81 |
| X | H | H | H | H | H | H | H |
| Y | CH₂ | CH₂ | 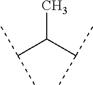 | CH₂ | CH₂ | CH₂ | CH₂ |
| No. | I.16-43 | I.16-44 | I.16-45 | I.16-46 | I.16-47 | I.16-48 | I.16-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 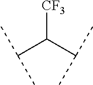 | CH₂ | CH₂ | CH₂ |
| No. | I.16-50 | I.16-51 | I.16-52 | I.16-53 | I.16-54 | I.16-55 | I.16-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.16-57 | I.16-58 | I.16-59 | I.16-60 | I.16-61 | I.16-62 | I.16-63 |
| Q | Q-51 | Q-134 | Q-109 | Q-139 | Q-140 | Q-149 | Q-141 |
| X | H | H | CH₃ | H | H | H | H |
| Y | CH₂ | 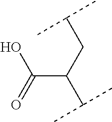 | CH₂ | CH₂ | CH₂ | CH₂ | CH₂ |

TABLE 17
with base structure I.17 and the radical definitions specified hereinafter:

| No. | I.17-1 | I.17-2 | I.17-3 | I. 17-4 | I.17-5 | I.17-6 | I.17-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.17-8 | I.17-9 | I.17-10 | I.17-11 | I.17-12 | I.17-13 | I.17-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.17-15 | I.17-16 | I.17-17 | I.17-18 | I.17-19 | I.17-20 | I.17-21 |
| Q | Q-1 | Q-6 | Q-1 | Q-1 | Q12 | Q-1 | Q-1 |
| X | H | H | c-Pr | 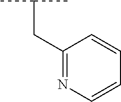 | CH₃ | CH₃ | H |
| Y | 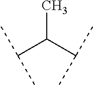 | 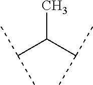 | CH₂ | CH₂ | CH₂ | 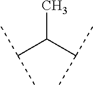 | 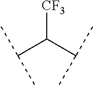 |
| No. | I.17-22 | I.17-23 | I.17-24 | I.17-25 | I.1-26 | I.17-27 | I.17-28 |
| Q | Q-1 | Q-17 | Q-8 | Q-37 | Q-38 | Q-1 | Q-25 |
| X | H | H | H | H | H | i-Pr | H |
| Y | 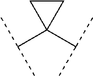 | CH₂ | CH₂ | CH₂ | CH₂ | CH₂ | CH₂ |
| No. | I.17-29 | I.17-30 | I.17-31 | I.17-32 | I.17-33 | I.17-34 | I.17-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.17-36 | I.17-37 | I.17-38 | I.17-39 | I.17-40 | I.17-41 | I.17-42 |
| Q | Q-33 | Q-34 | Q-31 | Q-52 | Q-79 | Q-80 | Q-81 |
| X | H | H | H | H | H | H | H |
| Y | CH₂ | CH₂ | 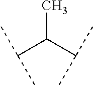 | CH₂ | CH₂ | CH₂ | CH₂ |
| No. | I.17-43 | I.17-44 | I.17-45 | I.17-46 | I.17-47 | I.17-48 | I.17-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 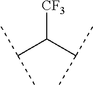 | CH₂ | CH₂ | CH₂ |
| No. | I.17-50 | I.17-51 | I.17-52 | I.17-53 | I.17-54 | I.17-55 | I.17-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.17-57 | I.17-58 | I.17-59 | I.17-60 | I.17-61 | I.17-62 | I.17-63 |
| Q | Q-51 | Q-134 | Q-109 | Q-139 | Q-140 | Q-149 | Q-141 |
| X | H | H | CH₃ | H | H | H | H |
| Y | CH₂ | 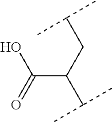 | CH₂ | CH₂ | CH₂ | CH₂ | CH₂ |

TABLE 18
with base structure I.18 and the radical definitions specified hereinafter:

| No. | I.18-1 | I.18-2 | I.18-3 | I.18-4 | I.18-5 | I.18-6 | I.18-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.18-8 | I.18-9 | I.18-10 | I.18-11 | I.18-12 | I.18-13 | I.18-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.18-15 | I.18-16 | I.18-17 | I.18-18 | I.18-19 | I.18-20 | I.18-21 |
| Q | Q-1 | Q-6 | Q-1 | Q-1 | Q12 | Q-1 | Q-1 |
| X | H | H | c-Pr | 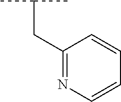 | CH₃ | CH₃ | H |
| Y | 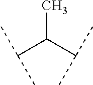 | 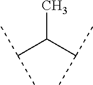 | CH₂ | CH₂ | CH₂ | 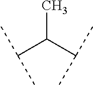 | 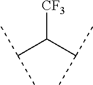 |
| No. | I.18-22 | I.18-23 | I.18-24 | I.18-25 | I.18-26 | I.18-27 | I.18-28 |
| Q | Q-1 | Q-17 | Q-8 | Q-37 | Q-38 | Q-1 | Q-25 |
| X | H | H | H | H | H | i-Pr | H |
| Y | 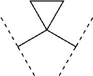 | CH₂ | CH₂ | CH₂ | CH₂ | CH₂ | CH₂ |
| No. | I.18-29 | I.18-30 | I.18-31 | I.18-32 | I.18-33 | I.18-34 | I.18-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.18-36 | I.18-37 | I.18-38 | I.18-39 | I.18-40 | I.18-41 | I.18-42 |
| Q | Q-33 | Q-34 | Q-31 | Q-52 | Q-79 | Q-80 | Q-81 |
| X | H | H | H | H | H | H | H |
| Y | CH₂ | CH₂ | 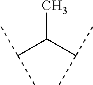 | CH₂ | CH₂ | CH₂ | CH₂ |
| No. | I.18-43 | I.18-44 | I.18-45 | I.18-46 | I.18-47 | I.18-48 | I.18-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 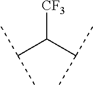 | CH₂ | CH₂ | CH₂ |
| No. | I.18-50 | I.18-51 | I.18-52 | I.18-53 | I.18-54 | I.18-55 | I.18-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.18-57 | I.18-58 | I.18-59 | I.18-60 | I.18-61 | I.18-62 | I.18-63 |
| Q | Q-51 | Q-134 | Q-109 | Q-139 | Q-140 | Q-149 | Q-141 |
| X | H | H | CH₃ | H | H | H | H |
| Y | CH₂ | 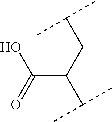 | CH₂ | CH₂ | CH₂ | CH₂ | CH₂ |

TABLE 19
with base structure I.19 and the radical definitions specified hereinafter:

| No. | I.19-1 | I.19-2 | I.19-3 | I.19-4 | I.19-5 | I.19-6 | I.19-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.19-8 | I.19-9 | I.19-10 | I.19-11 | I.19-12 | I.19-13 | I.19-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.19-15 | I.19-16 | I.19-17 | I.19-18 | I.19-19 | I.19-20 | I.19-21 |
| Q | Q-1 | Q-6 | Q-1 | Q-1 | Q12 | Q-1 | Q-1 |
| X | H | H | c-Pr | 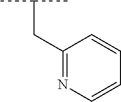 | CH₃ | CH₃ | H |
| Y | 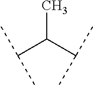 | 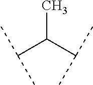 | CH₂ | CH₂ | CH₂ | 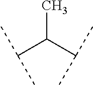 | 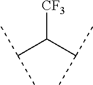 |
| No. | I.19-22 | I.19-23 | I.19-24 | I.19-25 | I.19-26 | I.19-27 | I.19-28 |
| Q | Q-1 | Q-17 | Q-8 | Q-37 | Q-38 | Q-1 | Q-25 |
| X | H | H | H | H | H | i-Pr | H |
| Y | 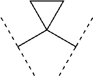 | CH₂ | CH₂ | CH₂ | CH₂ | CH₂ | CH₂ |
| No. | I.19-29 | I.19-30 | I.19-31 | I.19-32 | I.19-33 | I.19-34 | I.19-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.19-36 | I.19-37 | I.19-38 | I.19-39 | I.19-40 | I.19-41 | I.19-42 |
| Q | Q-33 | Q-34 | Q-31 | Q-52 | Q-79 | Q-80 | Q-81 |
| X | H | H | H | H | H | H | H |
| Y | CH₂ | CH₂ | 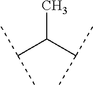 | CH₂ | CH₂ | CH₂ | CH₂ |
| No. | I.19-43 | I.19-44 | I.19-45 | I.19-46 | I.19-47 | I.19-48 | I.19-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 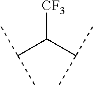 | CH₂ | CH₂ | CH₂ |
| No. | I.19-50 | I.19-51 | I.19-52 | I.19-53 | I.19-54 | I.19-55 | I.19-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.19-57 | I.19-58 | I.19-59 | I.19-60 | I.19-61 | I.19-62 | I.19-63 |
| Q | Q-51 | Q-134 | Q-109 | Q-139 | Q-140 | Q-149 | Q-141 |
| X | H | H | CH₃ | H | H | H | H |
| Y | CH₂ | 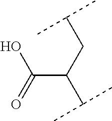 | CH₂ | CH₂ | CH₂ | CH₂ | CH₂ |

TABLE 20
with base structure I.20 and the radical definitions specified hereinafter:

| No. | I.20-1 | I.20-2 | I.20-3 | I.20-4 | I.20-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.20-6 | I.20-7 | I.20-8 | I.29-9 | I.20-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I-20-11 | I-20-12 | I.20-13 | I.20-14 | I.20-15 |
| Q | Q-1 | Q-12 | Q-13 | Q-14 | Q-1 |
| X | CH₃ | H | H | H | H |
| Y | CH₂ | CH₂ | CH₂ | CH₂ | 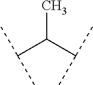 |
| No. | I.20-16 | I.20-17 | I.20-18 | I.20-19 | I.20-20 |
| Q | Q-6 | Q-1 | Q-1 | Q-12 | Q-1 |
| X | H | c-Pr | 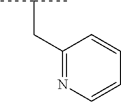 | CH₃ | CH₃ |
| Y | 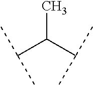 | CH₂ | CH₂ | CH₂ | 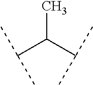 |
| No. | I.20-21 | I.20-22 | I.20-23 | I.20-24 | I.20-25 |
| Q | Q-1 | Q-1 | Q-17 | Q-8 | Q-37 |
| X | H | H | H | H | H |
| Y | 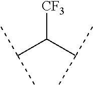 | 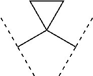 | CH₂ | CH₂ | CH₂ |
| No. | I.20-26 | I.20-27 | I.20-28 | I.20-29 | I.20-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.20-31 | I.20-32 | I.20-33 | I.20-34 | I.2-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.20-36 | I.20-37 | I.2-38 | I.20-39 | I.20-40 |
| Q | Q-33 | Q-34 | Q-31 | Q-52 | Q-79 |
| X | H | H | H | H | H |
| Y | CH₂ | CH₂ | 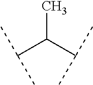 | CH₂ | CH₂ |
| No. | I.20-41 | I.20-42 | I-20-43 | I.20-44 | I.20-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.20-46 | I.20-47 | I.2-48 | I.20-49 | I.20-50 |
| Q | Q-79 | Q-57 | Q-58 | Q-60 | Q-64 |
| X | H | H | H | H | H |
| Y | 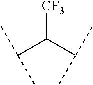 | CH₂ | CH₂ | CH₂ | CH₂ |
| No. | I.20-51 | I.20-52 | I.20-53 | I.2-54 | I.20-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.20-56 | I.2-57 | I.20-58 | I.20-59 | I.20-60 |
| Q | Q-110 | Q-51 | Q-134 | Q-109 | Q-139 |
| X | H | H | H | CH₃ | H |
| Y | CH₂ | CH₂ | 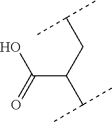 | CH₂ | CH₂ |
| | | No. | I.20-61 | I.20-62 | I.20-63 |
| | | Q | Q-140 | Q-149 | Q-141 |
| | | X | H | H | H |
| | | Y | CH₂ | CH₂ | CH₂ |

TABLE 21
with base structure I.21 and the radical definitions specified hereinafter:

| No. | I.21-1 | I.21-2 | I.21-3 | I.21-4 | I.21-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.21-6 | I.21-7 | I.21-8 | I.21-9 | I.21-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.21-11 | I.21-12 | I.21-13 | I.21-14 | I.21-15 |
| Q | Q-1 | Q-12 | Q-13 | Q-14 | Q-1 |
| X | CH₃ | H | H | H | H |
| Y | CH₂ | CH₂ | CH₂ | CH₂ | 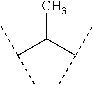 |
| No. | I.21-16 | I.21-17 | I.21-18 | I.21-19 | I.21-20 |
| Q | Q-6 | Q-1 | Q-1 | Q-12 | Q-1 |
| X | H | c-Pr | 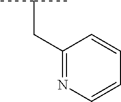 | CH₃ | CH₃ |
| Y | 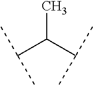 | CH₂ | CH₂ | CH₂ | 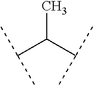 |
| No. | I.21-21 | I.21-22 | I.21-23 | I.21-24 | I.21-25 |
| Q | Q-1 | Q-1 | Q-17 | Q-8 | Q-37 |
| X | H | H | H | H | H |
| Y | 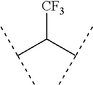 | 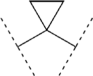 | CH₂ | CH₂ | CH₂ |
| No. | I.21-26 | I.21-17 | I.21-18 | I.21-29 | I.21-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.21-31 | I.21-32 | I.21-33 | I.21-34 | I.21-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.21-36 | I.21-37 | I.21-38 | I.21-39 | I.21-40 |
| Q | Q-33 | Q-34 | Q-31 | Q-52 | Q-79 |
| X | H | H | H | H | H |
| Y | CH₂ | CH₂ | 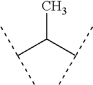 | CH₂ | CH₂ |
| No. | I.21-41 | I.21-42 | I.21-43 | I.21-44 | I.21-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.21-46 | I.21-47 | I.21-48 | I.21-49 | I.21-50 |
| Q | Q-79 | Q-57 | Q-58 | Q-60 | Q-64 |
| X | H | H | H | H | H |
| Y | 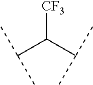 | CH₂ | CH₂ | CH₂ | CH₂ |
| No. | I.21-51 | I.21-52 | I.21-53 | I.21-54 | I.21-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.21-56 | I.21-57 | I.21-58 | I.21-59 | I.21-60 |
| Q | Q-110 | Q-51 | Q-134 | Q-109 | Q-139 |
| X | H | H | H | CH₃ | H |
| Y | CH₂ | CH₂ | 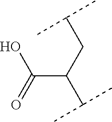 | CH₂ | CH₂ |
| | | No. | I.21-61 | I.21-62 | I.21-63 |
| | | Q | Q-140 | Q-149 | Q-141 |
| | | X | H | H | H |
| | | Y | CH₂ | CH₂ | CH₂ |

TABLE 22
with base structure I.22 and the radical definitions specified hereinafter:

| No. | I.22-1 | I.22-2 | I.22-3 | I.22-4 | I.22-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.22-6 | I.22-7 | I.22-8 | I.22-9 | I.22-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.22-11 | I.22-12 | I.22-13 | I.22-14 | I.22-15 |
| Q | Q-1 | Q-12 | Q-13 | Q-14 | Q-1 |
| X | CH₃ | H | H | H | H |
| Y | CH₂ | CH₂ | CH₂ | CH₂ | 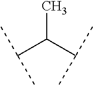 |
| No. | I.22-16 | I.22-17 | I.22-18 | I.22-19 | I.22-20 |
| Q | Q-6 | Q-1 | Q-1 | Q-12 | Q-1 |
| X | H | c-Pr | 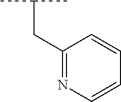 | CH₃ | CH₃ |
| Y | 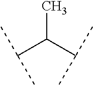 | CH₂ | CH₂ | CH₂ | 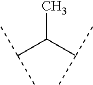 |
| No. | I.22-21 | I.22-22 | I.22-23 | I.22-24 | I.22-25 |
| Q | Q-1 | Q-1 | Q-17 | Q-8 | Q-37 |
| X | H | H | H | H | H |
| Y | 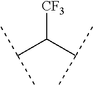 | 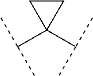 | CH₂ | CH₂ | CH₂ |
| No. | I.22-26 | I.22-27 | I.22-28 | I.22-29 | I.22-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.22-31 | I.22-32 | I.22-33 | I.22-34 | I.22-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.22-36 | I.22-37 | I.22-38 | I.22-39 | I.22-40 |
| Q | Q-33 | Q-34 | Q-31 | Q-52 | Q-79 |
| X | H | H | H | H | H |
| Y | CH₂ | CH₂ | 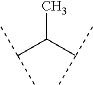 | CH₂ | CH₂ |
| No. | I.22-41 | I.22-42 | I.22-43 | I.22-44 | I.22-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.22-46 | I.22-47 | I.22-48 | I.22-49 | I.22-50 |
| Q | Q-79 | Q-57 | Q-58 | Q-60 | Q-64 |
| X | H | H | H | H | H |
| Y | 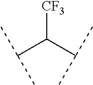 | CH₂ | CH₂ | CH₂ | CH₂ |
| No. | I.22-51 | I.22-52 | I.22-53 | I.22-54 | I.22-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.22-56 | I.22-57 | I.22-58 | I.22-59 | I.22-60 |
| Q | Q-110 | Q-51 | Q-134 | Q-109 | Q-139 |
| X | H | H | H | CH₃ | H |
| Y | CH₂ | CH₂ | 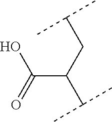 | CH₂ | CH₂ |
| | | No. | I.22-61 | I.22-62 | I.22-63 |
| | | Q | Q-140 | Q-149 | Q-141 |
| | | X | H | H | H |
| | | Y | CH₂ | CH₂ | CH₂ |

TABLE 23
with base structure I.23 and the radical definitions specified hereinafter:

| No. | I.23-1 | I.23-2 | I.23-3 | I.23-4 | I.23-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.23-6 | I.23-7 | I.23-8 | I.23-9 | I.23-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.23-11 | I.23-12 | I.23-13 | I.23-14 | I.23-15 |
| Q | Q-1 | Q-12 | Q-13 | Q-14 | Q-1 |
| X | CH₃ | H | H | H | H |
| Y | CH₂ | CH₂ | CH₂ | CH₂ | 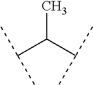 |
| No. | I.23-16 | I.23-17 | I.23-18 | I.23-19 | I.23-20 |
| Q | Q-6 | Q-1 | Q-1 | Q-12 | Q-1 |
| X | H | c-Pr | 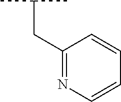 | CH₃ | CH₃ |
| Y | 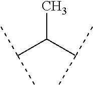 | CH₂ | CH₂ | CH₂ | 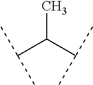 |
| No. | I.23-21 | I-23.22 | I.23-23 | I.23-24 | I.23-25 |
| Q | Q-1 | Q-2 | Q-17 | Q-8 | Q-37 |
| X | H | H | H | H | H |
| Y | 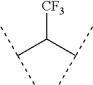 | 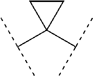 | CH₂ | CH₂ | CH₂ |
| No. | I.23-26 | I.23-27 | I.23-28 | I.23-29 | I.23-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.23-31 | I.23-32 | I.23-33 | I.23-34 | I.23-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.23-36 | I.23-27 | I.23-38 | I.23-39 | I.23-40 |
| Q | Q-33 | Q-34 | Q-31 | Q-52 | Q-79 |
| X | H | H | H | H | H |
| Y | CH₂ | CH₂ | 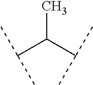 | CH₂ | CH₂ |
| No. | I.23-41 | I.23-42 | I.23-43 | I.23-44 | I.23-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.23-46 | I.23-47 | I.23-48 | I.23-49 | I.23-50 |
| Q | Q-79 | Q-57 | Q-58 | Q-60 | Q-64 |
| X | H | H | H | H | H |
| Y | 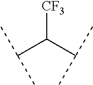 | CH₂ | CH₂ | CH₂ | CH₂ |
| No. | I.23-51 | I.23-52 | I.23-53 | I.23-54 | I.23-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.23-56 | I.23-57 | I.23-58 | I.23-59 | I.23-60 |
| Q | Q-110 | Q-51 | Q-134 | Q-109 | Q-139 |
| X | H | H | H | CH₃ | H |
| Y | CH₂ | CH₂ | 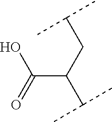 | CH₂ | CH₂ |
| | | No. | I.23-61 | I.23-62 | I.23-63 |
| | | Q | Q-140 | Q-149 | Q-141 |
| | | X | H | H | H |
| | | Y | CH₂ | CH₂ | CH₂ |

TABLE 24
with base structure I.24 and the radical definitions specified hereinafter:

| No. | I.24-1 | I.24-2 | I.24-3 | I.24-4 | I.24-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.24-6 | I.24-7 | I.24-8 | I.24-9 | I.24-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.24-11 | I.24-12 | I.24-13 | I.24-14 | I.24-15 |
| Q | Q-1 | Q-12 | Q-13 | Q-14 | Q-1 |
| X | CH₃ | H | H | H | H |
| Y | CH₂ | CH₂ | CH₂ | CH₂ | CH₂ |
| No. | I.24-16 | I.24-17 | I.24-18 | I.24-19 | I.24-20 |
| Q | Q-6 | Q-1 | Q-1 | Q-12 | Q-1 |
| X | H | c-Pr | 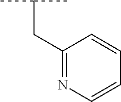 | CH₃ | CH₃ |
| Y | 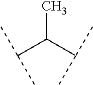 | CH₂ | CH₂ | CH₂ | 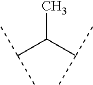 |
| No. | I.24-21 | I.24-22 | I.24-23 | I.24-24 | I.24-25 |
| Q | Q-1 | Q-1 | Q-17 | Q-8 | Q-37 |
| X | H | H | H | H | H |
| Y | 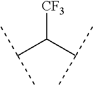 | 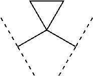 | CH₂ | CH₂ | CH₂ |
| No. | I.24-26 | I.24-27 | I.24-28 | I.24-29 | I.24-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.24-31 | I.24-32 | I.24-33 | I.24-34 | I.24-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.24-36 | I.24-37 | I.24-38 | I.24-39 | I.24-40 |
| Q | Q-33 | Q-34 | Q-31 | Q-52 | Q-79 |
| X | H | H | H | H | H |
| Y | CH₂ | CH₂ | 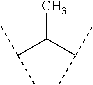 | CH₂ | CH₂ |
| No. | I.24-41 | I.24-42 | I.24-43 | I.24-44 | I.24-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.24-46 | I.24-47 | I.24-48 | I.24-49 | I.24-50 |
| Q | Q-79 | Q-57 | Q-58 | Q-60 | Q-64 |
| X | H | H | H | H | H |
| Y | 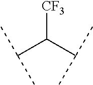 | CH₂ | CH₂ | CH₂ | CH₂ |
| No. | I.24-51 | I.24-52 | I.24-53 | I.24-54 | I.24-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.24-56 | I.24-57 | I.24-58 | I.24-59 | I.24-60 |
| Q | Q-110 | Q-51 | Q-134 | Q-109 | Q-139 |
| X | H | H | H | CH₃ | H |
| Y | CH₂ | CH₂ | 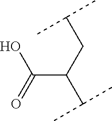 | CH₂ | CH₂ |
| | | No. | I.24-61 | I.24-62 | I.24-63 |
| | | Q | Q-140 | Q-149 | Q-141 |
| | | X | H | H | H |
| | | Y | CH₂ | CH₂ | CH₂ |

TABLE 25
with base structure I.25 and the radical definitions specified hereinafter:

| No. | I.25-1 | I.25-2 | I.25-3 | I.25-4 | I.25-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.25-6 | I.25-7 | I.25-8 | I.25-9 | I.25-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.25-11 | I.25-12 | I.25-13 | I.25-14 | I.25-15 |
| Q | Q-1 | Q-12 | Q-13 | Q-14 | Q-6 |
| X | CH₃ | H | H | H | H |
| Y | CH₂ | CH₂ | CH₂ | CH₂ | 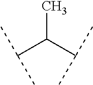 |
| No. | I.25-16 | I.25-17 | I.25-18 | I.25-19 | I.25-20 |
| Q | Q-6 | Q-1 | Q-1 | Q-12 | Q-1 |
| X | H | c-Pr | 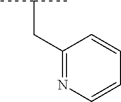 | CH₃ | CH₃ |
| Y | 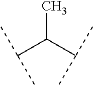 | CH₂ | CH₂ | CH₂ | 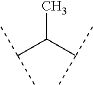 |
| No. | I.25-21 | I.25-22 | I.25-23 | I.25-24 | I.25-25 |
| Q | Q-1 | Q-1 | Q-17 | Q-8 | Q-37 |
| X | H | H | H | H | H |
| Y | 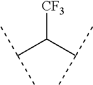 | 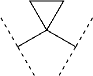 | CH₂ | CH₂ | CH₂ |
| No. | I.25-26 | I.25-27 | I.25-28 | I.25-29 | I.25-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.25-31 | I.25-32 | I.25-33 | I.25-34 | I.25-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.25-36 | I.25-37 | I.25-38 | I.25-39 | I.25-40 |
| Q | Q-33 | Q-34 | Q-31 | Q-52 | Q-79 |
| X | H | H | H | H | H |
| Y | CH₂ | CH₂ | 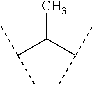 | CH₂ | CH₂ |
| No. | I.25-41 | I.25-42 | I.25-43 | I.25-44 | I.25-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I25-46 | I.25-47 | I.25-48 | I.25-49 | I.25-50 |
| Q | Q-79 | Q-57 | Q-58 | Q-60 | Q-64 |
| X | H | H | H | H | H |
| Y | 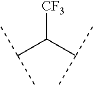 | CH₂ | CH₂ | CH₂ | CH₂ |
| No. | I.25-51 | I.25-52 | I.25-53 | I.25-54 | I.25-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.25-56 | I.25-57 | I.25-58 | I.25-59 | I.25-60 |
| Q | Q-110 | Q-51 | Q-134 | Q-109 | Q-139 |
| X | H | H | H | CH₃ | H |
| Y | CH₂ | CH₂ | 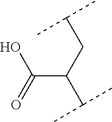 | CH₂ | CH₂ |
| | | No. | I.25-61 | I.25-62 | I.25-63 |
| | | Q | Q-140 | Q-149 | Q-141 |
| | | X | H | H | H |
| | | Y | CH₂ | CH₂ | CH₂ |

TABLE 26
with base structure I.26 and the radical definitions specified hereinafter:

| No. | I.26-1 | I.26-2 | I.26-3 | I.26-4 | I.26-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.26-6 | I.26-7 | I.26-8 | I.26-9 | I.26-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.26-11 | I.26-12 | I.26-13 | I.26-14 | I.26-15 |
| Q | Q-1 | Q-12 | Q-13 | Q-14 | Q-1 |
| X | CH₃ | H | H | H | H |
| Y | CH₂ | CH₂ | CH₂ | CH₂ | 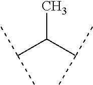 |
| No. | I.26-16 | I.26-17 | I.26-18 | I.26-19 | I.26-20 |
| Q | Q-6 | Q-1 | Q-1 | Q-12 | Q-1 |
| X | H | c-Pr | 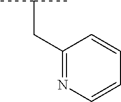 | CH₃ | CH₃ |
| Y | 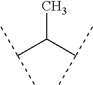 | CH₂ | CH₂ | CH₂ | 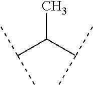 |
| No. | I.26-21 | I.26-22 | I.26-23 | I.26-24 | I.26-25 |
| Q | Q-1 | Q-1 | Q-17 | Q-8 | Q-37 |
| X | H | H | H | H | H |
| Y | 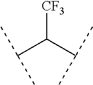 | 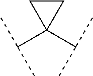 | CH₂ | CH₂ | CH₂ |
| No. | I.26-26 | I.26-27 | I.26-28 | I.26-29 | I.26-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.26-31 | I.26-32 | I.26-33 | I.26-34 | I.26-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.26-36 | I.26-37 | I.26-38 | I.26-39 | I.26-40 |
| Q | Q-33 | Q-34 | Q-31 | Q-52 | Q-79 |
| X | H | H | H | H | H |
| Y | CH₂ | CH₂ | 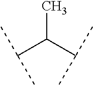 | CH₂ | CH₂ |
| No. | I.26-41 | I.26-42 | I.26-43 | I.26-44 | I.26-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.26-46 | I.26-47 | I.26-48 | I.26-49 | I.26-50 |
| Q | Q-79 | Q-57 | Q-58 | Q-60 | Q-64 |
| X | H | H | H | H | H |
| Y | 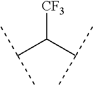 | CH₂ | CH₂ | CH₂ | CH₂ |
| No. | I.26-51 | I.26-52 | I.26-53 | I.26-54 | I.26-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.26-56 | I.26-57 | I.26-58 | I.26-59 | I.26-60 |
| Q | Q-110 | Q-51 | Q-134 | Q-109 | Q-139 |
| X | H | H | H | CH₃ | H |
| Y | CH₂ | CH₂ | 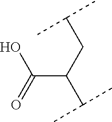 | CH₂ | CH₂ |
| | | No. | I.26-61 | I.26-62 | I.26-63 |
| | | Q | Q-140 | Q-149 | Q-141 |
| | | X | H | H | H |
| | | Y | CH₂ | CH₂ | CH₂ |

TABLE 27
with base structure I.27 and the radical definition specified hereinafter:

| No. | I.27-1 | I.27-2 | I.27-3 | I.27-4 | I.27-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.26-6 | I.27-7 | I.27-8 | I.27-9 | I.27-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.27-11 | I.27-12 | I.27-13 | I.27-14 | I.27-15 |
| Q | Q-1 | Q-12 | Q-13 | Q-14 | Q-1 |
| X | CH₃ | H | H | H | H |
| Y | CH₂ | CH₂ | CH₂ | CH₂ | 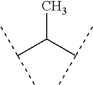 |
| No. | I.27-16 | I.27-17 | I.27-18 | I.27-19 | I.27-20 |
| Q | Q-6 | Q-1 | Q-1 | Q-12 | Q-1 |
| X | H | c-Pr | 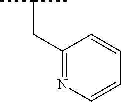 | CH₃ | CH₃ |
| Y | 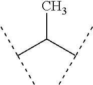 | CH₂ | CH₂ | CH₃ | 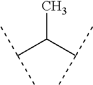 |
| No. | I.27-21 | I.27-22 | I.27-23 | I.27-24 | I.27-25 |
| Q | Q-1 | Q-1 | Q-17 | Q-8 | Q-37 |
| X | H | H | H | H | H |
| Y | 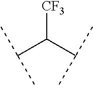 | 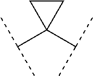 | CH₂ | CH₂ | CH₂ |
| No. | I.27-26 | I.27-27 | I.27-28 | I.27-29 | I.27-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.27-31 | I.27-32 | I.27-33 | I.27-34 | I.27-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.27-36 | I.27-37 | I.27-38 | I.27-39 | I.27-40 |
| Q | Q-33 | Q-34 | Q-31 | Q-52 | Q-79 |
| X | H | H | H | H | H |
| Y | CH₂ | CH₂ | 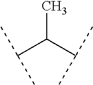 | CH₂ | CH₂ |
| No. | I.27-41 | I.27-42 | I.27-43 | I.27-44 | I.27-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.27-46 | I.27-47 | I.27-48 | I.27-49 | I.27-50 |
| Q | Q-79 | Q-57 | Q-58 | Q-60 | Q-64 |
| X | H | H | H | H | H |
| Y | 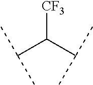 | CH₂ | CH₂ | CH₂ | CH₂ |
| No. | I.27-51 | I.27-52 | I.27-53 | I.27-54 | I.27-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.27-56 | I.27-57 | I.27-58 | I.27-59 | I.27-60 |
| Q | Q-110 | Q-51 | Q-134 | Q-109 | Q-139 |
| X | H | H | H | CH₃ | H |
| Y | CH₂ | CH₂ | 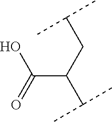 | CH₂ | CH₂ |
| | | No. | I.27-61 | I.27-62 | I.27-63 |
| | | Q | Q-140 | Q-149 | Q-141 |
| | | X | H | H | H |
| | | Y | CH₂ | CH₂ | CH₂ |

TABLE 28
with base structure I.28 and the radical definitions specified hereinafter:

| No. | I.28-1 | I.28-2 | I.28-3 | I.28-4 | I.28-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.28-6 | I.28-7 | I.28-8 | I.28-9 | I.28-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.28-11 | I.28-12 | I.28-13 | I.28-14 | I.28-15 |
| Q | Q-1 | Q-12 | Q-13 | Q-14 | Q-1 |
| X | CH₃ | H | H | H | H |
| Y | CH₂ | CH₂ | CH₂ | CH₂ | 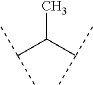 |
| No. | I.28-16 | I.28-17 | I.28-18 | I.28-19 | I.28-20 |
| Q | Q-6 | Q-1 | Q-1 | Q-12 | Q-1 |
| X | H | c-Pr | 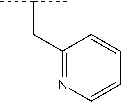 | CH₃ | CH₃ |
| Y | 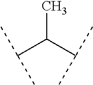 | CH₂ | CH₂ | CH₂ | 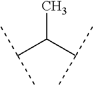 |
| No. | I.28-21 | I.28-22 | I.28-23 | I.28-24 | I.28-25 |
| Q | Q-1 | Q-1 | Q-17 | Q-8 | Q-37 |
| X | H | H | H | H | H |
| Y | 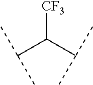 | 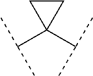 | CH₂ | CH₂ | CH₂ |
| No. | I.28-26 | I.28-27 | I.28-28 | I.28-29 | I.28-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.28-31 | I.28-32 | I.28-33 | I.28-34 | I.28-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.28-36 | I.28-37 | I.28-38 | I.28-39 | I.28-40 |
| Q | Q-33 | Q-34 | Q-31 | Q-52 | Q-79 |
| X | H | H | H | H | H |
| Y | CH₂ | CH₂ | 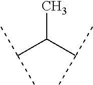 | CH₂ | CH₂ |
| No. | I.28-41 | I.28-42 | I.28-43 | I.28-44 | I.28-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.28-46 | I.28-47 | I.28-48 | I.28-49 | I.28-50 |
| Q | Q-79 | Q-57 | Q-58 | Q-60 | Q-64 |
| X | H | H | H | H | H |
| Y | 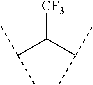 | CH₂ | CH₂ | CH₂ | CH₂ |
| No. | I.28-51 | I.28-52 | I.28-53 | I.28-54 | I.28-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.28-56 | I.28-57 | I.28-58 | I.28-59 | I.28-60 |
| Q | Q-110 | Q-51 | Q-134 | Q-109 | Q-139 |
| X | H | H | H | CH₃ | H |
| Y | CH₂ | CH₂ | 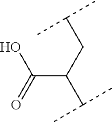 | CH₂ | CH₂ |
| | | No. | I.28-61 | I.28-62 | I.28-63 |
| | | Q | Q-140 | Q-149 | Q-141 |
| | | X | H | H | H |
| | | Y | CH₂ | CH₂ | CH₂ |

TABLE 29
with base structure I.29 and the radical definitions specified hereinafter:

| No. | I.29-1 | I.29-1 | I.29-3 | I.29-4 | I.29-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.29-6 | I.29-7 | I.29-8 | I.29-9 | I.29-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.29-11 | I.29-12 | I.29-13 | I.29-14 | I.29-15 |
| Q | Q-1 | Q-12 | Q-13 | Q-14 | Q-1 |
| X | CH₃ | H | H | H | H |
| Y | CH₂ | CH₂ | CH₂ | CH₂ | 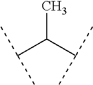 |
| No. | I.29-16 | I.29-17 | I.29-18 | I.29-19 | I.29-20 |
| Q | Q-8 | Q-1 | Q-1 | Q-12 | Q-1 |
| X | H | c-Pr | 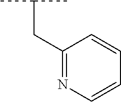 | CH₃ | CH₃ |
| Y | 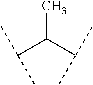 | CH₂ | CH₂ | CH₂ | 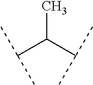 |
| No. | I.29-21 | I.29-22 | I.29-23 | I.29-24 | I.29-25 |
| Q | Q-1 | Q-1 | Q-17 | Q-8 | Q-37 |
| X | H | H | H | H | H |
| Y | 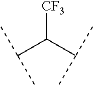 | 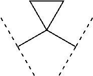 | CH₂ | CH₂ | CH₂ |
| No. | I.29-26 | I.29-27 | I.29-28 | I.29-29 | I.29-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.29-31 | I.29-32 | I.29-33 | I.29-34 | I.29-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.29-36 | I.29-37 | I.29-38 | I.29-39 | I.29-40 |
| Q | Q-33 | Q-34 | Q-31 | Q-52 | Q-79 |
| X | H | H | H | H | H |
| Y | CH₂ | CH₂ | 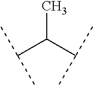 | CH₂ | CH₂ |
| No. | I.29-41 | I.29-42 | I.29-43 | I.29-44 | I.29-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.29-46 | I.29-47 | I.29-48 | I.29-49 | I.29-50 |
| Q | Q-79 | Q-57 | Q-58 | Q-60 | Q-64 |
| X | H | H | H | H | H |
| Y | 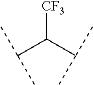 | CH₂ | CH₂ | CH₂ | CH₂ |
| No. | I.29-51 | I.29-52 | I.29-53 | I.29-54 | I.29-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.29-56 | I.29-57 | I.29-58 | I.29-59 | I.29-60 |
| Q | Q-110 | Q-51 | Q-134 | Q-109 | Q-139 |
| X | H | H | H | CH₃ | H |
| Y | CH₂ | CH₂ | 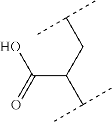 | CH₂ | CH₂ |
| | | No. | I.29-61 | I.29-62 | I.29-63 |
| | | Q | Q-140 | Q-149 | Q-141 |
| | | X | H | H | H |
| | | Y | CH₂ | CH₂ | CH₂ |

TABLE 30
with base structure I.30 and the radical definitions specified hereinafter:

| No. | I.30-1 | I.30-2 | I.30-3 | I.30-4 | I.30-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.30-6 | I.30-7 | I.30-8 | I.30-9 | I.30-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.30-11 | I.30-12 | I.30-13 | I.30-14 | I.30-15 |
| Q | Q-1 | Q-12 | Q-13 | Q-14 | Q-1 |
| X | CH₃ | H | H | H | H |
| Y | CH₂ | CH₂ | CH₂ | CH₂ | 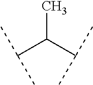 |
| No. | I.30-16 | I.30-17 | I.30-18 | I.30-19 | I.30-20 |
| Q | Q-6 | Q-1 | Q-1 | Q-12 | Q-1 |
| X | H | c-Pr | 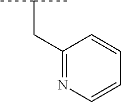 | CH₃ | CH₃ |
| Y | 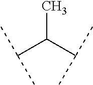 | CH₂ | CH₂ | CH₂ | 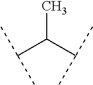 |
| No. | I.30-21 | I.30-22 | I.30-23 | I.30-24 | I.30-25 |
| Q | Q-1 | Q-1 | Q-17 | Q-8 | Q-37 |
| X | H | H | H | H | H |
| Y | 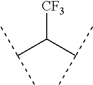 | 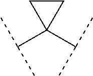 | CH₂ | CH₂ | CH₂ |
| No. | I.30-26 | I.30-27 | I.30-28 | I.30-29 | I.30-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.30-31 | I.30-32 | I.30-33 | I.30-34 | I.30-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.30-36 | I.30-37 | I.30-38 | I.30-39 | I.30-40 |
| Q | Q-33 | Q-34 | Q-31 | Q-52 | Q-79 |
| X | H | H | H | H | H |
| Y | CH₂ | CH₂ | 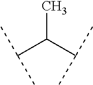 | CH₂ | CH₂ |
| No. | I.30-41 | I.30-42 | I.30-43 | I.30-44 | I.30-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.30-46 | I.30-47 | I.30-48 | I.30-49 | I.30-50 |
| Q | Q-79 | Q-57 | Q-58 | Q-60 | Q-64 |
| X | H | H | H | H | H |
| Y | 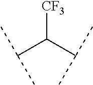 | CH₂ | CH₂ | CH₂ | CH₂ |
| No. | I.30-51 | I.30-52 | I.30-53 | I.30-54 | I.30-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.30-56 | I.30-57 | I.30-58 | I.30-59 | I.30-60 |
| Q | Q-110 | Q-51 | Q-134 | Q-109 | Q-139 |
| X | H | H | H | CH₃ | H |
| Y | CH₂ | CH₂ | 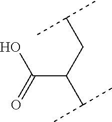 | CH₂ | CH₂ |
| | | No. | I.30-61 | I.30-62 | I.30-63 |
| | | Q | Q-140 | Q-149 | Q-141 |
| | | X | H | H | H |
| | | Y | CH₂ | CH₂ | CH₂ |

TABLE 31
with base structure I.31 and the radical definitions specified hereinafter:

| No. | I.31-1 | I.31-2 | I.31-3 | I.31-4 | I.31-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.31-6 | I.31-7 | I.31-8 | I.31-9 | I.31-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.31-11 | I.31-12 | I.31-13 | I.31-14 | I.31-15 |
| Q | Q-1 | Q-12 | Q-13 | Q-14 | Q-1 |
| X | CH₃ | H | H | H | H |
| Y | CH₂ | CH₂ | CH₂ | CH₂ | 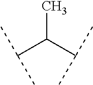 |
| No. | I.31-16 | I.31-17 | I.31-18 | I.31-19 | I.31-20 |
| Q | Q-6 | Q-1 | Q-1 | Q-12 | Q-1 |
| X | H | c-Pr | 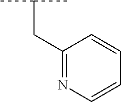 | CH₃ | CH₃ |
| Y | 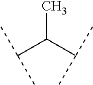 | CH₂ | CH₂ | CH₂ | 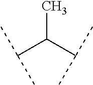 |
| No. | I.31-21 | I.31-22 | I.31-23 | I.31-24 | I.31-25 |
| Q | Q-1 | Q-1 | Q-17 | Q-8 | Q-37 |
| X | H | H | H | H | H |
| Y | 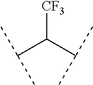 | 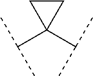 | CH₂ | CH₂ | CH₂ |
| No. | I.31-26 | I.31-27 | I.31-28 | I.31-29 | I.31-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.31-31 | I.31-32 | I.31-33 | I.31-34 | I.31-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.31-36 | I.31-37 | I.31-38 | I.31-39 | I.31-40 |
| Q | Q-33 | Q-34 | Q-31 | Q-52 | Q-79 |
| X | H | H | H | H | H |
| Y | CH₂ | CH₂ | 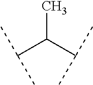 | CH₂ | CH₂ |
| No. | I.31-41 | I.31-42 | I.31-43 | I.31-44 | I.31-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.31-46 | I.31-47 | I.31-48 | I.31-49 | I.31-50 |
| Q | Q-79 | Q-57 | Q-58 | Q-60 | Q-64 |
| X | H | H | H | H | H |
| Y | 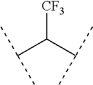 | CH₂ | CH₂ | CH₂ | CH₂ |
| No. | I.31-51 | I.31-52 | I.31-53 | I.31-54 | I.31-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.31-56 | I.31-57 | I.31-58 | I.31-59 | I.31-60 |
| Q | Q-110 | Q-51 | Q-134 | Q-109 | Q-139 |
| X | H | H | H | CH₃ | H |
| Y | CH₂ | CH₂ | 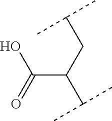 | CH₂ | CH₂ |
| | | No. | I.31-61 | I.31-62 | I.31-63 |
| | | Q | Q-140 | Q-149 | Q-141 |
| | | X | H | H | H |
| | | Y | CH₂ | CH₂ | CH₂ |

TABLE 32
with base structure I.32 and the radical definitions specified hereinafter:

| No. | I.32-1 | I.32-2 | I.32-3 | I.32-4 | I.32-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.32-6 | I.32-7 | I.32-8 | I.32-9 | I.32-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.32-11 | I.32-12 | I.32-13 | I.32-14 | I.32-15 |
| Q | Q-1 | Q-12 | Q-13 | Q-14 | Q-1 |
| X | CH₃ | H | H | H | H |
| Y | CH₂ | CH₂ | CH₂ | CH₂ | 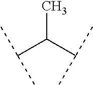 |
| No. | I.32-16 | I.32-17 | I.32-18 | I.32-19 | I.32-20 |
| Q | Q-6 | Q-1 | Q-1 | Q-12 | Q-1 |
| X | H | c-Pr | 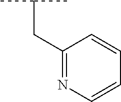 | CH₃ | CH₃ |
| Y | 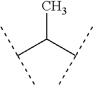 | CH₂ | CH₂ | CH₂ | 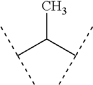 |
| No. | I.32-21 | I.32-22 | I.32-23 | I.32-24 | I.32-25 |
| Q | Q-1 | Q-1 | Q-17 | Q-8 | Q-37 |
| X | H | H | H | H | H |
| Y | 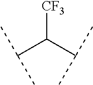 | 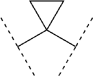 | CH₂ | CH₂ | CH₂ |
| No. | I.32-26 | I.32-27 | I.32-28 | I.32-29 | I.32-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.32-31 | I.32-32 | I.32-33 | I.32-34 | I.32-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.32-36 | I.32-37 | I.32-38 | I.32-39 | I.32-40 |
| Q | Q-33 | Q-34 | Q-31 | Q-52 | Q-79 |
| X | H | H | H | H | H |
| Y | CH₂ | CH₂ | 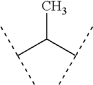 | CH₂ | CH₂ |
| No. | I.32-41 | I.32-42 | I.32-43 | I.32-44 | I.32-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.32-46 | I.32-47 | I.32-48 | I.32-49 | I.32-50 |
| Q | Q-79 | Q-57 | Q-58 | Q-60 | Q-64 |
| X | H | H | H | H | H |
| Y | 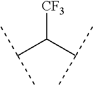 | CH₂ | CH₂ | CH₂ | CH₂ |
| No. | I.32-51 | I.32-52 | I.32-53 | I.32-54 | I.32-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.32-56 | I.32-57 | I.32-58 | I.32-59 | I.32-60 |
| Q | Q-110 | Q-51 | Q-134 | Q-109 | Q-139 |
| X | H | H | H | CH₃ | H |
| Y | CH₂ | CH₂ | 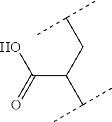 | CH₂ | CH₂ |
| | | No. | I.32-61 | I.32-62 | I.32-63 |
| | | Q | Q-140 | Q-149 | Q-141 |
| | | X | H | H | H |
| | | Y | CH₂ | CH₂ | CH₂ |

TABLE 33
with base structure I.33 and the radical definitions specified hereinafter:

| No. | I.33-1 | I.33-2 | I.33-3 | I.33-4 | I.33-5 | I.33-6 | I.33-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.33-8 | I.33-9 | I.33-10 | I.33-11 | I.33-12 | I.33-13 | I.33-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.33-15 | I.33-16 | I.33-17 | I.33-18 | I.33-19 | I.33-20 | I.33-21 |
| Q | Q-1 | Q-6 | Q-1 | Q-1 | Q-12 | Q-1 | Q-1 |
| X | H | H | c-Pr | 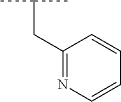 | CH₃ | CH₃ | H |
| Y | 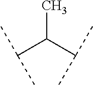 | 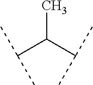 | CH₂ | CH₂ | CH₂ | 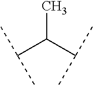 | 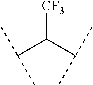 |
| No. | I.33-22 | I.33-23 | I.33-24 | I.33-25 | I.33-26 | I.33-27 | I.33-28 |
| Q | Q-1 | Q-17 | Q-8 | Q-37 | Q-38 | Q-1 | Q-25 |
| X | H | H | H | H | H | i-Pr | H |
| Y | 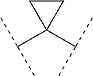 | CH₂ | CH₂ | CH₂ | CH₂ | CH₂ | CH₂ |
| No. | I.33-29 | I.33-30 | I.33-31 | I.33-32 | I.33-33 | I.33-34 | I.33-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.33-36 | I.33-37 | I.33-38 | I.33-39 | I.33-40 | I.33-41 | I.33-42 |
| Q | Q-33 | Q-34 | Q-31 | Q-52 | Q-79 | Q-80 | Q-81 |
| X | H | H | H | H | H | H | H |
| Y | CH₂ | CH₂ | 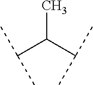 | CH₂ | CH₂ | CH₂ | CH₂ |
| No. | I.33-43 | I.33-44 | I.33-45 | I.33-46 | I.33-47 | I.33-48 | I.33-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 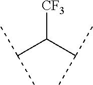 | CH₂ | CH₂ | CH₂ |
| No. | I.33-50 | I.33-51 | I.33-52 | I.33-53 | I.33-54 | I.33-55 | I.33-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.33-57 | I.33-58 | I.33-59 | I.33-60 | I.33-61 | I.33-62 | I.33-63 |
| Q | Q-51 | Q-134 | Q-109 | Q-139 | Q-140 | Q-149 | Q-141 |
| X | H | H | CH₃ | H | H | H | H |
| Y | CH₂ | 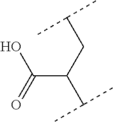 | CH₂ | CH₂ | CH₂ | CH₂ | CH₂ |

TABLE 34
with base structure I.34 and the radical definitions specified hereinafter:

| No. | I.34-1 | I.34-2 | I.34-3 | I.34-4 | I.34-5 | I.34-6 | I.34-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.34-8 | I.34-9 | I.34-10 | I.34-11 | I.34-12 | I.34-13 | I.34-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.34-15 | I.34-16 | I.34-17 | I.34-18 | I.34-19 | I.34-20 | I.34-21 |
| Q | Q-1 | Q-6 | Q-1 | Q-1 | Q-12 | Q-1 | Q-1 |
| X | H | H | c-Pr | 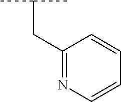 | CH₃ | CH₃ | H |
| Y | 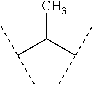 | 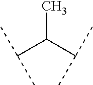 | CH₂ | CH₂ | CH₂ | 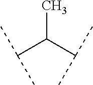 | 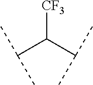 |
| No. | I.34-22 | I.34-23 | I.34-24 | I.34-25 | I.34-26 | I.34-27 | I.34-28 |
| Q | Q-1 | Q-17 | Q-8 | Q-37 | Q-38 | Q-1 | Q-25 |
| X | H | H | H | H | H | i-Pr | H |
| Y | 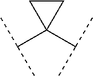 | CH₂ | CH₂ | CH₂ | CH₂ | CH₂ | CH₂ |
| No. | I.34-29 | I.34-30 | I.34-31 | I.34-32 | I.34-33 | I.34-34 | I.34-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.34-36 | I.34-37 | I.34-38 | I.34-39 | I.34-40 | I.34-41 | I.34-42 |
| Q | Q-33 | Q-34 | Q-31 | Q-52 | Q-79 | Q-80 | Q-81 |
| X | H | H | H | H | H | H | H |
| Y | CH₂ | CH₂ | 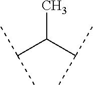 | CH₂ | CH₂ | CH₂ | CH₂ |
| No. | I.34-43 | I.34-44 | I.34-45 | I.34-46 | I.34-47 | I.34-48 | I.34-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 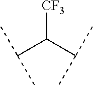 | CH₂ | CH₂ | CH₂ |
| No. | I.34-50 | I.34-51 | I.34-52 | I.34-53 | I.34-54 | I.34-55 | I.34-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.34-57 | I.34-58 | I.34-59 | I.34-60 | I.34-61 | I.34-62 | I.34-63 |
| Q | Q-51 | Q-134 | Q-109 | Q-139 | Q-140 | Q-149 | Q-141 |
| X | H | H | CH₃ | H | H | H | H |
| Y | CH₂ | 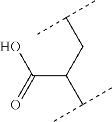 | CH₂ | CH₂ | CH₂ | CH₂ | CH₂ |

TABLE 35
with base structure I.35 and the radical definitions specified hereinafter:

| No. | I.35-1 | I.35-2 | I.35-3 | I.35-4 | I.35-5 | I.35-6 | I.35-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.35-8 | I.35-9 | I.35-10 | I.35-11 | I.35-12 | I.35-13 | I.35-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.35-15 | I.35-16 | I.35-17 | I.35-18 | I.35-19 | I.35-20 | I.35-21 |
| Q | Q-1 | Q-6 | Q-1 | Q-1 | Q-12 | Q-1 | Q-1 |
| X | H | H | c-Pr | 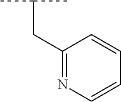 | CH₃ | CH₃ | H |
| Y | 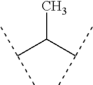 | 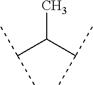 | CH₂ | CH₂ | CH₂ | 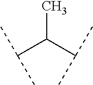 | 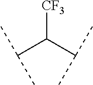 |
| No. | I.35-22 | I.35-23 | I.35-24 | I.35-25 | I.35-26 | I.35-27 | I.35-28 |
| Q | Q-1 | Q-17 | Q-8 | Q-37 | Q-38 | Q-1 | Q-25 |
| X | H | H | H | H | H | i-Pr | H |
| Y | 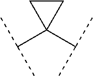 | CH₂ | CH₂ | CH₂ | CH₂ | CH₂ | CH₂ |
| No. | I.35-29 | I.35-30 | I.35-31 | I.35-32 | I.35-33 | I.35-34 | I.35-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.35-36 | I.35-37 | I.35-38 | I.35-39 | I.35-40 | I.35-41 | I.35-42 |
| Q | Q-33 | Q-34 | Q-31 | Q-52 | Q-79 | Q-80 | Q-81 |
| X | H | H | H | H | H | H | H |
| Y | CH₂ | CH₂ | 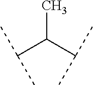 | CH₂ | CH₂ | CH₂ | CH₂ |
| No. | I.35-43 | I.35-44 | I.35-45 | I.35-46 | I.35-47 | I.35-48 | I.35-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 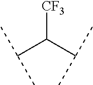 | CH₂ | CH₂ | CH₂ |
| No. | I.35-50 | I.35-51 | I.35-52 | I.35-53 | I.35-54 | I.35-55 | I.35-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.35-57 | I.35-58 | I.35-59 | I.35-60 | I.35-61 | I.35-62 | I.35-63 |
| Q | Q-51 | Q-134 | Q-109 | Q-139 | Q-140 | Q-149 | Q-141 |
| X | H | H | CH₃ | H | H | H | H |
| Y | CH₂ | 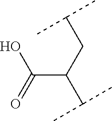 | CH₂ | CH₂ | CH₂ | CH₂ | CH₂ |

TABLE 36
with base structure I.36 and the radical definitions specified hereinafter:

| No. | I.36-1 | I.36-2 | I.36-3 | I.36-4 | I.36-5 | I.36-6 | I.36-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.36-8 | I.36-9 | I.36-10 | I.36-11 | I.36-12 | I.36-13 | I.36-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.36-15 | I.36-16 | I.36-17 | I.36-18 | I.36-19 | I.36-20 | I.36-21 |
| Q | Q-1 | Q-6 | Q-1 | Q-1 | Q-12 | Q-1 | Q-1 |
| X | H | H | c-Pr | 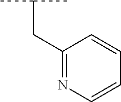 | CH₃ | CH₃ | H |
| Y | 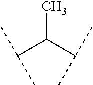 | 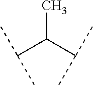 | CH₂ | CH₂ | CH₂ | 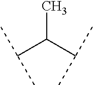 | 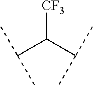 |
| No. | I.36-22 | I.36-23 | I.36-24 | I.36-25 | I.36-26 | I.36-27 | I.36-28 |
| Q | Q-1 | Q-17 | Q-8 | Q-37 | Q-38 | Q-1 | Q-25 |
| X | H | H | H | H | H | i-Pr | H |
| Y | 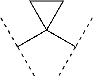 | CH₂ | CH₂ | CH₂ | CH₂ | CH₂ | CH₂ |
| No. | I.36-29 | I.36-30 | I.36-31 | I.36-32 | I.36-33 | I.36-34 | I.36-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.36-36 | I.36-37 | I.36-38 | I.36-39 | I.36-40 | I.36-41 | I.36-42 |
| Q | Q-33 | Q-34 | Q-31 | Q-52 | Q-79 | Q-80 | Q-81 |
| X | H | H | H | H | H | H | H |
| Y | CH₂ | CH₂ | 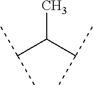 | CH₂ | CH₂ | CH₂ | CH₂ |
| No. | I.36-43 | I.36-44 | I.36-45 | I.36-46 | I.36-47 | I.36-48 | I.36-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 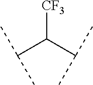 | CH₂ | CH₂ | CH₂ |
| No. | I.36-50 | I.36-51 | I.36-52 | I.36-53 | I.36-54 | I.36-55 | I.36-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.36-57 | I.36-58 | I.36-59 | I.36-60 | I.36-61 | I.36-62 | I.36-63 |
| Q | Q-51 | Q-134 | Q-109 | Q-139 | Q-140 | Q-149 | Q-141 |
| X | H | H | CH₃ | H | H | H | H |
| Y | CH₂ | 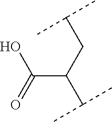 | CH₂ | CH₂ | CH₂ | CH₂ | CH₂ |

TABLE 37
with base structure I.37 and the radical definitions specified hereinafter:

| No. | I.37-1 | I.37-2 | I.37-3 | I.37-4 | I.37-5 | I.37-6 | I.37-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.37-8 | I.37-9 | I.37-10 | I.37-11 | I.37-12 | I.37-13 | I.37-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.37-15 | I.37-16 | I.37-17 | I.37-18 | I.37-19 | I.37-20 | I.37-21 |
| Q | Q-1 | Q-6 | Q-1 | Q-1 | Q-12 | Q-1 | Q-1 |
| X | H | H | c-Pr | 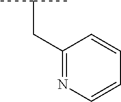 | CH₃ | CH₃ | H |
| Y | 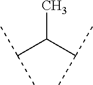 | 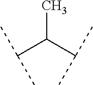 | CH₂ | CH₂ | CH₂ | 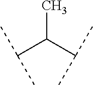 | 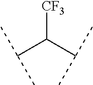 |
| No. | I.37-22 | I.37-23 | I.37-24 | I.37-25 | I.37-26 | I.37-27 | I.37-28 |
| Q | Q-1 | Q-17 | Q-8 | Q-37 | Q-38 | Q-1 | Q-25 |
| X | H | H | H | H | H | i-Pr | H |
| Y | 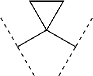 | CH₂ | CH₂ | CH₂ | CH₂ | CH₂ | CH₂ |
| No. | I.37-29 | I.37-30 | I.37-31 | I.37-32 | I.37-33 | I.37-34 | I.37-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.37-36 | I.37-37 | I.37-38 | I.37-39 | I.37-40 | I.37-41 | I.37-42 |
| Q | Q-33 | Q-34 | Q-31 | Q-52 | Q-79 | Q-80 | Q-81 |
| X | H | H | H | H | H | H | H |
| Y | CH₂ | CH₂ | 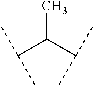 | CH₂ | CH₂ | CH₂ | CH₂ |
| No. | I.37-43 | I.37-44 | I.37-45 | I.37-46 | I.37-47 | I.37-48 | I.37-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 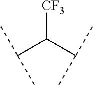 | CH₂ | CH₂ | CH₂ |
| No. | I.37-50 | I.37-51 | I.37-52 | I.37-53 | I.37-54 | I.37-55 | I.37-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.37-57 | I.37-58 | I.37-59 | I.37-60 | I.37-61 | I.37-62 | I.37-63 |
| Q | Q-51 | Q-134 | Q-109 | Q-139 | Q-140 | Q-149 | Q-141 |
| X | H | H | CH₃ | H | H | H | H |
| Y | CH₂ | 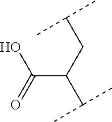 | CH₂ | CH₂ | CH₂ | CH₂ | CH₂ |

TABLE 38
with base structure I.38 and the radical definitions specified hereinafter:

| No. | I.38-1 | I.38-2 | I.38-3 | I.38-4 | I.38-5 | I.38-6 | I.38-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.38-8 | I.38-9 | I.38-10 | I.38-11 | I.38-12 | I.38-13 | I.38-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.38-15 | I.38-16 | I.38-17 | I.38-18 | I.38-19 | I.38-20 | I.38-21 |
| Q | Q-1 | Q-6 | Q-1 | Q-1 | Q-12 | Q-1 | Q-1 |
| X | H | H | c-Pr | 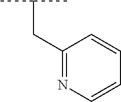 | CH₃ | CH₃ | H |
| Y | 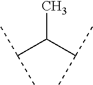 | 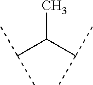 | CH₂ | CH₂ | CH₂ | 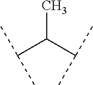 | 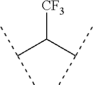 |
| No. | I.38-22 | I.38-23 | I.38-24 | I.38-25 | I.38-26 | I.38-27 | I.38-28 |
| Q | Q-1 | Q-17 | Q-8 | Q-37 | Q-38 | Q-1 | Q-25 |
| X | H | H | H | H | H | i-Pr | H |
| Y | 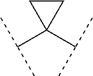 | CH₂ | CH₂ | CH₂ | CH₂ | CH₂ | CH₂ |
| No. | I.38-29 | I.38-30 | I.38-31 | I.38-32 | I.38-33 | I.38-34 | I.38-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.38-36 | I.38-37 | I.38-38 | I.38-39 | I.38-40 | I.38-41 | I.38-42 |
| Q | Q-33 | Q-34 | Q-31 | Q-52 | Q-79 | Q-80 | Q-81 |
| X | H | H | H | H | H | H | H |
| Y | CH₂ | CH₂ | 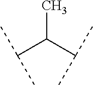 | CH₂ | CH₂ | CH₂ | CH₂ |
| No. | I.38-43 | I.38-44 | I.38-45 | I.38-46 | I.38-47 | I.38-48 | I.38-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 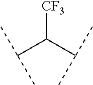 | CH₂ | CH₂ | CH₂ |
| No. | I.38-50 | I.38-51 | I.38-52 | I.38-53 | I.38-54 | I.38-55 | I.38-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.38-57 | I.38-58 | I.38-59 | I.38-60 | I.38-61 | I.38-62 | I.38-63 |
| Q | Q-51 | Q-134 | Q-109 | Q-139 | Q-140 | Q-149 | Q-141 |
| X | H | H | CH₃ | H | H | H | H |
| Y | CH₂ | 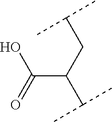 | CH₂ | CH₂ | CH₂ | CH₂ | CH₂ |

TABLE 39
with base structure I.39 and the radical definitions specified hereinafter:

| No. | I.39-1 | I.39-2 | I.39-3 | I.39-4 | I.39-5 | I.39-6 | I.39-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.39-8 | I.39-9 | I.39-10 | I.39-11 | I.39-12 | I.39-13 | I.39-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.39-15 | I.39-16 | I.39-17 | I.39-18 | I.39-19 | I.39-20 | I.39-21 |
| Q | Q-1 | Q-6 | Q-1 | Q-1 | Q-12 | Q-1 | Q-1 |
| X | H | H | c-Pr | 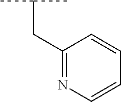 | CH₃ | CH₃ | H |
| Y | 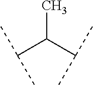 | 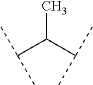 | CH₂ | CH₂ | CH₂ | 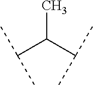 | 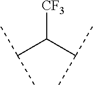 |
| No. | I.39-22 | I.39-23 | I.39-24 | I.39-25 | I.39-26 | I.39-27 | I.39-28 |
| Q | Q-1 | Q-17 | Q-8 | Q-37 | Q-38 | Q-1 | Q-25 |
| X | H | H | H | H | H | i-Pr | H |
| Y | 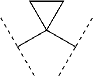 | CH₂ | CH₂ | CH₂ | CH₂ | CH₂ | CH₂ |
| No. | I.39-29 | I.39-30 | I.39-31 | I.39-32 | I.39-33 | I.39-34 | I.39-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.39-36 | I.39-37 | I.39-38 | I.39-39 | I.39-40 | I.39-41 | I.39-42 |
| Q | Q-33 | Q-34 | Q-31 | Q-52 | Q-79 | Q-80 | Q-81 |
| X | H | H | H | H | H | H | H |
| Y | CH₂ | CH₂ | 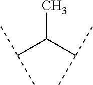 | CH₂ | CH₂ | CH₂ | CH₂ |
| No. | I.39-43 | I.39-44 | I.39-45 | I.39-46 | I.39-47 | I.39-48 | I.39-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 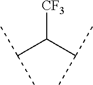 | CH₂ | CH₂ | CH₂ |
| No. | I.39-50 | I.39-51 | I.39-52 | I.39-53 | I.39-54 | I.39-55 | I.39-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.39-57 | I.39-58 | I.39-59 | I.39-60 | I.39-61 | I.39-62 | I.39-63 |
| Q | Q-51 | Q-134 | Q-109 | Q-139 | Q-140 | Q-149 | Q-141 |
| X | H | H | CH₃ | H | H | H | H |
| Y | CH₂ | 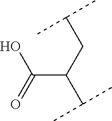 | CH₂ | CH₂ | CH₂ | CH₂ | CH₂ |

TABLE 40
with base structure I.40 and the radical definitions specified hereinafter:

| No. | I.40-1 | I.40-2 | I.40-3 | I.40-4 | I.40-5 | I.40-6 | I.40-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.40-8 | I.40-9 | I.40-10 | I.40-11 | I.40-12 | I.40-13 | I.40-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.40-15 | I.40-16 | I.40-17 | I.40-18 | I.40-19 | I.40-20 | I.40-21 |
| Q | Q-1 | Q-6 | Q-1 | Q-1 | Q-12 | Q-1 | Q-1 |
| X | H | H | c-Pr | 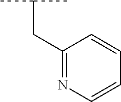 | CH₃ | CH₃ | H |
| Y | 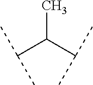 | 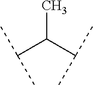 | CH₂ | CH₂ | CH₂ | 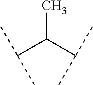 | 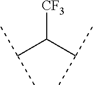 |
| No. | I.40-22 | I.40-23 | I.40-24 | I.40-25 | I.40-26 | I.40-27 | I.40-28 |
| Q | Q-1 | Q-17 | Q-8 | Q-37 | Q-38 | Q-1 | Q-25 |
| X | H | H | H | H | H | i-Pr | H |
| Y | 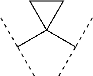 | CH₂ | CH₂ | CH₂ | CH₂ | CH₂ | CH₂ |
| No. | I.40-29 | I.40-30 | I.40-31 | I.40-32 | I.40-33 | I.40-34 | I.40-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.40-36 | I.40-37 | I.40-38 | I.40-39 | I.40-40 | I.40-41 | I.40-42 |
| Q | Q-33 | Q-34 | Q-31 | Q-52 | Q-79 | Q-80 | Q-81 |
| X | H | H | H | H | H | H | H |
| Y | CH₂ | CH₂ | 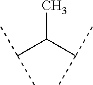 | CH₂ | CH₂ | CH₂ | CH₂ |
| No. | I.40-43 | I.40-44 | I.40-45 | I.40-46 | I.40-47 | I.40-48 | I.40-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 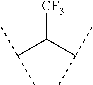 | CH₂ | CH₂ | CH₂ |
| No. | I.40-50 | I.40-51 | I.40-52 | I.40-53 | I.40-54 | I.40-55 | I.40-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.40-57 | I.40-58 | I.40-59 | I.40-60 | I.40-61 | I.40-62 | I.40-63 |
| Q | Q-51 | Q-134 | Q-109 | Q-139 | Q-140 | Q-149 | Q-141 |
| X | H | H | CH₃ | H | H | H | H |
| Y | CH₂ | 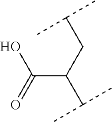 | CH₂ | CH₂ | CH₂ | CH₂ | CH₂ |

TABLE 41
with base structure I.41 and the radical definitions specified hereinafter:

| No. | I.41-1 | I.41-2 | I.41-3 | I.41-4 | I.41-5 | I.41-6 | I.41-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.41-8 | I.41-9 | I.41-10 | I.41-11 | I.41-12 | I.41-13 | I.41-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.41-15 | I.41-16 | I.41-17 | I.41-18 | I.41-19 | I.41-20 | I.41-21 |
| Q | Q-1 | Q-6 | Q-1 | Q-1 | Q-12 | Q-1 | Q-1 |
| X | H | H | c-Pr | 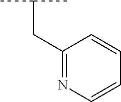 | CH₃ | CH₃ | H |
| Y | 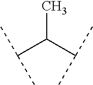 | 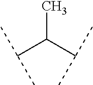 | CH₂ | CH₂ | CH₂ | 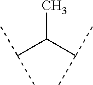 | 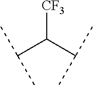 |
| No. | I.41-22 | I.41-23 | I.41-24 | I.41-25 | I.41-26 | I.41-27 | I.41-28 |
| Q | Q-1 | Q-17 | Q-8 | Q-37 | Q-38 | Q-1 | Q-25 |
| X | H | H | H | H | H | i-Pr | H |
| Y | 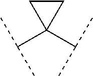 | CH₂ | CH₂ | CH₂ | CH₂ | CH₂ | CH₂ |
| No. | I.41-29 | I.41-30 | I.41-31 | I.41-32 | I.41-33 | I.41-34 | I.41-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.41-36 | I.41-37 | I.41-38 | I.41-39 | I.41-40 | I.41-41 | I.41-42 |
| Q | Q-33 | Q-34 | Q-31 | Q-52 | Q-79 | Q-80 | Q-81 |
| X | H | H | H | H | H | H | H |
| Y | CH₂ | CH₂ | 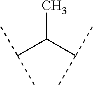 | CH₂ | CH₂ | CH₂ | CH₂ |
| No. | I.41-43 | I.41-44 | I.41-45 | I.41-46 | I.41-47 | I.41-48 | I.41-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 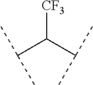 | CH₂ | CH₂ | CH₂ |
| No. | I.41-50 | I.41-51 | I.41-52 | I.41-53 | I.41-54 | I.41-55 | I.41-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.41-57 | I.41-58 | I.41-59 | I.41-60 | I.41-61 | I.41-62 | I.41-63 |
| Q | Q-51 | Q-134 | Q-109 | Q-139 | Q-140 | Q-149 | Q-141 |
| X | H | H | CH₃ | H | H | H | H |
| Y | CH₂ | 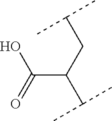 | CH₂ | CH₂ | CH₂ | CH₂ | CH₂ |

TABLE 42
with base structure I.42 and the radical definitions specified hereinafter:

| No. | I.42-1 | I.42-2 | I.42-3 | I.42-4 | I.42-5 | I.42-6 | I.42-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.42-8 | I.42-9 | I.42-10 | I.42-11 | I.42-12 | I.42-13 | I.42-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.42-15 | I.42-16 | I.42-17 | I.42-18 | I.42-19 | I.42-20 | I.42-21 |
| Q | Q-1 | Q-6 | Q-1 | Q-1 | Q-12 | Q-1 | Q-1 |
| X | H | H | c-Pr | 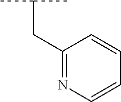 | CH₃ | CH₃ | H |
| Y | 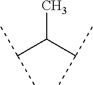 | 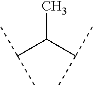 | CH₂ | CH₂ | CH₂ | 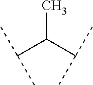 | 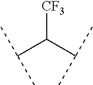 |
| No. | I.42-22 | I.42-23 | I.42-24 | I.42-25 | I.42-26 | I.42-27 | I.42-28 |
| Q | Q-1 | Q-17 | Q-8 | Q-37 | Q-38 | Q-1 | Q-25 |
| X | H | H | H | H | H | i-Pr | H |
| Y | 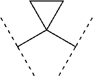 | CH₂ | CH₂ | CH₂ | CH₂ | CH₂ | CH₂ |
| No. | I.42-29 | I.42-30 | I.42-31 | I.42-32 | I.42-33 | I.42-34 | I.42-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.42-36 | I.42-37 | I.42-38 | I.42-39 | I.42-40 | I.42-41 | I.42-42 |
| Q | Q-33 | Q-34 | Q-31 | Q-52 | Q-79 | Q-80 | Q-81 |
| X | H | H | H | H | H | H | H |
| Y | CH₂ | CH₂ | 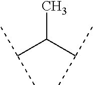 | CH₂ | CH₂ | CH₂ | CH₂ |
| No. | I.42-43 | I.42-44 | I.42-45 | I.42-46 | I.42-47 | I.42-48 | I.42-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 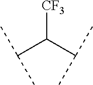 | CH₂ | CH₂ | CH₂ |
| No. | I.42-50 | I.42-51 | I.42-52 | I.42-53 | I.42-54 | I.42-55 | I.42-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.42-57 | I.42-58 | I.42-59 | I.42-60 | I.42-61 | I.42-62 | I.42-63 |
| Q | Q-51 | Q-134 | Q-109 | Q-139 | Q-140 | Q-149 | Q-141 |
| X | H | H | CH₃ | H | H | H | H |
| Y | CH₂ | 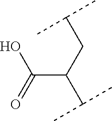 | CH₂ | CH₂ | CH₂ | CH₂ | CH₂ |

TABLE 43
with base structure I.43 and the radical definitions specified hereinafter:

| No. | I.43-1 | I.43-2 | I.43-3 | I.43-4 | I.43-5 | I.43-6 | I.43-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.43-8 | I.43-9 | I.43-10 | I.43-11 | I.43-12 | I.43-13 | I.43-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.43-15 | I.43-16 | I.43-17 | I.43-18 | I.43-19 | I.43-20 | I.43-21 |
| Q | Q-1 | Q-6 | Q-1 | Q-1 | Q-12 | Q-1 | Q-1 |
| X | H | H | c-Pr | 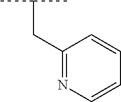 | CH₃ | CH₃ | H |
| Y | 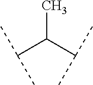 | 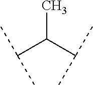 | CH₂ | CH₂ | CH₂ | 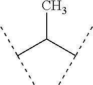 | 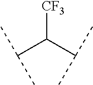 |
| No. | I.43-22 | I.43-23 | I.43-24 | I.43-25 | I.43-26 | I.43-27 | I.43-28 |
| Q | Q-1 | Q-17 | Q-8 | Q-37 | Q-38 | Q-1 | Q-25 |
| X | H | H | H | H | H | i-Pr | H |
| Y | 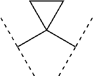 | CH₂ | CH₂ | CH₂ | CH₂ | CH₂ | CH₂ |
| No. | I.43-29 | I.43-30 | I.43-31 | I.43-32 | I.43-33 | I.43-34 | I.43-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.43-36 | I.43-37 | I.43-38 | I.43-39 | I.43-40 | I.43-41 | I.43-42 |
| Q | Q-33 | Q-34 | Q-31 | Q-52 | Q-79 | Q-80 | Q-81 |
| X | H | H | H | H | H | H | H |
| Y | CH₂ | CH₂ | 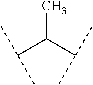 | CH₂ | CH₂ | CH₂ | CH₂ |
| No. | I.43-43 | I.43-44 | I.43-45 | I.43-46 | I.43-47 | I.43-48 | I.43-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 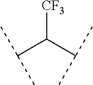 | CH₂ | CH₂ | CH₂ |
| No. | I.43-50 | I.43-51 | I.43-52 | I.43-53 | I.43-54 | I.43-55 | I.43-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.43-57 | I.43-58 | I.43-59 | I.43-60 | I.43-61 | I.43-62 | I.43-63 |
| Q | Q-51 | Q-134 | Q-109 | Q-139 | Q-140 | Q-149 | Q-141 |
| X | H | H | CH₃ | H | H | H | H |
| Y | CH₂ | 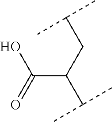 | CH₂ | CH₂ | CH₂ | CH₂ | CH₂ |

TABLE 44
with base structure I.44 and the radical definitions specified hereinafter:

| No. | I.44-1 | I.44-2 | I.44-3 | I.44-4 | I.44-5 | I.44-6 | I.44-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.44-8 | I.44-9 | I.44-10 | I.44-11 | I.44-12 | I.44-13 | I.44-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.44-15 | I.44-16 | I.44-17 | I.44-18 | I.44-19 | I.44-20 | I.44-21 |
| Q | Q-1 | Q-6 | Q-1 | Q-1 | Q-12 | Q-1 | Q-1 |
| X | H | H | c-Pr | 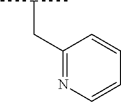 | CH₃ | CH₃ | H |
| Y | 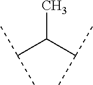 | 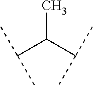 | CH₂ | CH₂ | CH₂ | 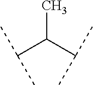 | 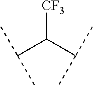 |
| No. | I.44-22 | I.44-23 | I.44-24 | I.44-25 | I.44-26 | I.44-27 | I.44-28 |
| Q | Q-1 | Q-17 | Q-8 | Q-37 | Q-38 | Q-1 | Q-25 |
| X | H | H | H | H | H | i-Pr | H |
| Y | 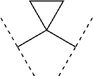 | CH₂ | CH₂ | CH₂ | CH₂ | CH₂ | CH₂ |
| No. | I.44-29 | I.44-30 | I.44-31 | I.44-32 | I.44-33 | I.44-34 | I.44-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.44-36 | I.44-37 | I.44-38 | I.44-39 | I.44-40 | I.44-41 | I.44-42 |
| Q | Q-33 | Q-34 | Q-31 | Q-52 | Q-79 | Q-80 | Q-81 |
| X | H | H | H | H | H | H | H |
| Y | CH₂ | CH₂ | 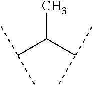 | CH₂ | CH₂ | CH₂ | CH₂ |
| No. | I.44-43 | I.44-44 | I.44-45 | I.44-46 | I.44-47 | I.44-48 | I.44-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 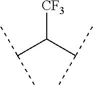 | CH₂ | CH₂ | CH₂ |
| No. | I.44-50 | I.44-51 | I.44-52 | I.44-53 | I.44-54 | I.44-55 | I.44-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.44-57 | I.44-58 | I.44-59 | I.44-60 | I.44-61 | I.44-62 | I.44-63 |
| Q | Q-51 | Q-134 | Q-109 | Q-139 | Q-140 | Q-149 | Q-141 |
| X | H | H | CH₃ | H | H | H | H |
| Y | CH₂ | 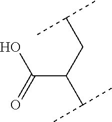 | CH₂ | CH₂ | CH₂ | CH₂ | CH₂ |

TABLE 45
with base structure I.45 and the radical definitions specified hereinafter:

| No. | I.45-1 | I.45-2 | I.45-3 | I.45-4 | I.45-5 | I.45-6 | I.45-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.45-8 | I.45-9 | I.45-10 | I.45-11 | I.45-12 | I.45-13 | I.45-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.45-15 | I.45-16 | I.45-17 | I.45-18 | I.45-19 | I.45-20 | I.45-21 |
| Q | Q-1 | Q-6 | Q-1 | Q-1 | Q-12 | Q-1 | Q-1 |
| X | H | H | c-Pr | 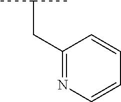 | CH₃ | CH₃ | H |
| Y | 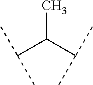 | 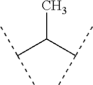 | CH₂ | CH₂ | CH₂ | 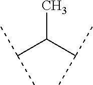 | 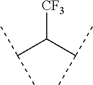 |
| No. | I.45-22 | I.45-23 | I.45-24 | I.45-25 | I.45-26 | I.45-27 | I.45-28 |
| Q | Q-1 | Q-17 | Q-8 | Q-37 | Q-38 | Q-1 | Q-25 |
| X | H | H | H | H | H | i-Pr | H |
| Y | 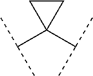 | CH₂ | CH₂ | CH₂ | CH₂ | CH₂ | CH₂ |
| No. | I.45-29 | I.45-30 | I.45-31 | I.45-32 | I.45-33 | I.45-34 | I.45-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.45-36 | I.45-37 | I.45-38 | I.45-39 | I.45-40 | I.45-41 | I.45-42 |
| Q | Q-33 | Q-34 | Q-31 | Q-52 | Q-79 | Q-80 | Q-81 |
| X | H | H | H | H | H | H | H |
| Y | CH₂ | CH₂ | 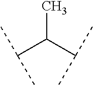 | CH₂ | CH₂ | CH₂ | CH₂ |
| No. | I.45-43 | I.45-44 | I.45-45 | I.45-46 | I.45-47 | I.45-48 | I.45-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 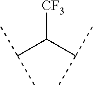 | CH₂ | CH₂ | CH₂ |
| No. | I.45-50 | I.45-51 | I.45-52 | I.45-53 | I.45-54 | I.45-55 | I.45-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.45-57 | I.45-58 | I.45-59 | I.45-60 | I.45-61 | I.45-62 | I.45-63 |
| Q | Q-51 | Q-134 | Q-109 | Q-139 | Q-140 | Q-149 | Q-141 |
| X | H | H | CH₃ | H | H | H | H |
| Y | CH₂ | 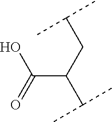 | CH₂ | CH₂ | CH₂ | CH₂ | CH₂ |

TABLE 46
with base structure I.46 and the radical definitions specified hereinafter:

| No. | I.46-1 | I.46-2 | I.46-3 | I.46-4 | I.46-5 | I.46-6 | I.46-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.46-8 | I.46-9 | I.46-10 | I.46-11 | I.46-12 | I.46-13 | I.46-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.46-15 | I.46-16 | I.46-17 | I.46-18 | I.46-19 | I.46-20 | I.46-21 |
| Q | Q-1 | Q-6 | Q-1 | Q-1 | Q-12 | Q-1 | Q-1 |
| X | H | H | c-Pr | 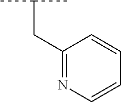 | CH₃ | CH₃ | H |
| Y | 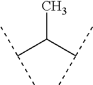 | 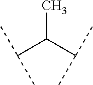 | CH₂ | CH₂ | CH₂ | 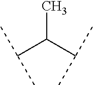 | 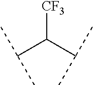 |
| No. | I.46-22 | I.46-23 | I.46-24 | I.46-25 | I.46-26 | I.46-27 | I.46-28 |
| Q | Q-1 | Q-17 | Q-8 | Q-37 | Q-38 | Q-1 | Q-25 |
| X | H | H | H | H | H | i-Pr | H |
| Y | 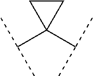 | CH₂ | CH₂ | CH₂ | CH₂ | CH₂ | CH₂ |
| No. | I.46-29 | I.46-30 | I.46-31 | I.46-32 | I.46-33 | I.46-34 | I.46-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.46-36 | I.46-37 | I.46-38 | I.46-39 | I.46-40 | I.46-41 | I.46-42 |
| Q | Q-33 | Q-34 | Q-31 | Q-52 | Q-79 | Q-80 | Q-81 |
| X | H | H | H | H | H | H | H |
| Y | CH₂ | CH₂ | 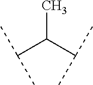 | CH₂ | CH₂ | CH₂ | CH₂ |
| No. | I.46-43 | I.46-44 | I.46-45 | I.46-46 | I.46-47 | I.46-48 | I.46-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 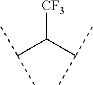 | CH₂ | CH₂ | CH₂ |
| No. | I.46-50 | I.46-51 | I.46-52 | I.46-53 | I.46-54 | I.46-55 | I.46-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.46-57 | I.46-58 | I.46-59 | I.46-60 | I.46-61 | I.46-62 | I.46-63 |
| Q | Q-51 | Q-134 | Q-109 | Q-139 | Q-140 | Q-149 | Q-141 |
| X | H | H | CH₃ | H | H | H | H |
| Y | CH₂ | 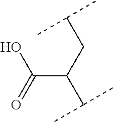 | CH₂ | CH₂ | CH₂ | CH₂ | CH₂ |

TABLE 47
with base structure I.47 and the radical definitions specified hereinafter:

| No. | I.47-1 | I.47-2 | I.47-3 | I.47-4 | I.47-5 | I.47-6 | I.47-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.47-8 | I.47-9 | I.47-10 | I.47-11 | I.47-12 | I.47-13 | I.47-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.47-15 | I.47-16 | I.47-17 | I.47-18 | I.47-19 | I.47-20 | I.47-21 |
| Q | Q-1 | Q-6 | Q-1 | Q-1 | Q-12 | Q-1 | Q-1 |
| X | H | H | c-Pr | 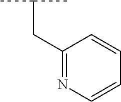 | CH₃ | CH₃ | H |
| Y | 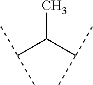 | 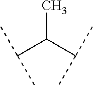 | CH₂ | CH₂ | CH₂ | 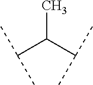 | 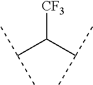 |
| No. | I.47-22 | I.47-23 | I.47-24 | I.47-25 | I.47-26 | I.47-27 | I.47-28 |
| Q | Q-1 | Q-17 | Q-8 | Q-37 | Q-38 | Q-1 | Q-25 |
| X | H | H | H | H | H | i-Pr | H |
| Y | 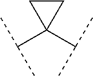 | CH₂ | CH₂ | CH₂ | CH₂ | CH₂ | CH₂ |
| No. | I.47-29 | I.47-30 | I.47-31 | I.47-32 | I.47-33 | I.47-34 | I.47-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.47-36 | I.47-37 | I.47-38 | I.47-39 | I.47-40 | I.47-41 | I.47-42 |
| Q | Q-33 | Q-34 | Q-31 | Q-52 | Q-79 | Q-80 | Q-81 |
| X | H | H | H | H | H | H | H |
| Y | CH₂ | CH₂ | 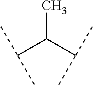 | CH₂ | CH₂ | CH₂ | CH₂ |
| No. | I.47-43 | I.47-44 | I.47-45 | I.47-46 | I.47-47 | I.47-48 | I.47-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 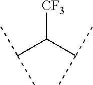 | CH₂ | CH₂ | CH₂ |
| No. | I.47-50 | I.47-51 | I.47-52 | I.47-53 | I.47-54 | I.47-55 | I.47-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.47-57 | I.47-58 | I.47-59 | I.47-60 | I.47-61 | I.47-62 | I.47-63 |
| Q | Q-51 | Q-134 | Q-109 | Q-139 | Q-140 | Q-149 | Q-141 |
| X | H | H | CH₃ | H | H | H | H |
| Y | CH₂ | 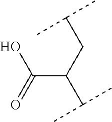 | CH₂ | CH₂ | CH₂ | CH₂ | CH₂ |

TABLE 48
with base structure I.48 and the radical definitions specified hereinafter:

| No. | I.48-1 | I.48-2 | I.48-3 | I.48-4 | I.48-5 | I.48-6 | I.48-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.48-8 | I.48-9 | I.48-10 | I.48-11 | I.48-12 | I.48-13 | I.48-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.48-15 | I.48-16 | I.48-17 | I.48-18 | I.48-19 | I.48-20 | I.48-21 |
| Q | Q-1 | Q-6 | Q-1 | Q-1 | Q-12 | Q-1 | Q-1 |
| X | H | H | c-Pr | 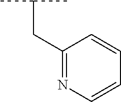 | CH₃ | CH₃ | H |
| Y | 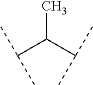 | 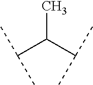 | CH₂ | CH₂ | CH₂ | 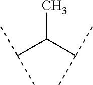 | 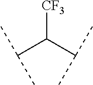 |
| No. | I.48-22 | I.48-23 | I.48-24 | I.48-25 | I.48-26 | I.48-27 | I.48-28 |
| Q | Q-1 | Q-17 | Q-8 | Q-37 | Q-38 | Q-1 | Q-25 |
| X | H | H | H | H | H | i-Pr | H |
| Y | 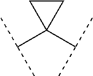 | CH₂ | CH₂ | CH₂ | CH₂ | CH₂ | CH₂ |
| No. | I.48-29 | I.48-30 | I.48-31 | I.48-32 | I.48-33 | I.48-34 | I.48-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.48-36 | I.48-37 | I.48-38 | I.48-39 | I.48-40 | I.48-41 | I.48-42 |
| Q | Q-33 | Q-34 | Q-31 | Q-52 | Q-79 | Q-80 | Q-81 |
| X | H | H | H | H | H | H | H |
| Y | CH₂ | CH₂ | 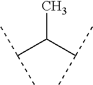 | CH₂ | CH₂ | CH₂ | CH₂ |
| No. | I.48-43 | I.48-44 | I.48-45 | I.48-46 | I.48-47 | I.48-48 | I.48-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 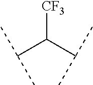 | CH₂ | CH₂ | CH₂ |
| No. | I.48-50 | I.48-51 | I.48-52 | I.48-53 | I.48-54 | I.48-55 | I.48-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.48-57 | I.48-58 | I.48-59 | I.48-60 | I.48-61 | I.48-62 | I.48-63 |
| Q | Q-51 | Q-134 | Q-109 | Q-139 | Q-140 | Q-149 | Q-141 |
| X | H | H | CH₃ | H | H | H | H |
| Y | CH₂ | 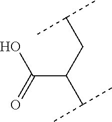 | CH₂ | CH₂ | CH₂ | CH₂ | CH₂ |

TABLE 49
with base structure I.49 and the radical definitions specified hereinafter:

| No. | I.49-1 | I.49-2 | I.49-3 | I.49-4 | I.49-5 | I.49-6 | I.49-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.49-8 | I.49-9 | I.49-10 | I.49-11 | I.49-12 | I.49-13 | I.49-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.49-15 | I.49-16 | I.49-17 | I.49-18 | I.49-19 | I.49-20 | I.49-21 |
| Q | Q-1 | Q-6 | Q-1 | Q-1 | Q-12 | Q-1 | Q-1 |
| X | H | H | c-Pr | 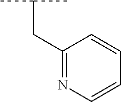 | CH₃ | CH₃ | H |
| Y | 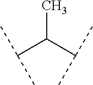 | 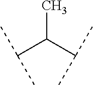 | CH₂ | CH₂ | CH₂ | 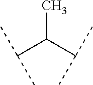 | 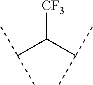 |
| No. | I.49-22 | I.49-23 | I.49-24 | I.49-25 | I.49-26 | I.49-27 | I.49-28 |
| Q | Q-1 | Q-17 | Q-8 | Q-37 | Q-38 | Q-1 | Q-25 |
| X | H | H | H | H | H | i-Pr | H |
| Y | 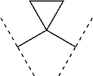 | CH₂ | CH₂ | CH₂ | CH₂ | CH₂ | CH₂ |
| No. | I.49-29 | I.49-30 | I.49-31 | I.49-32 | I.49-33 | I.49-34 | I.49-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.49-36 | I.49-37 | I.49-38 | I.49-39 | I.49-40 | I.49-41 | I.49-42 |
| Q | Q-33 | Q-34 | Q-31 | Q-52 | Q-79 | Q-80 | Q-81 |
| X | H | H | H | H | H | H | H |
| Y | CH₂ | CH₂ | 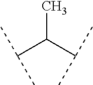 | CH₂ | CH₂ | CH₂ | CH₂ |
| No. | I.49-43 | I.49-44 | I.49-45 | I.49-46 | I.49-47 | I.49-48 | I.49-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 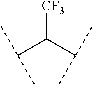 | CH₂ | CH₂ | CH₂ |
| No. | I.49-50 | I.49-51 | I.49-52 | I.49-53 | I.49-54 | I.49-55 | I.49-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.49-57 | I.49-58 | I.49-59 | I.49-60 | I.49-61 | I.49-62 | I.49-63 |
| Q | Q-51 | Q-134 | Q-109 | Q-139 | Q-140 | Q-149 | Q-141 |
| X | H | H | CH₃ | H | H | H | H |
| Y | CH₂ | 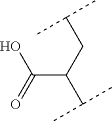 | CH₂ | CH₂ | CH₂ | CH₂ | CH₂ |

TABLE 50
with base structure I.50 and the radical definitions specified hereinafter:

| No. | I.50-1 | I.50-2 | I.50-3 | I.50-4 | I.50-5 | I.50-6 | I.50-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.50-8 | I.50-9 | I.50-10 | I.50-11 | I.50-12 | I.50-13 | I.50-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.50-15 | I.50-16 | I.50-17 | I.50-18 | I.50-19 | I.50-20 | I.50-21 |
| Q | Q-1 | Q-6 | Q-1 | Q-1 | Q-12 | Q-1 | Q-1 |
| X | H | H | c-Pr | 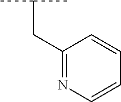 | CH₃ | CH₃ | H |
| Y | 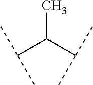 | 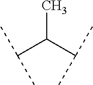 | CH₂ | CH₂ | CH₂ | 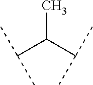 | 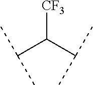 |
| No. | I.50-22 | I.50-23 | I.50-24 | I.50-25 | I.50-26 | I.50-27 | I.50-28 |
| Q | Q-1 | Q-17 | Q-8 | Q-37 | Q-38 | Q-1 | Q-25 |
| X | H | H | H | H | H | i-Pr | H |
| Y | 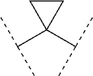 | CH₂ | CH₂ | CH₂ | CH₂ | CH₂ | CH₂ |
| No. | I.50-29 | I.50-30 | I.50-31 | I.50-32 | I.50-33 | I.50-34 | I.50-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.50-36 | I.50-37 | I.50-38 | I.50-39 | I.50-40 | I.50-41 | I.50-42 |
| Q | Q-33 | Q-34 | Q-31 | Q-52 | Q-79 | Q-80 | Q-81 |
| X | H | H | H | H | H | H | H |
| Y | CH₂ | CH₂ | 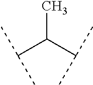 | CH₂ | CH₂ | CH₂ | CH₂ |
| No. | I.50-43 | I.50-44 | I.50-45 | I.50-46 | I.50-47 | I.50-48 | I.50-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 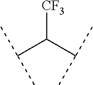 | CH₂ | CH₂ | CH₂ |
| No. | I.50-50 | I.50-51 | I.50-52 | I.50-53 | I.50-54 | I.50-55 | I.50-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.50-57 | I.50-58 | I.50-59 | I.50-60 | I.50-61 | I.50-62 | I.50-63 |
| Q | Q-51 | Q-134 | Q-109 | Q-139 | Q-140 | Q-149 | Q-141 |
| X | H | H | CH₃ | H | H | H | H |
| Y | CH₂ | 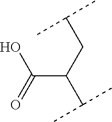 | CH₂ | CH₂ | CH₂ | CH₂ | CH₂ |

TABLE 51
with base structure I.51 and the radical definitions specified hereinafter:

| No. | I.51-1 | I.51-2 | I.51-3 | I.51-4 | I.51-5 | I.51-6 | I.51-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.51-8 | I.51-9 | I.51-10 | I.51-11 | I.51-12 | I.51-13 | I.51-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.51-15 | I.51-16 | I.51-17 | I.51-18 | I.51-19 | I.51-20 | I.51-21 |
| Q | Q-1 | Q-6 | Q-1 | Q-1 | Q-12 | Q-1 | Q-1 |
| X | H | H | c-Pr | 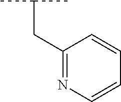 | CH₃ | CH₃ | H |
| Y | 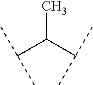 | 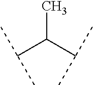 | CH₂ | CH₂ | CH₂ | 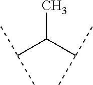 | 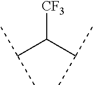 |
| No. | I.51-22 | I.51-23 | I.51-24 | I.51-25 | I.51-26 | I.51-27 | I.51-28 |
| Q | Q-1 | Q-17 | Q-8 | Q-37 | Q-38 | Q-1 | Q-25 |
| X | H | H | H | H | H | i-Pr | H |
| Y | 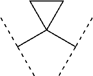 | CH₂ | CH₂ | CH₂ | CH₂ | CH₂ | CH₂ |
| No. | I.51-29 | I.51-30 | I.51-31 | I.51-32 | I.51-33 | I.51-34 | I.51-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.51-36 | I.51-37 | I.51-38 | I.51-39 | I.51-40 | I.51-41 | I.51-42 |
| Q | Q-33 | Q-34 | Q-31 | Q-52 | Q-79 | Q-80 | Q-81 |
| X | H | H | H | H | H | H | H |
| Y | CH₂ | CH₂ | 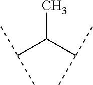 | CH₂ | CH₂ | CH₂ | CH₂ |
| No. | I.51-43 | I.51-44 | I.51-45 | I.51-46 | I.51-47 | I.51-48 | I.51-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 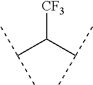 | CH₂ | CH₂ | CH₂ |
| No. | I.51-50 | I.51-51 | I.51-52 | I.51-53 | I.51-54 | I.51-55 | I.51-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.51-57 | I.51-58 | I.51-59 | I.51-60 | I.51-61 | I.51-62 | I.51-63 |
| Q | Q-51 | Q-134 | Q-109 | Q-139 | Q-140 | Q-149 | Q-141 |
| X | H | H | CH₃ | H | H | H | H |
| Y | CH₂ | 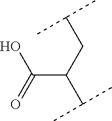 | CH₂ | CH₂ | CH₂ | CH₂ | CH₂ |

TABLE 52
with base structure I.52 and the radical definitions specified hereinafter:

| No. | I.52-1 | I.52-2 | I.52-3 | I.52-4 | I.52-5 | I.52-6 | I.52-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.52-8 | I.52-9 | I.52-10 | I.52-11 | I.52-12 | I.52-13 | I.52-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.52-15 | I.52-16 | I.52-17 | I.52-18 | I.52-19 | I.52-20 | I.52-21 |
| Q | Q-1 | Q-6 | Q-1 | Q-1 | Q-12 | Q-1 | Q-1 |
| X | H | H | c-Pr | 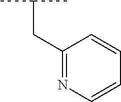 | CH₃ | CH₃ | H |
| Y | 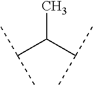 | 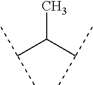 | CH₂ | CH₂ | CH₂ | 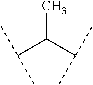 | 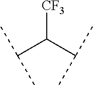 |
| No. | I.52-22 | I.52-23 | I.52-24 | I.52-25 | I.52-26 | I.52-27 | I.52-28 |
| Q | Q-1 | Q-17 | Q-8 | Q-37 | Q-38 | Q-1 | Q-25 |
| X | H | H | H | H | H | i-Pr | H |
| Y | 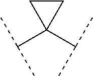 | CH₂ | CH₂ | CH₂ | CH₂ | CH₂ | CH₂ |
| No. | I.52-29 | I.52-30 | I.52-31 | I.52-32 | I.52-33 | I.52-34 | I.52-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.52-36 | I.52-37 | I.52-38 | I.52-39 | I.52-40 | I.52-41 | I.52-42 |
| Q | Q-33 | Q-34 | Q-31 | Q-52 | Q-79 | Q-80 | Q-81 |
| X | H | H | H | H | H | H | H |
| Y | CH₂ | CH₂ | 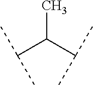 | CH₂ | CH₂ | CH₂ | CH₂ |
| No. | I.52-43 | I.52-44 | I.52-45 | I.52-46 | I.52-47 | I.52-48 | I.52-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 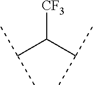 | CH₂ | CH₂ | CH₂ |
| No. | I.52-50 | I.52-51 | I.52-52 | I.52-53 | I.52-54 | I.52-55 | I.52-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.52-57 | I.52-58 | I.52-59 | I.52-60 | I.52-61 | I.52-62 | I.52-63 |
| Q | Q-51 | Q-134 | Q-109 | Q-139 | Q-140 | Q-149 | Q-141 |
| X | H | H | CH₃ | H | H | H | H |
| Y | CH₂ | 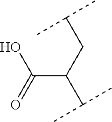 | CH₂ | CH₂ | CH₂ | CH₂ | CH₂ |

TABLE 53
with base structure I.53 and the radical definitions specified hereinafter:

| No. | I.53-1 | I.53-2 | I.53-3 | I.53-4 | I.53-5 | I.53-6 | I.53-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.53-8 | I.53-9 | I.53-10 | I.53-11 | I.53-12 | I.53-13 | I.53-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.53-15 | I.53-16 | I.53-17 | I.53-18 | I.53-19 | I.53-20 | I.53-21 |
| Q | Q-1 | Q-6 | Q-1 | Q-1 | Q-12 | Q-1 | Q-1 |
| X | H | H | c-Pr | 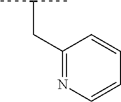 | CH₃ | CH₃ | H |
| Y | 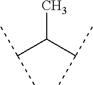 | 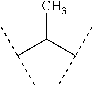 | CH₂ | CH₂ | CH₂ | 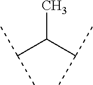 | 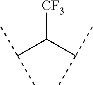 |
| No. | I.53-22 | I.53-23 | I.53-24 | I.53-25 | I.53-26 | I.53-27 | I.53-28 |
| Q | Q-1 | Q-17 | Q-8 | Q-37 | Q-38 | Q-1 | Q-25 |
| X | H | H | H | H | H | i-Pr | H |
| Y | 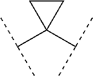 | CH₂ | CH₂ | CH₂ | CH₂ | CH₂ | CH₂ |
| No. | I.53-29 | I.53-30 | I.53-31 | I.53-32 | I.53-33 | I.53-34 | I.53-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.53-36 | I.53-37 | I.53-38 | I.53-39 | I.53-40 | I.53-41 | I.53-42 |
| Q | Q-33 | Q-34 | Q-31 | Q-52 | Q-79 | Q-80 | Q-81 |
| X | H | H | H | H | H | H | H |
| Y | CH₂ | CH₂ | 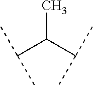 | CH₂ | CH₂ | CH₂ | CH₂ |
| No. | I.53-43 | I.53-44 | I.53-45 | I.53-46 | I.53-47 | I.53-48 | I.53-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 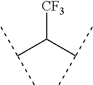 | CH₂ | CH₂ | CH₂ |
| No. | I.53-50 | I.53-51 | I.53-52 | I.53-53 | I.53-54 | I.53-55 | I.53-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.53-57 | I.53-58 | I.53-59 | I.53-60 | I.53-61 | I.53-62 | I.53-63 |
| Q | Q-51 | Q-134 | Q-109 | Q-139 | Q-140 | Q-149 | Q-141 |
| X | H | H | CH₃ | H | H | H | H |
| Y | CH₂ | 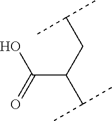 | CH₂ | CH₂ | CH₂ | CH₂ | CH₂ |

TABLE 54
with base structure I.54 and the radical definitions specified hereinafter:

| No. | I.54-1 | I.54-2 | I.54-3 | I.54-4 | I.54-5 | I.54-6 | I.54-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.54-8 | I.54-9 | I.54-10 | I.54-11 | I.54-12 | I.54-13 | I.54-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.54-15 | I.54-16 | I.54-17 | I.54-18 | I.54-19 | I.54-20 | I.54-21 |
| Q | Q-1 | Q-6 | Q-1 | Q-1 | Q-12 | Q-1 | Q-1 |
| X | H | H | c-Pr | 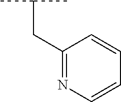 | CH₃ | CH₃ | H |
| Y | 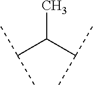 | 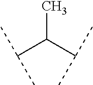 | CH₂ | CH₂ | CH₂ | 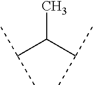 | 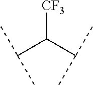 |
| No. | I.54-22 | I.54-23 | I.54-24 | I.54-25 | I.54-26 | I.54-27 | I.54-28 |
| Q | Q-1 | Q-17 | Q-8 | Q-37 | Q-38 | Q-1 | Q-25 |
| X | H | H | H | H | H | i-Pr | H |
| Y | 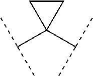 | CH₂ | CH₂ | CH₂ | CH₂ | CH₂ | CH₂ |
| No. | I.54-29 | I.54-30 | I.54-31 | I.54-32 | I.54-33 | I.54-34 | I.54-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.54-36 | I.54-37 | I.54-38 | I.54-39 | I.54-40 | I.54-41 | I.54-42 |
| Q | Q-33 | Q-34 | Q-31 | Q-52 | Q-79 | Q-80 | Q-81 |
| X | H | H | H | H | H | H | H |
| Y | CH₂ | CH₂ | 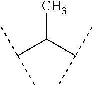 | CH₂ | CH₂ | CH₂ | CH₂ |
| No. | I.54-43 | I.54-44 | I.54-45 | I.54-46 | I.54-47 | I.54-48 | I.54-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 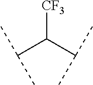 | CH₂ | CH₂ | CH₂ |
| No. | I.54-50 | I.54-51 | I.54-52 | I.54-53 | I.54-54 | I.54-55 | I.54-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.54-57 | I.54-58 | I.54-59 | I.54-60 | I.54-61 | I.54-62 | I.54-63 |
| Q | Q-51 | Q-134 | Q-109 | Q-139 | Q-140 | Q-149 | Q-141 |
| X | H | H | CH₃ | H | H | H | H |
| Y | CH₂ | 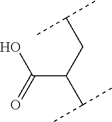 | CH₂ | CH₂ | CH₂ | CH₂ | CH₂ |

TABLE 55
with base structure I.55 and the radical definitions specified hereinafter:

| No. | I.55-1 | I.55-2 | I.55-3 | I.55-4 | I.55-5 | I.55-6 | I.55-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.55-8 | I.55-9 | I.55-10 | I.55-11 | I.55-12 | I.55-13 | I.55-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.55-15 | I.55-16 | I.55-17 | I.55-18 | I.55-19 | I.55-20 | I.55-21 |
| Q | Q-1 | Q-6 | Q-1 | Q-1 | Q-12 | Q-1 | Q-1 |
| X | H | H | c-Pr | 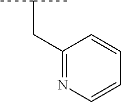 | CH₃ | CH₃ | H |
| Y | 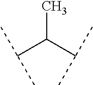 | 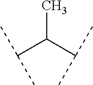 | CH₂ | CH₂ | CH₂ | 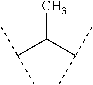 | 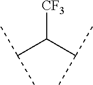 |
| No. | I.55-22 | I.55-23 | I.55-24 | I.55-25 | I.55-26 | I.55-27 | I.55-28 |
| Q | Q-1 | Q-17 | Q-8 | Q-37 | Q-38 | Q-1 | Q-25 |
| X | H | H | H | H | H | i-Pr | H |
| Y | 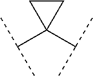 | CH₂ | CH₂ | CH₂ | CH₂ | CH₂ | CH₂ |
| No. | I.55-29 | I.55-30 | I.55-31 | I.55-32 | I.55-33 | I.55-34 | I.55-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.55-36 | I.55-37 | I.55-38 | I.55-39 | I.55-40 | I.55-41 | I.55-42 |
| Q | Q-33 | Q-34 | Q-31 | Q-52 | Q-79 | Q-80 | Q-81 |
| X | H | H | H | H | H | H | H |
| Y | CH₂ | CH₂ | 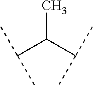 | CH₂ | CH₂ | CH₂ | CH₂ |
| No. | I.55-43 | I.55-44 | I.55-45 | I.55-46 | I.55-47 | I.55-48 | I.55-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 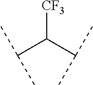 | CH₂ | CH₂ | CH₂ |
| No. | I.55-50 | I.55-51 | I.55-52 | I.55-53 | I.55-54 | I.55-55 | I.55-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.55-57 | I.55-58 | I.55-59 | I.55-60 | I.55-61 | I.55-62 | I.55-63 |
| Q | Q-51 | Q-134 | Q-109 | Q-139 | Q-140 | Q-149 | Q-141 |
| X | H | H | CH₃ | H | H | H | H |
| Y | CH₂ | 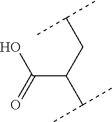 | CH₂ | CH₂ | CH₂ | CH₂ | CH₂ |

TABLE 56
with base structure I.56 and the radical definitions specified hereinafter:

| No. | I.56-1 | I.56-2 | I.56-3 | I.56-4 | I.56-5 | I.56-6 | I.56-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.56-8 | I.56-9 | I.56-10 | I.56-11 | I.56-12 | I.56-13 | I.56-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.56-15 | I.56-16 | I.56-17 | I.56-18 | I.56-19 | I.56-20 | I.56-21 |
| Q | Q-1 | Q-6 | Q-1 | Q-1 | Q-12 | Q-1 | Q-1 |
| X | H | H | c-Pr | 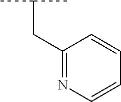 | CH₃ | CH₃ | H |
| Y | 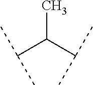 | 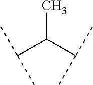 | CH₂ | CH₂ | CH₂ | 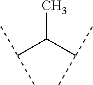 | 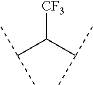 |
| No. | I.56-22 | I.56-23 | I.56-24 | I.56-25 | I.56-26 | I.56-27 | I.56-28 |
| Q | Q-1 | Q-17 | Q-8 | Q-37 | Q-38 | Q-1 | Q-25 |
| X | H | H | H | H | H | i-Pr | H |
| Y | 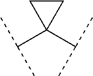 | CH₂ | CH₂ | CH₂ | CH₂ | CH₂ | CH₂ |
| No. | I.56-29 | I.56-30 | I.56-31 | I.56-32 | I.56-33 | I.56-34 | I.56-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.56-36 | I.56-37 | I.56-38 | I.56-39 | I.56-40 | I.56-41 | I.56-42 |
| Q | Q-33 | Q-34 | Q-31 | Q-52 | Q-79 | Q-80 | Q-81 |
| X | H | H | H | H | H | H | H |
| Y | CH₂ | CH₂ | 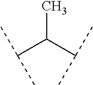 | CH₂ | CH₂ | CH₂ | CH₂ |
| No. | I.56-43 | I.56-44 | I.56-45 | I.56-46 | I.56-47 | I.56-48 | I.56-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 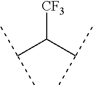 | CH₂ | CH₂ | CH₂ |
| No. | I.56-50 | I.56-51 | I.56-52 | I.56-53 | I.56-54 | I.56-55 | I.56-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.56-57 | I.56-58 | I.56-59 | I.56-60 | I.56-61 | I.56-62 | I.56-63 |
| Q | Q-51 | Q-134 | Q-109 | Q-139 | Q-140 | Q-149 | Q-141 |
| X | H | H | CH₃ | H | H | H | H |
| Y | CH₂ | 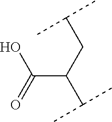 | CH₂ | CH₂ | CH₂ | CH₂ | CH₂ |

TABLE 57
with base structure I.57 and the radical definitions specified hereinafter:

| No. | I.57-1 | I.57-2 | I.57-3 | I.57-4 | I.57-5 | I.57-6 | I.57-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.57-8 | I.57-9 | I.57-10 | I.57-11 | I.57-12 | I.57-13 | I.57-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.57-15 | I.57-16 | I.57-17 | I.57-18 | I.57-19 | I.57-20 | I.57-21 |
| Q | Q-1 | Q-6 | Q-1 | Q-1 | Q-12 | Q-1 | Q-1 |
| X | H | H | c-Pr | 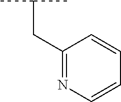 | CH₃ | CH₃ | H |
| Y | 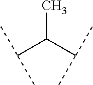 | 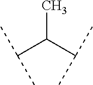 | CH₂ | CH₂ | CH₂ | 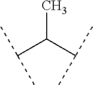 | 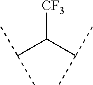 |
| No. | I.57-22 | I.57-23 | I.57-24 | I.57-25 | I.57-26 | I.57-27 | I.57-28 |
| Q | Q-1 | Q-17 | Q-8 | Q-37 | Q-38 | Q-1 | Q-25 |
| X | H | H | H | H | H | i-Pr | H |
| Y | 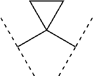 | CH₂ | CH₂ | CH₂ | CH₂ | CH₂ | CH₂ |
| No. | I.57-29 | I.57-30 | I.57-31 | I.57-32 | I.57-33 | I.57-34 | I.57-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.57-36 | I.57-37 | I.57-38 | I.57-39 | I.57-40 | I.57-41 | I.57-42 |
| Q | Q-33 | Q-34 | Q-31 | Q-52 | Q-79 | Q-80 | Q-81 |
| X | H | H | H | H | H | H | H |
| Y | CH₂ | CH₂ | 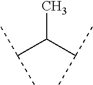 | CH₂ | CH₂ | CH₂ | CH₂ |
| No. | I.57-43 | I.57-44 | I.57-45 | I.57-46 | I.57-47 | I.57-48 | I.57-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 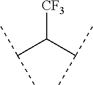 | CH₂ | CH₂ | CH₂ |
| No. | I.57-50 | I.57-51 | I.57-52 | I.57-53 | I.57-54 | I.57-55 | I.57-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.57-57 | I.57-58 | I.57-59 | I.57-60 | I.57-61 | I.57-62 | I.57-63 |
| Q | Q-51 | Q-134 | Q-109 | Q-139 | Q-140 | Q-149 | Q-141 |
| X | H | H | CH₃ | H | H | H | H |
| Y | CH₂ | 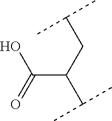 | CH₂ | CH₂ | CH₂ | CH₂ | CH₂ |

TABLE 58
with base structure I.58 and the radical definitions specified hereinafter:

| No. | I.58-1 | I.58-2 | I.58-3 | I.58-4 | I.58-5 | I.58-6 | I.58-7 |
|---|---|---|---|---|---|---|---|
| Q | Q-1 | Q-2 | Q-3 | Q-4 | Q-5 | Q-6 | Q-7 |
| X | H | H | H | H | H | H | H |
| Y | CH₂ | CH₂ | CH₂ | CH₂ | CH₂ | CH₂ | CH₂ |
| No. | I.58-8 | I.58-9 | I.58-10 | I.58-11 | I.58-12 | I.58-13 | I.58-14 |
| Q | Q-9 | Q-10 | Q-11 | Q-1 | Q-12 | Q-13 | Q-14 |
| X | H | H | H | CH₃ | H | H | H |
| Y | CH₂ | CH₂ | CH₂ | CH₂ | CH₂ | CH₂ | CH₂ |
| No. | I.58-15 | I.58-16 | I.58-17 | I.58-18 | I.58-19 | I.58-20 | I.58-21 |
| Q | Q-1 | Q-6 | Q-1 | Q-1 | Q-12 | Q-1 | Q-1 |
| X | H | H | c-Pr | 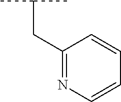 | CH₃ | CH₃ | H |
| Y | 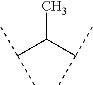 | 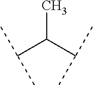 | CH₂ | CH₂ | CH₂ | 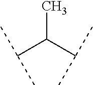 | 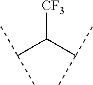 |
| No. | I.58-22 | I.58-23 | I.58-24 | I.58-25 | I.58-26 | I.58-27 | I.58-28 |
| Q | Q-1 | Q-17 | Q-8 | Q-37 | Q-38 | Q-1 | Q-25 |
| X | H | H | H | H | H | i-Pr | H |
| Y | 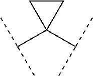 | CH₂ | CH₂ | CH₂ | CH₂ | CH₂ | CH₂ |
| No. | I.58-29 | I.58-30 | I.58-31 | I.58-32 | I.58-33 | I.58-34 | I.58-35 |
| Q | Q-81 | Q-136 | Q-26 | Q-27 | Q-29 | Q-31 | Q-32 |
| X | CH₃ | H | H | H | H | H | H |
| Y | CH₂ | CH₂ | CH₂ | CH₂ | CH₂ | CH₂ | CH₂ |
| No. | I.58-36 | I.58-37 | I.58-38 | I.58-39 | I.58-40 | I.58-41 | I.58-42 |
| Q | Q-33 | Q-34 | Q-31 | Q-52 | Q-79 | Q-80 | Q-81 |
| X | H | H | H | H | H | H | H |
| Y | CH₂ | CH₂ | 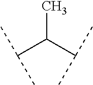 | CH₂ | CH₂ | CH₂ | CH₂ |
| No. | I.58-43 | I.58-44 | I.58-45 | I.58-46 | I.58-47 | I.58-48 | I.58-49 |
| Q | Q-82 | Q-83 | Q-84 | Q-79 | Q-57 | Q-58 | Q-60 |
| X | H | H | H | H | H | H | H |
| Y | CH₂ | CH₂ | CH₂ | 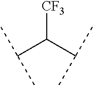 | CH₂ | CH₂ | CH₂ |
| No. | I.58-50 | I.58-51 | I.58-52 | I.58-53 | I.58-54 | I.58-55 | I.58-56 |
| Q | Q-64 | Q-78 | Q-77 | Q-113 | Q-114 | Q-109 | Q-110 |
| X | H | H | H | H | H | H | H |
| Y | CH₂ | CH₂ | CH₂ | CH₂ | CH₂ | CH₂ | CH₂ |
| No. | I.58-57 | I.58-58 | I.58-59 | I.58-60 | I.58-61 | I.58-62 | I.58-63 |
| Q | Q-51 | Q-134 | Q-109 | Q-139 | Q-140 | Q-149 | Q-141 |
| X | H | H | CH₃ | H | H | H | H |
| Y | CH₂ | 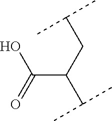 | CH₂ | CH₂ | CH₂ | CH₂ | CH₂ |

TABLE 59
with base structure I.59 and the radical definitions specified hereinafter:

| No. | I.59-1 | I.59-2 | I.59-3 | I.59-4 | I.59-5 | I.59-6 | I.59-7 | I.59-8 | I.59-9 | I.59-10 | I.59-11 | I.59-12 | I.59-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.59-14 | I.59-15 | I.59-16 | I.59-17 | I.59-18 | I.59-19 | I.59-20 |
|---|---|---|---|---|---|---|---|
| Q | Q-14 | Q-1 | Q-6 | Q-1 | Q-1 | Q-12 | Q-1 |
| X | H | H | H | c-Pr | 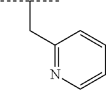 | CH₃ | CH₃ |
| Y | CH₂ | 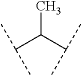 | 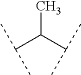 | CH₂ | CH₂ | CH₂ | 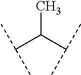 |

| No. | I.59-21 | I.59-22 | I.59-23 | I.59-24 | I.59-25 | I.59-26 | I.59-27 | I.59-28 | I.59-29 | I.59-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 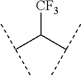 | 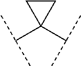 | CH₂ | CH₂ | CH₂ | CH₂ | CH₂ | CH₂ | CH₂ | CH₂ |

| No. | I.59-31 | I.59-32 | I.59-33 | I.59-34 | I.59-35 | I.59-36 | I.59-37 | I.59-38 | I.59-39 | I.59-40 | I.59-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 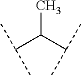 | CH₂ | CH₂ | CH₂ |

| No. | I.59-42 | I.59-43 | I.59-44 | I.59-45 | I.59-46 | I.59-47 | I.59-48 | I.59-49 | I.59-50 | I.59-51 | I.59-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 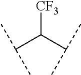 | CH₂ | CH₂ | CH₂ | CH₂ | CH₂ | CH₂ |

| No. | I.59-53 | I.59-54 | I.59-55 | I.59-56 | I.59-57 | I.59-58 | I.59-59 | I.59-60 | I.59-61 | I.59-62 | I.59-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 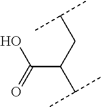 | CH₂ | CH₂ | CH₂ | CH₂ | CH₂ |

TABLE 60
with base structure I.60 and the radical definitions specified hereinafter:

| No. | I.60-1 | I.60-2 | I.60-3 | I.60-4 | I.60-5 | I.60-6 | I.60-7 | I.60-8 | I.60-9 | I.60-10 | I.60-11 | I.60-12 | I.60-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.60-14 | I.60-15 | I.60-16 | I.60-17 | I.60-18 | I.60-19 | I.60-20 |
|---|---|---|---|---|---|---|---|
| Q | Q-14 | Q-1 | Q-6 | Q-1 | Q-1 | Q-12 | Q-1 |
| X | H | H | H | c-Pr | 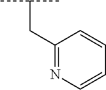 | CH₃ | CH₃ |
| Y | CH₂ | 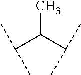 | 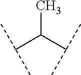 | CH₂ | CH₂ | CH₂ | 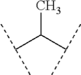 |

| No. | I.60-21 | I.60-22 | I.60-23 | I.60-24 | I.60-25 | I.60-26 | I.60-27 | I.60-28 | I.60-29 | I.60-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 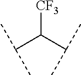 | 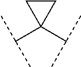 | CH₂ | CH₂ | CH₂ | CH₂ | CH₂ | CH₂ | CH₂ | CH₂ |

| No. | I.60-31 | I.60-32 | I.60-33 | I.60-34 | I.60-35 | I.60-36 | I.60-37 | I.60-38 | I.60-39 | I.60-40 | I.60-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 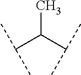 | CH₂ | CH₂ | CH₂ |

| No. | I.60-42 | I.60-43 | I.60-44 | I.60-45 | I.60-46 | I.60-47 | I.60-48 | I.60-49 | I.60-50 | I.60-51 | I.60-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 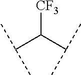 | CH₂ | CH₂ | CH₂ | CH₂ | CH₂ | CH₂ |

| No. | I.60-53 | I.60-54 | I.60-55 | I.60-56 | I.60-57 | I.60-58 | I.60-59 | I.60-60 | I.60-61 | I.60-62 | I.60-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 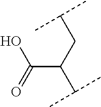 | CH₂ | CH₂ | CH₂ | CH₂ | CH₂ |

TABLE 61
with base structure I.61 and the radical definitions specified hereinafter:

| No. | I.61-1 | I.61-2 | I.61-3 | I.61-4 | I.61-5 | I.61-6 | I.61-7 | I.61-8 | I.61-9 | I.61-10 | I.61-11 | I.61-12 | I.61-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.61-14 | I.61-15 | I.61-16 | I.61-17 | I.61-18 | I.61-19 | I.61-20 |
|---|---|---|---|---|---|---|---|
| Q | Q-14 | Q-1 | Q-6 | Q-1 | Q-1 | Q-12 | Q-1 |
| X | H | H | H | c-Pr | 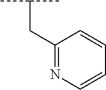 | CH₃ | CH₃ |
| Y | CH₂ | 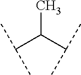 | 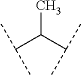 | CH₂ | CH₂ | CH₂ | 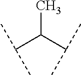 |

| No. | I.61-21 | I.61-22 | I.61-23 | I.61-24 | I.61-25 | I.61-26 | I.61-27 | I.61-28 | I.61-29 | I.61-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 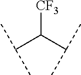 | 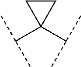 | CH₂ | CH₂ | CH₂ | CH₂ | CH₂ | CH₂ | CH₂ | CH₂ |

| No. | I.61-31 | I.61-32 | I.61-33 | I.61-34 | I.61-35 | I.61-36 | I.61-37 | I.61-38 | I.61-39 | I.61-40 | I.61-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 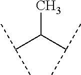 | CH₂ | CH₂ | CH₂ |

| No. | I.61-42 | I.61-43 | I.61-44 | I.61-45 | I.61-46 | I.61-47 | I.61-48 | I.61-49 | I.61-50 | I.61-51 | I.61-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 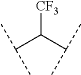 | CH₂ | CH₂ | CH₂ | CH₂ | CH₂ | CH₂ |

| No. | I.61-53 | I.61-54 | I.61-55 | I.61-56 | I.61-57 | I.61-58 | I.61-59 | I.61-60 | I.61-61 | I.61-62 | I.61-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 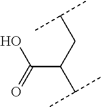 | CH₂ | CH₂ | CH₂ | CH₂ | CH₂ |

TABLE 62
with base structure I.62 and the radical definitions specified hereinafter:

| No. | I.62-1 | I.62-2 | I.62-3 | I.62-4 | I.62-5 | I.62-6 | I.62-7 | I.62-8 | I.62-9 | I.62-10 | I.62-11 | I.62-12 | I.62-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.62-14 | I.62-15 | I.62-16 | I.62-17 | I.62-18 | I.62-19 | I.62-20 |
|---|---|---|---|---|---|---|---|
| Q | Q-14 | Q-1 | Q-6 | Q-1 | Q-1 | Q-12 | Q-1 |
| X | H | H | H | c-Pr | 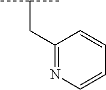 | CH₃ | CH₃ |
| Y | CH₂ | 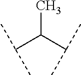 | 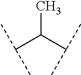 | CH₂ | CH₂ | CH₂ | 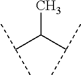 |

| No. | I.62-21 | I.62-22 | I.62-23 | I.62-24 | I.62-25 | I.62-26 | I.62-27 | I.62-28 | I.62-29 | I.62-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 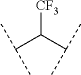 | 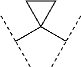 | CH₂ | CH₂ | CH₂ | CH₂ | CH₂ | CH₂ | CH₂ | CH₂ |

| No. | I.62-31 | I.62-32 | I.62-33 | I.62-34 | I.62-35 | I.62-36 | I.62-37 | I.62-38 | I.62-39 | I.62-40 | I.62-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 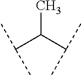 | CH₂ | CH₂ | CH₂ |

| No. | I.62-42 | I.62-43 | I.62-44 | I.62-45 | I.62-46 | I.62-47 | I.62-48 | I.62-49 | I.62-50 | I.62-51 | I.62-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 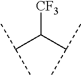 | CH₂ | CH₂ | CH₂ | CH₂ | CH₂ | CH₂ |

| No. | I.62-53 | I.62-54 | I.62-55 | I.62-56 | I.62-57 | I.62-58 | I.62-59 | I.62-60 | I.62-61 | I.62-62 | I.62-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 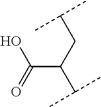 | CH₂ | CH₂ | CH₂ | CH₂ | CH₂ |

TABLE 63
with base structure I.63 and the radical definitions specified hereinafter:

| No. | I.63-1 | I.63-2 | I.63-3 | I.63-4 | I.63-5 | I.63-6 | I.63-7 | I.63-8 | I.63-9 | I.63-10 | I.63-11 | I.63-12 | I.63-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.63-14 | I.63-15 | I.63-16 | I.63-17 | I.63-18 | I.63-19 | I.63-20 |
|---|---|---|---|---|---|---|---|
| Q | Q-14 | Q-1 | Q-6 | Q-1 | Q-1 | Q-12 | Q-1 |
| X | H | H | H | c-Pr | 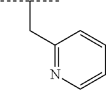 | CH₃ | CH₃ |
| Y | CH₂ | 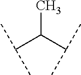 | 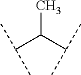 | CH₂ | CH₂ | CH₂ | 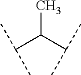 |

| No. | I.63-21 | I.63-22 | I.63-23 | I.63-24 | I.63-25 | I.63-26 | I.63-27 | I.63-28 | I.63-29 | I.63-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 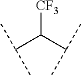 | 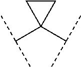 | CH₂ | CH₂ | CH₂ | CH₂ | CH₂ | CH₂ | CH₂ | CH₂ |

| No. | I.63-31 | I.63-32 | I.63-33 | I.63-34 | I.63-35 | I.63-36 | I.63-37 | I.63-38 | I.63-39 | I.63-40 | I.63-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 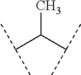 | CH₂ | CH₂ | CH₂ |

| No. | I.63-42 | I.63-43 | I.63-44 | I.63-45 | I.63-46 | I.63-47 | I.63-48 | I.63-49 | I.63-50 | I.63-51 | I.63-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 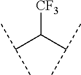 | CH₂ | CH₂ | CH₂ | CH₂ | CH₂ | CH₂ |

| No. | I.63-53 | I.63-54 | I.63-55 | I.63-56 | I.63-57 | I.63-58 | I.63-59 | I.63-60 | I.63-61 | I.63-62 | I.63-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 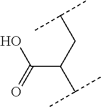 | CH₂ | CH₂ | CH₂ | CH₂ | CH₂ |

TABLE 64
with base structure I.64 and the radical definitions specified hereinafter:

| No. | I.64-1 | I.64-2 | I.64-3 | I.64-4 | I.64-5 | I.64-6 | I.64-7 | I.64-8 | I.64-9 | I.64-10 | I.64-11 | I.64-12 | I.64-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.64-14 | I.64-15 | I.64-16 | I.64-17 | I.64-18 | I.64-19 | I.64-20 |
|---|---|---|---|---|---|---|---|
| Q | Q-14 | Q-1 | Q-6 | Q-1 | Q-1 | Q-12 | Q-1 |
| X | H | H | H | c-Pr | 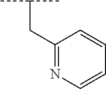 | CH₃ | CH₃ |
| Y | CH₂ | 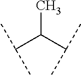 | 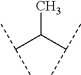 | CH₂ | CH₂ | CH₂ | 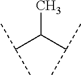 |

| No. | I.64-21 | I.64-22 | I.64-23 | I.64-24 | I.64-25 | I.64-26 | I.64-27 | I.64-28 | I.64-29 | I.64-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 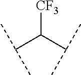 | 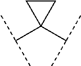 | CH₂ | CH₂ | CH₂ | CH₂ | CH₂ | CH₂ | CH₂ | CH₂ |

| No. | I.64-31 | I.64-32 | I.64-33 | I.64-34 | I.64-35 | I.64-36 | I.64-37 | I.64-38 | I.64-39 | I.64-40 | I.64-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 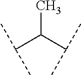 | CH₂ | CH₂ | CH₂ |

| No. | I.64-42 | I.64-43 | I.64-44 | I.64-45 | I.64-46 | I.64-47 | I.64-48 | I.64-49 | I.64-50 | I.64-51 | I.64-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 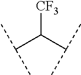 | CH₂ | CH₂ | CH₂ | CH₂ | CH₂ | CH₂ |

| No. | I.64-53 | I.64-54 | I.64-55 | I.64-56 | I.64-57 | I.64-58 | I.64-59 | I.64-60 | I.64-61 | I.64-62 | I.64-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 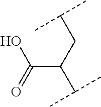 | CH₂ | CH₂ | CH₂ | CH₂ | CH₂ |

TABLE 65
with base structure I.65 and the radical definitions specified hereinafter:

| No. | I.65-1 | I.65-2 | I.65-3 | I.65-4 | I.65-5 | I.65-6 | I.65-7 | I.65-8 | I.65-9 | I.65-10 | I.65-11 | I.65-12 | I.65-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.65-14 | I.65-15 | I.65-16 | I.65-17 | I.65-18 | I.65-19 | I.65-20 |
|---|---|---|---|---|---|---|---|
| Q | Q-14 | Q-1 | Q-6 | Q-1 | Q-1 | Q-12 | Q-1 |
| X | H | H | H | c-Pr | 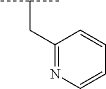 | CH₃ | CH₃ |
| Y | CH₂ | 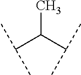 | 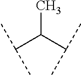 | CH₂ | CH₂ | CH₂ | 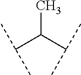 |

| No. | I.65-21 | I.65-22 | I.65-23 | I.65-24 | I.65-25 | I.65-26 | I.65-27 | I.65-28 | I.65-29 | I.65-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 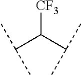 | 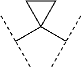 | CH₂ | CH₂ | CH₂ | CH₂ | CH₂ | CH₂ | CH₂ | CH₂ |

| No. | I.65-31 | I.65-32 | I.65-33 | I.65-34 | I.65-35 | I.65-36 | I.65-37 | I.65-38 | I.65-39 | I.65-40 | I.65-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 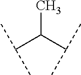 | CH₂ | CH₂ | CH₂ |

| No. | I.65-42 | I.65-43 | I.65-44 | I.65-45 | I.65-46 | I.65-47 | I.65-48 | I.65-49 | I.65-50 | I.65-51 | I.65-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 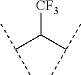 | CH₂ | CH₂ | CH₂ | CH₂ | CH₂ | CH₂ |

| No. | I.65-53 | I.65-54 | I.65-55 | I.65-56 | I.65-57 | I.65-58 | I.65-59 | I.65-60 | I.65-61 | I.65-62 | I.65-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 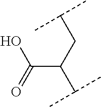 | CH₂ | CH₂ | CH₂ | CH₂ | CH₂ |

TABLE 66
with base structure I.66 and the radical definitions specified hereinafter:

| No. | I.66-1 | I.66-2 | I.66-3 | I.66-4 | I.66-5 | I.66-6 | I.66-7 | I.66-8 | I.66-9 | I.66-10 | I.66-11 | I.66-12 | I.66-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.66-14 | I.66-15 | I.66-16 | I.66-17 | I.66-18 | I.66-19 | I.66-20 |
|---|---|---|---|---|---|---|---|
| Q | Q-14 | Q-1 | Q-6 | Q-1 | Q-1 | Q-12 | Q-1 |
| X | H | H | H | c-Pr | 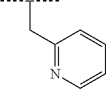 | CH₃ | CH₃ |
| Y | CH₂ | 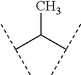 | 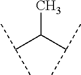 | CH₂ | CH₂ | CH₂ | 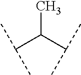 |

| No. | I.66-21 | I.66-22 | I.66-23 | I.66-24 | I.66-25 | I.66-26 | I.66-27 | I.66-28 | I.66-29 | I.66-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 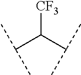 | 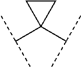 | CH₂ | CH₂ | CH₂ | CH₂ | CH₂ | CH₂ | CH₂ | CH₂ |

| No. | I.66-31 | I.66-32 | I.66-33 | I.66-34 | I.66-35 | I.66-36 | I.66-37 | I.66-38 | I.66-39 | I.66-40 | I.66-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 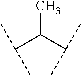 | CH₂ | CH₂ | CH₂ |

| No. | I.66-42 | I.66-43 | I.66-44 | I.66-45 | I.66-46 | I.66-47 | I.66-48 | I.66-49 | I.66-50 | I.66-51 | I.66-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 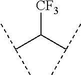 | CH₂ | CH₂ | CH₂ | CH₂ | CH₂ | CH₂ |

| No. | I.66-53 | I.66-54 | I.66-55 | I.66-56 | I.66-57 | I.66-58 | I.66-59 | I.66-60 | I.66-61 | I.66-62 | I.66-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 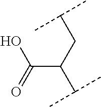 | CH₂ | CH₂ | CH₂ | CH₂ | CH₂ |

TABLE 67
with base structure I.67 and the radical definitions specified hereinafter:

| No. | I.67-1 | I.67-2 | I.67-3 | I.67-4 | I.67-5 | I.67-6 | I.67-7 | I.67-8 | I.67-9 | I.67-10 | I.67-11 | I.67-12 | I.67-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.67-14 | I.67-15 | I.67-16 | I.67-17 | I.67-18 | I.67-19 | I.67-20 |
|---|---|---|---|---|---|---|---|
| Q | Q-14 | Q-1 | Q-6 | Q-1 | Q-1 | Q-12 | Q-1 |
| X | H | H | H | c-Pr | 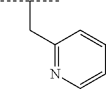 | CH₃ | CH₃ |
| Y | CH₂ | 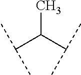 | 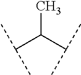 | CH₂ | CH₂ | CH₂ | 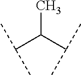 |

| No. | I.67-21 | I.67-22 | I.67-23 | I.67-24 | I.67-25 | I.67-26 | I.67-27 | I.67-28 | I.67-29 | I.67-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 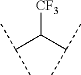 | 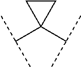 | CH₂ | CH₂ | CH₂ | CH₂ | CH₂ | CH₂ | CH₂ | CH₂ |

| No. | I.67-31 | I.67-32 | I.67-33 | I.67-34 | I.67-35 | I.67-36 | I.67-37 | I.67-38 | I.67-39 | I.67-40 | I.67-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 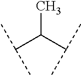 | CH₂ | CH₂ | CH₂ |

| No. | I.67-42 | I.67-43 | I.67-44 | I.67-45 | I.67-46 | I.67-47 | I.67-48 | I.67-49 | I.67-50 | I.67-51 | I.67-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 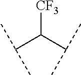 | CH₂ | CH₂ | CH₂ | CH₂ | CH₂ | CH₂ |

| No. | I.67-53 | I.67-54 | I.67-55 | I.67-56 | I.67-57 | I.67-58 | I.67-59 | I.67-60 | I.67-61 | I.67-62 | I.67-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 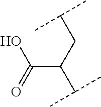 | CH₂ | CH₂ | CH₂ | CH₂ | CH₂ |

TABLE 68
with base structure I.68 and the radical definitions specified hereinafter:

| No. | I.68-1 | I.68-2 | I.68-3 | I.68-4 | I.68-5 | I.68-6 | I.68-7 | I.68-8 | I.68-9 | I.68-10 | I.68-11 | I.68-12 | I.68-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.68-14 | I.68-15 | I.68-16 | I.68-17 | I.68-18 | I.68-19 | I.68-20 |
|---|---|---|---|---|---|---|---|
| Q | Q-14 | Q-1 | Q-6 | Q-1 | Q-1 | Q-12 | Q-1 |
| X | H | H | H | c-Pr | 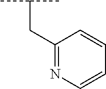 | CH₃ | CH₃ |
| Y | CH₂ | 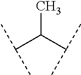 | 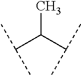 | CH₂ | CH₂ | CH₂ | 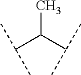 |

| No. | I.68-21 | I.68-22 | I.68-23 | I.68-24 | I.68-25 | I.68-26 | I.68-27 | I.68-28 | I.68-29 | I.68-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 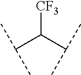 | 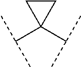 | CH₂ | CH₂ | CH₂ | CH₂ | CH₂ | CH₂ | CH₂ | CH₂ |

| No. | I.68-31 | I.68-32 | I.68-33 | I.68-34 | I.68-35 | I.68-36 | I.68-37 | I.68-38 | I.68-39 | I.68-40 | I.68-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 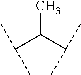 | CH₂ | CH₂ | CH₂ |

| No. | I.68-42 | I.68-43 | I.68-44 | I.68-45 | I.68-46 | I.68-47 | I.68-48 | I.68-49 | I.68-50 | I.68-51 | I.68-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 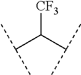 | CH₂ | CH₂ | CH₂ | CH₂ | CH₂ | CH₂ |

| No. | I.68-53 | I.68-54 | I.68-55 | I.68-56 | I.68-57 | I.68-58 | I.68-59 | I.68-60 | I.68-61 | I.68-62 | I.68-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 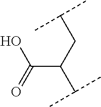 | CH₂ | CH₂ | CH₂ | CH₂ | CH₂ |

TABLE 69
with base structure I.69 and the radical definitions specified hereinafter:

| No. | I.69-1 | I.69-2 | I.69-3 | I.69-4 | I.69-5 | I.69-6 | I.69-7 | I.69-8 | I.69-9 | I.69-10 | I.69-11 | I.69-12 | I.69-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.69-14 | I.69-15 | I.69-16 | I.69-17 | I.69-18 | I.69-19 | I.69-20 |
|---|---|---|---|---|---|---|---|
| Q | Q-14 | Q-1 | Q-6 | Q-1 | Q-1 | Q-12 | Q-1 |
| X | H | H | H | c-Pr | 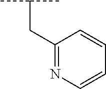 | CH₃ | CH₃ |
| Y | CH₂ | 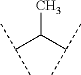 | 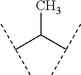 | CH₂ | CH₂ | CH₂ | 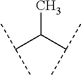 |

| No. | I.69-21 | I.69-22 | I.69-23 | I.69-24 | I.69-25 | I.69-26 | I.69-27 | I.69-28 | I.69-29 | I.69-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 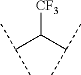 | 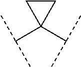 | CH₂ | CH₂ | CH₂ | CH₂ | CH₂ | CH₂ | CH₂ | CH₂ |

| No. | I.69-31 | I.69-32 | I.69-33 | I.69-34 | I.69-35 | I.69-36 | I.69-37 | I.69-38 | I.69-39 | I.69-40 | I.69-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 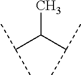 | CH₂ | CH₂ | CH₂ |

| No. | I.69-42 | I.69-43 | I.69-44 | I.69-45 | I.69-46 | I.69-47 | I.69-48 | I.69-49 | I.69-50 | I.69-51 | I.69-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 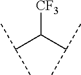 | CH₂ | CH₂ | CH₂ | CH₂ | CH₂ | CH₂ |

| No. | I.69-53 | I.69-54 | I.69-55 | I.69-56 | I.69-57 | I.69-58 | I.69-59 | I.69-60 | I.69-61 | I.69-62 | I.69-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 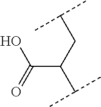 | CH₂ | CH₂ | CH₂ | CH₂ | CH₂ |

TABLE 70
with base structure I.70 and the radical definitions specified hereinafter:

| No. | I.70-1 | I.70-2 | I.70-3 | I.70-4 | I.70-5 | I.70-6 | I.70-7 | I.70-8 | I.70-9 | I.70-10 | I.70-11 | I.70-12 | I.70-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.70-14 | I.70-15 | I.70-16 | I.70-17 | I.70-18 | I.70-19 | I.70-20 |
|---|---|---|---|---|---|---|---|
| Q | Q-14 | Q-1 | Q-6 | Q-1 | Q-1 | Q-12 | Q-1 |
| X | H | H | H | c-Pr | 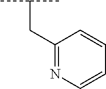 | CH₃ | CH₃ |
| Y | CH₂ | 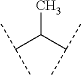 | 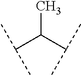 | CH₂ | CH₂ | CH₂ | 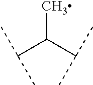 |

| No. | I.70-21 | I.70-22 | I.70-23 | I.70-24 | I.70-25 | I.70-26 | I.70-27 | I.70-28 | I.70-29 | I.70-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 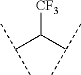 | 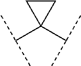 | CH₂ | CH₂ | CH₂ | CH₂ | CH₂ | CH₂ | CH₂ | CH₂ |

| No. | I.70-31 | I.70-32 | I.70-33 | I.70-34 | I.70-35 | I.70-36 | I.70-37 | I.70-38 | I.70-39 | I.70-40 | I.70-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 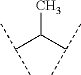 | CH₂ | CH₂ | CH₂ |

| No. | I.70-42 | I.70-43 | I.70-44 | I.70-45 | I.70-46 | I.70-47 | I.70-48 | I.70-49 | I.70-50 | I.70-51 | I.70-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 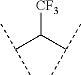 | CH₂ | CH₂ | CH₂ | CH₂ | CH₂ | CH₂ |

| No. | I.70-53 | I.70-54 | I.70-55 | I.70-56 | I.70-57 | I.70-58 | I.70-59 | I.70-60 | I.70-61 | I.70-62 | I.70-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 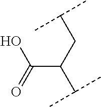 | CH₂ | CH₂ | CH₂ | CH₂ | CH₂ |

TABLE 71
with base structure I.71 and the radical definitions specified hereinafter:

| No. | I.71-1 | I.71-2 | I.71-3 | I.71-4 | I.71-5 | I.71-6 | I.71-7 | I.71-8 | I.71-9 | I.71-10 | I.71-11 | I.71-12 | I.71-13 |
|---|---|---|---|---|---|---|---|---|---|---|---|---|---|
| Q | Q-1 | Q-2 | Q-3 | Q-4 | Q-5 | Q-6 | Q-7 | Q-9 | Q-10 | Q-11 | Q-1 | Q-12 | Q-13 |
| X | H | H | H | H | H | H | H | H | H | H | CH₃ | H | H |
| Y | CH₂ | CH₂ | CH₂ | CH₂ | CH₂ | CH₂ | CH₂ | CH₂ | CH₂ | CH₂ | CH₂ | CH₂ | CH₂ |

| No. | I.71-14 | I.71-15 | I.71-16 | I.71-17 | I.71-18 | I.71-19 | I.71-20 |
|---|---|---|---|---|---|---|---|
| Q | Q-14 | Q-1 | Q-6 | Q-1 | Q-1 | Q-12 | Q-1 |
| X | H | H | H | c-Pr | 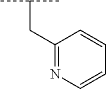 | CH₃ | CH₃ |
| Y | CH₂ | 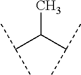 | 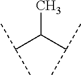 | CH₂ | CH₂ | CH₂ | 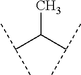 |

| No. | I.71-21 | I.71-22 | I.71-23 | I.71-24 | I.71-25 | I.71-26 | I.71-27 | I.71-28 | I.71-29 | I.71-30 |
|---|---|---|---|---|---|---|---|---|---|---|
| Q | Q-1 | Q-1 | Q-17 | Q-8 | Q-37 | Q-38 | Q-1 | Q-25 | Q-81 | Q-136 |
| X | H | H | H | H | H | H | i-Pr | H | CH₃ | H |
| Y | 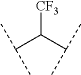 | 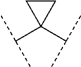 | CH₂ | CH₂ | CH₂ | CH₂ | CH₂ | CH₂ | CH₂ | CH₂ |

| No. | I.71-31 | I.71-32 | I.71-33 | I.71-34 | I.71-35 | I.71-36 | I.71-37 | I.71-38 | I.71-39 | I.71-40 | I.71-41 |
|---|---|---|---|---|---|---|---|---|---|---|---|
| Q | Q-26 | Q-27 | Q-29 | Q-31 | Q-32 | Q-33 | Q-34 | Q-31 | Q-52 | Q-79 | Q-80 |
| X | H | H | H | H | H | H | H | H | H | H | H |
| Y | CH₂ | CH₂ | CH₂ | CH₂ | CH₂ | CH₂ | CH₂ | 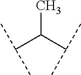 | CH₂ | CH₂ | CH₂ |

| No. | I.71-42 | I.71-43 | I.71-44 | I.71-45 | I.71-46 | I.71-47 | I.71-48 | I.71-49 | I.71-50 | I.71-51 | I.71-52 |
|---|---|---|---|---|---|---|---|---|---|---|---|
| Q | Q-81 | Q-82 | Q-83 | Q-84 | Q-79 | Q-57 | Q-58 | Q-60 | Q-64 | Q-78 | Q-77 |
| X | H | H | H | H | H | H | H | H | H | H | H |
| Y | CH₂ | CH₂ | CH₂ | CH₂ | 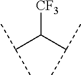 | CH₂ | CH₂ | CH₂ | CH₂ | CH₂ | CH₂ |

| No. | I.71-53 | I.71-54 | I.71-55 | I.71-56 | I.71-57 | I.71-58 | I.71-59 | I.71-60 | I.71-61 | I.71-62 | I.71-63 |
|---|---|---|---|---|---|---|---|---|---|---|---|
| Q | Q-113 | Q-114 | Q-109 | Q-110 | Q-51 | Q-134 | Q-109 | Q-139 | Q-140 | Q-149 | Q-141 |
| X | H | H | H | H | H | H | CH₃ | H | H | H | H |
| Y | CH₂ | CH₂ | CH₂ | CH₂ | CH₂ | 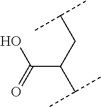 | CH₂ | CH₂ | CH₂ | CH₂ | CH₂ |

TABLE 72
with base structure I.72 and the radical definitions specified hereinafter:

| No. | I.72-1 | I.72-2 | I.72-3 | I.72-4 | I.72-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.72-6 | I.72-7 | I.72-8 | I.72-9 | I.72-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.72-11 | I.72-12 | I.72-13 | I.72-14 | I.72-15 |
| Q | Q-1 | Q-12 | Q-13 | Q-14 | Q-1 |
| X | CH₃ | H | H | H | H |
| Y | CH₂ | CH₂ | CH₂ | CH₂ | 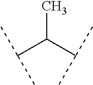 |
| No. | I.72-16 | I.71-17 | I.72-18 | I.72-19 | I.72-20 |
| Q | Q-6 | Q-1 | Q-1 | Q-12 | Q-1 |
| X | H | c-Pr | 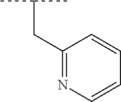 | CH₃ | CH₃ |
| Y | 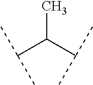 | CH₂ | CH₂ | CH₂ | 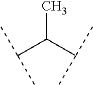 |
| No. | I.72-21 | I.72-22 | I.72-23 | I.72-24 | I.72-25 |
| Q | Q-1 | Q-1 | Q-17 | Q-8 | Q-37 |
| X | H | H | H | H | H |
| Y | 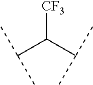 | 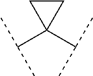 | CH₂ | CH₂ | CH₂ |
| No. | I.72-26 | I.72-27 | I.72-28 | I.72-29 | I.72-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.72-31 | I.72-32 | I.72-33 | I.72-34 | I.72-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.72-36 | I.72-37 | I.72-38 | I.72-39 | I.72-40 |
| Q | Q-33 | Q-34 | Q-31 | Q-52 | Q-79 |
| X | H | H | H | H | H |
| Y | CH₂ | CH₂ | 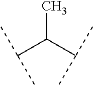 | CH₂ | CH₂ |
| No. | I.72-41 | I.72-42 | I.72-43 | I.72-44 | I.72-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.72-46 | I.72-47 | I.72-48 | I.72-49 | I.72-50 |
| Q | Q-79 | Q-57 | Q-58 | Q-60 | Q-64 |
| X | H | H | H | H | H |
| Y | 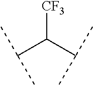 | CH₂ | CH₂ | CH₂ | CH₂ |
| No. | I.72-51 | I.72-52 | I.72-53 | I.72-54 | I.72-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.72-56 | I.72-57 | I.72-58 | I.72-59 | I.72-60 |
| Q | Q-110 | Q-51 | Q-134 | Q-109 | Q-139 |
| X | H | H | H | CH₃ | H |
| Y | CH₂ | CH₂ | 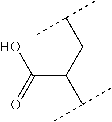 | CH₂ | CH₂ |
| | | No. | I.72-61 | I.72-62 | I.72-63 |
| | | Q | Q-140 | Q-149 | Q-141 |
| | | X | H | H | H |
| | | Y | CH₂ | CH₂ | CH₂ |

TABLE 73
with base structure I.73 and the radical definitions specified hereinafter:

| No. | I.73-1 | I.73-2 | I.73-3 | I.73-4 | I.73-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.73-6 | I.73-7 | I.73-8 | I.73-9 | I.73-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.73-11 | I.73-12 | I.73-13 | I.73-14 | I.73-15 |
| Q | Q-1 | Q-12 | Q-13 | Q-14 | Q-1 |
| X | CH₃ | H | H | H | H |
| Y | CH₂ | CH₂ | CH₂ | CH₂ | 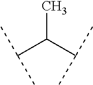 |
| No. | I.73-16 | I.73-17 | I.71-8 | I.73-19 | I.73-20 |
| Q | Q-6 | Q-1 | Q-1 | Q-12 | Q-1 |
| X | H | c-P | 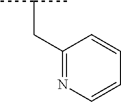 | CH₃ | CH₃ |
| Y | 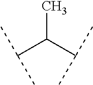 | CH₂ | CH₂ | CH₂ | 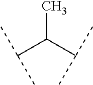 |
| No. | I.73-21 | I.73-22 | I.73-23 | I.73-24 | I.73-25 |
| Q | Q-1 | Q-1 | Q-17 | Q-8 | Q-37 |
| X | H | H | H | H | H |
| Y | 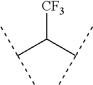 | 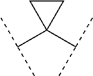 | CH₂ | CH₂ | CH₂ |
| No. | I.73-26 | I.73-37 | I.73-28 | I.73-29 | I.73-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.73-31 | I.73-32 | I.73-33 | I.73-34 | I.73-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.73-36 | I.73-37 | I.73-38 | I.73-39 | I.73-40 |
| Q | Q-33 | Q-34 | Q-31 | Q-52 | Q-79 |
| X | H | H | H | H | H |
| Y | CH₂ | CH₂ | 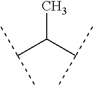 | CH₂ | CH₂ |
| No. | I.73-41 | I.73-42 | I.73-43 | I.73-44 | I.73-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.73-46 | I.73-47 | I.73-48 | I.73-49 | I.73-50 |
| Q | Q-79 | Q-57 | Q-58 | Q-60 | Q-64 |
| X | H | H | H | H | H |
| Y | 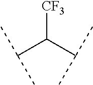 | CH₂ | CH₂ | CH₂ | CH₂ |
| No. | I.73-51 | I.73-52 | I.73-53 | I.73-54 | I.73-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.73-56 | I.73-57 | I.73-58 | I.73-59 | I.73-60 |
| Q | Q-110 | Q-51 | Q-134 | Q-109 | Q-139 |
| X | H | H | H | CH₃ | H |
| Y | CH₂ | CH₂ | 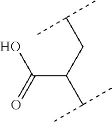 | CH₂ | CH₂ |
| | | No. | I.73-61 | I.73-62 | I.73-63 |
| | | Q | 1-140 | Q-149 | Q-141 |
| | | X | H | H | H |
| | | Y | CH₂ | CH₂ | CH₂ |

TABLE 74
with base structure I.74 and the radical definitions specified hereinafter:

| No. | I.74-1 | I.74-2 | I.74-3 | I.74-4 | I.74-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.74-6 | I.74-7 | I.74-8 | I.74-9 | I.74-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.74-11 | I.74-12 | I.74-13 | I.74-14 | I.74-15 |
| Q | Q-1 | Q-12 | Q-13 | Q-14 | Q-1 |
| X | CH₃ | H | H | H | H |
| Y | CH₂ | CH₂ | CH₂ | CH₂ | 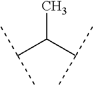 |
| No. | I.74-16 | I.74-17 | I.74-18 | I.74-19 | I.74-20 |
| Q | Q-6 | Q-1 | Q-1 | Q-12 | Q-1 |
| X | H | c-Pr | 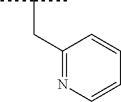 | CH₃ | CH₃ |
| Y | 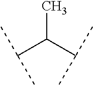 | CH₂ | CH₂ | CH₂ | 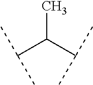 |
| No. | I.74-21 | I.74-22 | I.74-23 | I.74-24 | I.74-25 |
| Q | Q-1 | Q-1 | Q-17 | Q-8 | Q-37 |
| X | H | H | H | H | H |
| Y | 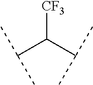 | 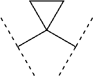 | CH₂ | CH₂ | CH₂ |
| No. | I.74-26 | I.74-27 | I.74-28 | I.74-29 | I.74-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.74-31 | I.74-32 | I.74-33 | I.74-34 | I.74-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.74-36 | I.74-37 | I.74-38 | I.74-39 | I.74-40 |
| Q | Q-33 | Q-34 | Q-31 | Q-52 | Q-79 |
| X | H | H | H | H | H |
| Y | CH₂ | CH₂ | 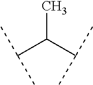 | CH₂ | CH₂ |
| No. | I.74-41 | I.74-42 | I.74-43 | I.74-44 | I.74-45 |
| Q | Q-81 | Q-81 | Q-82 | Q-83 | Q-84 |
| X | H | H | H | H | H |
| Y | CH₂ | CH₂ | CH₂ | CH₂ | CH₂ |
| No. | I.74-46 | I.74-47 | I.74-48 | I.74-49 | I.74-50 |
| Q | Q-79 | Q-57 | Q-58 | Q-60 | Q-64 |
| X | H | H | H | H | H |
| Y | 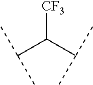 | CH₂ | CH₂ | CH₂ | CH₂ |
| No. | I.74-51 | I.74-52 | I.74-53 | I.74-54 | I.74-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.74-56 | I.74-57 | I.74-58 | I.74-59 | I.74-60 |
| Q | Q-110 | Q-51 | Q-134 | Q-109 | Q-139 |
| X | H | H | H | CH₃ | H |
| Y | CH₂ | CH₂ | 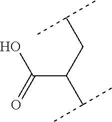 | CH₂ | CH₂ |
| | | No. | I.74-61 | I.74-62 | I.74-63 |
| | | Q | Q-140 | Q-149 | Q-141 |
| | | X | H | H | H |
| | | Y | CH₂ | CH₂ | CH₂ |

TABLE 75
with base structure I.75 and the radical definitions specified hereinafter:

| No. | I.75-1 | I.75-2 | I.75-3 | I.75-4 | I.75-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.75-6 | I.75-7 | I.75-8 | I.75-9 | I.75-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.75-11 | I.75-12 | I.75-13 | I.75-14 | I.75-15 |
| Q | Q-1 | Q-12 | Q-13 | Q-14 | Q-1 |
| X | CH₃ | H | H | H | H |
| Y | CH₂ | CH₂ | CH₂ | CH₂ | 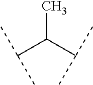 |
| No. | I.75-16 | I.75-17 | I.75-18 | I.75-19 | I.75-20 |
| Q | Q-6 | Q-1 | Q-1 | Q-12 | Q-1 |
| X | H | c-Pr | 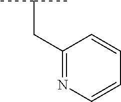 | CH₃ | CH₃ |
| Y | 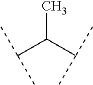 | CH₂ | CH₂ | CH₂ | 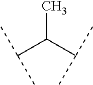 |
| No. | I.75-21 | I.75-22 | I.75-23 | I.75-24 | I.75-25 |
| Q | Q-1 | Q-1 | Q-17 | Q-8 | Q-37 |
| X | H | H | H | H | H |
| Y | 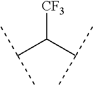 | 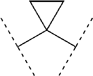 | CH₂ | CH₂ | CH₂ |
| No. | I.75-26 | I.75-27 | I.75-28 | I.75-29 | I.75-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.75-31 | I.75-32 | I.75-33 | I.75-34 | I.75-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.75-36 | I.75-37 | I.75-38 | I.75-39 | I.75-40 |
| Q | Q-33 | Q-34 | Q-31 | Q-52 | Q-79 |
| X | H | H | H | H | H |
| Y | CH₂ | CH₂ | 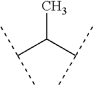 | CH₂ | CH₂ |
| No. | I.75-41 | I.75-42 | I.75-43 | I.75-44 | I.75-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.75-46 | I.75-47 | I.75-48 | I.75-49 | I.75-50 |
| Q | Q-79 | Q-57 | Q-58 | Q-60 | Q-64 |
| X | H | H | H | H | H |
| Y | 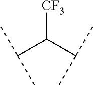 | CH₂ | CH₂ | CH₂ | CH₂ |
| No. | I.75-51 | I.75-52 | I.75-53 | I.75-54 | I.75-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.75-56 | I.75-57 | I.75-58 | I.75-59 | I.75-60 |
| Q | Q-110 | Q-51 | Q-134 | Q-109 | Q-139 |
| X | H | H | H | CH₃ | H |
| Y | CH₂ | CH₂ | 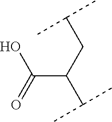 | CH₂ | CH₂ |
| | | No. | I.75-61 | I.75-62 | I.75-63 |
| | | Q | Q-140 | Q-149 | Q-141 |
| | | X | H | H | H |
| | | Y | CH₂ | CH₂ | CH₂ |

TABLE 76
with base structure I.76 and the radical definitions specified hereinafter:

| No. | I.76-1 | I.76-2 | I.76-3 | I.76-4 | I.76-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.76-6 | I.76-7 | I.76-8 | I.76-9 | I.76-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.76-11 | I.76-12 | I.76-13 | I.76-14 | I.76-15 |
| Q | Q-1 | Q-12 | Q-13 | Q-14 | Q-1 |
| X | CH₃ | H | H | H | H |
| Y | CH₂ | CH₂ | CH₂ | CH₂ | 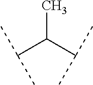 |
| No. | I.76-16 | I.76-17 | I.76-18 | I.76-19 | I.76-20 |
| Q | Q-6 | Q-1 | Q-1 | Q-12 | Q-1 |
| X | H | c-Pr | 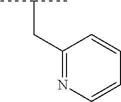 | CH₃ | CH₃ |
| Y | 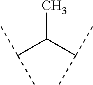 | CH₂ | CH₂ | CH₂ | 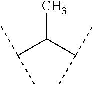 |
| No. | I.76-21 | I.76-22 | I.76-23 | I.76-24 | I.76-25 |
| Q | Q-1 | Q-1 | Q-17 | Q-8 | Q-37 |
| X | H | H | H | H | H |
| Y | 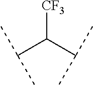 | 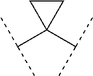 | CH₂ | CH₂ | CH₂ |
| No. | I.76-26 | I.76-27 | I.76-28 | I.76-29 | I.76-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.76-31 | I.76-32 | I.76-33 | I.76-34 | I.76-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.76-36 | I.76-37 | I.76-38 | I.76-39 | I.76-40 |
| Q | Q-33 | Q-34 | Q-31 | Q-52 | Q-79 |
| X | H | H | H | H | H |
| Y | CH₂ | CH₂ | 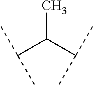 | CH₂ | CH₂ |
| No. | I.76-41 | I.76-42 | I.76-43 | I.76-44 | I.76-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.76-46 | I.76-47 | I.76-48 | I.76-49 | I.76-50 |
| Q | Q-79 | Q-57 | Q-58 | Q-60 | Q-64 |
| X | H | H | H | H | H |
| Y | 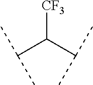 | CH₂ | CH₂ | CH₂ | CH₂ |
| No. | I.76-51 | I.76-52 | I.76-53 | I.76-54 | I.76-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.76-56 | I.76-57 | I.76-58 | I.76-59 | I.76-60 |
| Q | Q-110 | Q-51 | Q-134 | Q-109 | Q-139 |
| X | H | H | H | CH₃ | H |
| Y | CH₂ | CH₂ | 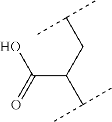 | CH₂ | CH₂ |
| | | No. | I.76-61 | I.76-62 | I.76-63 |
| | | Q | Q-140 | Q-149 | Q-141 |
| | | X | H | H | H |
| | | Y | CH₂ | CH₂ | CH₂ |

TABLE 77
with base structure I.77 and the radical definitions specified hereinafter:

| No. | I.77-1 | I.77-2 | I.77-3 | I.77-4 | I.77-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.77-6 | I.77-7 | I.77-8 | I.77-9 | I.77-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.77-11 | I.77-12 | I.77-13 | I.77-14 | I.77-15 |
| Q | Q-1 | Q-12 | Q-13 | Q-14 | Q-1 |
| X | CH₃ | H | H | H | H |
| Y | CH₂ | CH₂ | CH₂ | CH₂ | 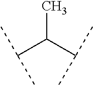 |
| No. | I.77-16 | I.77-17 | I.77-18 | I.77-19 | I.77-20 |
| Q | Q-6 | Q-1 | Q-1 | Q-12 | Q-1 |
| X | H | c-Pr | 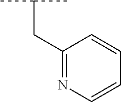 | CH₃ | CH₃ |
| Y | 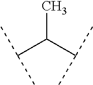 | CH₂ | CH₂ | CH₂ | 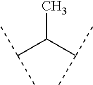 |
| No. | I.77-21 | I.77-22 | I.77-23 | I.77-24 | I.77-25 |
| Q | Q-1 | Q-1 | Q-17 | Q-8 | Q-37 |
| X | H | H | H | H | H |
| Y | 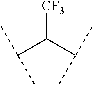 | 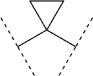 | CH₂ | CH₂ | CH₂ |
| No. | I.77-26 | I.77-27 | I.77-28 | I.77-29 | I.77-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.77-31 | I.77-32 | I.77-33 | I.77-34 | I.77-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.77-36 | I.77-37 | I.77-38 | I.77-39 | I.77-40 |
| Q | Q-33 | Q-34 | Q-31 | Q-52 | Q-79 |
| X | H | H | H | H | H |
| Y | CH₂ | CH₂ | 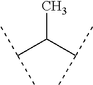 | CH₂ | CH₂ |
| No. | I.77-41 | I.77-42 | I.77-43 | I.77-44 | I.77-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.77-46 | I.77-47 | I.77-48 | I.77-49 | I.77-50 |
| Q | Q-79 | Q-57 | Q-58 | Q-60 | Q-64 |
| X | H | H | H | H | H |
| Y | 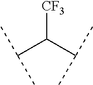 | CH₂ | CH₂ | CH₂ | CH₂ |
| No. | I.77-51 | I.77-52 | I.77-53 | I.77-54 | I.77-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.77-56 | I.77-57 | I.77-58 | I.77-59 | I.77-60 |
| Q | Q-110 | Q-51 | Q-134 | Q-109 | Q-139 |
| X | H | H | H | CH₃ | H |
| Y | CH₂ | CH₂ | 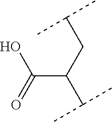 | CH₂ | CH₂ |
| | | No. | I.77-61 | I.77-62 | I.77-63 |
| | | Q | Q-140 | Q-149 | Q-141 |
| | | X | H | H | H |
| | | Y | CH₂ | CH₂ | CH₂ |

TABLE 78
with base structure I.78 and the radical definitions specified hereinafter:

| No. | I.78-1 | I.78-2 | I.78-3 | I.78-4 | I.78-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.78-6 | I.78-7 | I.78-8 | I.78-9 | I.78-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.78-11 | I.78-12 | I.78-13 | I.78-14 | I.78-15 |
| Q | Q-1 | Q-12 | Q-13 | Q-14 | Q-1 |
| X | CH₃ | H | H | H | H |
| Y | CH₂ | CH₂ | CH₂ | CH₂ | 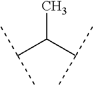 |
| No. | I.78-16 | I.78-17 | I.78-18 | I.78-19 | I.78-20 |
| Q | Q-6 | Q-1 | Q-1 | Q-12 | Q-1 |
| X | H | c-Pr | 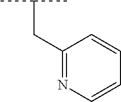 | CH₃ | CH₃ |
| Y | 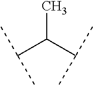 | CH₂ | CH₂ | CH₂ | 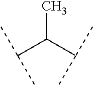 |
| No. | I.78-21 | I.78-22 | I.78-23 | I.78-24 | I.78-25 |
| Q | Q-1 | Q-1 | Q-17 | Q-8 | Q-37 |
| X | H | H | H | H | H |
| Y | 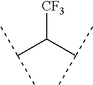 | 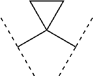 | CH₂ | CH₂ | CH₂ |
| No. | I.78-26 | I.78-27 | I.78-28 | I.78-29 | I.78-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.78-31 | I.78-32 | I.78-33 | I.78-34 | I.78-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.78-36 | I.78-37 | I.78-38 | I.78-39 | I.78-40 |
| Q | Q-33 | Q-34 | Q-31 | Q-52 | Q-79 |
| X | H | H | H | H | H |
| Y | CH₂ | CH₂ | 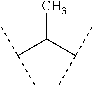 | CH₂ | CH₂ |
| No. | I.78-41 | I.78-42 | I.78-43 | I.78-44 | I.78-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.78-46 | I.78-47 | I.78-48 | I.78-49 | I.78-50 |
| Q | Q-79 | Q-57 | Q-58 | Q-60 | Q-64 |
| X | H | H | H | H | H |
| Y | 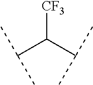 | CH₂ | CH₂ | CH₂ | CH₂ |
| No. | I.78-51 | I.78-52 | I.78-53 | I.78-54 | I.78-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.78-56 | I.78-57 | I.78-58 | I.78-59 | I.78-60 |
| Q | Q-110 | Q-51 | Q-134 | Q-109 | Q-139 |
| X | H | H | H | CH₃ | H |
| Y | CH₂ | CH₂ | 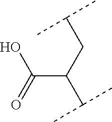 | CH₂ | CH₂ |
| | | No. | I.78-61 | I.78-62 | I.78-63 |
| | | Q | Q-140 | Q-149 | Q-141 |
| | | X | H | H | H |
| | | Y | CH₂ | CH₂ | CH₂ |

TABLE 79
with base structure I.79 and the radical definitions specified hereinafter:

| No. | I.79-1 | I.79-2 | I.79-3 | I.79-4 | I.79-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.79-6 | I.79-7 | I.79-8 | I.79-9 | I.79-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.79-11 | I.79-12 | I.79-13 | I.79-14 | I.79-15 |
| Q | Q-1 | Q-12 | Q-13 | Q-14 | Q-1 |
| X | CH₃ | H | H | H | H |
| Y | CH₂ | CH₂ | CH₂ | CH₂ | 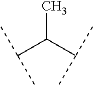 |
| No. | I.79-16 | I.79-17 | I.79-18 | I.79-19 | I.79-20 |
| Q | Q-6 | Q-1 | Q-1 | Q-12 | Q-1 |
| X | H | c-Pr | 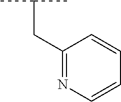 | CH₃ | CH₃ |
| Y | 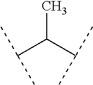 | CH₂ | CH₂ | CH₂ | 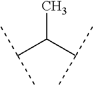 |
| No. | I.79-21 | I.79-22 | I.79-23 | I.79-24 | I.79-25 |
| Q | Q-1 | Q-1 | Q-17 | Q-8 | Q-37 |
| X | H | H | H | H | H |
| Y | 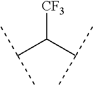 | 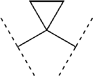 | CH₂ | CH₂ | CH₂ |
| No. | I.79-26 | I.79-27 | I.79-28 | I.79-29 | I.79-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.79-31 | I.79-32 | I.79-33 | I.79-34 | I.79-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.79-36 | I.79-37 | I.79-38 | I.79-39 | I.79-40 |
| Q | Q-33 | Q-34 | Q-31 | Q-52 | Q-79 |
| X | H | H | H | H | H |
| Y | CH₂ | CH₂ | 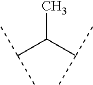 | CH₂ | CH₂ |
| No. | I.79-41 | I.79-42 | I.79-43 | I.79-44 | I.79-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.79-46 | I.79-47 | I.79-48 | I.79-49 | I.79-50 |
| Q | Q-79 | Q-57 | Q-58 | Q-60 | Q-64 |
| X | H | H | H | H | H |
| Y | 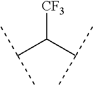 | CH₂ | CH₂ | CH₂ | CH₂ |
| No. | I.79-51 | I.79-52 | I.79-53 | I.79-54 | I.79-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.79-56 | I.79-57 | I.79-58 | I.79-59 | I.79-60 |
| Q | Q-110 | Q-51 | Q-134 | Q-109 | Q-139 |
| X | H | H | H | CH₃ | H |
| Y | CH₂ | CH₂ | 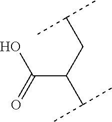 | CH₂ | CH₂ |
| | | No. | I.79-61 | I.79-62 | I.79-63 |
| | | Q | Q-140 | Q-149 | Q-141 |
| | | X | H | H | H |
| | | Y | CH₂ | CH₂ | CH₂ |

TABLE 80
with base structure I.80 and the radical definitions specified hereinafter:

| No. | I.80-1 | I.80-2 | I.80-3 | I.80-4 | I.80-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.80-6 | I.80-7 | I.80-8 | I.80-9 | I.80-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.80-11 | I.80-12 | I.80-13 | I.80-14 | I.80-15 |
| Q | Q-1 | Q-12 | Q-13 | Q-14 | Q-1 |
| X | CH₃ | H | H | H | H |
| Y | CH₂ | CH₂ | CH₂ | CH₂ | 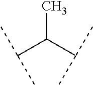 |
| No. | I.80-16 | I.80-17 | I.80-18 | I.80-19 | I.80-20 |
| Q | Q-6 | Q-1 | Q-1 | Q-12 | Q-1 |
| X | H | c-Pr | 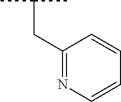 | CH₃ | CH₃ |
| Y | 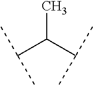 | CH₂ | CH₂ | CH₂ | 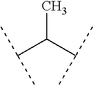 |
| No. | I.80-21 | I.80-22 | I.80-23 | I.80-24 | I.80-25 |
| Q | Q-1 | Q-1 | Q-17 | Q-8 | Q-37 |
| X | H | H | H | H | H |
| Y | 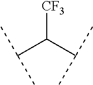 | 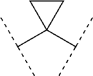 | CH₂ | CH₂ | CH₂ |
| No. | I.80-26 | I.80-27 | I.80-28 | I.80-29 | I.80-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.80-31 | I.80-32 | I.80-33 | I.80-34 | I.80-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.80-36 | I.80-37 | I.80-38 | I.80-39 | I.80-40 |
| Q | Q-33 | Q-34 | Q-31 | Q-52 | Q-79 |
| X | H | H | H | H | H |
| Y | CH₂ | CH₂ | 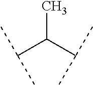 | CH₂ | CH₂ |
| No. | I.80-41 | I.80-42 | I.80-43 | I.80-44 | I.80-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.80-46 | I.80-47 | I.80-48 | I.80-49 | I.80-50 |
| Q | Q-79 | Q-57 | Q-58 | Q-60 | Q-64 |
| X | H | H | H | H | H |
| Y | 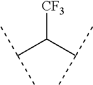 | CH₂ | CH₂ | CH₂ | CH₂ |
| No. | I.80-51 | I.80-52 | I.80-53 | I.80-54 | I.80-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.80-56 | I.80-57 | I.80-58 | I.80-59 | I.80-60 |
| Q | Q-110 | Q-51 | Q-134 | Q-109 | Q-139 |
| X | H | H | H | CH₃ | H |
| Y | CH₂ | CH₂ | 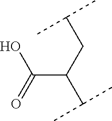 | CH₂ | CH₂ |
| | | No. | I.80-61 | I.80-62 | I.80-63 |
| | | Q | Q-140 | Q-149 | Q-141 |
| | | X | H | H | H |
| | | Y | CH₂ | CH₂ | CH₂ |

TABLE 81
with base structure I.81 and the radical definitions specified hereinafter:

| No. | I.81-1 | I.81-2 | I.81-3 | I.81-4 | I.81-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.81-6 | I.81-7 | I.81-8 | I.81-9 | I.81-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.81-11 | I.81-12 | I.81-13 | I.81-14 | I.81-15 |
| Q | Q-1 | Q-12 | Q-13 | Q-14 | Q-1 |
| X | H | H | H | H | H |
| Y | CH₂ | CH₂ | CH₂ | CH₂ | 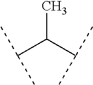 |
| No. | I.81-16 | I.81-17 | I.81-18 | I.81-19 | I.81-20 |
| Q | Q-6 | Q-1 | Q-1 | Q-12 | Q-1 |
| X | H | c-Pr | 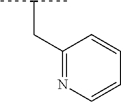 | CH₃ | CH₃ |
| Y | 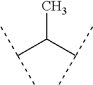 | CH₂ | CH₂ | CH₂ | 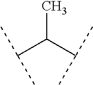 |
| No. | I.81-21 | I.81-22 | I.81-23 | I.81-24 | I.81-25 |
| Q | Q-1 | Q-1 | Q-17 | Q-8 | Q-37 |
| X | H | H | H | H | H |
| Y | 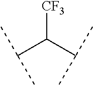 | 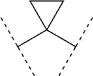 | CH₂ | CH₂ | CH₂ |
| No. | I.81-26 | I.81-27 | I.81-28 | I.81-29 | I.81-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.81-31 | I.81-32 | I.81-33 | I.81-34 | I.81-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.81-36 | I.81-37 | I.81-38 | I.81-39 | I.81-40 |
| Q | Q-33 | Q-34 | Q-31 | Q-52 | Q-79 |
| X | H | H | H | H | H |
| Y | CH₂ | CH₂ | 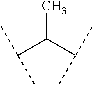 | CH₂ | CH₂ |
| No. | I.81-41 | I.81-42 | I.81-43 | I.81-44 | I.81-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.81-46 | I.81-47 | I.81-48 | I.81-49 | I.81-50 |
| Q | Q-79 | Q-57 | Q-58 | Q-60 | Q-64 |
| X | H | H | H | H | H |
| Y | 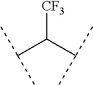 | CH₂ | CH₂ | CH₂ | CH₂ |
| No. | I.81-51 | I.81-52 | I.81-53 | I.81-54 | I.81-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.81-56 | I.81-57 | I.81-58 | I.81-59 | I.81-60 |
| Q | Q-110 | Q-51 | Q-134 | Q-109 | Q-139 |
| X | H | H | H | CH₃ | H |
| Y | CH₂ | CH₂ | 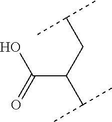 | CH₂ | CH₂ |
| | | No. | I.81-61 | I.81-62 | I.81-63 |
| | | Q | Q-140 | Q-149 | Q-141 |
| | | X | H | H | H |
| | | Y | CH₂ | CH₂ | CH₂ |

TABLE 82
with base structure I.82 and the radical definitions specified hereinafter:

| No. | I.82-1 | I.82-2 | I.82-3 | I.82-4 | I.82-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.82-6 | I.82-7 | I.82-8 | I.82-9 | I.82-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.82-11 | I.82-12 | I.82-13 | I.82-14 | I.82-15 |
| Q | Q-1 | Q-12 | Q-13 | Q-14 | Q-1 |
| X | CH₃ | H | H | H | H |
| Y | CH₂ | CH₂ | CH₂ | CH₂ | 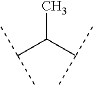 |
| No. | I.82-16 | I.82-17 | I.82-18 | I.82-19 | I.82-20 |
| Q | Q-6 | Q-1 | Q-1 | Q-12 | Q-1 |
| X | H | c-Pr | 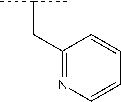 | CH₃ | CH₃ |
| Y | 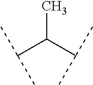 | CH₂ | CH₂ | CH₂ | 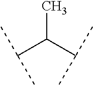 |
| No. | I.82-21 | I.82-22 | I.82-23 | I.82-24 | I.82-25 |
| Q | Q-1 | Q-1 | Q-17 | Q-8 | Q-37 |
| X | H | H | H | H | H |
| Y | 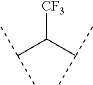 | 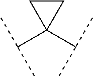 | CH₂ | CH₂ | CH₂ |
| No. | I.82-26 | I.82-27 | I.82-28 | I.82-29 | I.82-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.82-31 | I.82-32 | I.82-33 | I.82-34 | I.82-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.82-36 | I.82-37 | I.82-38 | I.82-39 | I.82-40 |
| Q | Q-33 | Q-34 | Q-31 | Q-52 | Q-79 |
| X | H | H | H | H | H |
| Y | CH₂ | CH₂ | 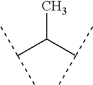 | CH₂ | CH₂ |
| No. | I.82-41 | I.82-42 | I.82-43 | I.82-44 | I.82-45 |
| Q | Q-80 | Q-81 | Q-82 | Q-83 | Q-84 |
| X | H | H | H | H | H |
| Y | CH₂ | CH₂ | CH₂ | CH₂ | CH₂ |
| No. | I.82-46 | I.82-47 | I.82-48 | I.82-49 | I.82-50 |
| Q | Q-79 | Q-57 | Q-58 | Q-60 | Q-64 |
| X | H | H | H | H | H |
| Y | 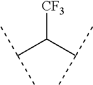 | CH₂ | CH₂ | CH₂ | CH₂ |
| No. | I.82-51 | I.82-52 | I.82-53 | I.82-54 | I.82-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.82-56 | I.82-57 | I.82-58 | I.82-59 | I.82-60 |
| Q | Q-110 | Q-51 | Q-134 | Q-109 | Q-139 |
| X | H | H | H | CH₃ | H |
| Y | CH₂ | CH₂ | 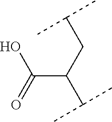 | CH₂ | CH₂ |
| | | No. | I.82-61 | I.82-62 | I.862-3 |
| | | Q | Q-140 | Q-149 | Q-141 |
| | | X | H | H | H |
| | | Y | CH₂ | CH₂ | CH₂ |

TABLE 83
with base structure I.83 and the radical definitions specified hereinafter:

| No. | I.83-1 | I.83-2 | I.83-3 | I.83-4 | I.83-5 |
|---|---|---|---|---|---|
| Q | Q-1 | Q-2 | Q-3 | Q-4 | Q-5 |
| X | H | H | H | H | H |
| Y | CH₂ | CH₂ | CH₂ | CH₂ | CH₂ |
| No. | I.83-6 | I.83-7 | I.83-8 | I.83-9 | I.83-10 |
| Q | Q-6 | Q-7 | Q-9 | Q-10 | Q-11 |
| X | H | H | H | H | H |
| Y | CH₂ | CH₂ | CH₂ | CH₂ | CH₂ |
| No. | I.83-11 | I.83-12 | I.83-13 | I.83-14 | I.83-15 |
| Q | Q-1 | Q-12 | Q-13 | Q-14 | Q-1 |
| X | CH₃ | H | H | H | H |
| Y | CH₂ | CH₂ | CH₂ | CH₂ | 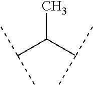 |
| No. | I.83-16 | I.83-17 | I.83-18 | I.83-19 | I.83-20 |
| Q | Q-6 | Q-1 | Q-1 | Q-12 | Q-1 |
| X | H | c-Pr | 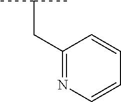 | CH₃ | CH₃ |
| Y | 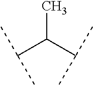 | CH₂ | CH₂ | CH₂ | 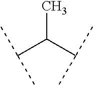 |
| No. | I.83-21 | I.83-22 | I.83-23 | I.83-24 | I.83-25 |
| Q | Q-1 | Q-1 | Q-17 | Q-8 | Q-37 |
| X | H | H | H | H | H |
| Y | 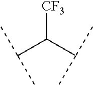 | 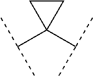 | CH₂ | CH₂ | CH₂ |
| No. | I.83-26 | I.83-27 | I.83-28 | I.83-29 | I.83-30 |
| Q | Q-38 | Q-1 | Q-25 | Q-81 | Q-136 |
| X | H | i-Pr | H | CH₃ | H |
| Y | CH₂ | CH₂ | CH₂ | CH₂ | CH₂ |
| No. | I.83-31 | I.83-32 | I.83-33 | I.83-34 | I.83-35 |
| Q | Q-26 | Q-27 | Q-29 | Q-31 | Q-32 |
| X | H | H | H | H | H |
| Y | CH₂ | CH₂ | CH₂ | CH₂ | CH₂ |
| No. | I.83-36 | I.83-37 | I.83-38 | I.83-39 | I.83-40 |
| Q | Q-33 | Q-34 | Q-31 | Q-52 | Q-79 |
| X | H | H | H | H | H |
| Y | CH₂ | CH₂ | 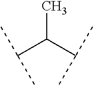 | CH₂ | CH₂ |
| No. | I.83-41 | I.83-42 | I.83-43 | I.83-44 | I.83-45 |
| X | Q-80 | Q-81 | Q-82 | Q-83 | Q-84 |
| Y | H | H | H | H | H |
| Y | CH₂ | CH₂ | CH₂ | CH₂ | CH₂ |
| No. | I.83-46 | I.83-47 | I.83-48 | I.83-49 | I.83-50 |
| Q | Q-79 | Q-57 | Q-58 | Q-60 | Q-64 |
| X | H | H | H | H | H |
| Y | 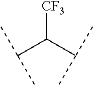 | CH₂ | CH₂ | CH₂ | CH₂ |
| No. | I.83-51 | I.83-52 | I.83-53 | I.83-54 | I.83-55 |
| Q | Q-78 | Q-77 | Q-113 | Q-114 | Q-109 |
| X | H | H | H | H | H |
| Y | CH₂ | CH₂ | CH₂ | CH₂ | CH₂ |
| No. | I.83-56 | I.83-57 | I.83-58 | I.83-59 | I.83-60 |
| Q | Q-110 | Q-51 | Q-134 | Q-109 | Q-139 |
| X | H | H | H | CH₃ | H |
| Y | CH₂ | CH₂ | 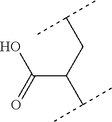 | CH₂ | CH₂ |
| | | No. | I.83-61 | I.83-62 | I.83-63 |
| | | Q | Q-140 | Q-149 | Q-141 |
| | | X | H | H | H |
| | | Y | CH₂ | CH₂ | CH₂ |

### Spectroscopic Data of the Chemical Examples:

The ¹H NMR, ¹³C NMR and ¹⁹F NMR spectroscopy data which are reported for the chemical examples described in the paragraphs which follow (400 MHz for ¹H NMR and 150 MHz for ¹³C NMR and 375 MHz for ¹⁹F NMR, solvent: CDCl₃, CD₃OD or d₆-DMSO, internal standard: tetramethylsilane δ=0.00 ppm), were obtained with a Bruker instrument, and the signals identified have the following definitions: br=broad; s=singlet, d=doublet, t=triplet, dd=double doublet, ddd=doublet of a double doublet, m=multiplet, q=quartet, quint=quintet, sext=sextet, sept=septet, t=triplet, dq=double quartet, dt=double triplet.

### Example No. I.1-1

¹H NMR (400 MHz, CDCl₃ δ, ppm) 8.44 (d, 1H), 7.71 (d, 2H), 7.62 (d, 1H), 7.57 (m, 2H), 7.17 (m, 2H), 6.05 (m, 1H, NH), 4.26 (d, 2H).

### Example No. I.1-4

¹H NMR (400 MHz, CDCl₃ δ, ppm) 8.09 (d, 1H), 7.97 (d, 1H), 7.74 (m, 4H), 7.52 (m, 3H), 7.21 (d, 1H), 6.46 (br. m, 1H, NH), 4.45 (s, 2H).

### Example No. I.1-6

¹H NMR (400 MHz, CDCl₃ δ, ppm) 8.70 (s, 1H), 7.87 (d, 1H), 7.70 (d, 2H), 7.59 (d, 2H), 7.32 (d, 1H), 5.81 (br. t, 1H, NH), 4.35 (d, 2H).

### Example No. I.1-7

¹H NMR (400 MHz, CDCl₃ δ, ppm) 8.62 (s, 1H), 7.88 (s, 1H), 7.74 (d, 2H), 7.58 (d, 2H), 6.15 (br. t, 1H, NH), 4.45 (d, 2H).

### Example No. I.1-9

¹H NMR (400 MHz, CDCl₃ δ, ppm) 8.21 (m, 1H), 7.72 (d, 2H), 7.59 (d, 2H), 7.15 (dd, 1H), 5.80 (br. t, 1H, NH), 4.33 (d, 2H).

### Example No. I.1-10

¹H NMR (400 MHz, CDCl₃ δ, ppm) 8.59 (s, 1H), 7.69 (d, 2H), 7.61 (d, 2H), 7.26 (s, 1H), 5.58 (br. t, 1H, NH), 4.34 (d, 2H).

### Example No. I.1-16

¹H NMR (400 MHz, CDCl₃ δ, ppm) 8.63 (s, 1H), 7.79 (d, 1H), 7.56 (d, 2H), 7.47 (d, 2H), 7.08 (d, 1H), 5.91 (br. d, 1H, NH), 4.59 (quint, 1H), 1.47 (d, 3H).

### Example No. I.1-31

¹H NMR (400 MHz, CDCl₃ δ, ppm) 8.31 (d, 1H), 7.68 (m, 3H), 7.61 (d, 2H), 7.19 (dd, 1H), 5.02 (br. t, 1H, NH), 4.28 (d, 2H).

### Example No. I.1-32

¹H NMR (400 MHz, CDCl₃ δ, ppm) 8.12 (d, 1H), 7.69 (d, 2H), 7.66 (d, 2H), 7.62 (dd, 1H), 7.31 (d, 1H), 4.74 (br. t, 1H, NH), 4.18 (d, 2H).

### Example No. I.1-38

¹H NMR (400 MHz, CDCl₃ δ, ppm) 8.49 (d, 2H), 7.58 (d, 2H), 7.55 (d, 2H), 7.11 (d, 2H), 4.88 (br. d, 1H, NH), 4.52 (quint, 1H), 1.41 (d, 3H).

### Example No. I.1-39

¹H NMR (400 MHz, CDCl₃ δ, ppm) 8.11 (d, 1H), 7.69 (d, 2H), 7.58 (d, 2H), 7.10 (dd, 1H), 7.06 (d, 1H), 5.82 (br. t, 1H, NH), 4.19 (d, 2H), 3.83 (s, 3H).

### Example No. I.1-46

¹H NMR (400 MHz, CDCl₃ δ, ppm) 7.64 (d, 2H), 7.58 (d, 2H), 7.31 (m, 1H), 6.30 (m, 2H), 5.36 (br. d, 1H, NH), 5.09 (m, 1H).

### Example No. I.1-52

¹H NMR (400 MHz, d₆-DMSO δ, ppm) 8.39 (br. t, 1H, NH), 7.77 (d, 2H), 7.66 (d, 2H), 5.99 (s, 1H), 4.03 (d, 2H), 2.30 (s, 3H).

### Example No. I.1-56

¹H NMR (400 MHz, CDCl₃ δ, ppm) 7.63 (d, 2H), 7.58 (d, 2H), 7.36 (s, 1H), 5.36 (br. t, 1H, NH), 4.38 (d, 2H).

### Example No. I.1-62

¹H NMR (400 MHz, d₆-DMSO, δ ppm) 8.71 (br. t, 1H, NH), 7.78 (m, 2H), 7.71 (d, 2H), 7.68 (d, 2H), 7.03 (m, 2H), 6.63 (s, 1H), 4.25 (d, 2H), 3.81 (s, 3H).

### Example No. I.2-1

¹H NMR (400 MHz, CDCl₃ δ, ppm) 8.69 (d, 1H), 8.26 (m, 2H), 7.98 (d, 1H), 7.87 (d, 1H), 7.67 (dd, 1H), 7.57 (dd, 1H), 7.44 (m, 2H), 6.99 (m, 2H), 6.17 (br. t, NH, 1H), 4.21 (d, 2H).

### Example No. I.2-6

¹H NMR (400 MHz, CDCl₃ δ, ppm) 8.63 (d, 1H), 8.39 (d, 1H), 8.22 (dd, 1H), 7.96 (d, 1H), 7.84 (d, 1H), 7.66 (m, 1H), 7.58 (m, 2H), 7.45 (dd, 1H), 7.04 (d, 1H), 6.00 (br. t, NH, 1H), 4.28 (d, 2H).

### Example No. I.2-7

¹H NMR (400 MHz, CDCl₃ δ, ppm) 8.67 (d, 1H), 8.28 (d, 1H), 8.19 (s, 1H), 7.92 (d, 1H), 7.81 (d, 1H), 7.68 (m, 1H), 7.60 (s, 1H), 7.56 (dd, 1H), 7.46 (dd, 1H), 6.28 (br. t, NH, 1H), 4.40 (d, 2H).

### Example No. I.2-11

¹H NMR (400 MHz, CDCl₃ δ, ppm) 8.78 (d, 1H), 8.49 (d, 1H), 8.23 (d, 1H), 8.08 (d, 1H), 7.94 (d, 1H), 7.66 (m, 2H), 7.61 (m, 1H), 7.55 (dd, 1H), 7.46 (d, 1H), 7.17 (dd, 1H), 4.53 (d, 2H), 2.82 (s, 3H).

### Example No. I.2-13

¹H NMR (400 MHz, CDCl₃ δ, ppm) 8.54 (d, 1H), 8.19 (m, 1H), 8.11 (d, 1H), 7.91 (d, 1H), 7.86 (d, 1H), 7.79 (dd, 1H), 7.48 (m, 2H), 6.90 (s, 1H), 6.70 (d, 1H), 5.75 (br. t, NH, 1H), 4.44 (d, 2H), 3.71 (s, 3H).

### Example No. I.2-14

¹H NMR (400 MHz, CDCl₃ δ, ppm) 8.62 (d, 1H), 8.22 (d, 1H), 7.99 (d, 1H), 7.87 (d, 1H), 7.69 (dd, 1H), 7.58 (dd, 1H), 7.46 (dd, 1H), 7.35 (dd, 1H), 6.97 (d, 1H), 6.88 (d, 1H), 5.89 (br. t, NH, 1H), 4.19 (d, 2H).

### Example No. I.2-15

¹H NMR (400 MHz, CDCl₃ δ, ppm) 8.59 (d, 1H), 8.30 (br. d, 1H, NH), 8.11 (d, 1H), 8.09 (m, 1H), 7.85 (d, 1H), 7.73 (m, 2H), 7.63 (dd, 1H), 7.56 (dd, 1H), 7.38 (dd, 1H), 7.18 (m, 2H), 4.69 (quint, 1H), 1.51 (d, 3H).

### Example No. I.2-16

¹H NMR (400 MHz, CDCl₃ δ, ppm) 8.55 (d, 1H), 8.13 (m, 2H), 7.83 (d, 1H), 7.75 (d, 1H), 7.63 (dd, 1H), 7.54 (dd, 1H), 7.37 (m, 2H), 6.75 (d, 1H), 6.08 (br. d, 1H, NH), 4.49 (quint, 1H), 1.41 (d, 3H).

### Example No. I.2-17

¹H NMR (400 MHz, CDCl₃ δ, ppm) 8.78 (d, 1H), 8.66 (m, 1H), 8.31 (d, 1H), 8.11 (d, 1H), 7.96 (m, 2H), 7.86 (m, 1H), 7.62 (dd, 1H), 7.58 (m, 2H), 7.46 (m, 1H), 4.81 (s, 2H), 2.32 (q, 1H), 0.52 (m, 4H).

### Example No. I.2-18

¹H NMR (400 MHz, CDCl₃ δ, ppm) 8.69 (d, 1H), 8.47 (d, 2H), 8.26 (d, 1H), 8.07 (d, 1H), 7.93 (d, 1H), 7.68 (m, 2H), 7.60 (m, 2H), 7.52 (dd, 1H), 7.41 (d, 2H), 7.26 (m, 2H), 4.84 (s, 4H).

### Example No. I.2-19

¹H NMR (400 MHz, CDCl₃ δ, ppm) 8.77 (d, 1H), 8.39 (m, 1H), 8.24 (d, 1H), 8.10 (d, 1H), 7.96 (d, 1H), 7.67 (dd, 1H), 7.60 (dd, 1H), 7.56 (dd, 1H), 7.43 (m, 1H), 7.21 (m, 1H), 4.51 (s, 2H), 2.85 (s, 3H).

### Example No. I.2-20

¹H NMR (400 MHz, CDCl₃ δ, ppm) 8.70 (d, 1H), 8.59 (m, 1H), 8.29 (d, 1H), 8.08 (d, 1H), 7.94 (d, 1H), 7.68 (dd, 1H), 7.63 (dd, 1H), 7.59 (dd, 1H), 7.55 (dd, 1H), 7.48 (m, 1H), 7.22 (m, 1H), 5.44 (q, 1H), 2.80 (s, 3H), 1.43 (d, 1H).

### Example No. I.2-21

¹H NMR (400 MHz, CDCl₃ δ, ppm) 8.64 (d, 1H), 8.20 (d, 1H), 8.16 (d, 1H), 7.91 (d, 1H), 7.79 (d, 1H), 7.66 (dd, 1H), 7.52 (dd, 1H), 7.40 (m, 2H), 7.05-6.97 (m, 3H), 4.86 (quint, 1H).

### Example No. I.2-25

¹H NMR (400 MHz, CDCl₃ δ, ppm) 8.56 (d, 1H), 8.23 (d, 1H), 7.98 (d, 1H), 7.86 (d, 1H), 7.62 (dd, 1H), 7.54 (dd, 1H), 7.46 (dd, 1H), 7.07-6.99 (m, 3H), 6.94 (m, 1H), 5.15 (br. t, 1H, NH), 4.20 (d, 2H).

### Example No. I.2-28

¹H NMR (400 MHz, CDCl₃ δ, ppm) 9.10 (m, 1H), 8.80 (m, 1H), 8.62 (m, 1H), 8.22 (m, 1H), 8.09 (d, 1H), 7.96 (d, 1H), 7.64 (m, 2H), 7.53 (m, 1H), 7.22 (m, 1H), 6.90 (m, 1H), 5.59 (br. t, 1H, NH), 4.66 (d, 2H).

### Example No. I.2-31

¹H NMR (400 MHz, CDCl₃ δ, ppm) 8.58 (d, 1H), 8.22 (d, 1H), 8.12 (d, 1H), 8.04 (d, 1H), 7.91 (d, 1H), 7.68 (dd, 1H), 7.60 (dd, 1H), 7.49 (m, 2H), 6.97 (m, 1H), 5.39 (br. t, 1H, NH), 4.21 (d, 2H).

### Example No. I.2-35

¹H NMR (400 MHz, CDCl₃ δ, ppm) 8.70 (m, 1H), 8.59 (d, 1H), 8.40 (m, 1H), 8.22 (d, 1H), 8.04 (d, 1H), 7.93 (d, 1H), 7.67 (dd, 1H), 7.62 (dd, 1H), 7.50 (dd, 1H), 7.30 (m, 1H), 5.59 (br. m, NH, 1H), 4.19 (4.22 (d, 2H).

### Example No. I.2-36

¹H NMR (400 MHz, CDCl₃ δ, ppm) 8.61 (d, 1H), 8.21 (d, 1H), 8.11 (d, 1H), 8.07 (d, 1H), 7.95 (d, 1H), 7.69 (dd, 1H), 7.61 (dd, 1H), 7.49 (dd, 1H), 6.97 (s, 1H), 6.90 (d, 1H), 5.39 (br. t, 1H, NH), 4.12 (d, 2H).

### Example No. I.2-38

¹H NMR (400 MHz, CDCl₃ δ, ppm) 8.56 (d, 1H), 8.16 (d, 2H), 8.06 (d, 1H), 7.96 (d, 1H), 7.88 (d, 1H), 7.64 (dd, 1H), 7.57 (dd, 1H), 7.36 (dd, 1H), 6.75 (d, 2H), 4.94 (br. d, 1H, NH), 4.43 (quint, 1H), 1.32 (d, 3H).

### Example No. I.2-39

¹H NMR (400 MHz, CDCl₃ δ, ppm) 8.68 (d, 1H), 8.23 (d, 1H), 7.98 (d, 1H), 7.91 (d, 1H), 7.88 (d, 1H), 7.66 (m, 1H), 7.57 (dd, 1H), 7.48 (dd, 1H), 6.96 (dd, 1H), 6.89 (d, 1H), 6.01 (br. t, 1H, NH), 4.15 (d, 2H), 3.76 (s, 3H).

### Example No. I.2-42

¹H NMR (400 MHz, CDCl₃ δ, ppm) 8.63 (d, 1H), 8.24 (d, 1H), 8.05 (d, 1H), 7.93 (d, 1H), 7.67 (dd, 1H), 7.58 (dd, 1H), 7.51 (dd, 1H), 4.96 (br. t, 1H, NH), 3.84 (m, 1H), 3.68 (m, 1H), 3.61 (m, 1H), 3.07 (m, 1H), 2.83 (m, 1H), 1.89-1.75 (m, 3H), 1.49 (m, 1H).

### Example No. I.2-48

¹H NMR (400 MHz, CDCl₃ δ, ppm) 8.64 (d, 1H), 8.27 (d, 1H), 8.04 (d, 1H), 7.91 (d, 1H), 7.63 (dd, 1H), 7.57 (dd, 1H), 7.50 (dd, 1H), 7.10 (d, 1H), 5.85 (d, 1H), 5.11 (br. t, 1H, NH), 4.12 (d, 2H), 3.82 (t, 2H), 1.68 (sext, 2H), 0.79 (t, 3H).

### Example No. I.2-49

¹H NMR (400 MHz, CDCl₃ δ, ppm) 8.59 (d, 1H), 8.28 (d, 1H), 8.00 (d, 1H), 7.87 (d, 1H), 7.63 (dd, 1H), 7.56 (dd, 1H), 7.49 (dd, 1H), 6.92 (s, 1H), 5.21 (br. t, 1H, NH), 4.12 (d, 2H), 3.50 (s, 3H).

### Example No. I.2-51

¹H NMR (400 MHz, CDCl₃ δ, ppm) 8.66 (d, 1H), 8.25 (d, 1H), 8.04 (d, 1H), 7.93 (d, 1H), 7.66 (dd, 1H), 7.56 (dd, 1H), 7.51 (dd, 1H), 3.02 (m, 1H), 2.88 (dd, 1H), 2.73 (dd, 1H), 2.38 (m, 1H), 2.13 (m, 1H), 1.99 (m, 1H), 1.84 (m, 1H), 1.68 (m, 1H), 1.65-1.48 (m, 4H), 0.75 (t, 3H).

### Example No. I.2-53

¹H NMR (400 MHz, CDCl₃ δ, ppm) 8.59 (d, 1H), 8.23 (d, 1H), 8.07 (d, 1H), 7.93 (d, 1H), 7.69 (dd, 1H), 7.60 (dd, 1H), 7.50 (dd, 1H), 5.40 (br. t, 1H, NH), 4.35 (d, 2H), 1.79 (m, 1H), 0.87 (m, 2H), 0.61 (m, 2H).

### Example No. I.2-55

¹H NMR (400 MHz, CDCl₃ δ, ppm) 8.57 (d, 1H), 8.20 (d, 1H), 7.99 (d, 1H), 7.88 (d, 1H), 7.64 (dd, 1H), 7.55 (dd, 1H), 7.45 (dd, 1H), 6.53 (s, 1H), 5.36 (br. t, 1H, NH), 4.18 (d, 2H), 2.34 (s, 3H).

### Example No. I.2-61

¹H NMR (400 MHz, CDCl₃ δ, ppm) 8.59 (d, 1H), 8.27 (d, 1H), 8.01 (d, 1H), 7.88 (d, 1H), 7.71 (dd, 1H), 7.60 (dd, 1H), 7.50 (dd, 1H), 7.48 (d, 2H), 7.38 (d, 2H), 6.00 (s, 1H), 5.25 (br. t, 1H, NH), 4.31 (d, 2H).

### Example No. I.2-62

¹H NMR (400 MHz, d₆-DMSO, δ ppm) 8.82 (br. t, 1H, NH), 8.64 (d, 1H), 8.17 (d, 1H), 8.13 (d, 1H), 8.04 (d, 1H), 7.72 (dd, 1H), 7.66 (dd, 1H), 7.60 (dd, 1H), 7.53 (d, 2H), 7.00 (d, 2H), 6.36 (s, 1H), 4.25 (d, 2H), 3.81 (s, 3H).

### Example No. I.3-1

¹H NMR (400 MHz, CDCl₃ δ, ppm) 8.72 (d, 1H), 8.31 (d, 1H), 8.22 (d, 1H), 8.08 (d, 1H), 7.80 (d, 1H), 7.69 (dd, 1H), 7.65 (dd, 1H), 7.44 (m, 1H), 7.01 (dd, 1H), 6.96 (d, 1H), 6.23 (br. t, 1H, NH), 4.22 (d, 2H).

### Example No. I.3-2

¹H NMR (400 MHz, CDCl₃ δ, ppm) 8.76 (d, 1H), 8.40 (d, 2H), 8.31 (d, 1H), 8.11 (d, 1H), 7.82 (d, 1H), 7.70 (dd, 1H), 7.65 (dd, 1H), 7.01 (t, 1H), 6.14 (br. t, 1H, NH), 4.37 (d, 2H).

### Example No. I.3-3

¹H NMR (400 MHz, CDCl₃ δ, ppm) 8.68 (d, 1H), 8.29 (d, 1H), 8.07 (d, 1H), 8.01 (d, 1H), 7.79 (d, 1H), 7.68 (dd, 1H), 7.64 (dd, 1H), 6.78 (d, 1H), 667 (s, 1H), 6.13 (br. t, 1H, NH), 4.16 (d, 2H), 2.18 (s, 3H).

### Example No. I.3-4

¹H NMR (400 MHz, CDCl₃ δ, ppm) 8.79 (d, 1H), 8.18 (d, 1H), 8.08 (d, 1H), 7.94 (d, 1H), 7.75 (d, 1H), 7.74-7.69 (m, 3H), 7.62 (dd, 1H), 7.54 (dd, 1H), 7.47 (dd, 1H), 7.03 (d, 1H), 6.68 (br. t, 1H, NH), 4.40 (d, 2H).

### Example No. I.3-5

¹H NMR (400 MHz, d₆-DMSO δ, ppm) 8.64 (m, 2H), 8.22 (d, 1H), 7.93 (s, 1H), 7.91 (d, 1H), 7.77 (m, 2H), 7.68 (s, 1H), 6.52 (m, 1H, NH), 4.10 (d, 2H), 3.69 (s, 3H), 3.40 (s, 3H).

### Example No. I.3-6

¹H NMR (400 MHz, CDCl₃ δ, ppm) 8.67 (d, 1H), 8.39 (s, 1H), 8.30 (d, 1H), 8.07 (d, 1H), 7.80 (d, 1H), 7.78-7.64 (m, 3H), 7.05 (d, 1H), 6.21 (br. t, 1H, NH), 4.28 (d, 2H).

### Example No. I.3-7

¹H NMR (400 MHz, CDCl₃ δ, ppm) 8.70 (d, 1H), 8.28 (d, 1H), 8.21 (s, 1H), 8.11 (d, 1H), 7.81 (d, 1H), 7.68 (m, 2H), 7.65 (s, 1H), 6.30 (br. t, 1H, NH), 4.39 (d, 2H).

### Example No. I.3-8

¹H NMR (400 MHz, CDCl₃ δ, ppm) 8.71 (d, 1H), 8.27 (d, 1H), 8.11 (d, 1H), 7.98 (m, 1H), 7.82 (d, 1H), 7.70 (dd, 1H), 7.66 (dd, 1H), 7.13 (dd, 1H), 7.02 (m, 1H), 6.25 (br. t, 1H, NH), 4.29 (d, 2H).

### Example No. I.3-9

¹H NMR (400 MHz, CDCl₃ δ, ppm) 8.66 (d, 1H), 8.30 (d, 1H), 8.09 (d, 1H), 7.81 (m, 2H), 7.66 (m, 2H), 6.88 (m, 1H), 5.98 (br. t, 1H, NH), 4.25 (d, 2H).

### Example No. I.3-10

¹H NMR (400 MHz, CDCl₃ δ, ppm) 8.66 (d, 1H), 8.34 (d, 1H), 8.26 (s; 1H), 8.06 (d, 1H), 7.80 (d, 1H), 7.71 (m, 2H), 7.16 (d, 1H), 5.81 (br. t, 1H, NH), 4.27 (d, 2H).

### Example No. I.3-11

¹H NMR (400 MHz, CDCl₃ δ, ppm) 8.81 (d, 1H), 8.40 (d, 1H), 8.09 (d, 1H), 7.89 (d, 1H), 7.78 (m, 1H), 7.70 (m, 4H), 7.18 (m, 2H), 4.58 (s, 2H), 2.85 (s, 3H).

### Example No. I.3-12

¹H NMR (400 MHz, CDCl₃ δ, ppm) 8.67 (d, 1H), 8.48 (dd, 1H), 8.30 (d, 1H), 8.18 (d, 1H), 8.08 (d, 1H), 7.84 (d, 1H), 7.69 (m, 2H), 7.04 (m, 1H), 6.04 (br. t, 1H, NH), 4.17 (d, 2H).

### Example No. I.3-14

¹H NMR (400 MHz, CDCl₃ δ, ppm) 8.65 (d, 1H), 8.30 (d, 1H), 8.04 (d, 1H), 7.78 (d, 1H), 7.73 (dd, 1H), 7.67 (dd, 1H), 7.34 (dd, 1H), 6.97 (d, 1H), 6.82 (d, 1H), 5.85 (br. t, 1H, NH), 4.18 (d, 2H).

### Example No. I.3-15

¹H NMR (400 MHz, CDCl₃ δ, ppm) 8.63 (d, 1H), 8.20 (d, 1H), 8.01 (m, 1H), 7.98 (d, 1H), 7.63 (m, 4H), 7.60 (dd, 1H), 7.10 (d, 1H), 6.97 (br. d, 1H, NH), 4.58 (quint, 1H) 1.47 (d, 3H).

### Example No. I.3-16

¹H NMR (400 MHz, CDCl₃ δ, ppm) 8.57 (d, 1H), 8.19 (d, 1H), 8.11 (s, 1H), 7.97 (d, 1H), 7.70 (m, 2H), 7.64 (dd, 1H), 7.44 (d, 1H), 6.75 (d, 1H), 6.06 (br. d, 1H, NH), 4.46 (quint, 1H) 1.39 (d, 3H).

### Example No. I.3-17

¹H NMR (400 MHz, CDCl₃ δ, ppm) 8.81 (d, 1H), 8.48 (d, 1H), 8.39 (d, 1H), 8.12 (d, 1H), 7.87 (m, 2H), 7.81 (m, 1H), 7.70 (m, 2H), 7.43 (m, 1H), 4.81 (s, 2H), 2.38 (m, 1H), 0.52 (m, 4H).

### Example No. I.3-18

¹H NMR (400 MHz, CDCl₃ δ, ppm) 8.71 (d, 1H), 8.42 (d, 2H), 8.38 (d, 1H), 8.09 (d, 1H), 7.82 (d, 1H), 7.69 (m, 2H), 7.63 (m, 2H), 7.36 (m, 2H), 7.20 (m, 2H), 4.81 (s, 4H).

### Example No. I.3-19

¹H NMR (400 MHz, CDCl₃ δ, ppm) 8.79 (d, 1H), 8.40 (d, 1H), 8.37 (d, 1H), 8.09 (d, 1H), 7.89 (d, 1H), 7.71 (m, 2H), 7.42 (m, 1H), 7.21 (m, 1H), 4.50 (s, 2H), 2.87 (s, 3H).

### Example No. I.3-20

¹H NMR (400 MHz, CDCl₃ δ, ppm) 8.73 (d, 1H), 8.52 (m, 1H), 8.38 (m, 1H), 8.10 (d, 1H), 7.87 (d, 1H), 7.70 (m, 2H), 7.64 (m, 1H), 7.38 (m, 1H), 7.21 (m, 1H), 5.39 (q, 1H), 2.78 (s, 3H), 1.44 (d, 3H).

### Example No. I.3-21

¹H NMR (400 MHz, CDCl₃ δ, ppm) 8.68 (d, 1H), 8.26 (d, 1H), 8.16 (d, 1H), 8.03 (d, 1H), 7.74 (d, 1H), 7.71 (dd, 1H), 7.65 (dd, 1H), 7.48 (dd, 1H), 7.07 (dd, 1H), 7.03 (br. d, 1H, NH), 7.01 (d, 1H), 4.88 (quint, 1H).

### Example No. I.3-25

¹H NMR (400 MHz, CDCl₃ δ, ppm) 8.57 (d, 1H), 8.29 (d, 1H), 8.03 (d, 1H), 7.77 (d, 1H), 7.66 (dd, 1H), 7.63 (dd, 1H), 7.29 (d, 1H), 7.20 (d, 1H), 6.98 (dd, 1H) 6.92 (dd, 1H), 5.25 (br. t, 1H, NH), 4.22 (d, 2H).

### Example No. I.3-26

¹H NMR (400 MHz, CDCl₃ δ, ppm) 8.60 (d, 1H), 8.32 (d, 1H), 8.03 (d, 1H), 7.78 (d, 1H), 7.72 (dd, 1H), 7.68 (dd, 1H), 7.31 (m, 2H), 7.22 (d, 1H), 7.18 (dd, 1H), 5.32 (br. t, 1H, NH), 4.33 (d, 2H).

### Example No. I.3-27

¹H NMR (400 MHz, CDCl₃ δ, ppm) 8.69 (d, 1H), 8.43 (d, 1H), 8.36 (d, 1H), 8.09 (d, 1H), 7.82 (d, 1H), 7.70 (m, 2H), 7.55 (dd, 1H), 7.51 (d, 1H), 7.11 (dd, 1H), 4.65 (s, 2H), 4.20 (sept, 1H), 0.93 (d, 6H).

### Example No. I.3-28

¹H NMR (400 MHz, CDCl₃ δ, ppm) 8.72 (d, 1H), 8.45 (d, 1H), 8.29 (dd, 1H), 8.21 (d, 1H), 8.12 (d, 1H), 7.82 (d, 1H), 7.72 (dd, 1H), 7.67 (m, 3H), 6.31 (br. t, 1H, NH), 4.39 (d, 2H).

### Example No. I.3-29

¹H NMR (400 MHz, CDCl₃ δ, ppm) 8.73 (m, 1H), 8.38 (m, 1H), 8.00 (d, 1H), 7.88 (d, 1H), 7.73-7.28 (m, 2H), 3.83-3.69 (m, 3H), 3.42 (dd, 1H), 3.22 (m, 1H), 3.09 (m, 1H), 2.82 (s, 3H), 2.51 (m, 1H), 1.97 (m, 1H), 1.60 (m 1H).

### Example No. I.3-31

¹H NMR (400 MHz, CDCl₃ δ, ppm) 8.58 (d, 1H), 8.33 (d, 1H), 8.18 (m, 1H), 8.06 (d, 1H), 7.83 (d, 1H), 7.71 (m, 2H), 7.42 (d, 1H), 7.00 (dd, 1H), 5.34 (br. t, 1H, NH), 4.21 (d, 2H).

### Example No. I.3-32

¹H NMR (400 MHz, CDCl₃ δ, ppm) 8.62 (d, 1H), 8.42 (d, 1H), 8.18 (m, 1H), 8.07 (d, 1H), 7.86 (d, 1H), 7.73 (m, 2H), 7.38 (d, 1H), 7.12 (dd, 1H), 5.04 (br. t, 1H, NH), 4.11 (d, 2H).

### Example No. I.3-33

¹H NMR (400 MHz, CDCl₃ δ, ppm) 8.62 (d, 1H), 8.40 (d, 1H), 8.38 (s, 1H), 8.04 (d, 1H), 7.83 (d, 1H), 7.72 (m, 2H), 7.59 (d, 1H), 7.43 (d, 1H), 5.11 (br. t, 1H, NH), 4.21 (d, 2H).

### Example No. I.3-34

¹H NMR (400 MHz, CDCl₃ δ, ppm) 9.04 (m, 2H), 8.68 (m, 1H), 8.42 (d, 1H), 8.08 (d, 1H), 7.86 (d, 1H), 7.73 (m, 2H), 7.19 (m, 2H), 5.48 (br. t, 1H, NH), 3.68 (d, 2H).

### Example No. I.3-35

¹H NMR (400 MHz, CDCl₃ δ, ppm) 8.60 (d, 1H), 8.55 (d, 1H), 8.37 (d, 1H), 8.29 (m, 1H), 8.06 (d, 1H), 7.84 (d, 1H), 7.72 (m, 2H), 7.51 (d, 1H), 5.31 (br. t, 1H, NH), 4.16 (d, 2H).

### Example No. I.3-36

¹H NMR (400 MHz, CDCl₃ δ, ppm) 8.63 (d, 1H), 8.40 (d, 1H), 8.15 (d, 1H), 8.05 (d, 1H), 7.85 (d, 1H), 7.74 (m, 2H), 6.98 (s, 1H), 6.92 (d, 1H), 5.06 (br. t, 1H, NH), 4.11 (d, 2H).

### Example No. I.3-37

¹H NMR (400 MHz, CDCl₃ δ, ppm) 8.87 (s, 1H), 8.70 (d, 1H), 8.51 (d, 1H), 8.36 (d, 1H), 8.09 (d, 1H), 7.83 (d, 1H), 7.72 (dd, 1H), 7.68 (dd, 1H), 7.07 (d, 1H), 5.97 (br. d, 1H, NH), 4.20 (d, 2H).

### Example No. I.3-38

¹H NMR (400 MHz, CDCl₃ δ, ppm) 8.61 (d, 1H), 8.34 (d, 1H), 8.29 (d, 2H), 7.92 (d, 1H), 7.71 (m, 3H), 6.99 (d, 2H), 5.60 (br. d, 1H, NH), 4.42 (quint, 1H), 1.33 (d, 3H).

### Example No. I.3-39

¹H NMR (400 MHz, CDCl₃ δ, ppm) 8.68 (d, 1H), 8.29 (d, 1H), 8.06 (d, 1H), 7.80 (m, 2H), 7.68 (m, 2H), 6.89 (d, 1H), 6.80 (d, 1H), 6.04 (br. d, 1H, NH), 4.16 (d, 2H), 3.75 (d, 3H).

### Example No. I.3-41

¹H NMR (400 MHz, CDCl₃ δ, ppm) 8.59 (d, 1H), 8.35 (d, 1H), 8.03 (d, 1H), 7.81 (d, 1H), 7.67 (m, 2H), 5.72 (d, 1H), 5.55 (d, 1H), 4.93 (br. t, 1H, NH), 4.10 (d, 2H), 1.84 (s, 3H).

### Example No. I.3-42

¹H NMR (400 MHz, CDCl₃ δ, ppm) 8.67 (d, 1H), 8.39 (d, 1H), 8.08 (d, 1H), 7.86 (d, 1H), 7.70 (m, 2H), 5.00 (br. t, 1H, NH), 3.85 (m, 1H), 3.68 (m, 1H), 3.60 (m, 1H), 3.06 (m, 1H), 2.82 (m, 1H), 1.78 (m, 3H), 1.48 (m, 1H).

### Example No. I.3-43

¹H NMR (400 MHz, CDCl₃ δ, ppm) 8.58 (d, 1H), 8.19 (d, 1H), 8.02 (d, 1H), 7.75 (d, 1H), 7.62 (dd, 1H), 7.51 (dd, 1H), 7.28-7.15 (m, 5H), 6.22 (d, 1H), 5.95 (d, 1H), 5.07 (br. t, 1H, NH), 4.23 (d, 2H).

### Example No. I.3-44 (Diastereomer Mixture)

¹H NMR (400 MHz, CDCl₃ δ, ppm) 8.61 (m, 1H), 8.36 (m, 1H), 8.02 (m, 1H), 7.80 (m, 1H), 7.70 (m, 1H), 7.68 (m, 1H), 5.80/5.76 (d, 1H), 5.11/5.00 (d, 1H), 4.91 (br. m, 1H, NH), 4.11 (m, 2H), 1.44/1.12 (m, 1H), 1.04/0.68 (d, 3H), 0.92/0.84 (m, 1H), 0.66/0.50 (m, 1H), 0.42/0.22 (m, 1H).

### Example No. I.3-45

¹H NMR (400 MHz, CDCl₃ δ, ppm) 8.54 (d, 1H), 8.33 (d, 1H), 8.04 (d, 1H), 7.82 (d, 1H), 7.70 (dd, 1H), 7.66 (dd, 1H), 5.93 (s, 1H), 5.01 (br. t, 1H, NH), 4.10 (d, 2H), 3.76 (s, 3H), 2.05 (s, 3H).

### Example No. I.3-46

¹H NMR (400 MHz, CDCl₃ δ, ppm) 8.53 (d, 1H), 8.34 (d, 1H), 8.02 (d, 1H), 7.82 (d, 1H), 7.71 (dd, 1H), 7.68 (dd, 1H), 6.94 (d, 1H), 6.02 (m, 2H), 5.48 (br. d, 1H, NH), 4.93 (quint, 1H).

### Example No. I.3-48

¹H NMR (400 MHz, CDCl₃ δ, ppm) 8.66 (d, 1H), 8.34 (d, 1H), 8.09 (d, 1H), 7.83 (d, 1H), 7.65 (m, 2H), 7.08 (d, 1H), 5.83 (d, 1H), 5.21 (br. t, 1H, NH), 4.13 (d, 2H), 3.78 (t, 2H), 1.65 (sext, 2H), 0.79 (t, 3H).

### Example No. I.3-49

¹H NMR (400 MHz, CDCl₃ δ, ppm) 8.61 (d, 1H), 8.32 (d, 1H), 8.10 (d, 1H), 7.83 (d, 1H), 7.66 (m, 2H), 6.88 (s, 1H), 5.26 (br. t, 1H, NH), 4.13 (d, 2H), 3.47 (s, 3H).

### Example No. I.3-50

¹H NMR (400 MHz, CDCl₃ δ, ppm) 8.60 (d, 1H), 8.41 (d, 1H), 8.04 (d, 1H), 7.86 (d, 1H), 7.74 (dd, 1H), 7.71 (dd, 1H), 7.16 (s, 1H), 4.95 (br. t, 1H, NH), 4.14 (d, 2H), 3.69 (s, 3H).

### Example No. I.3-51

¹H NMR (400 MHz, CDCl₃ δ, ppm) 8.68 (d, 1H), 8.38 (d, 1H), 8.09 (d, 1H), 7.86 (d, 1H), 7.68 (m, 2H), 3.03 (m, 1H), 2.87 (dd, 1H), 2.74 (dd, 1H), 2.40 (m, 1H), 2.17 (m, 1H), 2.01 (m, 1H), 1.88 (m, 1H), 1.68 (m, 1H), 1.65-1.45 (m, 4H), 0.78 (t, 3H).

### Example No. I.3-52

¹H NMR (400 MHz, CDCl₃ δ, ppm) 8.63 (d, 1H), 8.39 (d, 1H), 8.09 (d, 1H), 7.86 (d, 1H), 7.70 (m, 2H), 5.68 (s, 1H), 5.15 (br. t, 1H, NH), 4.12 (d, 2H), 1.54 (s, 3H).

### Example No. I.3-53

¹H NMR (400 MHz, CDCl₃ δ, ppm) 8.63 (d, 1H), 8.38 (d, 1H), 8.06 (d, 1H), 7.83 (d, 1H), 7.71 (m, 2H), 5.43 (br. t, 1H, NH), 4.36 (d, 2H), 1.79 (m, 1H), 0.88 (m, 2H), 0.57 (m, 2H).

### Example No. I.3-54

¹H NMR (400 MHz, CDCl₃ δ, ppm) 8.62 (d, 1H), 8.37 (d, 1H), 8.08 (d, 1H), 7.84 (d, 1H), 7.70 (m, 2H), 5.40 (br. t, 1H, NH), 4.39 (d, 2H), 2.43 (q, 2H), 1.09 (t, 3H).

### Example No. I.3-55

¹H NMR (400 MHz, CDCl₃ δ, ppm) 8.60 (d, 1H), 8.32 (d, 1H), 8.00 (d, 1H), 7.78 (d, 1H), 7.68 (m, 2H), 6.55 (s, 1H), 5.39 (br. t, 1H, NH), 4.19 (d, 2H), 2.29 (s, 3H).

### Example No. I.3-56

¹H NMR (400 MHz, CDCl₃ δ, ppm) 8.56 (d, 1H), 8.33 (d, 1H), 8.04 (d, 1H), 7.79 (d, 1H), 7.67 (m, 2H), 7.07 (s, 1H), 5.51 (br. t, 1H, NH), 4.31 (d, 2H).

### Example No. I.4-1

¹H NMR (400 MHz, CDCl₃ δ, ppm) 9.02 (d, 1H), 8.40 (d, 1H), 8.22 (m, 2H), 7.99 (d, 1H), 7.60 (dd, 1H), 7.52 (dd, 1H), 7.48 (dd, 1H), 7.25 (d, 1H), 7.13 (br. t, 1H, NH), 6.97 (dd, 1H), 4.22 (d, 2H).

### Example No. I.4-6

¹H NMR (400 MHz, CDCl₃ δ, ppm) 9.03 (d, 1H), 8.41 (d, 1H), 8.36 (dd, 1H), 8.22 (dd, 1H), 7.99 (d, 1H), 7.69 (dd, 1H), 7.69 (dd, 1H), 7.54 (dd, 1H), 7.39 (d, 1H), 7.18 (br. t, 1H, NH), 4.31 (d, 2H).

### Example No. I.4-7

¹H NMR (400 MHz, CDCl₃ δ, ppm) 9.04 (d, 1H), 8.44 (d, 1H), 8.22 (s, 1H), 8.17 (d, 1H), 7.96 (d, 1H), 7.58 (m, 3H), 7.51 (m, 3H), 7.39 (br. t, 1H, NH), 4.53 (d, 2H).

### Example No. I.4-39

¹H NMR (400 MHz, CDCl₃ δ, ppm) 9.01 (d, 1H), 8.38 (d, 1H), 8.21 (d, 1H), 7.98 (d, 1H), 7.84 (d, 1H), 7.59 (dd, 1H), 7.51 (dd, 1H), 7.11 (d, 1H), 7.02 (br. t, 1H, NH), 6.93 (dd, 1H), 4.17 (d, 2H), 3.74 (s, 3H).

### Example No. I.4-46

¹H NMR (400 MHz, CDCl₃ δ, ppm) 8.97 (d, 1H), 8.32 (d, 1H), 8.18 (d, 1H), 7.97 (d, 1H), 7.58 (dd, 1H), 7.50 (dd, 1H), 7.38 (br. d, 1H, NH), 6.69 (m, 1H), 5.89 (m, 1H), 5.79 (m, 1H), 5.06 (m, 1H).

### Example No. I.4-48

¹H NMR (400 MHz, CDCl₃ δ, ppm) 8.97 (d, 1H), 8.42 (d, 1H), 8.23 (d, 1H), 8.01 (d, 1H), 7.62 (dd, 1H), 7.50 (dd, 1H), 7.04 (d, 1H), 6.69 (br. t, 1H, NH), 5.94 (d, 1H), 4.12 (d, 2H), 3.76 (t, 2H), 1.62 (sext, 2H), 0.76 (t, 3H).

### Example No. I.4-49

¹H NMR (400 MHz, CDCl₃ δ, ppm) 8.99 (d, 1H), 8.43 (d, 1H), 8.20 (d, 1H), 7.97 (d, 1H), 7.60 (dd, 1H), 7.49 (dd, 1H), 6.84 (s, 1H), 6.75 (br. t, 1H, NH), 4.16 (d, 2H), 3.45 (s, 3H).

### Example No. I.4-50

¹H NMR (400 MHz, CDCl₃ δ, ppm) 8.98 (d, 1H), 8.39 (d, 1H), 8.26 (d, 1H), 8.05 (d, 1H), 7.63 (dd, 1H), 7.56 (dd, 1H), 6.99 (s, 1H), 6.84 (br. t, 1H, NH), 4.14 (d; 2H), 3.33 (s, 3H).

### Example No. I.4-51

¹H NMR (400 MHz, CDCl₃ δ, ppm) 9.04 (d, 1H), 8.44 (d, 1H), 8.26 (d, 1H), 8.03 (d, 1H), 7.63 (dd, 1H), 7.52 (dd, 1H), 6.50 (br. m, 1H, NH), 3.01 (m, 2H), 2.63 (dd, 1H), 2.43 (m, 1H), 2.28 (m, 1H), 2.00 (m, 1H), 1.92 (m, 1H), 1.67 (m, 1H), 1.65-1.45 (m, 3H), 0.81 (t, 3H).

### Example No. I.4-55

¹H NMR (400 MHz, CDCl₃ δ, ppm) 9.02 (d, 1H), 8.31 (d, 1H), 8.22 (d, 1H), 7.97 (d, 1H), 7.56 (dd, 1H), 7.51 (dd, 1H), 6.90 (br. t, 1H, NH), 6.74 (s, 1H), 4.22 (d, 2H), 2.25 (s, 3H).

### Example No. I.4-59

¹H NMR (400 MHz, CDCl₃ δ, ppm) 9.06 (d, 1H), 8.51 (d, 1H), 8.23 (d, 1H), 8.02 (d, 1H), 7.60 (dd, 1H), 7.51 (dd, 1H), 7.10 (s, 1H), 4.78 (d, 2H), 2.97 (s, 3H), 2.63 (s, 3H).

### Example No. I.5-1

¹H NMR (400 MHz, CDCl₃ δ, ppm) 8.73 (d, 1H), 8.32 (d, 1H), 8.30 (d, 1H), 8.22 (d, 1H), 8.17 (d, 1H), 7.54 (dd, 1H), 7.43 (dd, 1H), 7.31 (dd, 1H), 7.01 (dd, 1H), 6.91 (d, 1H), 6.18 (br. t, 1H, NH), 4.20 (d, 2H).

### Example No. I.5-3

¹H NMR (400 MHz, CDCl₃ δ, ppm) 8.70 (d, 1H), 8.32 (d, 1H), 8.30 (d, 1H), 8.16 (d, 1H), 8.05 (d, 1H), 7.54 (dd, 1H), 7.30 (dd, 1H), 6.79 (d, 1H), 6.63 (s, 1H), 6.20 (br. t, 1H, NH), 4.13 (d, 2H), 2.18 (s, 3H).

### Example No. I.5-9

¹H NMR (400 MHz, CDCl₃ δ, ppm) 8.69 (d, 1H), 8.34 (d, 1H), 8.32 (d, 1H), 8.18 (d, 1H), 7.86 (m, 1H), 7.55 (dd, 1H), 7.32 (dd, 1H), 6.89 (m, 1H), 5.98 (br. t, 1H, NH), 4.24 (d, 2H).

### Example No. I.5-48

¹H NMR (400 MHz, CDCl₃ δ, ppm) 8.69 (d, 1H), 8.36 (d, 1H), 8.32 (d, 1H), 8.18 (d, 1H), 7.56 (dd, 1H), 7.30 (dd, 1H), 7.08 (d, 1H), 5.82 (d, 1H), 5.21 (br. t, 1H, NH), 4.13 (d, 2H), 3.79 (t, 2H), 1.64 (sext, 2H), 0.78 (t, 3H).

### Example No. I.5-55

¹H NMR (400 MHz, CDCl₃ δ, ppm) 8.62 (d, 1H), 8.32 (d, 1H), 8.24 (d, 1H), 8.17 (d, 1H), 7.52 (dd, 1H), 7.31 (dd, 1H), 6.50 (s, 1H), 5.37 (br. t, 1H, NH), 4.17 (d, 2H), 2.32 (s, 3H).

### Example No. I.5-61

¹H NMR (400 MHz, CDCl₃ δ, ppm) 8.63 (d, 1H), 8.33 (m, 2H), 8.20 (d, 1H), 7.59 (dd, 1H), 7.44 (d, 2H), 7.41 (d, 2H), 7.37 (d, 1H), 5.85 (s, 1H), 5.22 (br. t, 1H, NH), 4.33 (d, 2H).

### Example No. I.6-6

¹H NMR (400 MHz, CDCl₃ δ, ppm) 8.64 (d, 1H), 8.41 (s, 1H), 8.21 (m, 1H), 8.13 (d, 1H), 7.72 (dd, 1H), 7.63 (m, 2H), 7.11 (m, 1H), 7.08 (d, 1H), 6.00 (br. t, 1H, NH), 4.29 (d, 2H).

### Example No. I.6-9

¹H NMR (400 MHz, CDCl₃ δ, ppm) 8.63 (d, 1H), 8.26 (m, 1H), 8.13 (d, 1H), 7.87 (d, 1H), 7.71 (dd, 1H), 7.63 (dd, 1H), 7.14 (m, 1H), 6.90 (m, 1H), 5.98 (br. t, 1H, NH), 4.26 (d, 2H).

### Example No. I.6-48

¹H NMR (400 MHz, CDCl₃ δ, ppm) 8.64 (d, 1H), 8.25 (m, 1H), 8.18 (d, 1H), 7.70 (dd, 1H), 7.63 (dd, 1H), 7.15 (m, 1H), 7.09 (d, 1H), 5.84 (d, 1H), 5.14 (br. t, 1H, NH), 4.12 (d, 2H), 3.80 (t, 2H), 1.66 (sext, 2H), 0.79 (t, 3H).

### Example No. I.6-51

¹H NMR (400 MHz, CDCl₃ δ, ppm) 8.66 (d, 1H), 8.21 (m, 2H), 7.72 (dd, 1H), 7.65 (dd, 1H), 7.17 (m, 1H), 3.02 (m, 1H), 2.86 (dd, 1H), 2.74 (dd, 1H), 2.39 (m, 1H), 2.17 (m, 1H), 2.00 (m, 1H), 1.88 (m, 1H), 1.69 (m, 1H), 1.65-1.48 (m, 4H), 0.77 (t, 3H).

### Example No. I.6-55

¹H NMR (400 MHz, CDCl₃ δ, ppm) 8.57 (d, 1H), 8.15 (m, 2H), 7.70 (dd, 1H), 7.62 (dd, 1H), 7.10 (m, 1H), 6.56 (s, 1H), 5.39 (br. t, 1H, NH), 4.18 (d, 2H), 2.33 (s, 3H).

### Example No. I.6-61

¹H NMR (400 MHz, d₆-DMSO, δ ppm) 8.82 (br. t, 1H, NH), 8.64 (d, 1H), 8.13 (m, 1H), 8.09 (d, 1H), 7.82 (dd, 1H), 7.75 (dd, 1H), 7.61 (d, 2H), 7.55 (d, 2H), 7.41 (dd, 1H), 6.48 (s, 1H), 4.29 (d, 2H).

### Example No. I.6-62

¹H NMR (400 MHz, d₆-DMSO, δ ppm) 8.91 (br. t, 1H, NH), 8.67 (d, 1H), 8.15 (m, 1H), 8.11 (d, 1H), 7.83 (dd, 1H), 7.76 (dd, 1H), 7.52 (d, 2H), 7.42 (dd, 1H), 7.00 (d, 2H), 6.37 (s, 1H), 4.26 (d, 2H), 3.81 (s, 3H).

### Example No. I.7-1

¹H NMR (400 MHz, d₆-DMSO δ, ppm) 10.14 (br. s, 1H, NH), 8.69 (d, 1H), 8.54 (br. t, 1H, NH), 8.31 (d, 1H), 8.28 (d, 1H), 8.10 (d, 1H), 7.91 (d, 1H), 7.69 (m, 2H), 7.59 (dd, 1H), 7.24 (d, 1H), 7.13 (dd, 1H), 4.11 (d, 2H), 2.23 (s, 3H).

### Example No. I.7-2

¹H NMR (400 MHz, d₆-DMSO δ, ppm) 10.12 (br. s, 1H, NH), 8.67 (d, 1H), 8.53 (br. t, 1H, NH), 8.47 (d, 2H), 8.22 (d, 1H), 8.06 (d, 1H), 7.84 (d, 1H), 7.65 (m, 2H), 7.18 (t, 1H), 4.22 (d, 2H), 2.21 (s, 3H).

### Example No. I.7-5

¹H NMR (400 MHz, d₆-DMSO δ, ppm) 10.11 (br. s, 1H, NH), 8.68 (d, 1H), 8.46 (br. t, 1H, NH), 8.28 (d, 1H), 8.07 (d, 1H), 7.91 (d, 1H), 7.81 (s, 1H), 7.67 (m, 2H), 6.58 (s, 1H), 4.07 (d, 2H), 3.71 (s, 3H), 3.39 (s, 3H), 2.22 (s, 3H).

### Example No. I.7-6

¹H NMR (400 MHz, CDCl₃ δ, ppm) 8.66 (d, 1H), 8.30 (s, 1H), 8.19 (d, 1H), 8.01 (br. s, 1H, NH), 7.79 (d, 1H), 7.63 (dd, 1H), 7.49 (m, 3H), 6.98 (d, 1H), 6.04 (br. t, 1H, NH), 4.26 (d, 2H), 2.32 (s, 3H).

### Example No. I.7-7

¹H NMR (400 MHz, d₆-DMSO δ, ppm) 10.07 (br. s, 1H, NH), 8.63 (br. t, 1H, NH), 8.59 (d, 1H), 8.26 (s, 1H), 8.19 (d, 1H), 8.06 (d, 1H), 7.97 (s, 1H), 7.85 (d, 1H), 7.63 (m, 2H), 4.34 (d, 2H), 2.22 (s, 3H).

### Example No. I.7-8

¹H NMR (400 MHz, d₆-DMSO δ, ppm) 10.12 (br. s, 1H, NH), 8.62 (d, 1H), 8.48 (br. t, 1H, NH), 8.22 (m, 1H), 8.04 (m, 2H), 7.85 (d, 1H), 7.64 (m, 2H), 7.37 (dd, 1H), 7.14 (m, 1H), 4.18 (d, 2H), 2.23 (s, 3H).

### Example No. I.7-9

¹H NMR (400 MHz, CDCl₃ δ, ppm) 8.69 (d, 1H), 8.26 (d, 1H), 8.05 (br. s, 1H, NH), 7.83 (m, 2H), 7.65 (dd, 1H), 7.50 (m, 2H), 6.91 (m, 1H), 5.96 (br. t, 1H, NH), 4.27 (d, 2H), 2.34 (s, 3H).

### Example No. I.7-10

¹H NMR (400 MHz, d₆-DMSO δ, ppm) 10.12 (br. s, 1H, NH), 8.69 (br. m, 1H, NH), 8.59 (d, 1H), 8.49 (s, 1H), 8.27 (d, 1H), 8.03 (d, 1H), 7.92 (d, 1H), 7.64 (m, 2H), 7.43 (S, 1H), 4.24 (d, 2H), 2.22 (s, 3H).

### Example No. I.7-11

¹H NMR (400 MHz, d₆-DMSO δ, ppm) 10.22 (br. s, 1H, NH), 8.68 (d, 1H), 8.50 (d, 1H), 8.33 (d, 1H), 8.17 (d, 1H), 8.01 (d, 1H), 7.77 (dd, 1H), 7.70 (m, 2H), 7.34 (d, 1H), 7.29 (dd, 1H), 4.51 (s, 2H), 2.81 (s, 3H), 2.26 (s, 3H).

### Example No. I.7-20

¹H NMR (400 MHz, d₆-DMSO δ, ppm) 10.21 (br. s, 1H, NH), 8.62 (d, 1H), 8.48 (d, 1H), 8.32 (d, 1H), 8.21 (d, 1H), 8.02 (d, 1H), 7.71 (dd, 1H), 7.68 (m, 2H), 7.22 (m, 2H), 5.23 (q, 1H), 2.68 (s, 3H), 2.25 (s, 3H), 1.33 (d, 3H).

### Example No. I.7-32

¹H NMR (400 MHz, CDCl₃ δ, ppm) 8.66 (d, 1H), 8.27 (d, 1H), 7.97 (m, 2H), 7.69 (m, 3H), 7.39 (d, 1H), 7.20 (m, 1H), 7.12 (d, 1H), 4.94 (br. t, NH, 1H), 4.10 (d, 2H), 2.38 (s, 3H).

### Example No. I.7-34

¹H NMR (400 MHz, CDCl₃ δ, ppm) 8.56 (d, 1H), 8.42 (m, 2H), 8.13 (d, 1H), 7.98 (m, 3H), 7.67 (dd, 1H), 7.63 (dd, 1H), 7.08 (d, 2H), 5.78 (br. t, NH, 1H), 4.31 (d, 2H), 2.38 (s, 3H).

### Example No. I.8-1

¹H NMR (400 MHz, CDCl₃ δ, ppm) 8.61 (d, 1H), 8.27 (m, 2H), 8.22 (d, 1H), 7.64 (dd, 1H), 7.53 (dd, 1H), 7.44 (m, 1H), 7.00 (m, 2H), 6.77 (d, 1H), 6.01 (br. t, 1H, NH), 4.16 (d, 2H), 4.05 (s, 3H).

### Example No. I.8-6

¹H NMR (400 MHz, CDCl₃ δ, ppm) 8.56 (d, 1H), 8.42 (s, 1H), 8.28 (d, 1H), 8.17 (d, 1H), 7.65 (dd, 1H), 7.662 (dd, 1H), 7.55 (dd, 1H), 7.08 (d, 1H), 6.73 (d, 1H), 5.88 (br. t, 1H, NH), 4.24 (d, 2H), 4.04 (s, 3H).

### Example No. I.8-7

¹H NMR (400 MHz, CDCl₃ δ, ppm) 8.59 (d, 1H), 8.24 (d, 1H), 8.22 (m, 2H), 7.66 (dd, 1H), 7.60 (s, 1H), 7.52 (dd, 1H), 6.74 (d, 1H), 6.22 (br. t, 1H, NH), 4.35 (d, 2H), 4.04 (s, 3H).

### Example No. I.8-11

¹H NMR (400 MHz, d₆-DMSO δ, ppm) 8.61 (d, 1H), 8.50 (d, 1H), 8.31 (d, 1H), 8.18 (d, 1H), 7.76 (m, 2H), 7.67 (dd, 1H), 7.32 (d, 1H), 7.29 (dd, 1H), 7.12 (d, 1H), 4.46 (s, 2H), 4.10 (s, 3H), 2.75 (s, 3H).

### Example No. I.8-29

¹H NMR (400 MHz, CDCl₃ δ, ppm) 8.60 (d, 1H), 8.34 (d, 1H), 8.18 (d, 1H), 7.64 (m, 1H), 7.56 (m, 1H), 6.82 (d, 1H), 4.08 (s, 3H), 3.78-3.65 (m, 3H), 3.37 (dd, 1H), 3.18 (m, 1H), 3.07 (m, 1H), 2.80 (s, 3H), 2.50 (m, 1H), 1.94 (m, 1H), 1.56 (m, 1H).

### Example No. I.8-52

¹H NMR (400 MHz, d₆-DMSO δ, ppm) 8.57 (d, 1H), 8.42 (br. t, 1H, NH), 8.28 (d, 1H), 8.07 (d, 1H), 7.72 (dd, 1H), 7.64 (dd, 1H), 7.07 (d, 1H), 5.76 (s, 1H), 4.06 (s, 3H), 3.95 (d, 2H), 2.18 (s, 3H).

### Example No. I.9-1

¹H NMR (400 MHz, d₆-DMSO δ, ppm) 8.60 (br. t, 1H, NH), 8.46 (d, 1H), 7.83 (d, 1H), 7.73 (m, 1H), 7.62 (d, 1H), 7.43 (d, 1H), 7.40 (d, 1H), 7.25 (dd, 1H), 7.10 (dd, 1H), 6.61 (d, 1H), 4.31 (d, 2H), 4.16 (s, 3H).

### Example No. I.9-2

¹H NMR (400 MHz, d₆-DMSO δ, ppm) 8.63 (d, 2H), 8.51 (br. t, 1H, NH), 7.86 (d, 1H), 7.64 (d, 1H), 7.41 (d, 1H), 7.29 (t, 1H), 7.04 (dd, 1H), 6.58 (d, 1H), 4.38 (d, 2H), 4.18 (s, 3H).

### Example No. I.9-5

¹H NMR (400 MHz, d₆-DMSO δ, ppm) 8.47 (br. t, 1H, NH), 7.99 (s, 1H), 7.80 (d, 1H), 7.64 (d, 1H), 7.44 (d, 1H), 7.09 (dd, 1H), 6.89 (s, 1H), 6.60 (d, 1H), 4.23 (d, 2H), 4.18 (s, 3H), 3.79 (s, 3H), 3.67 (s, 3H).

### Example No. I.9-6

¹H NMR (400 MHz, d₆-DMSO δ, ppm) 8.78 (s, 1H), 8.71 (br. t, 1H, NH), 8.10 (d, 1H), 7.78 (d, 1H), 7.59 (d, 1H), 7.42 (d, 1H), 7.08 (dd, 1H), 6.59 (d, 1H), 4.42 (d, 2H), 4.16 (s, 3H).

### Example No. I.9-7

¹H NMR (400 MHz, d₆-DMSO δ, ppm) 8.58 (m, 2H), 8.18 (s, 1H), 7.70 (d, 1H), 7.63 (d, 1H), 7.38 (d, 1H), 7.00 (dd, 1H), 6.52 (d, 1H), 4.48 (d, 2H), 4.18 (s, 3H).

### Example No. I.9-8

¹H NMR (400 MHz, d₆-DMSO δ, ppm) 8.49 (br. t, 1H, NH), 8.27 (d, 1H), 7.76 (d, 1H), 7.60 (d, 1H), 7.52 (m, 1H), 7.40 (d, 1H), 7.29 (m, 1H), 7.04 (dd, 1H), 6.57 (d, 1H), 4.37 (d, 2H), 4.13 (s, 3H).

### Example No. I.9-9

¹H NMR (400 MHz, d₆-DMSO δ, ppm) 8.52 (br. t, 1H, NH), 8.27 (d, 1H), 7.76 (d, 1H), 7.60 (d, 1H), 7.52 (m, 1H), 7.39 (d, 1H), 7.03 (dd, 1H), 6.58 (d, 1H), 4.32 (d, 2H), 4.16 (s, 3H).

### Example No. I.9-10

¹H NMR (400 MHz, d₆-DMSO δ, ppm) 8.69 (br. t, 1H, NH), 8.67 (s, 1H), 7.76 (d, 1H), 7.62 (s, 1H), 7.60 (d, 1H), 7.40 (d, 1H), 7.04 (dd, 1H), 6.56 (d; 1H), 4.38 (d, 2H), 4.16 (s, 3H).

### Example No. I.9-11

¹H NMR (400 MHz, d₆-DMSO δ, ppm) 8.56 (d, 1H), 7.88 (d, 1H), 7.80 (m, 1H), 7.54 (d, 1H), 7.47 (d, 1H), 7.39 (d, 1H), 7.33 (dd, 1H), 7.15 (dd, 1H), 6.65 (d, 1H), 4.64 (s, 2H), 4.12 (s, 3H), 3.00 (s, 3H).

### Example No. I.9-20

¹H NMR (400 MHz, d₆-DMSO δ, ppm) 8.57 (d, 1H), 7.87 (d, 1H), 7.82 (m, 1H), 7.54 (d, 1H), 7.44 (d, 1H), 7.32 (dd, 1H), 7.18 (d, 1H), 7.12 (dd, 1H), 6.64 (d, 1H), 5.12 (q, 1H), 4.16 (s, 3H), 2.86 (s, 3H), 1.62 (d, 3H).

### Example No. I.9-62

¹H NMR (400 MHz, d₆-DMSO, δ ppm) 8.84 (br. t, 1H, NH), 7.83 (d, 1H), 7.72 (d, 2H), 7.60 (d, 1H), 7.46 (d, 1H), 7.11 (dd, 1H), 7.04 (d, 2H), 6.70 (s, 1H), 6.62 (d, 1H), 4.42 (d, 2H), 4.15 (s, 3H), 3.82 (s, 3H).

### Example No. I.10-1

¹H NMR (400 MHz, d₆-DMSO δ, ppm) 8.69 (d, 1H), 8.52 (br. t, 1H, NH), 8.31 (d, 1H), 8.13 (d, 1H), 8.00 (d, 1H), 7.69 (m, 2H), 7.57 (dd, 1H), 7.45 (d, 1H), 7.22 (d, 1H), 7.13 (dd, 1H), 4.09 (d, 2H), 2.71 (s, 3H).

### Example No. I.10-2

¹H NMR (400 MHz, d₆-DMSO δ, ppm) 8.68 (d, 1H), 8.51 (br. t, 1H, NH), 8.45 (d, 2H), 8.10 (d, 1H), 7.96 (d, 1H), 7.68 (m, 2H), 7.40 (d, 1H), 7.17 (t, 1H), 4.21 (d, 2H), 2.69 (s, 3H).

### Example No. I.10-4

¹H NMR (400 MHz, CDCl₃ δ, ppm) 8.82 (d, 1H), 8.16 (d, 1H), 7.93 (d, 2H), 7.78 (d, 1H), 7.66 (m, 3H), 7.50 (m, 2H), 7.28 (d, 1H), 7.07 (d, 1H), 6.57 (br. m, 1H, NH), 4.38 (d, 2H), 2.64 (s, 3H).

### Example No. I.10-5

¹H NMR (400 MHz, d₆-DMSO δ, ppm) 8.68 (d, 1H), 8.45 (br. t, 1H, NH), 8.11 (d, 1H), 7.96 (d, 1H), 7.77 (s, 1H), 7.69 (m, 2H), 7.39 (d, 1H), 6.52 (s, 1H), 4.05 (d, 2H), 3.71 (s, 3H), 3.37 (s, 3H), 2.68 (s, 3H).

### Example No. I.10-6

¹H NMR (400 MHz, d₆-DMSO δ, ppm) 8.69 (br. t, 1H, NH), 8.63 (d, 1H), 8.52 (s, 1H), 8.09 (d, 1H), 7.94 (d, 1H), 7.83 (dd, 1H), 7.67 (m, 2H), 7.40 (d, 1H), 7.34 (d, 1H), 4.21 (d, 2H), 2.69 (s, 3H).

### Example No. I.10-7

¹H NMR (400 MHz, d₆-DMSO δ, ppm) 8.66 (br. t, 1H, NH), 8.62 (d, 1H), 8.18 (s, 1H), 7.99 (m, 2H), 7.94 (d, 1H), 7.65 (m, 2H), 7.35 (d, 1H), 4.34 (d, 2H), 2.62 (s, 3H).

### Example No. I.10-8

¹H NMR (400 MHz, d₆-DMSO δ, ppm) 8.63 (m, 1H), 8.47 (br. t, 1H, NH), 8.09 (d, 1H), 8.02 (d, 1H), 7.97 (d, 1H), 7.66 (m, 2H), 7.41 (d, 1H), 7.16 (dd, 1H), 4.16 (d, 2H), 2.69 (s, 3H).

### Example No. I.10-9

¹H NMR (400 MHz, d₆-DMSO δ, ppm) 8.60 (d, 1H), 8.53 (br. t, 1H, NH), 8.09 (d, 1H), 7.99 (d, 1H), 7.94 (d, 1H), 7.64 (m, 2H), 7.50 (m, 1H), 7.40 (d, 1H), 4.14 (d, 2H), 2.69 (s, 3H).

### Example No. I.10-10

¹H NMR (400 MHz, d₆-DMSO δ, ppm) 8.71 (br. t, 1H, NH), 8.58 (d, 1H), 8.40 (s, 1H), 8.06 (d, 1H), 7.89 (d, 1H), 7.66 (m, 2H), 7.34 (d, 1H), 7.29 (s, 1H), 4.26 (d, 2H), 2.65 (s, 3H).

### Example No. I.10-11

¹H NMR (400 MHz, d₆-DMSO δ, ppm) 8.68 (d, 1H), 8.50 (d, 1H), 8.20 (d, 1H), 8.08 (d, 1H), 7.73 (m, 3H), 7.54 (d, 1H), 7.34 (d, 1H), 7.29 (dd, 1H), 4.49 (s, 2H), 2.79 (s, 3H), 2.76 (s, 3H).

### Example No. I.10-20

¹H NMR (400 MHz, d₆-DMSO δ, ppm) 8.62 (d, 1H), 8.47 (s, 1H), 8.18 (d, 1H), 8.12 (d, 1H), 7.69 (m, 3H), 7.53 (d, 1H), 7.23 (m, 2H), 5.25 (q, 1H), 2.74 (s, 3H), 2.68 (s, 3H), 1.32 (d, 3H).

### Example No. I.10-34

¹H NMR (400 MHz, CDCl₃ δ, ppm) 8.66 (m, 1H), 8.58 (d, 1H), 8.38 (m, 1H), 7.92 (m, 3H), 7.50 (m, 1H), 7.42 (m, 2H), 7.21 (d, 1H), 7.04 (br. m, NH, 1H), 4.10 (d, 2H), 2.59 (s, 3H).

### Example No. I.10-39

¹H NMR (400 MHz, CDCl₃ δ, ppm) 8.68 (d, 1H), 8.13 (d, 1H), 8.03 (d, 1H), 7.90 (d, 1H), 7.62 (m, 1H), 7.58 (dd, 1H), 7.22 (d, 1H), 6.91 (dd, 1H), 6.87 (d, 1H), 5.88 (br. t, NH, 1H), 4.12 (d, 2H), 3.75 (s, 3H), 2.72 (s, 3H).

### Example No. I.12-1

¹H NMR (400 MHz, d₆-DMSO δ, ppm) 8.56 (d, 1H), 8.46 (br. m, 1H, NH), 8.32 (m, 3H), 7.99 (d, 1H), 7.80 (d, 1H), 7.37 (dd, 1H), 7.32 (dd, 1H), 7.24 (dd, 1H), 6.77 (d, 1H), 5.89 (br. s, 2H, NH), 4.20 (d, 2H).

### Example No. I.12-7

¹H NMR (400 MHz, d₆-DMSO δ, ppm) 8.45 (s, 1H), 8.42 (br. t, 1H, NH), 8.26 (d, 1H), 8.12 (s, 1H), 7.98 (d, 1H), 7.74 (d, 1H), 7.33 (dd, 1H), 7.29 (dd, 1H), 6.74 (d, 1H), 5.90 (br. s, 2H, NH), 4.31 (d, 2H).

### Example No. I.12-8

¹H NMR (400 MHz, d₆-DMSO δ, ppm) 8.42 (br. t, 1H, NH), 8.29 (m, 1H), 8.13 (m, 1H), 8.00 (d, 1H), 7.77 (d, 1H), 7.48 (m, 1H), 7.35 (dd, 1H), 7.30 (dd, 1H), 7.22 (m, 1H), 6.75 (d, 1H), 5.88 (br. s, 2H, NH), 4.13 (d, 2H).

### Example No. I.12-9

¹H NMR (400 MHz, d₆-DMSO δ, ppm) 8.38 (br. t, 1H, NH), 8.30 (d, 1H), 8.11 (m, 1H), 7.97 (d, 1H), 7.72 (d, 1H), 7.59 (m, 1H), 7.38 (dd, 1H), 7.29 (dd, 1H), 6.75 (d, 1H), 5.90 (br. s, 2H, NH), 4.12 (d, 2H).

### Example No. I.12-10

¹H NMR (400 MHz, d₆-DMSO δ, ppm) 8.49 (s, 1H), 8.43 (br. t, 1H, NH), 8.30 (d, 1H), 8.08 (m, 1H), 7.72 (d, 1H), 7.62 (dd, 1H), 7.53 (dd, 1H), 7.36 (s, 1H), 6.76 (d, 1H), 5.92 (br. s, 2H, NH), 4.28 (d, 2H).

### Example No. I.12-11

¹H NMR (400 MHz, d₆-DMSO δ, ppm) 8.51 (d, 1H), 8.41 (d, 1H), 8.08 (d, 1H), 7.80 (d, 1H), 7.77 (d, 1H), 7.47 (dd, 1H), 7.38 (dd, 1H), 7.35 (dd, 1H), 7.30 (dd, 1H), 6.80 (d, 1H), 5.95 (br. s, 2H, NH), 4.47 (s, 2H), 2.78 (s, 3H).

### Example No. I.12-20

¹H NMR (400 MHz, d₆-DMSO δ, ppm) 8.49 (d, 1H), 8.40 (d, 1H), 8.12 (d, 1H), 7.73 (d, 1H), 7.68 (m, 1H), 7.46 (dd, 1H), 7.35 (dd, 1H), 7.25 (m, 2H), 6.79 (d, 1H), 6.02 (br. s, 2H, NH), 5.19 (q, 1H), 2.66 (s, 3H), 1.30 (d, 3H).

### Example No. I.12-34

¹H NMR (400 MHz, CDCl₃ δ, ppm) 8.39 (d, 2H), 8.23 (d, 1H), 8.10 (d, 1H), 8.03 (d, 1H), 7.50 (dd, 1H), 7.47 (dd, 1H), 7.28 (br. m, 2H, NH), 7.03 (d, 2H), 6.90 (d, 1H), 5.11 (br. t, 1H, NH), 4.09 (d, 2H).

### Example No. I.12-61

¹H NMR (400 MHz, CDCl₃ δ, ppm) 8.26 (d, 1H), 7.98 (m, 2H), 7.47 (m, 4H), 7.40 (d, 2H), (d, 1H), 7.32 (br. m, 2H, NH), 6.89 (d, 1H), 5.93 (s, 1H), 5.20 (br. t, 1H, NH), 4.28 (d, 2H), 3.84 (s, 3H).

### Example No. I.13-1

¹H NMR (400 MHz, d₆-DMSO δ, ppm) 8.53 (br. t, 1H, NH), 8.41 (d, 1H), 8.30 (m, 2H), 8.09 (d, 1H), 7.54 (m, 3H), 7.24 (d, 1H), 7.20 (d, 1H), 7.12 (dd, 1H), 4.12 (d, 2H), 2.82 (s, 6H).

### Example No. I.13-2

¹H NMR (400 MHz, d₆-DMSO δ, ppm) 8.52 (br. t, 1H, NH), 8.45 (d, 2H), 8.30 (d, 1H), 8.05 (d, 1H), 7.52 (m, 3H), 7.23 (d, 1H), 6.16 (t, 1H), 4.24 (d, 2H), 2.82 (s, 6H).

### Example No. I.13-5

¹H NMR (400 MHz, d₆-DMSO δ, ppm) 8.50 (br. t, 1H, NH), 8.36 (d, 1H), 8.31 (d, 1H), 8.03 (d, 1H), 7.80 (s, 1H), 7.59 (dd, 1H), 7.50 (dd, 1H), 7.22 (d, 1H), 6.53 (s, 1H), 4.11 (d, 2H), 3.71 (s, 3H), 2.81 (s, 6H), 2.54 (s, 3H).

### Example No. I.13-6

¹H NMR (400 MHz, d₆-DMSO δ, ppm) 8.71 (br. t, 1H, NH), 8.62 (d, 1H), 8.39 (d, 1H), 8.28 (d, 1H), 8.07 (d, 1H), 7.92 (dd, 1H), 7.56 (dd, 1H), 7.52 (dd, 1H), 7.41 (d, 1H), 7.23 (d, 1H), 4.24 (d, 2H), 2.81 (s, 6H).

### Example No. I.13-7

¹H NMR (400 MHz, d₆-DMSO δ, ppm) 8.64 (br. t, 1H, NH), 8.37 (d, 1H), 8.34 (d, 1H), 8.25 (d, 1H), 8.10 (d, 1H), 8.06 (d, 1H), 7.52 (dd, 1H), 7.49 (dd, 1H), 7.20 (d, 1H), 4.39 (d, 2H), 2.79 (s, 6H).

### Example No. I.13-8

¹H NMR (400 MHz, d₆-DMSO δ, ppm) 8.51 (br. t, 1H, NH), 8.36 (d, 1H), 8.26 (d, 1H), 8.06 (d, 1H), 8.01 (d, 1H), 7.53 (m, 2H), 7.38 (m, 1H), 7.24 (d, 1H), 7.13 (dd, 1H), 4.19 (d, 2H), 2.83 (s, 6H).

### Example No. I.13-9

¹H NMR (400 MHz, d₆-DMSO δ, ppm) 8.58 (br. t, 1H, NH), 8.36 (d, 1H), 8.29 (d, 1H), 8.03 (d, 1H), 7.98 (d, 1H), 7.50 (m, 2H), 7.48 (m, 1H), 7.20 (d, 1H), 4.18 (d, 2H), 2.80 (s, 6H).

### Example No. I.13-10

¹H NMR (400 MHz, d₆-DMSO δ, ppm) 8.77 (br. t, 1H, NH), 8.43 (s, 1H), 8.34 (d, 1H), 8.21 (d, 1H), 8.02 (d, 1H), 7.55 (dd, 1H), 7.48 (dd, 1H), 7.39 (s, 1H), 7.21 (d, 1H), 4.29 (d, 2H), 2.79 (s, 6H).

### Example No. I.13-11

¹H NMR (400 MHz, d₆-DMSO δ, ppm) 8.50 (m, 2H), 8.30 (d, 1H), 8.15 (d, 1H), 7.78 (dd, 1H), 7.61 (m, 2H), 7.34 (d, 1H), 7.29 (dd, 1H), 7.26 (d, 1H), 4.52 (s, 2H), 2.83 (s, 6H), 2.54 (s, 3H).

### Example No. I.13-20

¹H NMR (400 MHz, d₆-DMSO δ, ppm) 8.49 (m, 2H), 8.22 (d, 1H), 8.20 (d, 1H), 7.69 (dd, 1H), 7.62 (dd, 1H), 7.59 (dd, 1H), 7.34 (m, 2H), 7.29 (d, 1H), 5.26 (q, 1H), 2.83 (s, 6H), 2.70 (s, 3H), 1.36 (d, 3H).

### Example No. I.13-61

¹H NMR (400 MHz, d₆-DMSO, δ ppm) 8.82 (br. t, 1H, NH), 8.37 (d, 1H), 8.26 (d, 1H), 8.12 (d, 1H), 7.61 (d, 2H), 7.58 (dd, 1H), 7.54 (dd, 1H), 7.52 (d, 2H), 7.21 (d, 1H), 6.40 (s, 1H), 4.28 (d, 2H), 2.73 (s, 6H).

### Example No. I.13-62

¹H NMR (400 MHz, CDCl₃ δ, ppm) 8.50 (d, 1H), 8.27 (m, 2H), 7.59 (dd, 1H), 7.50 (dd, 1H), 7.47 (d, 2H), 7.20 (d, 1H), 6.90 (d, 2H), 5.96 (s, 1H), 5.24 (br. t, 1H, NH), 4.29 (d, 2H), 3.84 (s, 3H), 2.85 (s, 6H).

### Example No. I.14-16

¹H NMR (400 MHz, CDCl₃ δ, ppm) 8.20 (s, 1H), 8.09 (d, 1H), 7.81 (d, 1H), 7.73 (d, 1H), 7.60 (d, 1H), 7.39 (d, 1H), 7.19 (dd, 1H), 7.15 (dd, 1H), 6.71 (d, 1H), 6.06 (br. d, NH, 1H), 4.43 (quint, 1H), 4.20 (m, 2H), 1.52 (t, 3H), 1.40 (d, 3H).

### Example No. I.14-17

¹H NMR (400 MHz, CDCl₃ δ, ppm) 8.28 (m, 1H), 8.09 (m, 1H), 7.99 (d, 1H), 7.87 (m, 1H), 7.82 (d, 1H), 7.77 (m, 1H), 7.47 (m, 1H), 7.40 (dd, 1H), 7.21 (m, 1H), 6.74 (d, 1H), 4.77 (m, 2H), 4.18 (q, 2H), 2.38 (m, 1H), 1.49 (t, 3H), 0.48 (m, 4H).

### Example No. I.14-18

¹H NMR (400 MHz, CDCl₃ δ, ppm) 8.46 (d, 2H), 8.22 (d, 1H), 7.98 (d, 1H), 7.94 (d, 1H), 7.80 (d, 1H), 7.63 (dd, 2H), 7.33 (m, 3H), 7.21 (m, 2H), 7.11 (m, 1H), 4.82 (s, 4H), 4.18 (q, 2H), 1.48 (t, 3H).

### Example No. I.14-19

¹H NMR (400 MHz, CDCl₃ δ, ppm) 8.46 (m, 1H), 8.19 (d, 1H), 8.07 (d, 1H), 8.00 (d, 1H), 7.83 (d, 1H), 7.48 (m, 1H), 7.39 (dd, 1H), 7.23 (m, 2H), 4.54 (s, 2H), 4.16 (q, 2H), 1.49 (t, 3H).

### Example No. I.14-20

¹H NMR (400 MHz, CDCl₃ δ, ppm) 8.61 (m, 1H), 8.23 (d, 1H), 7.99 (d, 1H), 7.97 (d, 1H), 7.81 (d, 1H), 7.69 (dd, 1H), 7.40 (m, 1H), 7.37 (dd, 1H), 7.21 (m, 2H), 5.39 (q, 1H), 4.15 (q, 2H), 2.81 (s, 3H), 1.49 (t, 3H), 1.47 (d, 3H).

### Example No. I.14-35

¹H NMR (400 MHz, CDCl₃ δ, ppm) 8.35 (s, 1H), 8.19 (d, 1H), 8.02 (d, 1H), 7.93 (d, 1H), 7.86 (d, 1H), 7.78 (d, 1H), 7.32 (dd, 1H), 7.22 (m, 1H), 6.72 (d, 1H), 5.21 (br. t, NH, 1H), 4.16 (m, 4H), 1.50 (t, 3H).

### Example No. I.14-61

¹H NMR (400 MHz, CDCl₃ δ, ppm) 8.21 (d, 1H), 7.89 (m, 2H), 7.72 (m, 2H), 7.42 (d, 2H), 7.30 (m, 3H), 6.02 (s, 1H), 5.17 (br. t, 1H, NH), 4.30 (d, 2H), 4.22 (q, 2H), 1.49 (t, 3H).

### Example No. I.14-62

¹H NMR (400 MHz, CDCl₃ δ, ppm) 8.22 (d, 1H), 7.91 (m, 2H), 7.76 (d, 1H), 7.49 (d, 2H), 7.34 (dd, 1H), 7.22 (d, 1H), 6.92 (d, 2H), 5.99 (s, 1H), 5.14 (br. t, 1H, NH), 4.28 (d, 2H), 4.21 (q, 2H), 3.86 (s, 3H), 1.48 (t, 3H).

### Example No. I.15-14

¹H NMR (400 MHz, CDCl₃ δ, ppm) 8.21 (d, 1H), 7.98 (m, 2H), 7.55 (dd, 1H), 7.38 (m, 2H), 7.01 (d, 1H), 6.93 (d, 1H), 6.72 (d, 1H), 5.81 (br. t, NH, 1H), 4.13 (d, 2H), 3.03 (s, 3H).

### Example No. I.15-15

¹H NMR (400 MHz, CDCl₃ δ, ppm) 8.20 (m, 1H), 8.10 (d, 1H), 8.02 (m, 2H), 7.84 (m, 2H), 7.52 (m, 1H), 7.31 (m, 2H), 7.22 (m, 1H), 6.81 (d, 1H), 5.79 (br. d, NH, 1H), 4.68 (q, 1H), 3.05 (d, 3H), 1.58 (d, 3H).

### Example No. I.15-16

¹H NMR (400 MHz, CDCl₃ δ, ppm) 8.13 (d, 1H), 8.10 (d, 1H), 7.82 (m, 2H), 7.49 (dd, 1H), 7.38 (dd, 1H), 7.29 (dd, 1H), 6.71 (d, 1H), 6.62 (d, 1H), 6.06 (br. d, NH, 1H), 4.44 (m, 1H), 2.99 (d, 3H), 1.39 (d, 3H).

### Example No. I.15-17

¹H NMR (400 MHz, CDCl₃ δ, ppm) 8.70 (d, 1H), 8.30 (d, 1H), 8.18 (dd, 1H), 8.12 (m, 3H), 8.05 (d, 1H), 7.63 (m, 1H), 7.54 (dd, 1H), 7.50 (dd, 1H), 6.72 (d, 1H), 4.91 (s, 2H), 3.04 (d, 3H), 2.30 (m, 1H), 0.56 (m, 2H), 0.48 (m, 2H).

### Example No. I.15-18

¹H NMR (400 MHz, CDCl₃ δ, ppm) 10.12 (br. m, 1H, NH), 8.49 (m, 2H), 8.23 (d, 1H), 8.06 (d, 1H), 7.98 (d, 1H), 7.72 (dd, 2H), 7.51 (dd, 1H), 7.42 (m, 4H), 7.29 (m, 1H), 6.71 (d, 1H), 4.82 (s, 4H), 3.04 (d, 3H).

### Example No. I.15-19

¹H NMR (400 MHz, CDCl₃ δ, ppm) 10.01 (br. m, 1H, NH), 8.46 (d, 1H), 8.24 (d, 1H), 8.09 (m, 2H), 7.56 (m, 2H), 7.49 (dd, 1H), 7.32 (m, 1H), 6.73 (d, 1H), 4.56 (s, 2H), 3.05 (d, 3H), 2.85 (s, 3H).

### Example No. I.15-20

¹H NMR (400 MHz, CDCl₃ δ, ppm) 10.04 (br. m, 1H, NH), 8.61 (d, 1H), 8.28 (d, 1H), 8.04 (m, 2H), 7.71 (m, 1H), 7.51 (dd, 1H), 7.49 (d, 1H), 7.47 (dd, 1H), 7.26 (m, 1H), 6.70 (d, 1H), 5.40 (q, 1H), 3.04 (d, 3H), 2.76 (s, 3H), 1.43 (d, 3H).

### Example No. I.15-34

¹H NMR (400 MHz, CDCl₃ δ, ppm) 8.40 (d, 2H), 8.16 (d, 1H), 8.06 (d, 1H), 7.87 (d, 1H), 7.55 (m, 2H), 7.39 (m, 2H), 7.10 (d, 1H), 6.73 (d, 1H), 5.62 (br. t, NH, 1H), 4.30 (d, 2H), 3.02 (s, 3H).

### Example No. I.15-35

¹H NMR (400 MHz, CDCl₃ δ, ppm) 8.28 (m, 1H), 8.19 (d, 1H), 8.02 (d, 1H), 7.84 (d, 1H), 7.52 (dd, 1H), 7.39 (dd, 1H), 7.22 (br. m, NH, 1H), 6.92 (m, 2H), 6.71 (d, 1H), 5.79 (br. t, NH, 1H), 4.26 (d, 2H), 3.02 (d, 3H).

### Example No. I.15-62

¹H NMR (400 MHz, CDCl₃ δ, ppm) 8.35 (d, 1H), 7.96 (d, 1H), 7.89 (d, 1H), 7.56 (dd, 1H), 7.44 (d, 2H), 7.42 (dd, 1H), 7.29 (br. m, 1H, NH), 6.91 (d, 2H), 6.68 (d, 1H), 5.81 (s, 1H), 5.20 (br. t, 1H, NH), 4.25 (d, 2H), 3.86 (s, 3H), 2.96 (d, 3H).

### Example No. I.16-14

¹H NMR (400 MHz, CDCl₃ δ, ppm) 8.52 (d, 1H), 8.26 (d, 1H), 8.05 (d, 1H), 7.73 (d, 1H), 7.66 (dd, 1H), 7.51 (dd, 1H), 7.48 (br. s, NH, 1H), 7.38 (dd, 1H), 7.03 (d, 1H), 6.85 (d, 1H), 5.90 (br. t, NH, 1H), 4.19 (d, 2H), 2.38 (s, 3H).

### Example No. I.16-15

¹H NMR (400 MHz, CDCl₃ δ, ppm) 8.55 (d, 1H), 8.08 (m, 1H), 7.92 (d, 1H), 7.82 (m, 2H), 7.74 (m, 1H), 7.55 (m, 3H), 7.18 (m, 1H), 7.11 (m, 1H), 5.82 (br. d, NH, 1H), 4.66 (q, 1H), 2.36 (s, 3H), 1.61 (d, 3H).

### Example No. I.16-16

¹H NMR (400 MHz, CDCl₃ δ, ppm) 8.37 (d, 1H), 8.28 (s, 1H), 8.09 (d, 1H), 7.86 (d, 1H), 7.70 (d, 1H), 7.61 (br. s, NH, 1H), 7.50 (m, 2H), 7.29 (dd, 1H), 6.83 (d, 1H), 6.20 (br. d, NH, 1H), 4.51 (q, 1H), 2.30 (s, 3H), 1.39 (d, 3H).

### Example No. I.16-17

¹H NMR (400 MHz, CDCl₃ δ, ppm) 8.76 (m, 1H), 8.60 (d, 1H), 8.22 (m, 2H), 8.19 (d, 1H), 8.02 (m, 2H), 7.73 (d, 1H), 7.68 (br. s, NH, 1H), 7.61 (dd, 1H), 7.54 (dd, 1H), 4.88 (s, 2H), 2.35 (s, 3H), 2.29 (m, 1H), 0.62 (m, 2H), 0.59 (m, 2H).

### Example No. I.16-18

¹H NMR (400 MHz, CDCl₃ δ, ppm) 8.71 (m, 1H), 8.35 (m, 2H), 8.26 (d, 1H), 8.09 (d, 1H), 8.00 (d, 1H), 7.78 (m, 3H), 7.60 (d, 1H), 7.31 (m, 5H), 4.84 (s, 4H), 2.30 (s, 3H).

### Example No. I.16-19

¹H NMR (400 MHz, CDCl₃ δ, ppm) 8.59 (d, 1H), 8.41 (m, 1H), 8.17 (d, 1H), 8.09 (d, 1H), 7.81 (br. s, NH, 1H), 7.72 (d, 1H), 7.57 (dd, 1H), 7.49 (m, 2H), 7.25 (m, 1H), 4.51 (s, 2H), 2.82 (s, 3H), 2.31 (s, 3H).

### Example No. I.16-20

¹H NMR (400 MHz, CDCl₃ δ, ppm) 8.68 (m, 1H), 8.39 (d, 1H), 8.20 (d, 1H), 8.09 (m, 2H), 7.94 (m, 1H), 7.65 (d, 1H), 7.48 (m, 4H), 5.49 (m, 1H), 2.83 (s, 3H), 2.32 (s, 3H), 1.50 (d, 1H).

### Example No. I.16-28

¹H NMR (400 MHz, CDCl₃ δ, ppm) 9.12 (m, 1H), 8.88 (m, 1H), 8.49 (m, 1H), 8.28 (m, 1H), 8.21 (d, 1H), 8.10 (d, 1H), 7.92 (d, 1H), 7.81 (br. s, NH, 1H), 7.76 (d, 1H), 7.53 (m, 1H), 7.49 (m, 1H), 6.16 (br. t, NH, 1H), 4.27 (d, 2H), 2.38 (s, 3H).

### Example No. I.16-31

¹H NMR (400 MHz, CDCl₃ δ, ppm) 8.47 (d, 1H), 8.20 (d, 1H), 8.16 (d, 1H), 8.08 (d, 1H), 7.78 (d, 1H), 7.63 (m, 2H), 7.51 (m, 2H), 7.06 (d, 1H), 5.39 (br. t, NH, 1H), 4.27 (d, 2H), 2.39 (s, 3H).

### Example No. I.16-32

¹H NMR (400 MHz, CDCl₃ δ, ppm) 8.50 (d, 1H), 8.27 (d, 1H), 8.16 (d, 1H), 8.02 (s, 1H), 7.88 (d, 1H), 7.68 (dd, 1H), 7.57 (dd, 1H), 7.47 (br. s, NH, 1H), 7.39 (m, 1H), 7.09 (d, 1H), 5.02 (br. t, NH, 1H), 4.12 (d, 2H), 2.38 (s, 3H).

### Example No. I.16-34

¹H NMR (400 MHz, CDCl₃ δ, ppm) 8.46 (d, 1H), 8.10 (d, 1H), 8.07 (d, 2H), 8.03 (d, 1H), 7.89 (d, 1H), 7.70 (m, 1H), 7.30 (m, 1H), 7.08 (d, 2H), 5.85 (br. t, NH, 1H), 4.38 (d, 2H), 2.37 (s, 3H).

### Example No. I.16-35

¹H NMR (400 MHz, CDCl₃ δ, ppm) 8.46 (d, 1H), 8.29 (m, 1H), 8.21 (d, 1H), 8.10 (d, 1H), 7.87 (d, 1H), 7.76 (br. s, NH, 1H), 7.65 (dd, 1H), 7.49 (dd, 1H), 7.12 (m, 2H), 5.42 (br. t, NH, 1H), 4.24 (d, 2H), 2.36 (s, 3H).

### Example No. I.17-1

¹H NMR (400 MHz, CDCl₃ δ, ppm) 8.71 (d, 1H), 8.46 (d, 1H), 8.31 (d, 1H), 8.24 (d, 1H), 7.86 (d, 1H), 7.57 (dd, 1H), 7.49 (dd, 1H), 7.42 (m, 1H), 7.01 (dd, 1H), 6.93 (d, 1H), 6.23 (br. t, NH, 1H), 4.21 (d, 2H).

### Example No. I.17-6

¹H NMR (400 MHz, CDCl₃ δ, ppm) 8.67 (d, 1H), 8.47 (m, 2H), 8.31 (d, 1H), 7.89 (d, 1H), 7.67 (dd, 1H), 7.59 (dd, 1H), 7.51 (dd, 1H), 7.08 (d, 1H), 6.07 (br. t, NH, 1H), 4.28 (d, 2H).

### Example No. I.17-7

¹H NMR (400 MHz, CDCl₃ δ, ppm) 8.72 (d, 1H), 8.46 (d, 1H), 8.35 (d, 1H), 8.32 (s, 1H), 7.88 (d, 1H), 7.69 (s, 1H), 7.59 (dd, 1H), 7.50 (dd, 1H), 6.33 (br. t, NH, 1H), 4.39 (d, 2H).

### Example No. I.17-29

¹H NMR (400 MHz, CDCl₃ δ, ppm) 8.72 (d, 1H), 8.56 (d, 1H), 8.22 (d, 1H), 7.64 (t, 1H), 7.48 (t, 1H), 3.83-3.68 (m, 3H), 3.42 (dd, 1H), 3.21 (m, 1H), 3.10 (m, 1H), 2.85 (s, 3H), 2.51 (m, 1H), 1.97 (m, 1H), 1.62 (m, 1H).

### Example No. I.17-39

¹H NMR (400 MHz, CDCl₃ δ, ppm) 8.67 (d, 1H), 8.46 (d, 1H), 8.29 (d, 1H), 7.87 (m, 2H), 7.57 (dd, 1H), 7.47 (dd, 1H), 6.89 (dd, 1H), 7.78 (d, 1H), 6.06 (br. t, NH, 1H), 4.14 (d, 2H), 3.76 (s, 3H).

### Example No. I.17-57

¹H NMR (400 MHz, CDCl₃ δ, ppm) 8.73 (d, 1H), 8.45 (d, 1H), 8.30 (d, 1H), 8.19 (s, 1H), 7.89 (d, 1H), 7.71 (d, 1H), 7.58-7.51 (m, 2H), 6.38 (t, 1H), 4.38 (d, 2H).

### Example No. I.17-62

¹H NMR (400 MHz, CDCl₃ δ, ppm) 8.60 (d, 1H), 8.48 (d, 1H), 8.33 (d, 1H), 7.90 (d, 1H), 7.60 (dd, 1H), 7.52 (dd, 1H), 7.47 (d, 2H), 6.92 (d, 2H), 5.87 (s, 1H), 5.23 (br. t, 1H, NH), 4.33 (d, 2H), 3.85 (s, 3H).

### Example No. I.18-13

¹H NMR (400 MHz, CDCl₃ δ, ppm) 8.47 (d, 1H), 7.24 (d, 1H), 7.17 (br. t, NH, 1H), 7.07 (dd, 1H), 4.48 (d, 2H), 4.06 (s, 3H), 2.94 (s, 2H), 2.50 (s, 3H), 2.46 (s, 3H), 2.08 (s, 3H), 1.46 (s, 6H).

### Example No. I.18-14

¹H NMR (400 MHz, CDCl₃ δ, ppm) 7.50 (dd, 1H), 7.13 (d, 1H), 7.06 (d, 1H), 5.60 (br. t, NH, 1H), 4.19 (d, 2H), 2.95 (s, 2H), 2.58 (s, 3H), 2.50 (s, 3H), 2.08 (s, 3H), 1.47 (s, 6H).

### Example No. I.18-15

¹H NMR (400 MHz, CDCl₃ δ, ppm) 8.56 (d, 1H), 7.70 (dd, 1H), 7.29 (m, 1H), 7.19 (d, 1H), 6.41 (br. d, 1H, NH), 4.56 (quint, 1H), 2.91 (s, 2H), 2.49 (s, 3H), 2.44 (s, 3H), 2.01 (s, 3H), 1.50 (d, 3H), 1.45 (s, 6H).

### Example No. I.18-16

¹H NMR (400 MHz, CDCl₃ δ, ppm) 8.67 (s, 1H), 7.68 (dd, 1H), 7.09 (d, 1H), 5.73 (br. d, 1H, NH), 4.52 (quint, 1H), 2.88 (s, 2H), 2.48 (s, 3H), 2.44 (s, 3H), 1.98 (s, 3H), 1.46 (d, 3H), 1.43 (s, 6H)

### Example No. I.18-17

¹H NMR (400 MHz, CDCl₃ δ, ppm) 8.72 (m, 1H), 7.99 (m., 2H), 7.45 (m, 1H), 4.88 (s, 2H), 2.99 (s, 2H), 2.48 (s, 3H), 2.47 (s, 3H), 2.39 (m, 1H), 2.12 (s, 3H), 1.49 (s, 6H), 0.49 (m, 2H), 0.33 (m, 2H).

### Example No. I.18-18

¹H NMR (400 MHz, CDCl₃ δ, ppm) 8.55 (m, 2H), 7.73 (dd, 2H), 7.41 (d, 2H), 7.29 (m, 2H), 4.69 (s, 4H), 2.96 (s, 2H), 2.49 (s, 6H), 2.07 (s, 3H), 1.49 (s, 6H).

### Example No. I.18-19

¹H NMR (400 MHz, CDCl₃ δ, ppm) 8.40 (d, 1H), 7.32 (s, 1H), 7.20 (dd, 1H), 4.41 (d, 2H), 2.99 (s, 2H), 2.74 (s, 3H), 2.52 (s, 3H), 2.50 (s, 3H), 2.11 (s, 3H), 1.49 (s, 6H).

### Example No. I.18-20

¹H NMR (400 MHz, CDCl₃ δ, ppm) 8.61 (d, 1H), 7.70 (m, 1H), 7.38 (d, 1H), 7.24 (m, 1H), 5.22 (q, 1H), 2.98 (s, 2H), 2.66 (s, 3H), 2.48 (s, 3H), 2.47 (s, 3H), 2.10 (s, 3H), 1.64 (d, 3H), 1.48 (s, 6H).

### Example No. I.18-28

¹H NMR (400 MHz, CDCl₃ δ, ppm) 9.06 (d, 1H), 8.82 (d, 1H), 8.27 (d, 1H), 7.78 (dd, 1H), 6.18 (br. t, 1H, NH), 4.27 (d, 2H), 2.96 (s, 2H), 2.50 (s, 3H), 2.44 (s, 3H), 2.08 (s, 3H), 1.48 (s, 6H).

### Example No. I.18-32

¹H NMR (400 MHz, CDCl₃ δ, ppm) 8.16 (m, 1H), 7.53 (dd, 1H), 7.19 (d, 1H), 4.90 (br. t, NH, 1H), 4.11 (d, 2H), 2.97 (s, 2H), 2.52 (s, 3H), 2.46 (s, 3H), 2.10 (s, 3H), 1.50 (s, 6H).

### Example No. I.18-34

¹H NMR (400 MHz, CDCl₃ δ, ppm) 8.72 (m, 2H), 7.72 (m, 2H), 6.06 (br. m, NH, 1H), 4.32 (d, 2H), 2.98 (s, 2H), 2.54 (s, 3H), 2.43 (s, 3H), 2.12 (s, 3H), 1.50 (s, 6H).

### Example No. I.18-35

¹H NMR (400 MHz, CDCl₃ δ, ppm) 8.50 (m, 1H), 8.38 (d, 1H), 7.32 (d, 1H), 5.21 (br. t, NH, 1H), 4.21 (d, 2H), 2.94 (s, 2H), 2.53 (s, 3H), 2.46 (s, 3H), 2.09 (s, 3H), 1.48 (s, 6H).

### Example No. I.18-61

¹H NMR (400 MHz, CDCl₃ δ, ppm) 7.59 (d, 2H), 7.41 (d, 2H), 6.17 (s, 1H), 5.00 (br. t, 1H, NH), 4.31 (d, 2H), 2.91 (s, 2H), 2.54 (s, 3H), 2.49 (s, 3H), 2.06 (s, 3H), 1.38 (s, 6H).

### Example No. I.18-62

¹H NMR (400 MHz, CDCl₃ δ, ppm) 7.58 (d, 2H), 6.94 (d, 2H), 6.12 (s, 1H), 4.97 (br. t, 1H, NH), 4.29 (d, 2H), 3.85 (s, 3H), 2.91 (s, 2H), 2.54 (s, 3H), 2.49 (s, 3H), 2.06 (s, 3H), 1.38 (s, 6H).

### Example No. I.19-1

¹H NMR (400 MHz, d₆-DMSO δ, ppm) 8.38 (d, 1H), 7.94 (br. t, 1H, NH), 7.64 (dd, 1H), 7.25 (d, 1H), 7.17 (dd, 1H), 6.73 (s, 1H), 4.04 (d, 2H), 3.80 (s, 3H), 2.54 (s, 3H), 2.50 (s, 3H), 2.48 (s, 3H).

### Example No. I.19-2

¹H NMR (400 MHz, d₆-DMSO δ, ppm) 8.57 (d, 2H), 7.91 (br. t, 1H, NH), 7.22 (t, 1H), 6.65 (s, 1H), 4.17 (d, 2H), 3.78 (s, 3H), 2.53 (s, 3H), 2.50 (s, 3H), 2.47 (s, 3H).

### Example No. I.19-5

¹H NMR (400 MHz, d₆-DMSO δ, ppm) 7.97 (s, 1H), 7.90 (br. t, 1H, NH), 6.84 (s, 1H), 6.70 (s, 1H), 4.06 (d, 2H), 3.80 (s, 3H), 3.79 (s, 3H), 3.73 (s, 3H), 2.54 (s, 3H), 2.51 (s, 3H), 2.45 (s, 3H).

### Example No. I.19-6

¹H NMR (400 MHz, d₆-DMSO δ, ppm) 8.69 (d, 1H), 8.12 (br. t, 1H, NH), 8.00 (dd, 1H), 7.43 (d, 1H), 6.65 (s, 1H), 4.18 (d, 2H), 3.78 (s, 3H), 2.53 (s, 3H), 2.50 (s, 3H), 2.42 (s, 3H).

### Example No. I.19-7

¹H NMR (400 MHz, d₆-DMSO δ, ppm) 8.63 (d, 1H), 8.22 (d, 1H), 8.11 (br. t, 1H, NH), 6.60 (s, 1H), 4.34 (d, 2H), 3.75 (s, 3H), 2.52 (s, 3H), 2.50 (s, 3H), 2.42 (s, 3H).

### Example No. I.19-8

¹H NMR (400 MHz, d₆-DMSO δ, ppm) 8.21 (d, 1H), 7.90 (br. t, 1H, NH), 7.46 (dd, 1H), 7.23 (m, 1H), 6.66 (s, 1H), 4.14 (d, 2H), 3.79 (s, 3H), 2.55 (s, 3H), 2.51 (s, 3H), 2.40 (s, 3H).

### Example No. I.19-9

¹H NMR (400 MHz, d₆-DMSO δ, ppm) 8.26 (d, 1H), 7.98 (br. t, 1H, NH), 7.62 (m, 1H), 6.64 (s, 1H), 4.13 (d, 2H), 3.79 (s, 3H), 2.53 (s, 3H), 2.50 (s, 3H), 2.38 (s, 3H).

### Example No. I.19-10

¹H NMR (400 MHz, d₆-DMSO δ, ppm) 8.65 (s, 1H), 8.18 (br. t, 1H, NH), 7.45 (s, 1H), 6.57 (s, 1H), 4.25 (d, 2H), 3.76 (s, 3H), 2.51 (s, 3H), 2.49 (s, 3H), 2.39 (s, 3H).

### Example No. I.19-11

¹H NMR (400 MHz, d₆-DMSO δ, ppm) 8.50 (d, 1H), 7.76 (m, 1H), 7.29 (dd, 1H), 7.26 (d, 1H), 6.86 (s, 1H), 4.35 (s, 2H), 3.85 (s, 3H), 2.63 (s, 3H), 2.62 (s, 3H), 2.51 (s, 3H), 2.47 (s, 3H).

### Example No. I.19-12

¹H NMR (400 MHz, d₆-DMSO δ, ppm) 8.33 (d, 1H), 8.06 (br. t, 1H, NH), 7.28 (dd, 1H), 7.17 (d, 1H), 6.69 (s, 1H), 4.10 (d, 2H), 3.80 (s, 3H), 2.53 (s, 3H), 2.50 (s, 3H), 2.43 (s, 3H).

### Example No. I.19-13

¹H NMR (400 MHz, d₆-DMSO δ, ppm) 8.54 (d, 1H), 8.17 (br. t, 1H, NH), 7.28 (dd, 1H), 7.17 (d, 1H), 6.51 (s, 1H), 4.25 (d, 2H), 3.80 (s, 3H), 3.72 (s, 3H), 2.54 (s, 3H), 2.50 (s, 3H), 2.43 (s, 3H).

### Example No. I.19-14

¹H NMR (400 MHz, d₆-DMSO δ, ppm) 8.03 (br. t, 1H, NH), 7.67 (dd, 1H), 7.24 (d, 1H), 7.21 (d, 1H), 6.69 (s, 1H), 4.04 (d, 2H), 3.80 (s, 3H), 2.54 (s, 3H), 2.50 (s, 3H), 2.45 (s, 3H).

### Example No. I.19-15

¹H NMR (400 MHz, d₆-DMSO δ, ppm) 8.33 (br. d, 1H, NH), 7.91 (d, 1H), 7.57 (dd, 1H), 7.20 (d, 1H), 7.11 (dd, 1H), 6.67 (s, 1H), 4.28 (dq, 1H), 3.78 (s, 3H), 2.53 (s, 3H), 2.50 (s, 3H), 2.42 (s, 3H), 1.27 (d, 3H).

### Example No. I.19-16

¹H NMR (400 MHz, d₆-DMSO δ, ppm) 8.62 (d, 1H), 8.08 (br. d, 1H, NH), 7.92 (dd, 1H), 7.36 (d, 1H), 6.58 (s, 1H), 4.39 (dq, 1H), 3.75 (s, 3H), 2.51 (s, 3H), 2.50 (s, 3H), 2.38 (s, 3H), 1.33 (d, 3H).

### Example No. I.19-17

¹H NMR (400 MHz, d₆-DMSO δ, ppm) 8.34 (d, 1H), 7.60 (dd, 1H), 7.32 (d, 1H), 7.12 (dd, 1H), 6.66 (s, 1H), 4.41 (s, 2H), 3.67 (s, 3H), 2.42 (s, 3H), 2.31 (s, 3H), 2.28 (m, 1H), 2.24 (s, 3H), 0.21 (m, 2H), 0.01 (m, 2H).

### Example No. I.19-18

¹H NMR (400 MHz, d₆-DMSO δ, ppm) 8.42 (d, 2H), 7.62 (dd, 2H), 7.21 (dd, 2H), 7.08 (d, 2H), 6.76 (s, 1H), 4.53 (s, 4H), 3.82 (s, 3H), 2.62 (s, 3H), 2.50 (s, 3H), 2.38 (s, 3H).

### Example No. I.19-19

¹H NMR (400 MHz, d₆-DMSO δ, ppm) 8.42 (d, 1H), 7.43 (dd, 1H), 7.22 (d, 1H), 6.84 (s, 1H), 4.37 (s, 2H), 3.85 (s, 3H), 2.71 (s, 3H), 2.60 (s, 3H), 2.49 (s, 3H), 2.43 (s, 3H).

### Example No. I.19-20

¹H NMR (400 MHz, d₆-DMSO δ, ppm) 8.52 (d, 1H), 7.72 (dd, 1H), 7.28 (dd, 1H), 7.13 (d, 1H), 6.85 (s, 1H), 4.96 (q, 1H), 3.85 (s, 3H), 2.60 (s, 3H), 2.58 (s, 3H), 2.50 (s, 3H), 2.41 (s, 3H), 1.48 (d, 3H).

### Example No. I.19-28

¹H NMR (400 MHz, d₆-DMSO δ, ppm) 8.39 (dd, 1H), 8.35 (d, 1H), 7.97 (br. t, 1H, NH), 7.58 (d, 1H), 7.25 (dd, 1H), 6.75 (s, 1H), 4.00 (d, 2H), 3.81 (s, 3H), 2.57 (s, 3H), 2.51 (s, 3H), 2.44 (s, 3H).

### Example No. I.19-32

¹H NMR (400 MHz, CDCl₃ δ, ppm) 8.12 (m, 1H), 7.49 (dd, 1H), 7.18 (d, 1H); 6.55 (s, 1H), 4.99 (br. t, NH, 1H), 4.10 (d, 2H), 3.86 (s, 3H), 2.64 (s, 3H), 2.54 (s, 3H), 2.12 (s, 3H).

### Example No. I.19-34

¹H NMR (400 MHz, CDCl₃ δ, ppm) 8.70 (m, 2H), 7.79 (m, 2H), 6.63 (br. m, NH, 1H), 6.55 (s, 1H), 4.29 (d, 2H), 3.85 (s, 3H), 2.59 (s, 3H), 2.54 (s, 3H), 2.12 (s, 3H).

### Example No. I.19-35

¹H NMR (400 MHz, d₆-DMSO δ, ppm) 8.48 (s, 1H), 8.40 (d, 1H), 8.13 (br. t, 1H, NH), 7.37 (d, 1H), 6.75 (s, 1H), 4.09 (d, 2H), 3.81 (s, 3H), 2.55 (s, 3H), 2.50 (s, 3H), 2.48 (s, 3H).

### Example No. I.19-62

¹H NMR (400 MHz, CDCl₃ δ, ppm) 7.56 (d, 2H), 6.93 (d, 2H), 6.48 (s, 1H), 6.04 (s, 1H), 5.03 (br. t, 1H, NH), 4.29 (d, 2H), 3.86 (s, 3H), 3.68 (s, 3H), 2.67 (s, 3H), 2.56 (s, 3H), 2.07 (s, 3H).

### Example No. I.20-1

¹H NMR (400 MHz, d₆-DMSO δ, ppm) 9.12 (d, 1H), 9.08 (d, 1H), 8.87 (br. t, 1H, NH), 8.20 (d, 1H), 8.09 (d, 1H), 8.01 (d, 1H), 7.82 (dd, 1H), 7.52 (m, 1H), 7.13 (d, 1H), 7.09 (dd, 1H), 4.17 (d, 2H).

### Example No. I.20-2

¹H NMR (400 MHz, d₆-DMSO δ, ppm) 9.10 (d, 2H), 8.91 (br. t, 1H, NH), 8.34 (d, 2H), 8.04 (d, 1H), 7.98 (d, 1H), 7.80 (m, 1H), 7.12 (t, 1H), 4.30 (d, 2H).

### Example No. I.20-5

¹H NMR (400 MHz, d₆-DMSO δ, ppm) 9.10 (d, 1H), 9.02 (d, 1H), 8.83 (br. t, 1H, NH), 8.07 (d, 1H), 8.02 (d, 1H), 7.81 (dd, 1H), 7.79 (s, 1H), 6.81 (s, 1H), 4.19 (d, 2H), 3.76 (s, 3H), 3.59 (s, 3H).

### Example No. I.20-6

¹H NMR (400 MHz, d₆-DMSO δ, ppm) 9.11 (d, 1H), 9.02 (d, 1H), 8.99 (br. t, 1H, NH), 8.49 (d, 1H), 8.04 (d, 1H), 7.97 (d, 1H), 7.89 (dd, 1H), 7.82 (dd, 1H), 7.35 (d, 1H), 4.28 (d, 2H).

### Example No. I.20-7

¹H NMR (400 MHz, d₆-DMSO δ, ppm) 9.10 (d, 1H), 9.06 (d, 1H), 9.03 (br. t, 1H, NH), 8.25 (s, 1H), 8.10 (s, 1H), 8.01 (d, 1H), 7.93 (d, 1H), 7.80 (dd, 1H), 4.43 (d, 2H).

### Example No. I.20-8

¹H NMR (400 MHz, d₆-DMSO δ, ppm) 9.09 (d, 1H), 9.03 (d, 1H), 8.87 (br. t, 1H, NH), 8.04 (d, 1H), 7.99 (d, 1H), 7.89 (d, 1H), 7.79 (dd, 1H), 7.32 (dd, 1H), 7.10 (m, 1H), 4.25 (d, 2H).

### Example No. I.20-9

¹H NMR (400 MHz, d₆-DMSO δ, ppm) 9.10 (d, 1H), 9.02 (d, 1H), 8.91 (br. t, 1H, NH), 8.05 (d, 1H), 8.01 (d, 1H), 7.97 (d, 1H), 7.78 (dd, 1H), 7.57 (m, 1H), 4.23 (d, 2H).

### Example No. I.20-10

¹H NMR (400 MHz, d₆-DMSO δ, ppm) 9.11 (d, 1H), 9.02 (d, 1H), 9.00 (br. t, 1H, NH), 8.45 (s, 1H), 8.02 (d, 1H), 7.96 (s, 1H), 7.81 (dd, 1H), 7.37 (s, 1H), 4.33 (d, 2H).

### Example No. I.20-11

¹H NMR (400 MHz, d₆-DMSO δ, ppm) 9.15 (d, 1H), 9.09 (d, 1H), 8.41 (d, 1H), 8.21 (d, 1H), 8.12 (d, 1H), 7.84 (dd, 1H), 7.75 (m, 1H), 7.33 (d, 1H), 7.27 (dd, 1H), 4.52 (d, 2H), 2.85 (s, 3H).

### Example No. I.20-12

¹H NMR (400 MHz, d₆-DMSO δ, ppm) 9.10 (d, 1H), 9.06 (d, 1H), 8.91 (br. t, 1H, NH), 8.11 (d, 1H), 8.08 (d, 1H), 7.99 (d, 1H), 7.82 (dd, 1H), 7.16 (m, 1H), 7.07 (d, 1H), 4.21 (d, 2H).

### Example No. I.20-13

¹H NMR (400 MHz, d₆-DMSO δ, ppm) 9.24 (d, 1H), 9.12 (d, 1H), 9.06 (d, 1H), 8.92 (br. t, 1H, NH), 8.09 (d, 1H), 8.06 (d, 1H), 7.93 (d, 1H), 7.86 (dd, 1H), 7.67 (dd, 1H), 4.22 (d, 2H), 3.71 (s, 3H).

### Example No. I.20-14

¹H NMR (400 MHz, d₆-DMSO δ, ppm) 9.11 (d, 1H), 9.06 (d, 1H), 8.91 (br. t, 1H, NH), 8.06 (d, 1H), 7.98 (d, 1H), 7.82 (dd, 1H), 7.55 (dd, 1H), 7.12 (m, 2H), 4.17 (d, 2H).

### Example No. I.20-15

¹H NMR (400 MHz, d₆-DMSO δ, ppm) 9.07 (d, 1H), 9.04 (d, 1H), 8.77 (br. d, 1H, NH), 8.04 (d, 1H), 7.99 (d, 1H), 7.92 (d, 1H), 7.77 (dd, 1H), 7.32 (m, 1H), 6.99 (d, 1H), 6.92 (dd, 1H), 4.40 (quint, 1H), 1.24 (d, 3H).

### Example No. I.20-16

¹H NMR (400 MHz, d₆-DMSO δ, ppm) 9.09 (d, 1H), 9.02 (d, 1H), 8.93 (br. d, 1H, NH), 8.32 (s, 1H), 7.95 (d, 1H), 7.89 (d, 1H), 7.78 (dd, 1H), 7.70 (dd, 1H), 7.20 (d, 1H), 4.53 (quint, 1H), 1.29 (d, 3H).

### Example No. I.20-17

¹H NMR (400 MHz, d₆-DMSO δ, ppm) 9.14 (m, 2H), 8.33 (m, 1H), 8.20 (d, 1H), 8.11 (d, 1H), 7.83 (dd, 1H), 7.74 (m, 1H), 7.39 (d, 1H), 7.23 (dd, 1H), 4.61 (s, 2H), 2.43 (m, 1H), 0.59 (m, 2H), 0.53 (m, 2H).

### Example No. I.20-18

¹H NMR (400 MHz, d₆-DMSO δ, ppm) 9.10 (d, 1H), 9.01 (d, 1H), 8.24 (m, 3H), 8.02 (d, 1H), 7.77 (dd, 1H), 7.59 (m, 2H), 7.14 (m, 4H), 4.72 (s, 4H).

### Example No. I.20-19

¹H NMR (400 MHz, d₆-DMSO δ, ppm) 9.16 (d, 1H), 9.07 (d, 1H), 8.36 (d, 1H), 8.21 (d, 1H), 8.12 (d, 1H), 7.84 (dd, 1H), 7.41 (d, 1H), 7.35 (d, 1H), 4.54 (s, 2H), 2.91 (s, 3H).

### Example No. I.20-20

¹H NMR (400 MHz, d₆-DMSO δ, ppm) 9.14 (d, 1H), 9.03 (d, 1H), 8.37 (d, 1H), 8.23 (d, 1H), 8.11 (d, 1H), 7.82 (dd, 1H), 7.69 (m, 1H), 7.22 (m, 2H), 5.28 (q, 1H), 2.74 (s, 3H), 1.35 (d, 3H).

### Example No. I.20-28

¹H NMR (400 MHz, d₆-DMSO δ, ppm) 9.13 (d, 1H), 9.03 (d, 1H), 8.84 (br. t, 1H, NH), 8.37 (d, 1H), 8.34 (d, 1H), 8.11 (d, 1H), 8.05 (d, 1H), 7.83 (dd, 1H), 7.63 (d, 1H), 7.27 (dd, 1H), 4.14 (d, 2H).

### Example No. I.20-31

¹H NMR (400 MHz, CDCl₃ δ, ppm) 9.12 (d, 1H), 8.98 (d, 1H), 8.18 (m, 2H), 7.88 (d, 1H), 7.64 (dd, 1H), 7.45 (d, 1H), 7.01 (dd, 1H), 5.51 (br. t, NH, 1H), 4.26 (d, 2H).

### Example No. I.20-32

¹H NMR (400 MHz, CDCl₃ δ, ppm) 9.18 (d, 1H), 9.06 (d, 1H), 8.18 (m, 2H), 7.92 (d, 1H), 7.67 (dd, 1H), 7.48 (d, 1H), 7.19 (d, 1H), 5.58 (br. t, NH, 1H), 4.17 (d, 2H).

### Example No. I.20-34

¹H NMR (400 MHz, CDCl₃ δ, ppm) 9.20 (d, 1H), 9.11 (d, 1H), 8.77 (d, 2H), 8.21 (d, 1H), 7.96 (d, 1H), 7.83 (d, 2H), 7.72 (dd, 1H), 6.51 (br. t, NH, 1H), 4.37 (d, 2H).

### Example No. I.20-35

¹H NMR (400 MHz, d₆-DMSO δ, ppm) 9.14 (d, 1H), 9.08 (d, 1H), 8.96 (br. t, 1H, NH), 8.40 (s, 1H), 8.27 (d, 1H), 8.11 (d, 1H), 8.04 (d, 1H), 7.85 (dd, 1H), 7.28 (d, 1H), 4.19 (d, 2H).

### Example No. I.22-1

¹H NMR (400 MHz, d₆-DMSO δ, ppm) 8.66 (br. t, 1H, NH), 8.37 (d, 1H), 7.69 (m, 1H), 7.66 (d, 1H), 7.63 (d, 1H), 7.52 (dd, 1H), 7.33 (d, 1H), 7.21 (dd, 1H), 4.22 (d, 2H).

### Example No. I.22-2

¹H NMR (400 MHz, d₆-DMSO δ, ppm) 8.67 (br. t, 1H, NH), 8.61 (d, 2H), 7.71 (d, 1H), 7.58 (dd, 1H), 7.49 (d, 1H), 7.30 (t, 1H), 4.37 (d, 2H).

### Example No. I.22-5

¹H NMR (400 MHz, d₆-DMSO δ, ppm) 8.65 (br. t, 1H, NH), 7.99 (s, 1H), 7.73 (dd, 1H), 7.60 (m, 1H), 7.54 (dd, 1H), 7.07 (s, 1H), 4.21 (d, 2H), 3.83 (s, 3H), 3.82 (s, 3H).

### Example No. I.22-6

¹H NMR (400 MHz, d₆-DMSO δ, ppm) 8.83 (br. t, 1H, NH), 8.72 (d, 1H), 8.12 (dd, 1H), 7.71 (dd, 1H), 7.58 (dd, 1H), 7.55 (d, 1H), 7.49 (d, 1H), 4.34 (d, 2H).

### Example No. I.22-7

¹H NMR (400 MHz, d₆-DMSO δ, ppm) 8.78 (br. t, 1H, NH), 8.66 (d, 1H), 8.34 (d, 1H), 7.68 (dd, 1H), 7.53 (dd, 1H), 7.44 (d, 1H), 4.49 (d, 2H).

### Example No. I.22-8

¹H NMR (400 MHz, d₆-DMSO δ, ppm) 8.63 (br. t, 1H, NH), 8.19 (d, 1H), 8.69 (dd, 1H), 7.60 (d, 1H), 7.57 (dd, 1H), 7.50 (dd, 1H), 7.31 (m, 1H), 4.31 (d, 2H).

### Example No. I.22-9

¹H NMR (400 MHz, d₆-DMSO δ, ppm) 8.69 (br. t, 1H, NH), 8.28 (d, 1H), 7.79 (m, 1H), 7.71 (dd, 1H), 7.57 (dd, 1H), 7.51 (dd, 1H), 4.28 (d, 2H).

### Example No. I.22-10

¹H NMR (400 MHz, d₆-DMSO δ, ppm) 8.87 (br. t, 1H, NH), 8.72 (s, 1H), 7.69 (m, 2H), 7.56 (dd, 1H), 7.49 (dd, 1H), 4.36 (d, 2H).

### Example No. I.22-11

¹H NMR (400 MHz, d₆-DMSO δ, ppm) 8.45 (d, 1H), 7.88 (d, 1H), 7.79 (m, 1H), 7.75 (dd, 1H), 7.63 (dd, 1H), 7.36 (d, 1H), 7.29 (dd, 1H), 4.43 (s, 2H), 2.83 (s, 3H).

### Example No. I.22-12

¹H NMR (400 MHz, d₆-DMSO δ, ppm) 8.75 (br. t, 1H, NH), 8.34 (d, 1H), 7.73 (dd, 1H), 7.62 (dd, 1H), 7.52 (dd, 1H), 7.36 (m, 2H), 4.25 (d, 2H).

### Example No. I.22-14

¹H NMR (400 MHz, d₆-DMSO δ, ppm) 8.74 (br. t, 1H, NH), 7.76 (d, 1H), 7.73 (dd, 1H), 7.60 (d, 1H), 7.51 (dd, 1H), 7.31 (m, 2H), 4.21 (d, 2H).

### Example No. I.22-15

¹H NMR (400 MHz, d₆-DMSO δ, ppm) 8.65 (br. d, 1H, NH), 8.31 (d, 1H), 7.62 (m, 2H), 7.53 (dd, 1H), 7.44 (dd, 1H), 7.28 (d, 1H), 7.14 (dd, 1H), 4.45 (quint, 1H), 1.33 (d, 3H).

### Example No. I.22-16

¹H NMR (400 MHz, d₆-DMSO δ, ppm) 8.84 (br. d, 1H, NH), 8.66 (d, 1H), 8.05 (dd, 1H), 7.60 (dd, 1H), 7.52 (d, 1H), 7.47 (d, 1H), 7.40 (dd, 1H), 4.57 (quint, 1H), 1.38 (d, 3H).

### Example No. I.22-17

¹H NMR (400 MHz, d₆-DMSO δ, ppm) 8.42 (d, 1H), 7.86 (dd, 1H), 7.75 (m, 2H), 7.64 (dd, 1H), 7.39 (d, 1H), 7.29 (dd, 1H), 4.57 (s, 2H), 2.49 (m, 1H), 0.58 (m, 4H).

### Example No. I.22-18

¹H NMR (400 MHz, d₆-DMSO δ, ppm) 8.34 (m, 2H), 7.76 (dd, 1H), 7.69 (m, 3H), 7.51 (dd, 1H), 7.21 (m, 4H), 4.64 (s,

### Example No. I.22-19

¹H NMR (400 MHz, d₆-DMSO δ, ppm) 8.43 (d, 1H), 7.87 (dd, 1H), 7.74 (dd, 1H), 7.64 (dd, 1H), 7.45 (d, 1H), 7.43 (d, 1H), 4.47 (s, 2H), 2.87 (s, 3H).

### Example No. I.22-20

¹H NMR (400 MHz, d₆-DMSO δ, ppm) 8.44 (d, 1H), 7.82 (dd, 1H), 7.75 (m, 2H), 7.60 (d, 1H), 7.26 (m, 2H), 5.13 (q, 1H), 2.78 (s, 3H), 1.39 (d, 3H).

### Example No. I.22-28

¹H NMR (400 MHz, d₆-DMSO δ, ppm) 8.68 (br. t, 1H), 8.39 (m, 2H), 7.73 (dd, 1H), 7.67 (dd, 1H), 7.61 (dd, 1H), 7.56 (dd, 1H), 7.25 (dd, 1H), 4.16 (d, 2H).

### Example No. I.22-35

¹H NMR (400 MHz, d₆-DMSO δ, ppm) 8.87 (br. t, 1H, NH), 8.52 (s, 1H), 8.46 (d, 1H), 7.78 (dd, 1H), 7.69 (dd, 1H), 7.58 (d, 1H), 7.43 (d, 1H), 4.26 (d, 2H).

### Example No. I.22-61

¹H NMR (400 MHz, CDCl₃ δ, ppm) 7.70 (dd, 1H), 7.63 (d, 2H), 7.43 (d, 2H), 7.34 (m, 2H), 6.33 (s, 1H), 5.31 (br. t, 1H, NH), 4.45 (d, 2H).

### Example No. I.22-62

¹H NMR (400 MHz, CDCl₃ δ, ppm) 7.69 (dd, 1H), 7.60 (d, 2H), 7.35 (d, 1H), 7.33 (m, 1H), 6.95 (d, 2H), 6.27 (s, 1H), 5.36 (br. t, 1H, NH), 4.43 (d, 2H), 3.85 (s, 3H).

### Example No. I.23-1

¹H NMR (400 MHz, d₆-DMSO δ, ppm) 8.64 (br. t, 1H, NH), 8.39 (d, 1H), 8.10 (d, 1H), 8.03 (d, 1H), 7.96 (d, 1H), 7.72 (dd, 1H), 7.32 (d, 1H), 7.22 (dd, 1H), 4.24 (d, 2H).

### Example No. I.23-2

¹H NMR (400 MHz, d₆-DMSO δ, ppm) 8.62 (d, 2H), 8.58 (br. t, 1H, NH), 8.08 (d, 1H), 7.99 (dd, 1H), 7.95 (d, 1H), 7.32 (t, 1H), 4.38 (d, 2H).

### Example No. I.23-5

¹H NMR (400 MHz, d₆-DMSO δ, ppm) 8.55 (br. t, 1H, NH), 8.07 (d, 1H), 8.01 (dd, 1H), 7.95 (s, 1H), 7.89 (d, 1H), 6.93 (s, 1H), 4.18 (d, 2H), 3.80 (s, 3H), 3.76 (s, 3H).

### Example No. I.23-6

¹H NMR (400 MHz, d₆-DMSO δ, ppm) 8.81 (br. t, 1H, NH), 8.77 (d, 1H), 8.14 (dd, 1H), 8.09 (d, 1H), 8.00 (dd, 1H), 7.91 (d, 1H), 7.55 (d, 1H), 4.36 (d, 2H).

### Example No. I.23-7

¹H NMR (400 MHz, d₆-DMSO δ, ppm) 8.70 (d, 1H), 8.67 (br. t, 1H, NH), 8.37 (d, 1H), 8.07 (d, 1H), 7.97 (dd, 1H), 7.89 (d, 1H), 4.51 (d, 2H).

### Example No. I.23-8

¹H NMR (400 MHz, d₆-DMSO δ, ppm) 8.60 (br. t, 1H, NH), 8.21 (d, 1H), 8.08 (d, 1H), 8.00 (dd, 1H), 7.91 (d, 1H), 7.59 (m, 1H), 7.34 (dd, 1H), 4.34 (d, 2H).

### Example No. I.23-9

¹H NMR (400 MHz, d₆-DMSO δ, ppm) 8.67 (br. t, 1H, NH), 8.32 (d, 1H), 8.09 (d, 1H), 8.02 (dd, 1H), 7.88 (d, 1H), 7.82 (m, 1H), 4.31 (d, 2H).

### Example No. I.23-10

¹H NMR (400 MHz, d₆-DMSO δ, ppm) 8.79 (br. t, 1H, NH), 8.76 (s, 1H), 8.07 (d, 1H), 7.98 (dd, 1H), 7.86 (d, 1H), 7.64 (s, 1H), 4.40 (d, 2H).

### Example No. I.23-11

¹H NMR (400 MHz, d₆-DMSO δ, ppm) 8.51 (d, 1H), 8.20 (d, 1H), 8.11 (dd, 1H), 8.04 (d, 1H), 7.81 (m, 1H), 7.39 (d, 1H), 7.33 (dd, 1H), 4.57 (s, 2H), 2.88 (s, 3H).

### Example No. I.23-12

¹H NMR (400 MHz, d₆-DMSO δ, ppm) 8.71 (br. t, 1H, NH), 8.38 (d, 1H), 8.10 (d, 1H), 8.02 (dd, 1H), 7.93 (d, 1H), 7.37 (dd, 1H), 7.33 (d, 1H), 4.27 (d, 2H).

### Example No. I.23-13

¹H NMR (400 MHz, d₆-DMSO δ, ppm) 8.75 (br. t, 1H, NH), 8.42 (d, 1H), 8.12 (d, 1H), 8.09 (dd, 1H), 7.98 (m, 1H), 7.82 (d, 1H), 7.07 (d, 1H), 4.30 (d, 2H), 3.90 (s, 3H).

### Example No. I.23-14

¹H NMR (400 MHz, d₆-DMSO δ, ppm) 8.67 (br. t, 1H, NH), 8.09 (d, 1H), 8.00 (dd, 1H), 7.92 (d, 1H), 7.77 (dd, 1H), 7.32 (m, 2H), 4.25 (d, 2H).

### Example No. I.23-15

¹H NMR (400 MHz, d₆-DMSO δ, ppm) 8.55 (br. d, 1H, NH), 8.35 (d, 1H), 8.00 (d, 1H), 7.94 (dd, 1H), 7.86 (d, 1H), 7.63 (m, 1H), 7.24 (d, 1H), 7.16 (dd, 1H), 4.49 (quint, 1H), 1.36 (d, 3H).

### Example No. I.23-16

¹H NMR (400 MHz, d₆-DMSO δ, ppm) 8.74 (br. d, 1H, NH), 8.72 (d, 1H), 8.07 (dd, 1H), 8.00 (d, 1H), 7.92 (dd, 1H), 7.82 (d, 1H), 7.49 (d, 1H), 4.60 (quint, 1H), 1.40 (d, 3H).

### Example No. I.23-17

¹H NMR (400 MHz, d₆-DMSO δ, ppm) 8.58 (d, 1H), 8.19 (d, 1H), 8.12 (dd, 1H), 8.08 (d, 1H), 7.81 (dd, 1H), 7.42 (d, 1H), 7.30 (dd, 1H), 4.65 (s, 2H), 2.53 (m, 1H), 0.54 (m, 4H).

### Example No. I.23-18

¹H NMR (400 MHz, d₆-DMSO δ, ppm) 8.40 (m, 2H), 8.11 (m, 2H), 8.00 (dd, 1H), 7.69 (m, 2H), 7.21 (m, 4H), 4.68 (s, 4H).

### Example No. I.23-19

¹H NMR (400 MHz, d₆-DMSO δ, ppm) 8.49 (d, 1H), 8.20 (d, 1H), 8.11 (dd, 1H), 8.02 (d, 1H), 7.49 (dd, 1H), 7.43 (d, 1H), 4.60 (s, 2H), 2.92 (s, 3H).

### Example No. I.23-20

¹H NMR (400 MHz, d₆-DMSO δ, ppm) 8.50 (d, 1H), 8.17 (d, 1H), 8.10 (dd, 1H), 8.06 (d, 1H), 7.77 (m, 1H), 7.31 (m, 2H), 5.19 (q, 1H), 2.77 (s, 3H), 1.46 (d, 3H).

### Example No. I.23-28

¹H NMR (400 MHz, d₆-DMSO δ, ppm) 8.66 (br. t, 1H), 8.42 (m, 2H), 8.11 (d, 1H), 8.07 (dd, 1H), 7.95 (d, 1H), 7.63 (dd, 1H), 7.29 (dd, 1H), 4.18 (d, 2H).

### Example No. I.23-35

¹H NMR (400 MHz, d₆-DMSO δ, ppm) 8.84 (br. t, 1H, NH), 8.54 (s, 1H), 8.48 (d, 1H), 8.14 (d, 1H), 8.09 (dd, 1H), 7.98 (d, 1H), 7.43 (d, 1H), 4.27 (d, 2H).

### Example No. I.23-61

¹H NMR (400 MHz, CDCl₃ δ, ppm) 8.09 (d, 1H), 7.98 (d, 1H), 7.76 (d, 1H), 7.68 (d, 2H), 7.56 (d, 2H), 6.78 (s, 1H), 5.53 (br. t, 1H, NH), 4.40 (d, 2H).

### Example No. I.23-62

¹H NMR (400 MHz, CDCl₃ δ, ppm) 8.00 (d, 1H), 7.97 (s, 1H), 7.74 (d, 1H), 7.62 (d, 2H), 6.96 (d, 2H), 6.31 (s, 1H), 5.25 (br. t, 1H, NH), 4.42 (d, 2H), 3.86 (s, 3H).

### Example No. I.24-1

¹H NMR (400 MHz, d₆-DMSO δ, ppm) 8.38 (d, 1H), 8.16 (br. t, 1H, NH), 7.73 (d, 1H), 7.67 (m, 1H), 7.52 (m, 2H), 7.30 (dd, 1H), 7.27 (d, 1H), 7.20 (dd, 1H), 6.86 (d, 1H), 4.01 (d, 2H), 3.86 (s, 3H).

### Example No. I.24-2

¹H NMR (400 MHz, d₆-DMSO δ, ppm) 8.60 (d, 2H), 8.06 (br. t, 1H, NH), 7.69 (d, 1H), 7.49 (m, 2H), 7.28 (t, 1H), 7.22 (dd, 1H), 6.84 (d, 1H), 4.12 (d, 2H), 3.84 (s, 3H).

### Example No. I.24-5

¹H NMR (400 MHz, d₆-DMSO δ, ppm) 8.10 (br. t, 1H, NH), 7.89 (s, 1H), 7.70 (d, 1H), 7.52 (d, 1H), 7.48 (d, 1H), 7.22 (dd, 1H), 6.87 (d, 1H), 6.62 (s, 1H), 3.98 (d, 2H), 3.85 (s, 3H), 3.75 (s, 3H), 3.49 (s, 3H).

### Example No. I.24-6

¹H NMR (400 MHz, d₆-DMSO δ, ppm) 8.69 (d, 1H), 8.33 (br. t, 1H, NH), 7.99 (dd, 1H), 7.69 (d, 1H), 7.51 (d, 1H), 7.48 (m, 2H), 7.22 (dd, 1H), 6.81 (d, 1H), 4.13 (d, 2H), 3.84 (s, 3H).

### Example No. I.24-7

¹H NMR (400 MHz, d₆-DMSO δ, ppm) 8.49 (d, 1H), 8.19 (br. t, 1H, NH), 8.16 (d, 1H), 7.62 (d, 1H), 7.47 (m, 2H), 7.18 (dd, 1H), 6.60 (d, 1H), 4.29 (d, 2H), 3.80 (s, 3H).

### Example No. I.24-8

¹H NMR (400 MHz, d₆-DMSO δ, ppm) 8.20 (d, 1H), 8.06 (br. t, 1H, NH), 7.69 (d, 1H), 7.51 (m, 3H), 7.29 (dd, 1H), 7.24 (dd, 1H), 6.85 (d, 1H), 4.08 (d, 2H), 3.79 (s, 3H).

### Example No. I.24-9

¹H NMR (400 MHz, d₆-DMSO δ, ppm) 8.22 (d, 1H), 8.12 (br. t, 1H, NH), 7.70 (d, 1H), 7.68 (dd, 1H), 7.48 (m, 2H), 7.22 (dd, 1H), 6.82 (d, 1H), 4.07 (d, 2H), 3.84 (s, 3H).

### Example No. I.24-10

¹H NMR (400 MHz, d₆-DMSO δ, ppm) 8.53 (s, 1H), 8.40 (br. t, 1H, NH), 7.61 (d, 1H), 7.49 (d, 1H), 7.38 (d, 1H), 7.30 (s, 1H), 7.12 (dd, 1H), 6.75 (d, 1H), 4.22 (d, 2H), 3.81 (s, 3H).

### Example No. I.24-11

¹H NMR (400 MHz, d₆-DMSO δ, ppm) 8.48 (d, 1H), 7.82 (d, 1H), 7.79 (dd, 1H), 7.58 (m, 2H), 7.38 (dd, 1H), 7.34 (d, 1H), 7.30 (dd, 1H), 6.84 (d, 1H), 4.27 (s, 2H), 3.89 (s, 3H), 2.65 (s, 3H).

### Example No. I.24-12

¹H NMR (400 MHz, d₆-DMSO δ, ppm) 8.30 (d, 1H), 8.27 (br. t, 1H, NH), 7.69 (d, 1H), 7.54 (d, 1H), 7.46 (d, 1H), 7.26 (dd, 1H), 7.20 (dd, 1H), 7.13 (d, 1H), 6.83 (d, 1H), 4.08 (d, 2H), 3.85 (s, 3H).

### Example No. I.24-14

¹H NMR (400 MHz, d₆-DMSO δ, ppm) 8.26 (br. t, 1H, NH), 7.69 (m, 2H), 7.51 (m, 2H), 7.26 (m, 3H), 6.83 (d, 1H), 4.01 (d, 2H), 3.86 (s, 3H).

### Example No. I.24-15

¹H NMR (400 MHz, d₆-DMSO δ, ppm) 8.33 (d, 1H), 8.11 (br. d, 1H, NH), 7.67 (d, 1H), 7.55 (m, 1H), 7.50 (d, 1H), 7.47 (d, 1H), 7.22 (dd, 1H), 7.19 (d, 1H), 7.11 (dd, 1H), 6.85 (d, 1H), 4.32 (quint, 1H), 3.84 (s, 3H), 1.09 (d, 3H).

### Example No. I.24-16

¹H NMR (400 MHz, d₆-DMSO δ, ppm) 8.56 (d, 1H), 8.27 (br. d, 1H, NH), 7.81 (dd, 1H), 7.60 (d, 1H), 7.47 (d, 1H), 7.42 (d, 1H), 7.34 (d, 1H), 7.14 (dd, 1H), 6.79 (d, 1H), 4.41 (quint, 1H), 3.80 (s, 3H), 1.18 (d, 3H).

### Example No. I.24-17

¹H NMR (400 MHz, d₆-DMSO δ, ppm) 8.46 (d, 1H), 7.82 (d, 1H), 7.77 (m, 1H), 7.57 (m, 2H), 7.41 (d, 1H), 7.34 (dd, 1H), 7.27 (dd, 1H), 6.85 (d, 1H), 4.44 (s, 2H), 3.89 (s, 3H), 2.19 (m, 1H), 0.50 (m, 4H).

### Example No. I.24-18

¹H NMR (400 MHz, d₆-DMSO δ, ppm) 8.32 (d, 2H), 7.79 (d, 1H), 7.56 (m, 4H), 7.29 (dd, 1H), 7.14 (m, 4H), 6.81 (d, 1H), 4.52 (s, 4H), 3.88 (s, 3H).

### Example No. I.24-19

¹H NMR (400 MHz, d₆-DMSO δ, ppm) 8.47 (d, 1H), 7.84 (d, 1H), 7.56 (m, 2H), 7.44 (d, 1H), 7.33 (m, 2H), 6.82 (d, 1H), 4.32 (s, 2H), 3.88 (s, 3H), 2.72 (s, 3H).

### Example No. I.24-20

¹H NMR (400 MHz, d₆-DMSO δ, ppm) 8.48 (d, 1H), 7.80 (d, 1H), 7.74 (m, 1H), 7.57 (m, 2H), 7.33 (d, 1H), 7.30 (dd, 1H), 7.27 (dd, 1H), 6.81 (d, 1H), 5.25 (q, 1H), 3.88 (s, 3H), 2.64 (s, 3H), 1.08 (d, 3H).

### Example No. I.24-28

¹H NMR (400 MHz, d₆-DMSO δ, ppm) 8.37 (d, 1H), 8.33 (d, 1H), 8.16 (br. t, 1H, NH), 7.73 (d, 1H), 7.54 (m, 3H), 7.28 (dd, 1H), 7.22 (dd, 1H), 6.85 (d, 1H), 3.96 (d, 2H), 3.86 (s, 3H).

### Example No. I.24-32

¹H NMR (400 MHz, CDCl₃ δ, ppm) 8.19 (d, 1H), 7.69 (d, 1H), 7.62 (d, 1H), 7.53 (d, 1H), 7.29 (m, 1H), 7.22 (d, 1H), 7.06 (dd, 1H), 6.86 (d, 1H), 5.13 (br. t, NH, 1H), 4.19 (d, 2H), 3.86 (s, 3H).

### Example No. I.24-35

¹H NMR (400 MHz, d₆-DMSO δ, ppm) 8.46 (s, 1H), 8.35 (d, 1H), 8.32 (br. t, 1H, NH), 7.73 (d, 1H), 7.53 (m, 2H), 7.35 (d, 1H), 7.26 (dd, 1H), 6.83 (d, 1H), 4.05 (d, 2H), 3.86 (s, 3H).

### Example No. I.24-61

¹H NMR (400 MHz, CDCl₃ δ, ppm) 7.73 (d, 1H), 7.55 (d, 2H), 7.49 (d, 1H), 7.41 (d, 2H), 7.28 (dd, 1H), 7.23 (d, 1H), 6.87 (d, 1H), 6.05 (s, 1H), 5.06 (br. t, 1H, NH), 4.28 (d, 2H), 3.77 (s, 3H).

### Example No. I.24-62

¹H NMR (400 MHz, CDCl₃ δ, ppm) 7.72 (d, 1H), 7.53 (d, 2H), 7.47 (d, 1H), 7.29 (dd, 1H), 7.22 (d, 1H), 6.93 (d, 2H), 6.86 (d, 1H), 5.96 (s, 1H), 5.04 (br. t, 1H, NH), 4.26 (d, 2H), 3.86 (s, 3H), 3.75 (s, 3H).

### Example No. I.26-61

¹H NMR (400 MHz, CDCl₃ δ, ppm) 8.10 (s, 1H), 7.82 (m, 2H), 7.61 (d, 2H), 7.44 (d, 2H), 6.35 (s, 1H), 5.18 (br. t, 1H, NH), 4.44 (d, 2H).

### Example No. I.26-62

¹H NMR (400 MHz, d₆-DMSO, δ ppm) 8.75 (br. t, 1H, NH), 8.06 (m, 2H), 7.91 (dd, 1H), 7.66 (d, 2H), 7.03 (d, 2H), 6.65 (s, 1H), 4.34 (d, 2H), 3.81 (s, 3H).

### Example No. I.27-62

¹H NMR (400 MHz, CDCl₃ δ, ppm) 8.02 (m, 1H), 7.82 (m, 1H), 7.60 (d, 2H), 7.55 (m, 2H), 6.94 (d, 2H), 6.84 (br. t, 1H, NH), 6.34 (s, 1H), 4.45 (q, 2H), 4.40 (d, 2H), 3.85 (s, 3H), 1.42 (t, 3H).

### Example No. I.29-61

¹H NMR (400 MHz, CDCl₃ δ, ppm) 7.86 (m, 2H), 7.63 (d, 2H), 7.42 (d, 2H), 7.15 (m, 2H), 6.38 (s, 1H), 4.98 (br. t, 1H, NH), 4.39 (d, 2H).

### Example No. I.29-62

¹H NMR (400 MHz, CDCl₃ δ, ppm) 7.87 (m, 2H), 7.62 (d, 2H), 7.16 (m, 2H), 6.95 (d, 2H), 6.32 (s, 1H), 5.00 (br. t, 1H, NH), 4.37 (d, 2H), 3.86 (s, 3H).

### Example No. I.30-62

¹H NMR (400 MHz, d₆-DMSO, δ ppm) 8.70 (br. t, 1H, NH), 7.93 (d, 1H), 7.80 (d, 1H), 7.71 (dd, 1H), 7.68 (d, 2H), 7.03 (d, 2H), 6.68 (s, 1H), 4.31 (d, 2H), 3.81 (s, 3H).

### Example No. I.31-62

¹H NMR (400 MHz, CDCl₃ δ, ppm) 7.97 (d, 1H), 7.59 (d, 2H), 7.48 (d, 1H), 7.32 (dd, 1H), 6.97 (d, 2H), 6.23 (s, 1H), 5.60 (br. t, 1H, NH), 4.38 (d, 2H), 3.86 (s, 3H).

### Example No. I.32-38

¹H NMR (400 MHz, CDCl₃ δ, ppm) 8.52 (d, 2H), 7.66 (d, 2H), 7.39 (d, 2H), 7.15 (d, 2H), 4.95 (br. d, 1H, NH), 4.53 (quint, 1H), 1.42 (d, 3H).

### Example No. I.32-46

¹H NMR (400 MHz, CDCl₃ δ, ppm) 7.72 (d, 2H), 7.42 (d, 2H), 7.31 (s, 1H), 6.31 (m, 2H), 5.32 (br. d, 1H, NH), 5.08 (m, 1H).

### Example No. I.32-56

¹H NMR (400 MHz, CDCl₃ δ, ppm) 7.71 (d, 2H), 7.49 (d, 2H), 7.32 (s, 1H), 5.18 (br. t, 1H, NH), 4.37 (d, 2H).

### Example No. I.32-60

¹H NMR (400 MHz, d₆-DMSO δ, ppm) 8.69 (br. t, 1H, NH), 7.79 (d, 2H), 7.74 (m, 2H), 7.61 (d, 2H), 7.49 (m, 3H), 6.70 (s, 1H), 4.30 (d, 2H).

### Example No. I.32-61

¹H NMR (400 MHz, d₆-DMSO δ, ppm) 8.60 (br. t, 1H, NH), 7.80 (d, 4H), 7.60 (d, 2H), 7.58 (d, 2H), 6.74 (s, 1H), 4.30 (d, 2H).

### Example No. I.32-63

¹H NMR (400 MHz, d₆-DMSO δ, ppm) 8.60 (br. t, 1H, NH), 7.79 (d, 2H), 7.60 (d, 2H), 7.52 (m, 3H), 7.50 (d, 1H), 6.59 (s, 1H), 4.36 (d, 2H).

### Example No. I.34-1

¹H NMR (400 MHz, CDCl₃ δ, ppm) 8.52 (d, 1H), 7.96 (dd, 1H), 7.69 (d, 1H), 7.60 (d, 1H), 7.54 (d, 1H), 7.50 (dd, 1H), 7.47 (dd, 1H), 6.81 (br. t, 1H, NH), 4.39 (d, 2H), 2.42 (s, 3H).

### Example No. I.34-2

¹H NMR (400 MHz, CDCl₃ δ, ppm) 8.61 (d, 2H), 7.72 (d, 1H), 7.58 (d, 1H), 7.53 (dd, 1H), 7.18 (t, 1H), 5.89 (br. t, 1H, NH), 4.42 (d, 2H), 2.41 (s, 3H).

### Example No. I.34-3

¹H NMR (400 MHz, d₆-DMSO δ, ppm) 8.32 (br. t, 1H, NH), 8.24 (d, 1H), 7.74 (d, 1H), 7.65 (d, 1H), 7.47 (dd, 1H), 7.04 (m, 2H), 4.08 (d, 2H), 2.36 (s, 3H), 2.22 (s, 3H).

### Example No. I.34-5

¹H NMR (400 MHz, d₆-DMSO δ, ppm) 8.26 (br. t, 1H, NH), 7.92 (s, 1H), 7.71 (d, 1H), 7.58 (d, 1H), 7.44 (dd, 1H), 6.84 (d, 1H), 4.05 (d, 2H), 3.78 (s, 3H), 3.72 (s, 3H), 2.33 (s, 3H).

### Example No. I.34-6

¹H NMR (400 MHz, CDCl₃ δ, ppm) 8.71 (s, 1H), 7.85 (d, 1H), 7.67 (s, 1H), 7.61 (d, 1H), 7.50 (d, 1H), 7.32 (d, 1H), 5.77 (br. t, 1H, NH), 4.34 (d, 2H), 2.41 (s, 3H).

### Example No. I.34-7

¹H NMR (400 MHz, d₆-DMSO δ, ppm) 8.72 (s, 1H), 8.52 (br. t, 1H, NH), 8.36 (s, 1H), 7.68 (d, 1H), 7.59 (d, 1H), 7.42 (dd, 1H), 4.38 (d, 2H), 2.34 (s, 3H).

### Example No. I.34-9

¹H NMR (400 MHz, CDCl₃ δ, ppm) 8.20 (m, 1H), 7.69 (d, 1H), 7.60 (d, 1H), 7.50 (dd, 1H), 7.12 (m, 1H), 5.81 (br. t, 1H, NH), 4.32 (d, 2H), 2.42 (s, 3H).

### Example No. I.34-11

¹H NMR (400 MHz, d₆-DMSO δ, ppm) 8.49 (d, 1H), 7.84 (d, 1H), 7.82 (dd, 1H), 7.80 (d, 1H), 7.58 (dd, 1H), 7.42 (d, 1H), 7.32 (dd, 1H), 4.29 (s, 2H), 2.70 (s, 3H), 2.45 (s, 3H).

### Example No. I.34-15

¹H NMR (400 MHz, d₆-DMSO δ, ppm) 8.36 (d, 1H), 8.34 (br. d, 1H, NH), 7.64 (d, 1H), 7.60 (dd, 1H), 7.51 (d, 1H), 7.38 (dd, 1H), 7.23 (d, 1H), 7.14 (dd, 1H), 4.40 (q, 1H), 2.30 (s, 3H), 1.26 (d, 3H).

### Example No. I.34-16

¹H NMR (400 MHz, d₆-DMSO δ, ppm) 8.69 (d, 1H), 8.53 (br. d, 1H, NH), 8.00 (dd, 1H), 7.61 (d, 1H), 7.44 (d, 1H), 7.42 (d, 1H), 7.31 (dd, 1H), 4.53 (q, 1H), 2.27 (s, 3H), 1.32 (d, 3H).

### Example No. I.34-62

¹H NMR (400 MHz, d₆-DMSO δ, ppm) 7.78 (br. t, 1H, NH), 7.76 (d, 1H), 7.70 (d, 1H), 7.68 (d, 2H), 7.51 (dd, 1H), 7.05 (d, 2H), 6.64 (s, 1H), 4.24 (d, 2H), 3.81 (s, 3H), 2.33 (s, 3H).

### Example No. I.36-1

¹H NMR (400 MHz, CDCl₃ δ, ppm) 8.43 (d, 1H), 7.98 (d, 2H), 7.70 (d, 2H), 7.60 (m, 1H), 7.16 (dd, 1H), 7.12 (d, 1H), 5.50 (br. m, 1H, NH), 4.30 (s, 2H)

### Example No. I.36-61

¹H NMR (400 MHz, CDCl₃ δ, ppm) 7.97 (d, 2H), 7.74 (d, 2H), 7.60 (d, 2H), 7.41 (d, 2H), 6.34 (s, 1H), 5.21 (br. t, 1H, NH), 4.43 (d_{;} 2H).

### Example No. I.36-62

¹H NMR (400 MHz, CDCl₃ δ, ppm) 7.99 (d, 2H), 7.73 (d, 2H), 7.59 (d, 2H), 6.94 (d, 2H), 6.29 (s, 1H), 5.35 (br. t, 1H, NH), 4.41 (d, 2H), 3.85 (s, 3H).

### Example No. I.37-4

¹H NMR (400 MHz, CDCl₃ δ, ppm) 8.08 (d, 1H), 7.98 (d, 1H), 7.83 (d, 2H), 7.78 (d, 1H), 7.70 (m, 1H), 7.52 (dd, 1H), 7.21 (d, 1H), 6.86 (d, 2H), 6.27 (br. t, 1H, NH), 4.41 (d, 2H), 3.78 (s, 3H).

### Example No. I.37-6

¹H NMR (400 MHz, CDCl₃ δ, ppm) 8.71 (s, 1H), 7.83 (d, 1H), 7.76 (d, 2^{\}H), 7.33 (d, 1H), 6.89 (d, 2H), 5.68 (br. t, 1H, NH), 4.32 (d, 2H), 3.84 (s, 3H).

### Example No. I.37-7

¹H NMR (400 MHz, CDCl₃ δ, ppm) 8.60 (s, 1H), 7.83 (s, 1H), 7.79 (d, 2H), 6.88 (d, 2H), 6.02 (br. t, 1H, NH), 4.42 (d, 1H), 3.82 (s, 3H).

### Example No. I.37-38

¹H NMR (400 MHz, CDCl₃ δ, ppm) 8.53 (d, 2H), 7.68 (d, 2H), 7.35 (d, 2H), 6.89 (d, 2H), 5.03 (br. d, 1H, NH), 4.52 (quint, 1H), 3.84 (s, 3H), 1.41 (s, 3H).

### Example No. I.37-39

¹H NMR (400 MHz, CDCl₃ δ, ppm) 8.13 (d, 1H), 7.77 (d, 2H), 7.11 (dd, 1H), 7.09 (d, 1H), 6.90 (d, 2H), 5.59 (br. t, 1H, NH), 4.16 (d, 2H), 3.84 (s, 3H), 3.83 (s, 3H).

### Example No. I.37-46

¹H NMR (400 MHz, CDCl₃ δ, ppm) 7.73 (d, 2H), 7.29 (m, 1H), 6.89 (d, 2H), 6.28 (m, 2H), 5.29 (br. d, 1H, NH), 5.05 (m, 1H), 3.85 (s, 3H).

### Example No. I.37-52

¹H NMR (400 MHz, d₆-DMSO δ, ppm) 8.09 (br. t, 1H, NH), 7.69 (d, 2H), 7.09 (d, 2H), 6.02 (s, 1H), 3.94 (d, 2H), 3.84 (s, 3H), 2.31 (s, 3H).

### Example No. I.37-56

¹H NMR (400 MHz, CDCl₃ δ, ppm) 7.82 (d, 2H), 7.33 (s, 1H), 6.92 (d, 2H), 5.42 (br. t, 1H, NH), 4.35 (d, 2H), 3.85 (s, 3H).

### Example No. I.37-63

¹H NMR (400 MHz, d₆-DMSO δ, ppm) 8.31 (br. t, 1H, NH), 7.71 (d, 2H), 7.61 (d, 1H), 7.57 (dd, 1H), 7.51 (dd, 1H), 7.48 (d, 1H), 7.06 (d, 2H), 6.57 (s, 1H), 4.27 (d, 2H), 3.80 (s, 3H).

### Example No. I.38-1

¹H NMR (400 MHz, CDCl₃ δ, ppm) 8.45 (d, 1H), 7.74 (d, 2H), 7.60 (dd, 1H), 7.23 (d, 2H), 7.15 (m, 2H), 5.86 (br. t, 1H, NH), 4.24 (d, 2H), 2.39 (s, 3H)

### Example No. I.38-4

¹H NMR (400 MHz, CDCl₃ δ, ppm) 8.08 (d, 1H), 7.97 (d, 1H), 7.78 (m, 3H), 7.69 (m, 1H), 7.53 (dd, 1H), 7.22 (d, 1H), 7.19 (d, 2H), 6.31 (br. t, 1H, NH), 4.42 (d, 2H), 2.33 (s, 3H).

### Example No. I.38-6

¹H NMR (400 MHz, CDCl₃ δ, ppm) 8.69 (d, 1H), 7.82 (dd, 1H), 7.69 (d, 2H), 7.32 (d, 1H), 7.22 (d, 2H), 5.71 (br. t, 1H, NH), 4.33 (d, 2H), 2.38 (s, 3H).

### Example No. I.38-7

¹H NMR (400 MHz, CDCl₃ δ, ppm) 8.59 (s, 1H), 7.83 (s, 1H), 7.73 (d, 2H), 7.22 (d, 2H), 6.05 (br. t, 1H, NH), 4.44 (d, 2H), 2.36 (s, 3H).

### Example No. I.38-39

¹H NMR (400 MHz, CDCl₃ δ, ppm) 8.13 (d, 1H), 7.73 (d, 2H), 7.24 (d, 2H), 7.11 (d, 1H), 7.09 (d, 1H), 5.63 (br. t, 1H, NH), 4.17 (d, 2H), 3.82 (s, 3H), 2.39 (s, 3H).

### Example No. I.38-52

¹H NMR (400 MHz, d₆-DMSO δ, ppm) 8.17 (br. t, 1H, NH), 7.65 (d, 2H), 7.36 (d, 2H), 6.01 (s, 1H), 3.95 (d, 2H), 2.38 (s, 3H), 2.31 (s, 3H).

### Example No. I.42-1

¹H NMR (400 MHz, CDCl₃ δ, ppm) 8.63 (d, 1H), 8.42 (d, 1H), 7.98 (dd, 1H), 7.86 (d, 1H), 7.60 (m, 1H), 7.19 (d, 1H), 7.15 (m, 1H), 5.92 (br. m, 1H, NH), 4.46 (d, 2H).

### Example No. I.43-61

¹H NMR (400 MHz, d₆-DMSO δ, ppm) 8.90 (br. t, 1H, NH), 7.83 (s, 1H), 7.77 (d, 2H), 7.60 (m, 2H), 7.57 (d, 2H), 6.80 (s, 1H), 4.40 (d, 2H).

### Example No. I.43-63

¹H NMR (400 MHz, d₆-DMSO δ, ppm) 8.93 (br. t, 1H, NH), 7.87 (s, 1H), 7.60 (m, 2H), 7.51 (m, 3H), 7.48 (d, 1H), 6.62 (s, 1H), 4.48 (d, 2H).

### Example No. I.44-60

¹H NMR (400 MHz, d₆-DMSO δ, ppm) 8.70 (br. t, 1H, NH), 7.96 (d, 1H), 7.72 (m, 2H), 7.52 (m, 2H), 7.47 (m, 4H), 6.68 (s, 1H), 4.38 (d, 2H).

### Example No. I.45-61

¹H NMR (400 MHz, d₆-DMSO δ, ppm) 8.58 (br. t, 1H, NH), 7.72 (d, 2H), 7.57 (d, 2H), 7.40 (d, 1H), 7.32 (dd, 1H), 7.27 (d, 1H), 6.69 (s, 1H), 4.37 (d, 2H), 2.62 (s, 3H).

### Example No. I.48-62

¹H NMR (400 MHz, CDCl₃ δ, ppm) 7.53 (d, 2H), 7.13 (dd, 1H), 6.94 (m, 3H), 6.80 (d, 1H), 6.22 (s, 1H), 6.01 (br. t, 1H, NH), 4.44 (d, 2H), 3.98 (s, 3H), 3.86 (s, 3H).

### Example No. I.49-60

¹H NMR (400 MHz, d₆-DMSO δ, ppm) 8.07 (br. t, 1H, NH), 7.73 (m, 2H), 7.49 (m, 3H), 7.21 (s, 1H), 7.06 (s, 2H), 6.63 (s, 1H), 4.29 (d, 2H), 3.81 (s, 3H), 3.69 (s, 3H)

### Example No. I.50-60

¹H NMR (400 MHz, d₆-DMSO δ, ppm) 8.99 (br. t, 1H, NH), 7.98 (s, 2H), 7.71 (m, 2H), 7.48 (m, 3H), 6.71 (s, 1H), 4.40 (d, 2H).

### Example No. I.50-62

¹H NMR (400 MHz, d₆-DMSO δ, ppm) 8.96 (br. t, 1H, NH), 7.99 (s, 1H), 7.97 (s, 1H), 7.68 (d, 2H), 7.03 (d, 2H), 6.68 (s, 1H), 4.39 (d, 2H), 3.81 (s, 3H).

### Example No. I.51-1

¹H NMR (400 MHz, CDCl₃ δ, ppm) 8.93 (d, 1H), 8.73 (d, 1H), 8.43 (d, 1H), 8.19 (m, 1H), 7.61 (m, 1H), 7.29 (m, 1H), 7.15 (m, 1H), 7.13 (d, 1H), 5.85 (br. m, 1H, NH), 4.37 (d, 2H).

### Example No. I.51-34

¹H NMR (400 MHz, CDCl₃ δ, ppm) 8.69 (d, 2H), 8.00 (d, 1H), 7.79 (dd, 1H), 7.76 (dd, 1H), 7.61 (d, 2H), 7.42 (dd, 1H), 5.52 (br. m, 1H, NH), 4.35 (d, 2H).

### Example No. I.52-60

¹H NMR (400 MHz, d₆-DMSO δ, ppm) 8.89 (br. t, 1H, NH), 7.93 (d, 1H), 7.80 (d, 1H), 7.71 (m, 2H), 7.48 (m, 4H), 6.64 (s, 1H), 4.38 (d, 2H).

### Example No. I.53-1

¹H NMR (400 MHz, CDCl₃ δ, ppm) 8.56 (d, 1H), 8.01 (m, 1H), 7.63 (dd, 1H), 7.56 (dd, 1H), 7.28 (d, 1H), 7.15 (m, 1H), 6.81 (m, 1H), 5.82 (br. m, 1H, NH), 3.99 (d, 2H).

### Example No. I.54-1

¹H NMR (400 MHz, CDCl₃ δ, ppm) 9.11 (d, 1H), 8.40 (d, 1H), 8.28 (dd, 1H), 7.72 (d, 1H), 7.60 (m, 1H), 7.14 (m, 1H), 7.11 (d, 1H), 6.07 (br. m, 1H, NH), 4.38 (d, 2H).

### Example No. I.55-1

¹H NMR (400 MHz, CDCl₃ δ, ppm) 8.93 (d, 1H), 8.74 (d, 1H), 8.43 (d, 1H), 8.21 (m, 1H), 7.62 (m, 1H), 7.15 (m, 1H), 7.12 (d, 1H), 5.89 (br. m, 1H, NH), 4.37 (d, 2H).

### Example No. I.56-1

¹H NMR (400 MHz, CDCl₃ δ, ppm) 9.05 (d, 1H), 8.72 (d, 1H), 8.44 (d, 1H), 8.12 (m, 1H), 7.64 (m, 1H), 7.36 (dd, 1H), 7.20 (m, 1H), 7.18 (d, 1H), 6.27 (br. t, 1H, NH), 4.35 (d, 2H).

### Example No. I.56-34

¹H NMR (400 MHz, CDCl₃ δ, ppm) 9.09 (d, 1H), 8.83 (d, 1H), 8.67 (d, 2H), 8.17 (m, 1H), 7.61 (d, 2H), 7.49 (dd, 1H), 6.23 (br. t, 1H, NH), 4.36 (d, 2H).

### Example No. I.57-1

¹H NMR (400 MHz, CDCl₃ δ, ppm) 9.23 (d, 1H), 8.66 (d, 1H), 8.38 (d, 1H), 8.13 (d, 1H), 7.74 (m, 2H), 7.62 (dd, 1H), 7.51 (dd, 1H), 7.10 (d, 1H), 7.04 (dd, 1H), 5.76 (br. m, 1H, NH), 4.38 (d, 2H).

### Example No. I.58-1

¹H NMR (400 MHz, CDCl₃ δ, ppm) 8.62 (d, 1H), 8.46 (d, 1H), 8.00 (d, 1H), 7.86 (m, 1H), 7.61 (m, 1H), 7.42 (m, 1H), 7.22 (d, 1H), 7.15 (m, 1H), 6.11 (br. m, 1H, NH), 4.46 (d, 2H).

### Example No. I.59-1

¹H NMR (400 MHz, CDCl₃ δ, ppm) 8.69 (d, 1H), 8.42 (d, 1H), 8.12 (d, 1H), 7.62 (m, 1H), 7.17 (m, 1H), 7.12 (d, 1H), 5.92 (br. m, 1H, NH), 4.37 (d, 2H).

### Example No. I.60-1

¹H NMR (400 MHz, CDCl₃ δ, ppm) 8.71 (d, 1H), 8.43 (d, 1H), 8.26 (d, 1H), 7.61 (m, 1H), 7.19 (m, 1H), 7.13 (d, 1H), 5.95 (br. m, 1H, NH), 4.37 (d, 2H).

### Example No. I.61-1

¹H NMR (400 MHz, CDCl₃ δ, ppm) 8.44 (d, 1H), 8.11 (d, 1H), 7.78 (d, 1H), 7.68 (dd, 1H), 7.59 (m, 2H), 7.16 (d, 1H), 7.13 (dd, 1H), 6.38 (br. m, NH, 1H), 4.39 (d, 2H).

### Example No. I.61-15

¹H NMR (400 MHz, CDCl₃ δ, ppm) 8.40 (d, 1H), 8.00 (d, 1H), 7.68 (d, 1H), 7.59 (dd, 1H), 7.51 (m, 2H), 7.08 (dd, 1H), 7.04 (d, 1H), 6.49 (br. m, NH, 1H), 4.63 (q, 1H), 1.50 (d, 3H).

### Example No. I.61-16

¹H NMR (400 MHz, CDCl₃ δ, ppm) 8.64 (s, 1H), 7.97 (d, 1H), 7.78 (dd, 1H), 7.70 (d, 1H), 7.55 (m, 2H), 7.24 (d, 1H), 6.23 (br. d, NH, 1H), 4.75 (q, 1H), 1.52 (d, 3H).

### Example No. I.62-11

¹H NMR (400 MHz, CDCl₃ δ, ppm) 8.48 (d, 1H), 7.90 (d, 1H), 7.72 (m, 2H), 7.47 (d, 1H), 7.39 (m, 1H), 7.21 (dd, 1H), 4.60 (s, 2H), 2.93 (s, 3H).

### Example No. I.63-1

¹H NMR (400 MHz, CDCl₃ δ, ppm) 8.49 (d, 1H), 7.59 (m, 1H), 7.52 (d, 1H), 7.43 (dd, 1H), 7.21 (d, 1H), 7.13 (dd, 1H), 7.07 (d, 1H), 6.36 (br. t, NH, 1H), 4.33 (d, 2H), 3.09 (s, 6H).

### Example No. I.64-11

¹H NMR (400 MHz, CDCl₃ δ, ppm) 8.50 (d, 1H), 7.90 (s, 1H), 7.77 (d, 1H), 7.71 (m, 1H), 7.40 (m, 2H), 7.21 (dd, 1H), 4.58 (s, 2H), 2.90 (s, 3H), 2.51 (s, 3H).

### Example No. I.65-11

¹H NMR (400 MHz, CDCl₃ δ, ppm) 8.53 (d, 1H), 7.73 (m, 2H), 7.52 (m, 3H), 7.21 (dd, 1H), 4.69 (s, 2H), 2.93 (s, 3H), 2.76 (s, 3H).

### Example No. I.66-11

¹H NMR (400 MHz, CDCl₃ δ, ppm) 8.50 (d, 1H), 7.94 (d, 1H), 7.71 (m, 1H), 7.60 (dd, 1H), 7.57 (dd, 1H), 7.53 (d, 1H), 7.21 (dd, 1H), 4.59 (s, 2H), 2.89 (s, 3H), 2.68 (s, 3H).

### Example No. I.67-11

¹H NMR (400 MHz, CDCl₃ δ, ppm) 8.50 (d, 1H), 7.71 (m, 1H), 7.62 (dd, 1H), 7.59 (dd, 1H), 7.48 (m, 2H), 7.40 (m, 3H), 7.38 (m, 1H), 7.20 (m, 2H), 5.31 (s, 2H), 4.59 (s, 2H), 2.90 (s, 3H).

### Example No. I.68-11

¹H NMR (400 MHz, CDCl₃ δ, ppm) 8.50 (d, 1H), 7.94 (d, 1H), 7.74 (m, 2H), 7.59 (d, 1H), 7.52 (d, 1H), 7.25 (m, 1H), 6.91-6.57 (t, 1H), 4.63 (s, 2H), 2.95 (s, 3H).

### Example No. I.69-1

¹H NMR (400 MHz, CDCl₃ δ, ppm) 8.41 (d, 1H), 7.99 (d, 1H), 7.83 (d, 1H), 7.63 (dd, 1H), 7.59 (dd, 1H), 7.27 (d, 1H), 7.17 (dd, 1H), 6.67 (br. t, NH, 1H), 4.45 (d, 2H).

### Example No. I.70-1

¹H NMR (400 MHz, CDCl₃ δ, ppm) 8.47 (d, 1H), 8.12 (d, 1H), 7.66 (d, 1H), 7.57 (dd, 1H), 7.42 (dd, 1H), 7.35 (dd, 1H), 7.13 (m, 2H), 6.14 (br. t, 1H, NH), 4.24 (d, 2H).

### Example No. I.70-3

¹H NMR (400 MHz, d₆-DMSO δ, ppm) 8.46 (br. t, 1H, NH), 8.23 (d, 1H), 7.93 (d, 1H), 7.79 (d, 1H), 7.52 (dd, 1H), 7.48 (dd, 1H), 7.11 (s, 1H), 7.02 (d, 1H), 4.17 (d, 2H), 2.22 (s, 3H).

### Example No. I.70-6

¹H NMR (400 MHz, d₆-DMSO δ, ppm) 8.76 (s, 1H), 8.62 (br. t, 1H, NH), 8.13 (dd, 1H), 7.92 (d, 1H), 7.78 (d, 1H), 7.60 (d, 1H), 7.49 (m, 2H), 4.32 (d, 2H).

### Example No. I.70-9

¹H NMR (400 MHz, d₆-DMSO δ, ppm) 8.48 (br. t, 1H, NH), 8.28 (d, 1H), 7.87 (d, 1H), 7.78 (m 1H), 7.74 (d, 1H), 7.47 (m, 2H), 4.27 (d, 2H).

### Example No. I.70-10

¹H NMR (400 MHz, d₆-DMSO δ, ppm) 8.71 (br. t, 1H, NH), 8.69 (s, 1H), 7.87 (d, 1H), 7.72 (d, 1H), 7.70 (s, 1H), 7.46 (m, 2H), 4.36 (d, 2H).

### Example No. I.70-42

¹H NMR (400 MHz, d₆-DMSO δ, ppm) 8.07 (br. t, 1H, NH), 7.98 (d, 1H), 7.87 (d, 1H), 7.58 (dd, 1H), 7.53 (dd, 1H), 3.61 (m, 1H), 3.57 (m, 2H), 2.82 (m, 2H), 2.27 (m, 1H), 1.83 (m, 1H), 1.78 (m, 1H), 1.48 (m, 1H).

### Example No. I.70-52

¹H NMR (400 MHz, d₆-DMSO δ, ppm) 8.58 (br. t, 1H, NH), 7.94 (d, 1H), 7.82 (d, 1H), 7.52 (m, 2H), 6.01 (s, 1H), 4.12 (d, 2H), 2.29 (s, 3H).

### Example No. I.70-61

¹H NMR (400 MHz, CDCl₃ δ, ppm) 8.09 (d, 1H), 7.69 (d, 1H), 7.59 (d, 2H), 7.42 (m, 3H), 7.34 (dd, 1H), 6.31 (s, 1H), 5.81 (br. t, 1H, NH), 4.38 (d, 2H).

### Example No. I.70-62

¹H NMR (400 MHz, CDCl₃ δ, ppm) 8.10 (d, 1H), 7.68 (d, 1H), 7.58 (d, 2H), 7.40 (dd, 1H), 7.33 (dd, 1H), 6.94 (d, 2H), 6.24 (s, 1H), 5.77 (br. t, 1H, NH), 4.36 (d, 2H), 3.86 (s, 3H).

### Example No. I.71-1

¹H NMR (400 MHz, CDCl₃ δ, ppm) 8.45 (d, 1H), 7.78 (d, 1H), 7.52 (m, 1H), 7.44 (dd, 1H), 7.29 (d, 1H), 7.11 (d, 1H), 7.00 (dd, 1H), 6.87 (d, 1H), 6.19 (br. t, 1H, NH), 4.20 (d, 2H), 3.91 (s, 3H).

### Example No. I.71-61

¹H NMR (400 MHz, CDCl₃ δ, ppm) 7.84 (d, 1H), 7.54 (d, 2H), 7.40 (m, 3H), 6.99 (dd, 1H), 6.88 (d, 1H), 6.12 (s, 1H), 5.56 (br. t, 1H, NH), 4.34 (d, 2H), 3.95 (s, 3H).

### Example No. I.71-62

¹H NMR (400 MHz, CDCl₃ δ, ppm) 7.86 (d, 1H), 7.53 (d, 2H), 7.39 (m, 1H), 6.99 (dd, 1H), 6.92 (d, 2H), 6.87 (d, 1H), 6.06 (s, 1H), 5.58 (br. t, 1H, NH), 4.31 (d, 2H), 3.93 (s, 3H), 3.85 (s, 3H).

### Example No. I.71-63

¹H NMR (400 MHz, CDCl₃ δ, ppm) 7.87 (d, 1H), 7.55 (d, 2H), 7.41 (dd, 1H), 7.01 (dd, 1H), 6.98 (d, 2H), 6.88 (d, 1H), 6.05 (s, 1H), 5.58 (br. t, 1H, NH), 4.33 (d, 2H), 3.93 (s, 3H), 3.85 (s, 1H)

### Example No. I.72-1

¹H NMR (400 MHz, CDCl₃ δ, ppm) 8.45 (d, 1H), 7.79 (d, 2H), 7.60 (m, 3H), 7.50 (br. s, 1H, NH), 7.15 (d, 2H), 5.77 (br. t, 1H, NH), 4.24 (d, 2H), 2.20 (s, 3H).

### Example No. I.72-61

¹H NMR (400 MHz, CDCl₃ δ, ppm) 8.21 (br. s, 1H, NH), 7.74 (d, 2H), 7.68 (d, 2H), 7.42 (d, 2H), 6.63 (d, 2H), 6.44 (s, 1H), 5.93 (br. t, 1H, NH), 4.04 (d, 2H), 2.17 (s, 3H).

### Example No. I.72-62

¹H NMR (400 MHz, d₆-DMSO δ, ppm) 10.21 (br. s, 1H, NH), 8.29 (br. t, 1H, NH), 7.72 (d, 4H), 7.68 (d, 2H), 7.02 (d, 2H), 6.59 (s, 1H), 4.20 (d, 2H), 3.81 (s, 3H), 2.08 (s, 3H).

### Example No. I.73-60

¹H NMR (400 MHz, d₆-DMSO δ, ppm) 8.46 (br. t, 1H, NH), 7.79 (m, 2H), 7.75 (m, 2H), 7.52 (m, 3H), 7.49 (m, 3H), 6.71 (s, 1H), 4.25 (d, 2H).

### Example No. I.73-61

¹H NMR (400 MHz, d₆-DMSO δ, ppm) 8.47 (br. t, 1H, NH), 7.80 (m, 4H), 7.56 (m, 5H), 6.77 (s, 1H), 4.26 (d, 2H).

### Example No. I.73-62

¹H NMR (400 MHz, d₆-DMSO δ, ppm) 8.42 (br. t, 1H, NH), 7.70 (d, 2H), 7.70 (d, 2H), 7.56 (m, 3H), 7.03 (m, 2H), 6.64 (s, 1H), 4.22 (d, 2H), 3.82 (s, 3H).

### Example No. I.77-1

¹H NMR (400 MHz, CDCl₃ δ, ppm) 8.59 (s, 1H), 8.40 (d, 1H), 8.06 (d, 1H), 7.97 (m, 1H), 7.54 (m, 1H), 7.12 (d, 1H), 7.09 (m, 1H), 6.22 (br. t, 1H, NH), 4.35 (d, 2H).

### Example No. I.77-9

¹H NMR (400 MHz, CDCl₃ δ, ppm) 8.61 (s, 1H), 8.16 (m, 1H), 8.08 (d, 1H), 7.98 (dd, 1H), 7.09 (m, 1H), 5.99 (br. t, 1H, NH), 4.41 (d, 2H).

### Example No. I.77-51

¹H NMR (400 MHz, CDCl₃ δ, ppm) 8.59 (s, 1H), 8.44 (br. t, 1H, NH), 8.13 (d, 1H), 7.97 (d, 1H), 3.27 (m, 1H), 3.09 (dd, 1H), 3.03 (dd, 1H), 2.81 (m, 1H), 2.70 (m, 1H), 2.29 (m, 2H), 1.91 (m, 1H), 1.77 (m, 2H), 1.68 (m, 1H), 1.08 (t, 3H).

### Example No. I.78-1

¹H NMR (400 MHz, CDCl₃ δ, ppm) 8.50 (d, 1H), 8.19 (d, 1H), 7.90 (m, 1H), 7.74 (m, 2H), 7.46 (d, 1H), 7.42 (dd, 1H), 7.04 (br. t, 1H, NH), 4.40 (d, 2H), 3.96 (s, 3H).

### Example No. I.78-7

¹H NMR (400 MHz, CDCl₃ δ, ppm) 8.62 (s, 1H), 8.24 (d, 1H), 7.88 (s, 1H), 7.78 (dd, 1H), 7.73 (d, 1H), 6.19 (br. t, 1H, NH), 4.47 (d, 2H), 3.96 (s, 3H).

### Example No. I.78-9

¹H NMR (400 MHz, CDCl₃ δ, ppm) 8.21 (d, 1H), 8.19 (d, 1H), 7.75 (m, 2H), 7.13 (m, 1H), 5.87 (br. t, 1H, NH), 4.35 (d, 2H), 3.96 (s, 3H).

### Example No. I.78-41

¹H NMR (400 MHz, CDCl₃ δ, ppm) 8.18 (d, 1H), 7.74 (d, 1H), 7.69 (dd, 1H), 5.97 (d, 1H), 5.74 (d, 1H), 4.91 (br. t, 1H, NH), 4.18 (d, 2H), 3.96 (s, 3H), 2.06 (s, 3H).

### Example No. I.78-51

¹H NMR (400 MHz, CDCl₃ δ, ppm) 8.77 (br. t, 1H, NH), 8.59 (s, 1H), 8.26 (s, 1H), 7.82 (m, 2H), 3.96 (s, 3H), 3.79 (m, 1H), 3.64 (dd, 1H), 3.61 (dd, 1H), 3.22 (m, 2H), 3.08 (m, 1H), 2.93 (m, 1H), 2.21 (m, 1H), 2.08 (m, 2H), 1.83 (m, 1H), 1.40 (t, 3H).

### Example No. I.79-1

¹H NMR (400 MHz, CDCl₃ δ, ppm) 8.48 (d, 1H), 8.45 (m, 2H), 8.17 (d, 1H), 8.03 (d, 1H), 7.80 (m, 1H), 7.53 (dd, 1H), 7.32 (dd, 1H), 6.76 (br. t, 1H, NH), 4.38 (d, 2H), 3.95 (s, 3H).

### Example No. I.79-7

¹H NMR (400 MHz, CDCl₃ δ, ppm) 8.60 (s, 1H), 8.49 (d, 1H), 8.16 (d, 1H), 8.06 (d, 1H), 7.84 (s, 1H), 7.55 (dd, 1H), 6.19 (br. t, 1H, NH), 4.48 (d, 2H), 3.95 (s, 3H).

### Example No. I.79-9

¹H NMR (400 MHz, CDCl₃ δ, ppm) 8.47 (d, 1H), 8.18 (m, 2H), 8.04 (d, 1H), 7.55 (dd, 1H), 7.10 (dd, 1H), 5.87 (br. t, 1H, NH), 4.36 (d, 2H), 3.96 (s, 3H).

### Example No. I.79-41

¹H NMR (400 MHz, CDCl₃ δ, ppm) 8.45 (d, 1H), 8.19 (d, 1H), 7.98 (dd, 1H), 7.55 (dd, 1H), 5.94 (d, 1H), 5.71 (d, 1H), 4.85 (br. t, 1H, NH), 4.19 (d, 2H), 3.96 (s, 3H), 2.07 (s, 3H).

### Example No. I.79-51

¹H NMR (400 MHz, CDCl₃ δ, ppm) 8.50 (m, 2H), 8.23 (d, 1H), 8.08 (d, 1H), 7.61 (dd, 1H), 3.95 (s, 3H), 3.80 (m, 1H), 3.68 (m, 2H), 3.25 (m, 2H), 3.08 (m, 1H), 2.92 (m, 1H), 2.21 (m, 1H), 2.08 (m, 2H), 1.82 (m, 1H), 1.41 (t, 3H).

### Example No. I.80-1

¹H NMR (400 MHz, CDCl₃ δ, ppm) 8.52 (d, 1H), 8.09 (d, 1H), 8.08 (s, 1H), 7.96 (dd, 1H), 7.79 (d, 1H), 7.61 (dd, 1H), 7.53 (d, 1H), 7.47 (m, 1H), 6.72 (br. t, 1H, NH), 4.42 (d, 2H).

### Example No. I.80-7

¹H NMR (400 MHz, CDCl₃ δ, ppm) 8.64 (s, 1H), 8.18 (d, 1H), 8.10 (dd, 1H), 7.91 (s, 1H), 7.79 (d, 1H), 7.61 (dd, 1H), 6.24 (br. t, 1H, NH), 4.49 (d, 2H).

### Example No. I.80-9

¹H NMR (400 MHz, CDCl₃ δ, ppm) 8.19 (d, 1H), 8.15 (s, 1H), 8.09 (dd, 1H), 7.79 (d, 1H), 7.59 (dd, 1H), 7.18 (m, 1H), 5.93 (br. t, 1H, NH), 4.38 (d, 2H).

### Example No. I.80-51

¹H NMR (400 MHz, CDCl₃ δ, ppm) 8.94 (br. t, 1H, NH), 8.15 (s, 1H), 8.11 (d, 1H), 7.84 (d, 1H), 7.68 (dd, 1H), 3.79 (m, 1H), 3.65 (m, 2H), 3.26 (m, 2H), 3.09 (m, 1H), 2.94 (m, 1H), 2.23 (m, 1H), 2.09 (m, 2H), 1.82 (m, 1H), 1.42 (t, 3H):

### Example No. I.81-1

¹H NMR (400 MHz, CDCl₃ δ, ppm) 8.49 (d, 1H), 8.14 (d, 1H), 8.08 (d, 1H), 7.87 (dd, 1H), 7.58 (dd, 1H), 7.43 (d, 1H), 7.39 (m, 1H), 7.03 (br. t, 1H, NH), 4.44 (q, 2H), 4.39 (d, 2H), 1.42 (t, 3H).

### Example No. I.81-7

¹H NMR (400 MHz, CDCl₃ δ, ppm) 8.61 (s, 1H), 8.18 (d, 1H), 8.06 (d, 1H), 7.88 (s, 1H), 7.60 (dd, 1H), 6.24 (br. t, 1H, NH), 4.47 (d, 2H), 4.40 (q, 2H), 1.43 (t, 3H).

### Example No. I.81-9

¹H NMR (400 MHz, CDCl₃ δ, ppm) 8.59 (m, 1H), 8.16 (d, 1H), 8.07 (d, 1H), 7.63 (dd, 1H), 7.38 (m, 1H), 4.69 (br. t, 1H, NH), 4.40 (d, 2H), 4.15 (q, 2H), 1.43 (t, 3H).

### Example No. I.81-41

¹H NMR (400 MHz, CDCl₃ δ, ppm) 8.13 (d, 1H), 8.07 (d, 1H), 7.51 (dd, 1H), 5.98 (m, 1H), 5.74 (m, 1H), 4.86 (br. t, 1H, NH), 4.44 (q, 2H), 4.19 (d, 2H), 2.07 (s, 3H), 1.41 (t, 3H).

### Example No. I.81-51

¹H NMR (400 MHz, CDCl₃ δ, ppm) 8.71 (br. t, 1H, NH), 8.21 (d, 1H), 8.14 (d, 1H), 7.61 (dd, 1H), 4.43 (q, 2H), 3.80 (m, 1H), 3.66 (m, 2H), 3.29 (m, 1H), 3.21 (m, 1H), 3.09 (m, 1H), 2.92 (m, 1H), 2.22 (m, 1H), 2.09 (m, 2H), 1.83 (m, 1H), 1.42 (m, 6H).

### Example No. I.82-1

¹H NMR (400 MHz, CDCl₃ δ, ppm) 8.45 (d, 1H), 8.38 (d, 1H), 8.20 (dd, 1H), 7.74 (m, 1H), 7.61 (d, 1H), 7.31 (m, 1H), 6.45 (br. t, 1H, NH), 5.36 (s, 2H), 4.35 (d, 2H).

### Example No. I.82-7

¹H NMR (400 MHz, CDCl₃ δ, ppm) 8.63 (s, 1H), 8.44 (d, 1H), 8.23 (d, 1H), 7.89 (s, 1H), 7.61 (d, 1H), 6.28 (br. t, 1H, NH), 5.36 (s, 2H), 4.49 (d, 2H).

### Example No. I.82-9

¹H NMR (400 MHz, CDCl₃ δ, ppm) 8.42 (d, 1H), 8.21 (m, 2H), 7.61 (d, 1H), 7.17 (m, 1H), 5.91 (br. t, 1H, NH), 5.37 (s, 2H), 4.37 (d, 2H).

### Example No. I.82-41

¹H NMR (400 MHz, CDCl₃ δ, ppm) 8.32 (d, 1H), 8.13 (dd, 1H), 7.60 (d, 1H), 5.96 (m, 1H), 5.73 (m, 1H), 5.38 (s, 2H), 5.09 (br. t, 1H, NH), 4.20 (d, 2H), 2.07 (s, 3H).

### Example No. I.82-51

¹H NMR (400 MHz, CDCl₃ δ, ppm) 8.75 (br. t, 1H, NH), 8.39 (d, 1H), 8.24 (dd, 1H), 7.68 (d, 1H), 5.40 (s, 2H), 3.78 (m, 1H), 3.69 (m, 2H), 3.25 (m, 2H), 3.10 (m, 1H), 2.94 (m, 1H), 2.25 (m, 1H), 2.10 (m, 2H), 1.84 (m, 1H), 1.41 (t, 3H).

### Example No. I.83-1

¹H NMR (400 MHz, CDCl₃ δ, ppm) 8.54 (d, 1H), 8.33 (d, 1H), 8.04 (dd, 1H), 7.83 (dd, 1H), 7.64 (d, 1H), 7.51 (dd, 1H), 7.35 (d, 1H), 7.06 (br. t, 1H, NH), 4.43 (d, 2H), 3.92 (s, 3H), 2.64 (s, 3H).

### Example No. I.83-7

¹H NMR (400 MHz, CDCl₃ δ, ppm) 8.58 (s, 1H), 8.34 (d, 1H), 7.85 (dd, 1H), 7.83 (s, 1H), 7.30 (d, 1H), 6.14 (br. t, 1H, NH), 4.46 (d, 2H), 3.91 (s, 3H), 2.60 (s, 3H).

### Example No. I.83-9

¹H NMR (400 MHz, CDCl₃ δ, ppm) 8.35 (d, 1H), 8.18 (d, 1H), 7.84 (dd, 1H), 7.34 (d, 1H), 7.10 (m, 1H), 5.82 (br. t, 1H, NH), 4.33 (d, 2H), 3.91 (s, 3H), 2.63 (s, 3H).

### Example No. I.83-41

¹H NMR (400 MHz, CDCl₃ δ, ppm) 8.33 (d, 1H), 7.80 (dd, 1H), 7.34 (d, 1H), 5.96 (m, 1H), 5.74 (m, 1H), 4.81 (br. t, 1H, NH), 4.14 (d, 2H), 3.92 (s, 3H), 2.66 (s, 3H), 2.08 (s, 3H).

### Example No. I.83-51

¹H NMR (400 MHz, CDCl₃ δ, ppm) 8.38 (d, 1H), 8.24 (br. t, 1H, NH), 7.88 (dd, 1H), 7.40 (d, 1H), 3.92 (s, 3H), 3.81 (m, 1H), 3.69 (m, 2H), 3.29 (m, 1H), 3.21 (m, 1H), 3.08 (m, 1H), 2.93 (m, 1H), 2.67 (s, 3H), 2.21 (m, 1H), 2.09 (m, 2H), 1.84 (m, 1H), 1.42 (t, 3H).

The present invention accordingly provides for the use of at least one compound selected from the group consisting of substituted sulfonamides of the formula (I), and of any desired mixtures of these inventive substituted sulfonamides of the formula (I), alone or in combination with active agrochemical ingredients corresponding to the definition below, for increasing the resistance of plants to abiotic stress factors, especially for boosting plant growth and/or for increasing plant yield.

The present invention provides for application to plants which have been genetically modified (are transgenic) with regard to the abscisic acid signaling chain (ABA signaling chain), and also to those plants which have not been genetically modified with regard to the ABA signaling chain, preference being given to application to plants which have not been genetically modified with regard to the ABA signaling chain.

The present invention further provides a spray solution for treatment of plants, comprising an amount, effective for enhancing the resistance of plants to abiotic stress factors, of at least one compound selected from the group of the compounds of the formula (I). Abiotic stress conditions which can be relativized may include, for example, drought, cold and hot conditions, osmotic stress, waterlogging, elevated soil salinity, elevated exposure to minerals, ozone conditions, strong light conditions, limited availability of nitrogen nutrients, limited availability of phosphorus nutrients.

In one embodiment, it is possible, for example, that the compounds envisaged in accordance with the invention, the substituted sulfonamides of the formula (I), are applied by spray application to appropriate plants or plant parts to be treated. The use of the inventive compounds (I) envisaged in accordance with the invention is effected preferably with a dosage between 0.0005 and 3 kg/ha, more preferably between 0.001 and 2 kg/ha, especially preferably between 0.005 and 1 kg/ha. When, in the context of the present invention, abscisic acid is used simultaneously with aryl- or hetaryl-substituted sulfonamides of the formula (I), for example in the context of a joint preparation or formulation, abscisic acid is preferably added in a dosage between 0.001 and 3 kg/ha, more preferably between 0.005 and 2 kg/ha, especially preferably between 0.01 and 1 kg/ha.

The term “resistance to abiotic stress” is understood in the context of the present invention to mean various kinds of advantages for plants. Such advantageous properties are manifested, for example, in the following improved plant characteristics: improved root growth with regard to surface area and depth, increased stolon and tiller formation, stronger and more productive stolons and tillers, improvement in shoot growth, increased lodging resistance, increased shoot base diameter, increased leaf area, higher yields of nutrients and constituents, for example carbohydrates, fats, oils, proteins, vitamins, minerals, essential oils, dyes, fibers, better fiber quality, earlier flowering, increased number of flowers, reduced content of toxic products such as mycotoxins, reduced content of residues or disadvantageous constituents of any kind, or better digestibility, improved storage stability of the harvested material, improved tolerance to disadvantageous temperatures, improved tolerance to drought and aridity, and also oxygen deficiency as a result of waterlogging, improved tolerance to elevated salt contents in soil and water, enhanced tolerance to ozone stress, improved compatibility with respect to herbicides and other crop treatment compositions, improved water absorption and photosynthesis performance, advantageous plant properties, for example acceleration of ripening, more homogeneous ripening, greater attractiveness to beneficial animals, improved pollination, or other advantages well known to a person skilled in the art.

More particularly, the inventive use of compounds of the formula (I) exhibits the advantages described in spray application to plants and plant parts. Combinations of the corresponding aryl- or hetaryl-substituted sulfonamides of the formula (I) with substances including insecticides, acaricides, attractants, nematicides, fungicides, herbicides, growth regulators, safeners, substances which influence plant maturity, and bactericides can likewise be employed in the control of plant disorders in the context of the present invention. In addition, the combined use of corresponding aryl or hetaryl-substituted sulfonamides of the formula (I) with genetically modified cultivars, or those modified by mutation, with a view to increased tolerance to abiotic stress is likewise possible.

As is known, some of the various kinds of advantages for plants, which have been mentioned above, can be combined, and generally applicable terms can be used to describe them. Such terms are, for example, the following names: phytotonic effect, resistance to stress factors, less plant stress, plant health, healthy plants, plant fitness, plant wellness, plant concept, vigor effect, stress shield, protective shield, crop health, crop health properties, crop health products, crop health management, crop health therapy, plant health, plant health properties, plant health products, plant health management, plant health therapy, greening effect or regreening effect, freshness, or other terms with which a person skilled in the art is quite familiar.

In the context of the present invention, a good effect on resistance to abiotic stress is understood to mean, without limitation,
at least an emergence improved by generally 3%, especially more than 5%, preferably more than 10%,
at least a yield enhanced by generally 3%, especially more than 5%, preferably more than 10%,
at least a root development improved by generally 3%, especially more than 5%, preferably more than 10%,
at least a shoot size rising by generally 3%, especially more than 5%, preferably more than 10%,
at least a leaf area increased by generally 3%, especially more than 5%, preferably more than 10%,
at least an emergence improved by generally 3%, especially more than 5%, preferably more than 10%,
at least a photosynthesis performance improved by generally 3%, especially more than 5%, preferably more than 10%, and/or
at least a flower formation improved by generally 3%, especially more than 5%, preferably more than 10%,
   and the effects may occur individually or else in any combination of two or more effects.

The present invention further provides a spray solution for treatment of plants, comprising an amount, effective for enhancement of the resistance of plants to abiotic stress factors, of at least one compound of the formula (I). The spray solution may comprise other customary constituents, such as solvents, formulation aids, especially water. Further constituents may include active agrochemical ingredients described below.

The present invention further provides for the use of corresponding spray solutions for increasing the resistance of plants to abiotic stress factors. The remarks which follow apply both to the inventive use of the compounds of the formula (I) per se and to the corresponding spray solutions.

In accordance with the invention, it has additionally been found that the application, to plants or in their environment, of the compounds of the formula (I) in combination with at least one fertilizer as defined below is possible.

Fertilizers which can be used in accordance with the invention together with the compounds of the formula (I) elucidated in detail above are generally organic and inorganic nitrogen-containing compounds, for example ureas, urea/formaldehyde condensation products, amino acids, ammonium salts and ammonium nitrates, potassium salts (preferably chlorides, sulfates, nitrates), salts of phosphoric acid and/or salts of phosphorous acid (preferably potassium salts and ammonium salts). In this context, particular mention may be made of the NPK fertilizers, i.e. fertilizers which contain nitrogen, phosphorus and potassium, calcium ammonium nitrate, i.e. fertilizers which additionally contain calcium, or ammonia nitrate sulfate (formula (NH₄)₂SO₄ NH₄NO₃), ammonium phosphate and ammonium sulfate. These fertilizers are common knowledge to those skilled in the art; see also, for example, Ullmann's Encyclopedia of Industrial Chemistry, 5th edition, Vol. A 10, pages 323 to 431, Verlagsgesellschaft, Weinheim, 1987.

The fertilizers may also contain salts of micronutrients (preferably calcium, sulfur, boron, manganese, magnesium, iron, boron, copper, zinc, molybdenum and cobalt) and phytohormones (for example vitamin B1 and indole-3-acetic acid) or mixtures thereof. Fertilizers used in accordance with the invention may also contain other salts such as monoammonium phosphate (MAP), diammonium phosphate (DAP), potassium sulfate, potassium chloride, magnesium sulfate. Suitable amounts of the secondary nutrients, or trace elements, are amounts of 0.5 to 5% by weight, based on the overall fertilizer. Further possible ingredients are crop protection compositions, insecticides or fungicides, growth regulators or mixtures thereof. This will be explained in more detail below.

The fertilizers can be used, for example, in the form of powders, granules, prills or compactates. However, the fertilizers can also be used in liquid form, dissolved in an aqueous medium. In this case, dilute aqueous ammonia can also be used as a nitrogen fertilizer. Further possible ingredients for fertilizers are described, for example, in Ullmann's Encyclopedia of Industrial Chemistry, 5th edition, 1987, volume A 10, pages 363 to 401, DE-A 41 28 828, DE-A 19 05 834 and DE-A 196 31 764. The general composition of the fertilizers which, within the context of the present invention, may take the form of straight and/or compound fertilizers, for example composed of nitrogen, potassium or phosphorus, may vary within a wide range. In general, a content of 1 to 30% by weight of nitrogen (preferably 5 to 20% by weight), 1 to 20% by weight of potassium (preferably 3 to 15% by weight) and a content of 1 to 20% by weight of phosphorus (preferably 3 to 10% by weight) is advantageous. The microelement content is usually in the ppm range, preferably in the range from 1 to 1000 ppm.

In the context of the present invention, the fertilizer and the compounds of the formula (I) may be administered simultaneously, i.e. synchronously. However, it is also possible first to apply the fertilizer and then a compound of the formula (I), or first to apply a compound of the formula (I) and then the fertilizer. In the case of nonsynchronous application of a compound of the formula (I) and the fertilizer, the application in the context of the present invention is, however, effected in a functional relationship, especially within a period of generally 24 hours, preferably 18 hours, more preferably 12 hours, specifically 6 hours, more specifically 4 hours, even more specifically within 2 hours. In very particular embodiments of the present invention, the inventive compound of the formula (I) and the fertilizer are applied within a time frame of less than 1 hour, preferably less than 30 minutes, more preferably less than 15 minutes.

The active ingredients of the formula (I) to be used in accordance with the invention, if appropriate in combination with fertilizers, can preferably be used on the following plants, though the enumeration which follows is not limiting.

Preferred plants are those from the group of the useful plants, ornamental plants, turfgrass types, commonly used trees which are employed as ornamentals in public and domestic areas, and forestry trees. Forestry trees include trees for the production of timber, cellulose, paper and products made from parts of the trees. The term “useful plants” as used here refers to crop plants which are employed as plants for obtaining foods, animal feeds, fuels or for industrial purposes.

The useful plants include, for example, the following types of plants: triticale, durum (hard wheat), turf, vines, cereals, for example wheat, barley, rye, oats, hops, rice, corn and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao beans and peanuts; cucurbits, for example pumpkin/squash, cucumbers and melons; fiber plants, for example cotton, flax, hemp and jute; citrus fruit, for example, oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or also plants such as tobacco, nuts, coffee, aubergine, sugarcane, tea, pepper, grapevines, hops, bananas, latex plants and ornamentals, for example flowers, shrubs, deciduous trees and coniferous trees. This enumeration is not a limitation.

The following plants are considered to be particularly suitable target crops for the application of the method according to the invention: oats, rye, triticale, durum, cotton, aubergine, turf, pome fruit, stone fruit, soft fruit, corn, wheat, barley, cucumber, tobacco, vines, rice, cereals, pear, peppers, beans, soybeans, oilseed rape, tomato, bell pepper, melons, cabbage, potatoes and apples.

Examples of trees which can be improved in accordance with the method according to the invention include: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved by the method according to the invention are: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiate, P. ponderosa, P. contorta, P. sylvestre, P. elliottii, P. montecola, P. albicaulis, P. resinosa, P. palustris, P. taeda, P. flexilis, P. jeffregi, P. baksiana, P. strobes; from the tree species Eucalyptus: E. grandis, E. globulus, E. camadentis, E. nitens, E. obliqua, E. regnans, E. pilularus.

Particularly preferred trees which can be improved by the method according to the invention are: from the tree species Pinus: P. radiate, P. ponderosa, P. contorta, P. sylvestre, P. strobes; from the tree species Eucalyptus: E. grandis, E. globulus and E. camadentis.

Particularly preferred trees which can be improved by the method according to the invention are: horse chestnut, Platanaceae, linden tree, maple tree.

The present invention can also be applied to any turfgrass types, including cool-season turfgrasses and warm-season turfgrasses. Examples of cold-season turfgrasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);
ryegrasses (Lolium spp.), such as annual ryegrass (Lolium multiflorum Lam.), perennial ryegrass (Lolium perenne L.) and italian ryegrass (Lolium multiflorum Lam.);
and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turfgrasses are beachgrass (Ammophila breviligulata Fern.), smooth bromegrass (Bromus inermis Leyss.), cattails such as Timothy (Phleum pratense L.), sand cattail (Phleum subulatum L.), orchard grass (Dactylis glomerata L.), weeping alkaligrass (Puccinellia distans (L.) Parl.) and crested dog's-tail (Cynosurus cristatus L.).

Examples of warm-season turfgrasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpet grass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), Blue gramma (Bouteloua gracilis (H.B.K.) Lag. ex Griffiths), seashore paspalum (Paspalum vaginatum Swartz) and sideoats grama (Bouteloua curtipendula (Michx.) Torr.). Cool-season turfgrasses are generally preferred for the use in accordance with the invention. Particular preference is given to bluegrass, bentgrass and redtop, fescues and ryegrasses. Bentgrass is especially preferred.

More preferably, plants of the plant cultivars which are commercially available or are in use are treated in accordance with the invention. Plant cultivars are understood to mean plants which have new properties (“traits”) and which have been obtained by conventional breeding, by mutagenesis or with the aid of recombinant DNA techniques. Crop plants may accordingly be plants which can be obtained by conventional breeding and optimization methods or by biotechnological and genetic engineering methods or combinations of these methods, including the transgenic plants and including the plant varieties which are protectable and non-protectable by plant breeders' rights.

The inventive treatment method can thus also be used for the treatment of genetically modified organisms (GMOs), e.g. plants or seeds. Genetically modified plants (or transgenic plant's) are plants in which a heterologous gene has been integrated stably into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example antisense technology, cosuppression technology or RNAi technology [RNA interference]). A heterologous gene present in the genome is also called a transgene. A transgene that is defined by its specific presence in the plant genome is called a transformation or transgenic event.

Plants and plant varieties which are preferably treated in accordance with the invention include all plants which have genetic material which imparts particularly advantageous, useful traits to these plants (whether obtained by breeding and/or biotechnological means).

Plants and plant varieties which can likewise be treated in accordance with the invention are those plants which are resistant to one or more abiotic stress factors. The abiotic stress conditions may include, for example, drought, cold and hot conditions, osmotic stress, waterlogging, elevated soil salinity, elevated exposure to minerals, ozone conditions, strong light conditions, limited availability of nitrogen nutrients, limited availability of phosphorus nutrients or avoidance of shade.

Plants and plant varieties which can likewise be treated in accordance with the invention are those plants which are characterized by enhanced yield characteristics. Enhanced yield in these plants may be the result of, for example, improved plant physiology, improved plant growth and development, such as water use efficiency, water retention efficiency, improved nitrogen use, enhanced carbon assimilation, improved photosynthesis, increased germination efficiency and accelerated maturation. Yield can also be affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants which can likewise be treated in accordance with the invention are hybrid plants that already express the characteristics of heterosis, or hybrid vigour, which generally results in higher yield, increased vigour, better health and better resistance towards biotic and abiotic stress factors. Such plants are typically produced by crossing an inbred male-sterile parent line (the female parent) with another inbred male-fertile parent line (the male parent). The hybrid seed is typically harvested from the male-sterile plants and sold to growers. Male-sterile plants can sometimes (for example in corn) be produced by detasseling (i.e. mechanical removal of the male reproductive organs or male flowers); however, it is more typical for male sterility to be the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmatic male sterility (CMS) have been described, for example, for Brassica species (WO 1992/005251, WO 1995/009910, WO 1998/27806, WO 2005/002324, WO 2006/021972 and U.S. Pat. No. 6,229,072). However, genetic determinants for male sterility can also be located in the nuclear genome. Male-sterile plants can also be obtained by plant biotechnology methods such as genetic engineering. A particularly useful means of obtaining male-sterile plants is described in WO 89/10396, in which, for example, a ribonuclease such as a barnase is selectively expressed in the tapetum cells in the stamens. Fertility can then be restored by expression in the tapetum cells of a ribonuclease inhibitor such as barstar (e.g. WO 1991/002069).

Plants or plant cultivars (obtained by plant biotechnology methods such as genetic engineering) which can likewise be treated in accordance with the invention are herbicide-tolerant plants, i.e. plants which have been made tolerant to one or more given herbicides. Such plants can be obtained either by genetic transformation, or by selection of plants containing a mutation imparting such herbicide tolerance.

Herbicide-tolerant plants are, for example, glyphosate-tolerant plants, i.e. plants which have been made tolerant to the herbicide glyphosate or salts thereof. For example, glyphosate-tolerant plants can be obtained by transforming the plant with a gene which encodes the enzyme 5-enolpyruvylshikimate-3-phosphate synthase (EPSPS). Examples of such EPSPS genes are the AroA gene (mutant CT7) of the bacterium Salmonella typhimurium (Comai et al., Science (1983), 221, 370-371), the CP4 gene of the bacterium Agrobacterium sp. (Barry et al., Curr. Topics Plant Physiol. (1992), 7, 139-145), the genes encoding a petunia EPSPS (Shah et al., Science (1986), 233, 478-481), a tomato EPSPS (Gasser et al., J. Biol. Chem. (1988), 263, 4280-4289) or an Eleusine EPSPS (WO 2001/66704). The gene may also be a mutated EPSPS, as described, for example, in EP-A 0837944, WO 2000/066746, WO 2000/066747 or WO 2002/026995. Glyphosate-tolerant plants can also be obtained by expressing a gene that encodes a glyphosate oxidoreductase enzyme as described in U.S. Pat. No. 5,776,760 and U.S. Pat. No. 5,463,175. Glyphosate-tolerant plants can also be obtained by expressing a gene that encodes a glyphosate acetyltransferase enzyme as described, for example, in WO 2002/036782, WO 2003/092360, WO 2005/012515 and WO 2007/024782. Glyphosate-tolerant plants can also be obtained by selecting plants containing naturally occurring mutations of the above-mentioned genes, as described, for example, in WO 2001/024615 or WO 2003/013226.

Other herbicide-resistant plants are, for example, plants that have been made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species). Plants expressing an exogenous phosphinothricin acetyltransferase are described, for example, in U.S. Pat. No. 5,561,236; U.S. Pat. No. 5,648,477; U.S. Pat. No. 5,646,024; U.S. Pat. No. 5,273,894; U.S. Pat. No. 5,637,489; U.S. Pat. No. 5,276,268; U.S. Pat. No. 5,739,082; U.S. Pat. No. 5,908,810 and U.S. Pat. No. 7,112,665.

Further herbicide-tolerant plants are also plants that have been made tolerant to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is converted to homogentisate. Plants tolerant to HPPD-inhibitors can be transformed with a gene encoding a naturally-occurring resistant HPPD enzyme, or a gene encoding a mutated HPPD enzyme according to WO 1996/038567, WO 1999/024585 and WO 1999/024586. Tolerance to HPPD inhibitors can also be obtained by transforming plants with genes encoding certain enzymes enabling the formation of homogentisate despite the inhibition of the native HPPD enzyme by the HPPD inhibitor. Such plants and genes are described in WO 1999/034008 and WO 2002/36787. Tolerance of plants to HPPD inhibitors can also be improved by transforming plants with a gene encoding an enzyme prephenate dehydrogenase in addition to a gene encoding an HPPD-tolerant enzyme, as described in WO 2004/024928.

Further herbicide-resistant plants are plants that have been made tolerant to acetolactate synthase (ALS) inhibitors. The known ALS inhibitors include, for example, sulfonylurea, imidazolinone, triazolopyrimidines, pyrimidinyl oxy(thio)benzoates and/or sulfonylaminocarbonyltriazolinone herbicides. It is known that different mutations in the ALS enzyme (also known as acetohydroxy acid synthase, AHAS) confer tolerance to different herbicides and groups of herbicides, as described, for example, in Tranel and Wright, Weed Science (2002), 50, 700-712, but also in U.S. Pat. No. 5,605,011, U.S. Pat. No. 5,378,824, U.S. Pat. No. 5,141,870 and U.S. Pat. No. 5,013,659. The production of sulfonylurea-tolerant plants and imidazolinone-tolerant plants has been described in U.S. Pat. No. 5,605,011; U.S. Pat. No. 5,013,659; U.S. Pat. No. 5,141,870; U.S. Pat. No. 5,767,361; U.S. Pat. No. 5,731,180; U.S. Pat. No. 5,304,732; U.S. Pat. No. 4,761,373; U.S. Pat. No. 5,331,107; U.S. Pat. No. 5,928,937; and U.S. Pat. No. 5,378,824; and in the international publication WO 1996/033270. Further imidazolinone-tolerant plants have also been described, for example, in WO 2004/040012, WO 2004/106529, WO 2005/020673, WO 2005/093093, WO 2006/007373, WO 2006/015376, WO 2006/024351 and WO 2006/060634. Further sulfonylurea- and imidazolinone-tolerant plants have also been described, for example in WO 2007/024782.

Further plants tolerant to imidazolinone and/or sulfonylurea can be obtained by induced mutagenesis, by selection in cell cultures in the presence of the herbicide or by mutation breeding, as described, for example, for soybeans in U.S. Pat. No. 5,084,082, for rice in WO 1997/41218, for sugarbeet in U.S. Pat. No. 5,773,702 and WO 1999/057965, for lettuce in U.S. Pat. No. 5,198,599 or for sunflower in WO 2001/065922.

Plants or plant cultivars (obtained by plant biotechnology methods such as genetic engineering) which can also be treated in accordance with the invention are insect-resistant transgenic plants, i.e. plants which have been made resistant to attack by certain target insects. Such plants can be obtained by genetic transformation, or by selection of plants containing a mutation which imparts such insect resistance.

In the present context, the term “insect-resistant transgenic plant” includes any plant containing at least one transgene comprising a coding sequence which encodes the following:

1) an insecticidal crystal protein from Bacillus thuringiensis or an insecticidal portion thereof, such as the insecticidal crystal proteins compiled by Crickmore et al., Microbiology and Molecular Biology Reviews (1998), 62, 807-813, updated by Crickmore et al. (2005) in the Bacillus thuringiensis toxin nomenclature, (online at: http://www.lifesci.sussex.ac.uk/Home/Neil_Crickmore/Bt/), or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein from Bacillus thuringiensis or a portion thereof, such as the binary toxin made up of the Cy34 and Cy35 crystal proteins (Moellenbeck et al., Nat. Biotechnol. (2001), 19, 668-72; Schnepf et al., Applied Environm. Microb. (2006), 71, 1765-1774); or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by corn event MON98034 (WO 2007/027777); or
4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in corn events MON863 or MON88017, or the Cry3A protein in corn event MIR 604; or
5) an insecticidal secreted protein from Bacillus thuringiensis or Bacillus cereus, or an insecticidal portion thereof, such as the vegetative insecticidal proteins (VIPs) listed under the following link, for example proteins from the VIP3Aa protein class: http://www.lifesci.sussex.ac.uk/Home/Neil_Crickmore/Bt/vip.html; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 1994/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the abovementioned classes 1 to 8. In one embodiment, an insect-resistant plant contains more than one transgene encoding a protein of any one of the abovementioned classes 1 to 8, to expand the range of target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:

a. plants which contain a transgene capable of reducing the expression and/or the activity of the poly(ADP-ribose)polymerase (PARP) gene in the plant cells or plants, as described in WO 2000/004173 or EP 04077984.5 or EP 06009836.5.
b. plants which contain a stress tolerance-enhancing transgene capable of reducing the expression and/or the activity of the PARG encoding genes of the plants or plant cells, as described, for example, in WO 2004/090140;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase, as described, for example, in EP 04077624.7 or WO 2006/133827 or PCT/EP07/002,433.

Plants or plant cultivars (obtained by plant biotechnology methods such as genetic engineering) which may also be treated according to the invention show altered quantity, quality and/or storage stability of the harvested product and/or altered properties of specific ingredients of the harvested product such as:

1) Transgenic plants which synthesize a modified starch which is altered with respect to its chemophysical traits, in particular the amylose content or the amylose/amylopectin ratio, the degree of branching, the average chain length, the distribution of the side chains, the viscosity behavior, the gel resistance, the grain size and/or grain morphology of the starch in comparison to the synthesized starch in wild-type plant cells or plants, such that this modified starch is better suited for certain applications. These transgenic plants synthesizing a modified starch are described, for example, in EP 0571427, WO 1995/004826, EP 0719338, WO 1996/15248, WO 1996/19581, WO 1996/27674, WO 1997/11188, WO 1997/26362, WO 1997/32985, WO 1997/42328, WO 1997/44472, WO 1997/45545, WO 1998/27212, WO 1998/40503, WO 99/58688, WO 1999/58690, WO 1999/58654, WO 2000/008184, WO 2000/008185, WO 2000/28052, WO 2000/77229, WO 2001/12782, WO 2001/12826, WO 2002/101059, WO 2003/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 2000/22140, WO 2006/063862, WO 2006/072603, WO 2002/034923, EP 06090134.5, EP 06090228.5, EP 06090227.7, EP 07090007.1, EP 07090009.7, WO 2001/14569, WO 2002/79410, WO 2003/33540, WO 2004/078983, WO 2001/19975, WO 1995/26407, WO 1996/34968, WO 1998/20145, WO 1999/12950, WO 1999/66050, WO 1999/53072, U.S. Pat. No. 6,734,341, WO 2000/11192, WO 1998/22604, WO 1998/32326, WO 2001/98509, WO 2001/98509, WO 2005/002359, U.S. Pat. No. 5,824,790, U.S. Pat. No. 6,013,861, WO 1994/004693, WO 1994/009144, WO 1994/11520, WO 1995/35026 and WO 1997/20936.
2) Transgenic plants which synthesize non-starch carbohydrate polymers or which synthesize non-starch carbohydrate polymers with altered properties in comparison to wild-type plants without genetic modification. Examples are plants producing polyfructose, especially of the inulin and levan type, as described in EP 0663956, WO 1996/001904, WO 1996/021023, WO 1998/039460 and WO 1999/024593, plants producing alpha-1,4-glucans, as described in WO 1995/031553, US 2002/031826, U.S. Pat. No. 6,284,479, U.S. Pat. No. 5,712,107, WO 1997/047806, WO 1997/047807, WO 1997/047808 and WO 2000/14249, plants producing alpha-1,6-branched alpha-1,4-glucans, as described in WO 2000/73422, and plants producing alternan, as described in WO 2000/047727, EP 06077301.7, U.S. Pat. No. 5,908,975 and EP 0728213.
3) Transgenic plants which produce hyaluronan, as for example described in WO 2006/032538, WO 2007/039314, WO 2007/039315, WO 2007/039316, JP 2006/304779 and WO 2005/012529.

Plants or plant cultivars (obtained by plant biotechnology methods such as genetic engineering) which may also be treated according to the invention are plants, such as cotton plants, with altered fiber characteristics. Such plants can be obtained by genetic transformation, or by selection of plants containing a mutation imparting such altered fiber characteristics and include:

a) plants, such as cotton plants, which contain an altered form of cellulose synthase genes, as described in WO 1998/000549;
b) plants, such as cotton plants, which contain an altered form of rsw2 or rsw3 homologous nucleic acids, as described in WO 2004/053219;
c) plants, such as cotton plants, with an increased expression of sucrose phosphate synthase, as described in WO 2001/017333;
d) plants, such as cotton plants, with an increased expression of sucrose synthase, as described in WO 02/45485;
e) plants, such as cotton plants, wherein the timing of the plasmodesmatal gating at the basis of the fiber cell is altered, for example through downregulation of fiber-selective β-1,3-glucanase as described in WO 2005/017157;
f) plants, such as cotton plants, which have fibers with altered reactivity, for example through the expression of the N-acetylglucosaminetransferase gene including nodC and chitin synthase genes, as described in WO 2006/136351.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants containing a mutation imparting such altered oil characteristics and include:

a) plants, such as oilseed rape plants, which produce oil having a high oleic acid content, as described, for example, in U.S. Pat. No. 5,969,169, U.S. Pat. No. 5,840,946 or U.S. Pat. No. 6,323,392 or U.S. Pat. No. 6,063,947;
b) plants, such as oilseed rape plants, which produce oil having a low linolenic acid content, as described in U.S. Pat. No. 6,270,828, U.S. Pat. No. 6,169,190 or U.S. Pat. No. 5,965,755;
c) plants, such as oilseed rape plants, which produce oil having a low level of saturated fatty acids, as described, for example, in U.S. Pat. No. 5,434,283.

Particularly useful transgenic plants which may be treated according to the invention are plants which comprise one or more genes which encode one or more toxins and are the transgenic plants which are sold under the following trade names: YIELD GARD® (for example corn, cotton, soybeans), KnockOut® (for example corn), BiteGard® (for example corn), BT-Xtra® (for example corn), StarLink® (for example corn), Bollgard® (cotton), Nucotn® (cotton), Nucotn 33B® (cotton), NatureGard® (for example corn), Protecta® and NewLeaf® (potato). Examples of herbicide-tolerant plants which should be mentioned are corn varieties, cotton varieties and soybean varieties which are available under the following trade names: Roundup Ready® (tolerance to glyphosate, for example corn, cotton, soybeans), Liberty Link® (tolerance to phosphinothricin, for example oilseed rape), IMI® (tolerance to imidazolinone) and SCS® (tolerance to sulfonylurea, for example corn). Herbicide-resistant plants (plants bred in a conventional manner for herbicide tolerance) which should be mentioned include the varieties sold under the Clearfield® name (for example corn).

Particularly useful transgenic plants which may be treated according to the invention are plants containing transformation events, or a combination of transformation events, and that are listed for example in the databases for various national or regional regulatory agencies.

The compounds of the formula (I) to be used in accordance with the invention can be converted to customary formulations, such as solutions: emulsions, wettable powders, water- and oil-based suspensions, powders, dusts, pastes, soluble powders, soluble granules, granules for broadcasting, suspoemulsion concentrates, natural compounds impregnated with active ingredient, synthetic substances impregnated with active ingredient, fertilizers, and also microencapsulations in polymeric substances. In the context of the present invention, it is especially preferred when the compounds of the formula (I) are used in the form of a spray formulation.

The present invention therefore additionally also relates to a spray formulation for enhancing the resistance of plants to abiotic stress. A spray formulation is described in detail hereinafter:

The formulations for spray application are produced in a known manner, for example by mixing the compounds of the formula (I) for use in accordance with the invention with extenders, i.e. liquid solvents and/or solid carriers, optionally with use of surfactants, i.e. emulsifiers and/or dispersants and/or foam formers. Further customary additives, for example customary extenders and solvents or diluents, dyes, wetting agents, dispersants, emulsifiers, antifoams, preservatives, secondary thickeners, stickers, gibberellins and also water, can optionally also be used. The formulations are prepared either in suitable facilities or else before or during application.

The auxiliaries used may be those substances which are suitable for imparting, to the composition itself and/or to preparations derived therefrom (for example spray liquors), particular properties such as particular technical properties and/or else special biological properties. Typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which may optionally also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender utilized is water, it is also possible to use, for example, organic solvents as auxiliary solvents. Useful liquid solvents essentially include: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethyl sulfoxide, and also water.

It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

Useful wetting agents which may be present in the formulations usable in accordance with the invention are all substances which promote wetting and which are conventionally used for the formulation of active agrochemical ingredients.

Preference is given to using alkyl naphthalenesulfonates, such as diisopropyl or diisobutyl naphthalenesulfonates.

Useful dispersants and/or emulsifiers which may be present in the formulations usable in accordance with the invention are all nonionic, anionic and cationic dispersants conventionally used for the formulation of active agrochemical ingredients. Usable with preference are nonionic or anionic dispersants or mixtures of nonionic or anionic dispersants. Suitable nonionic dispersants are especially ethylene oxide/propylene oxide block polymers, alkylphenol polyglycol ethers and tristryrylphenol polyglycol ether, and the phosphated or sulfated derivatives thereof. Suitable anionic dispersants are especially lignosulfonates, polyacrylic acid salts and arylsulfonate/formaldehyde condensates.

Antifoams which may be present in the formulations usable in accordance with the invention are all foam-inhibiting substances conventionally used for the formulation of active agrochemical ingredients. Silicone antifoams and magnesium stearate can be used with preference.

Preservatives which may be present in the formulations usable in accordance with the invention are all substances usable for such purposes in agrochemical compositions. Examples include dichlorophene and benzyl alcohol hemiformal.

Secondary thickeners which may be present in the formulations usable in accordance with the invention are all substances usable for such purposes in agrochemical compositions. Preferred examples include cellulose derivatives, acrylic acid derivatives, xanthan, modified clays and finely divided silica.

Stickers which may be present in the formulations usable in accordance with the invention include all customary binders usable in seed-dressing products. Preferred examples include polyvinylpyrrolidone, polyvinyl acetate, polyvinyl alcohol and tylose. Gibberellins which may be present in the formulations usable in accordance with the invention may preferably be gibberellins A1, A3 (=gibberellic acid), A4 and A7; particular preference is given to using gibberellic acid. The gibberellins are known (cf. R. Wegler “Chemie der Pflanzenschutz- and Schädlingsbekämpfungsmittel” [Chemistry of the Crop Protection Compositions and Pesticides], vol. 2, Springer Verlag, 1970, p. 401-412).

Further additives may be fragrances, mineral or vegetable, optionally modified oils, waxes and nutrients (including trace nutrients), such as salts of iron, manganese, boron, copper, cobalt, molybdenum and zinc. Additionally present may be stabilizers, such as cold stabilizers, antioxidants, light stabilizers or other agents which improve chemical and/or physical stability.

The formulations contain generally between 0.01 and 98% by weight, preferably between 0.5 and 90%, of the compound of the formula (I).

The inventive active ingredient may be present in its commercially available formulations and in the use forms, prepared from these formulations, as a mixture with other active ingredients, such as insecticides, attractants, sterilants, bactericides, acaricides, nematicides, fungicides, growth regulators, herbicides, safeners, fertilizers or semiochemicals.

In addition, the described positive effect of the compounds of the formula (I) on the plants' own defenses can be supported by an additional treatment with active insecticidal, fungicidal or bactericidal ingredients.

Preferred times for the application of compounds of the formula (I) for enhancing resistance to abiotic stress are treatments of the soil, stems and/or leaves with the approved application rates.

The active ingredients of the formula (I) may generally additionally be present in their commercial formulations and in the use forms prepared from these formulations in mixtures with other active ingredients, such as insecticides, attractants, sterilants, acaricides, nematicides, fungicides, bactericides, growth regulators, substances which influence plant maturity, safeners or herbicides. Particularly favorable mixing partners are, for example, the active ingredients of the different classes, specified below in groups, without any preference resulting from the sequence thereof:

### Fungicides:

F1) nucleic acid synthesis inhibitors, for example benalaxyl, benalaxyl-M, bupirimate, chiralaxyl, clozylacon, dimethirimol, ethirimol, furalaxyl, hymexazole, metalaxyl, metalaxyl-M, ofurace, oxadixyl, oxolinic acid;
F2) mitosis and cell division inhibitors, for example benomyl, carbendazim, diethofencarb, fuberidazole, fluopicolid, pencycuron, thiabendazole, thiophanate-methyl, zoxamide and chloro-7-(4-methylpiperidin-1-yl)-6-(2,4,6-trifluorophenyl)[1,2,4]triazolo[1,5-a]pyrimidine;
F3) respiratory chain complex I/II inhibitors, for example diflumetorim, bixafen, boscalid, carboxin, diflumethorim, fenfuram, fluopyram, flutolanil, furametpyr, mepronil, oxycarboxin, penflufen, penthiopyrad, thifluzamide, N-[2-(1,3-dimethylbutyl)phenyl]-5-fluoro-1,3-dimethyl-1H-pyrazole-4-carboxamide, isopyrazam, sedaxan, 3-(difluoromethyl)-1-methyl-N-(3′,4′,5′-trifluorobiphenyl-2-yl)-1H-pyrazole-4-carboxamide, 3-(difluoromethyl)-1-methyl-N-[2-(1,1,2,2-tetrafluoroethoxy)phenyl]-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide and corresponding salts;
F4) respiratory chain complex III inhibitors, for example amisulbrom, azoxystrobin, cyazofamid, dimoxystrobin, enestrobin, famoxadone, fenamidone, fluoxastrobin, kresoxim-methyl, metominostrobin, orysastrobin, pyraclostrobin, pyribencarb, picoxystrobin, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(ethoxyimino)-N-methyl-2-(2-{[({(1E)-1-[3-(trifluoromethyl)phenyl]ethylidene}amino)oxy]methyl}phenyl)ethanamide and corresponding salts, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, 2-methyl {2-[({cyclopropyl[(4-methoxyphenyl)imino]methyl}sulfanyl)methyl]phenyl}-3-methoxyacrylate, N-(3-ethyl-3,5,5-trimethylcyclohexyl)-3-(formylamino)-2-hydroxybenzamide and corresponding salts;
F5) decouplers, for example dinocap, fluazinam;
F6) ATP production inhibitors, for example fentin acetate, fentin chloride, fentin hydroxide, silthiofam
F7) amino acid and protein biosynthesis inhibitors, for example andoprim, blasticidin-S, cyprodinil, kasugamycin, kasugamycin hydrochloride hydrate, mepanipyrim, pyrimethanil
F8) signal transduction inhibitors, for example fenpiclonil, fludioxonil, quinoxyfen
F9) lipid and membrane synthesis inhibitors, for example chlozolinate, iprodione, procymidone, vinclozolin, ampropylfos, potassium-ampropylfos, edifenphos, iprobenfos (IBP), isoprothiolane, pyrazophos, tolclofos-methyl, biphenyl, iodocarb, propamocarb, propamocarb hydrochloride
F10) ergosterol biosynthesis inhibitors, for example fenhexamid, azaconazole, bitertanol, bromuconazole; diclobutrazole, difenoconazole, diniconazole, diniconazole-M, etaconazole, fenbuconazole, fluquinconazole, flusilazole, flutriafol, furconazole, furconazole-cis, hexaconazole, imibenconazole, ipconazole, metconazole, myclobutanil, paclobutrazole, penconazole, propiconazole, prothioconazole, simeconazole, spiroxamine, tebuconazole, triadimefon, triadimenol, triticonazole, uniconazole, voriconazole, imazalil, imazalil sulfate, oxpoconazole, fenarimol, flurprimidol, nuarimol, pyrifenox, triforin, pefurazoate, prochloraz, triflumizole, viniconazole, aldimorph, dodemorph, dodemorph acetate, fenpropimorph, tridemorph, fenpropidin, naftifin, pyributicarb, terbinafin, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-{1-[(4-methoxyphenoxy)methyl]-2,2-dimethylpropyl}-1H-imidazole-1-carbothioate;
F11) cell wall synthesis inhibitors, for example benthiavalicarb, bialaphos, dimethomorph, flumorph, iprovalicarb, polyoxins, polyoxorim, validamycin A
F12) melanine biosynthesis inhibitors, for example capropamide, diclocymet, fenoxanil, phthalide, pyroquilon, tricyclazole
F13) resistance induction, for example acibenzolar-S-methyl, probenazole, tiadinil
F14) multisite, for example captafol, captan, chlorothalonil, copper salts such as: copper hydroxide, copper naphthenate, copper oxychloride, copper sulfate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulfur and sulfur preparations containing calcium polysulfide, thiram, tolylfluanid, zineb, ziram
F15) unknown mechanism, for example amibromdol, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methyl sulfate, diphenylamine, ethaboxam, ferimzone, flumetover, flusulfamide, fluopicolide, fluoroimide, fosatyl-AI, hexachlorobenzene, 8-hydroxyquinoline sulfate, iprodione, irumamycin, isotianil, methasulfocarb, metrafenone, methyl isothiocyanate, mildiomycin, natamycin, nickel dimethyldithiocarbamate, nitrothal-isopropyl, octhilinone, oxamocarb, oxyfenthiin, pentachlorophenol and salts, 2-phenylphenol and salts, piperalin, propanosine-sodium, proquinazid, pyrrolnitrin, quintozene, tecloftalam, tecnazene, triazoxide, trichlamide, zarilamid and 2,3,5,6-tetrachloro-4-(methylsulfonyl)pyridine, N-(4-chloro-2-nitrophenyl)-N-ethyl-4-methylbenzenesulfonamide, 2-amino-4-methyl-N-phenyl-5-thiazolecarboxamide, 2-chloro-N-(2,3-dihydro-1,1,3-trimethyl-1H-inden-4-yl)-3-pyridinecarboxamide, 3-[5-(4-chlorophenyl)-2,3-dimethylisoxazolidin-3-yl]pyridine, cis-1-(4-chlorophenyl)-2-(1H-1,2,4-triazol-1-yl)cycloheptanol, 2,4-dihydro-5-methoxy-2-methyl-4-[[[[1-[3-(trifluoromethyl)phenyl]ethylidene]amino]oxy]methyl]phenyl]-3H-1,2,3-triazol-3-one (185336-79-2), methyl 1-(2,3-dihydro-2,2-dimethyl-1H-inden-1-yl)-1H-imidazole-5-carboxylate, 3,4,5-trichloro-2,6-pyridinedicarbonitrile, methyl 2-[[[cyclopropyl[(4-methoxyphenyl)imino]methyl]thio]methyl]-.alpha.-(methoxymethylene)benzacetate, 4-chloro-alpha-propynyloxy-N-[2-[3-methoxy-4-(2-propynyloxy)phenyl]ethyl]benzacetamide, (2S)—N-[2-[4-[[3-(4-chlorophenyl)-2-propynyl]oxy]-3-methoxyphenyl]ethyl]-3-methyl-2-[(methylsulfonyl)amino]butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidine-7-amine, 5-chloro-N-[(1R)-1,2-dimethylpropyl]-6-(2,4,6-trifluorophenyl)[1,2,4]triazolo[1,5-a]pyrimidine-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propylbenzopyranon-4-one, N-{(Z)-[(cyclopropylmethoxy)imino][6-(difluoromethoxy)-2,3-difluorophenyl]methyl}-2-benzacetamide, N-(3-ethyl-3,5,5-trimethylcyclohexyl)-3-formylamino-2-hydroxybenzamide, 2-[[[[1-[3(1-fluoro-2-phenylethyl)oxy]phenyl]ethylidene]amino]oxy]methyl]-alpha-(methoxyimino)-N-methyl-alphaE-benzacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid; O-[1-[(4-methoxyphenoxy)methyl]-2,2-dimethylpropyl]-1H-imidazole-1-carbothioic acid, 2-(2-{[6-(3-chloro-2-methylphenoxy)-5-fluoropyrimidin-4-yl]oxy}phenyl)-2-(methoxyimino)-N-methylacetamide.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

### Insecticides/Acaricides/Nematicides:

I1) acetylcholine esterase (AChE) inhibitors, a) from the substance group of the carbamates, for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, fenoxycarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate, b) from the group of the organophosphates, for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S-methyl, demeton-S-methylsulfone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion
I2) sodium channel modulators/voltage-dependent sodium channel blockers, a) from the group of the pyrethroids, for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, eflusilanate, empenthrin (1R isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, pyrethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum), b) DDT, c) oxadiazines, for example indoxacarb, d) semicarbazones, for example metaflumizone (BAS3201)
I3) acetylcholine receptor agonists/antagonists, a) from the group of the chloronicotinyls, for example acetamiprid, AKD 1022, clothianidin, dinotefuran, imidacloprid, imidaclothiz, nitenpyram, nithiazine, thiacloprid, thiamethoxam, b) nicotine, bensultap, cartap;
I4) acetylcholine receptor modulators from the group of the spinosyns, for example spinosad
I5) GABA-controlled chloride channel antagonists, a) from the group of the organochlorines, for example camphechlor, chlordane, endosulfan, gamma-HCH, HCH, heptachlor, lindane, methoxychlor, b) fiproles, for example acetoprole, ethiprole, fipronil, pyrafluprole, pyriprole, vaniliprole;
I6) chloride channel activators, for example abamectin, emamectin, emamectin benzoate, ivermectin, lepimectin, milbemycin;
I7) juvenile hormone mimetics, for example diofenolan, epofenonane, fenoxycarb, hydroprene, kinoprene, methoprene, pyriproxifen, triprene;
I8) ecdysone agonists/disruptors, for example chromafenozide, halofenozide, methoxyfenozide, tebufenozide;
I9) chitin biosynthesis inhibitors, for example bistrifluoron, chiofluazuron, diflubenzuron, fluazuron, flucycloxuron, flufenoxuron, hexaflumuron, lufenuron, novaluron, noviflumuron, penfluoron, teflubenzuron, triflumuron, buprofezin, cyromazine;
I10) inhibitors of oxidative phosphorylation, a) ATP disruptors, for example diafenthiuron, b) organotin compounds, for example azocyclotin, cyhexatin, fenbutatin oxide;
I111) decouplers of oxidative phosphorylation by interruption of the H-proton gradient, a) from the group of the pyrroles, for example chlorfenapyr, b) from the class of the dinitrophenols, for example binapacyrl, dinobuton, dinocap, DNOC, meptyldinocap;
I12) site I electron transport inhibitors, for example METIs, especially, as examples, fenazaquin, fenpyroximate, pyrimidifen, pyridaben, tebufenpyrad, tolfenpyrad or else hydramethylnon, dicofol
I13) site II electron transport inhibitors, for example rotenone
I14) site III electron transport inhibitors, for example acequinocyl, fluacrypyrim
I15) microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins, for example Cry1Ab, Cry1Ac, Cry1Fa, Cry2Ab, mCry3A, Cry3Ab, Cry3Bb, Cry34/35Ab1.
I16) lipid synthesis inhibitors, a) from the group of the tetronic acids, for example spirodiclofen, spiromesifen, b) from the class of the tetramic acids, for example spirotetramat, cis-3-(2,5-dimethylphenyl)-4-hydroxy-8-methoxy-1-azaspiro[4.5]dec-3-en-2-one
I17) octopaminergic agonists, for example amitraz
I18) inhibitors of magnesium-stimulated ATPase, for example propargite
I19) nereistoxin analogs, for example thiocyclam hydrogen oxalate, thiosultap-sodium
I20) ryanodine receptor agonists, a) from the group of the benzenedicarboxamides, for example flubendiamide, b) from the group of the anthranilamides, for example rynaxypyr (3-bromo-N-{4-chloro-2-methyl-6-[(methylamino)carbonyl]phenyl}-1-(3-chloropyridin-2-yl)-1H-pyrazole-5-carboxamide), cyazypyr (ISO-proposed) (3-bromo-N-{4-cyano-2-methyl-6-[(methylamino)carbonyl]phenyl}-1-(3-chloropyridin-2-yl)-1H-pyrazole-5-carboxamide) (known from WO 2004067528) and 3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934) or methyl 2-[3,5-dibromo-2-({[3-bromo-1-(3-chloropyridin-2-yl)-1H-pyrazol-5-yl]carbonyl}amino)benzoyl]-1,2-dimethylhydrazinecarboxylate (known from WO2007/043677)
I21) biologics, hormones or pheromones, for example azadirachtin, Bacillus spec., Beauveria spec., codlemone, Metarrhizium spec., Paecilomyces spec., thuringiensin, Verticillium spec.
I22) active ingredients with unknown or nonspecific mechanisms of action, a) fumigants, for example aluminum phosphide, methyl bromide, sulfuryl fluoride, b) antifeedants, for example cryolite, flonicamide, pymetrozine, c) mite growth inhibitors, for example clofentezine, etoxazole, hexythiazox, d) amidoflumet, benclothiaz, benzoximate, bifenazate, bromopropylate, buprofezin, chinomethionat, chlorodimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulfluramid, tetradifon, tetrasul, triarathene, verbutin and the following known active compounds: 4-{[(6-bromopyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(2-chloro-1,3-thiazol-5-yl)methyl](2-fluoroethyl)amino}furan-2(5H)-one (known from WO 2007/115644), 4-{[(6-chloropyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(6-chloro-5-fluoropyrid-3-yl)methyl](methyl)amino}furan-2(5H)-one (known from WO 2007/115643), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](cyclopropyl)amino}furan-2(5H)-one (known from WO 2007/115643), 4-{[(6-chloropyrid-3-yl)methyl](cyclopropyl)amino}furan-2(5H)-one (known from EP0539588), 4-{[(6-chloropyrid-3-yl)methyl](methyl)amino}furan-2(5H)-one (known from EP0539588), [1-(6-chloropyridin-3-yl)ethyl](methyl)oxido-λ⁴-sulfanylidenecyanamide (known from WO 2007/149134) and the diastereomers thereof {[(1R)-1-(6-chloropyridin-3-yl)ethyl](methyl)oxido-lambda⁶-sulfanylidene}cyanamide and {[(1S)-1-(6-chloropyridin-3-yl)ethyl](methyl)oxido-lambda⁶-sulfanylidene}cyanamide (likewise known from WO 2007/149134) and sulfoxaflor (likewise known from WO 2007/149134), 1-[2-fluoro-4-methyl-5-[(2,2,2-trifluoroethyl)sulfinyl]phenyl]-3-(trifluoromethyl)-1H-1,2,4-triazol-5-amine (known from WO 2006/043635), [(3S,4aR,12R,12aS,12bS)-3-[(cyclopropylcarbonyl)oxy]-6,12-dihydroxy-4,12b-dimethyl-11-oxo-9-(pyridin-3-yl)-1,3,4,4a,5,6,6a,12,12a,12b-decahydro-2H,11H-benzo[f]pyrano[4,3-b]chromen-4-yl]methyl cyclopropane-carboxylate (known from WO 2006/129714), 2-cyano-3-(difluoromethoxy)-N,N-dimethylbenzenesulfonamide (known from WO2006/056433), 2-cyano-3-(difluoromethoxy)-N-methylbenzenesulfonamide (known from WO2006/100288), 2-cyano-3-(difluoromethoxy)-N-ethylbenzenesulfonamide (known from WO2005/035486), 4-(difluoromethoxy)-N-ethyl-N-methyl-1,2-benzothiazole-3-amine 1,1-dioxide (known from WO2007/057407), N-[1-(2,3-dimethylphenyl)-2-(3,5-dimethylphenyl)ethyl]-4,5-dihydro-1,3-thiazole-2-amine (known from WO2008/104503), {1′-[(2E)-3-(4-chlorophenyl)prop-2-en-1-yl]-5-fluorospiro[indole-3,4′-piperidin]-1(2H)-yl}(2-chloropyridin-4-yl)methanone (known from WO2003106457), 3-(2,5-dimethylphenyl)-4-hydroxy-8-methoxy-1,8-diazaspiro[4.5]dec-3-en-2-one (known from WO2009049851), 3-(2,5-dimethylphenyl)-8-methoxy-2-oxo-1,8-diazaspiro[4.5]dec-3-en-4-ylethyl carbonate (known from WO2009049851), 4-(but-2-yn-1-yloxy)-6-(3,5-dimethylpiperidin-1-yl)-5-fluoropyrimidine (known from WO2004099160), (2,2,3,3,4,4,5,5-octafluoropentyl) (3,3,3-trifluoropropyl)malononitrile (known from WO2005063094), (2,2,3,3,4,4,5,5-octafluoropentyl) (3,3,4,4,4-pentafluorobutyl)malononitrile (known from WO2005063094), 8-[2-(cyclopropylmethoxy)-4-(trifluoromethyl)phenoxy]-3-[6-(trifluoromethyl)pyridazin-3-yl]-3-azabicyclo[3.2.1]octane (known from WO2007040280/282), 2-ethyl-7-methoxy-3-methyl-6-[(2,2,3,3-tetrafluoro-2,3-dihydro-1,4-benzodioxin-6-yl)oxy]quinolin-4-ylmethyl carbonate (known from JP2008110953), 2-ethyl-7-methoxy-3-methyl-6-[(2,2,3,3-tetrafluoro-2,3-dihydro-1,4-benzodioxin-6-yl)oxy]quinolin-4-yl acetate (known from JP2008110953), PF1364 (Chemical Abstracts No. 1204776-60-2, known from JP2010018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO2007075459), 5-[5-(2-chloropyridin-4-yl)-5-(trifluoromethyl)-4,5-dihydro-1,2-oxazol-3-yl]-2-(1H-1,2,4-triazol-1-yl)benzonitrile (known from WO2007075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005085216).

Safeners are preferably selected from the group consisting of:
S1) compounds of the formula (S1)

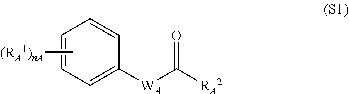

where the symbols and indices are each defined as follows:
n_{A} is a natural number from 0 to 5, preferably from 0 to 3;
R_{A}¹ is halogen, (C₁-C₄)alkyl, (C₁-C₄)alkoxy, nitro or (C₁-C₄)haloalkyl;

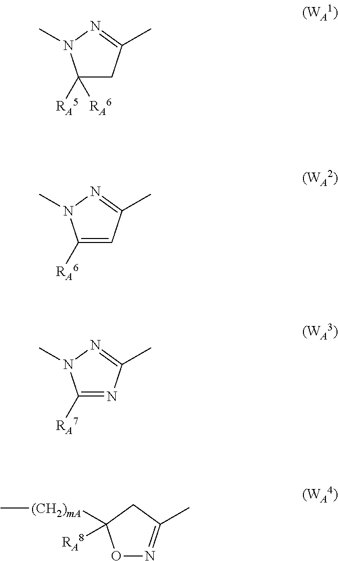

W_{A} is an unsubstituted or substituted divalent heterocyclic radical from the group of the partially unsaturated or aromatic five-membered heterocycles having 1 to 3 ring heteroatoms of the N or O type, where at least one nitrogen atom and at most one oxygen atom is present in the ring, preferably a radical from the group of (W_{A}¹) to (W_{A}¹)
m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group of O and S, which is joined to the carbonyl group in (S1) via the nitrogen atom and is unsubstituted or substituted by radicals from the group of (C₁-C₄)alkyl, (C₁-C₄)alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbyl radical preferably having a total of 1 to 18 carbon atoms;
R_{A}⁴ is hydrogen, (C₁-C₆)alkyl, (C₁-C₆)alkoxy or substituted or unsubstituted phenyl;
R_{A}⁵ is H, (C₁-C₈)alkyl, (C₁-C₈)haloalkyl, (C₁-C₄)alkoxy(C₁-C₈)alkyl, cyano or COOR_{A}⁹ in which R_{A}⁹ is hydrogen, (C₁-C₈)alkyl, (C₁-C₈)haloalkyl, (C₁-C₄)alkoxy-(C₁-C₄)alkyl, (C₁-C₆)hydroxyalkyl, (C₃-C₁₂)cycloalkyl or tri-(C₁-C₄)-alkylsilyl;
R_{A}⁶, R_{A}⁷, R_{A}⁸ are the same or different and are each hydrogen, (C₁-C₈)alkyl, (C₁-C₆)haloalkyl, (C₃-C₁₂)cycloalkyl or substituted or unsubstituted phenyl;
   preferably:
a) compounds of the dichlorophenylpyrazoline-3-carboxylic acid (S1^{a}) type, preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874; derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;
d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.
S2) Quinoline derivatives of the formula (S2)

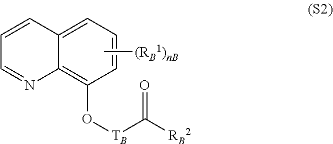

where the symbols and indices are each defined as follows:
R_{B}¹ is halogen, (C₁-C₄)alkyl, (C₁-C₄)alkoxy, nitro or (C₁-C₄)haloalkyl;
n_{B} is a natural number from 0 to 5, preferably from 0 to 3;
R_{B}² is OR_{B}³, SR_{B}³ or NR_{B}³R_{B}⁴ or a saturated
   or unsaturated 3- to 7-membered heterocycle having at least one nitrogen atom and up to 3 heteroatoms, preferably from the group of O and S, which is joined to the carbonyl group in (S2) via the nitrogen atom and is unsubstituted or substituted by radicals from the group of (C₁-C₄)alkyl, (C₁-C₄)alkoxy or optionally substituted phenyl, preferably a radical of the formula OR_{B}³, NHR_{B}⁴ or N(CH₃)₂, especially of the formula OR_{B}³;
R_{B}³ is hydrogen or an unsubstituted or substituted aliphatic hydrocarbyl radical preferably having a total of 1 to 18 carbon atoms;
R_{B}⁴ is hydrogen, (C₁-C₆)alkyl, (C₁-C₆)alkoxy or substituted or unsubstituted phenyl;
T_{B} is a (C₁ or C₂)-alkanediyl chain which is unsubstituted or substituted by one or two (C₁-C₄)alkyl radicals or by [(C₁-C₃)-alkoxy]carbonyl;
   preferably:
a) compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Compounds of the formula (S3)

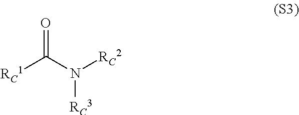

where the symbols and indices are each defined as follows:
R_{C}¹ is (C₁-C₄)alkyl, (C₁-C₄)haloalkyl, (C₂-C₄)alkenyl, (C₂-C₄)haloalkenyl, (C₃-C₇)cycloalkyl, preferably dichloromethyl;
R_{C}², R_{C}³ are the same or different and are each hydrogen, (C₁-C₄)alkyl, (C₂-C₄)alkenyl,
   (C₂-C₄)alkynyl, (C₁-C₄)haloalkyl, (C₂-C₄)haloalkenyl, (C₁-C₄)alkylcarbamoyl-(C₁-C₄)alkyl, (C₂-C₄)alkenylcarbamoyl-(C₁-C₄)alkyl, (C₁-C₄)alkoxy-(C₁-C₄)alkyl, dioxolanyl-(C₁-C₄)alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring; preferably: active ingredients of the dichloroacetamide type, which are frequently used as pre-emergence safeners (soil-acting safeners), for example “dichlormid” (N,N-diallyl-2,2-dichloroacetamide) (S3-1), “R-29148” (3-dichloroacetyl-2,2,5-trimethyl-1,3-oxazolidine) from Stauffer (S3-2), “R-28725” (3-dichloroacetyl-2,2-dimethyl-1,3-oxazolidine) from Stauffer (S3-3), “benoxacor” (4-dichloroacetyl-3,4-dihydro-3-methyl-2H-1,4-benzoxazine) (S3-4), “PPG-1292” (N-allyl-N-[(1,3-dioxolan-2-yl)methyl]dichloroacetamide) from PPG Industries (S3-5), “DKA-24” (N-allyl-N-[(allylaminocarbonyl)methyl]dichloroacetamide) from Sagro-Chem (S3-6), “AD-67” or “MON 4660” (3-dichloroacetyl-1-oxa-3-azaspiro[4,5]decane) from Nitrokemia or Monsanto (S3-7), “TI-35” (1-dichloroacetylazepane) from TRI-Chemical RT (S3-8), “diclonon” (dicyclonone) or “BAS145138” or “LAB145138” (S3-9) ((RS)-1-dichloroacetyl-3,3,8a-trimethylperhydropyrrolo[1,2-a]pyrimidin-6-one) from BASF, “furilazole” or “MON 13900” ((RS)-3-dichloroacetyl-5-(2-furyl)-2,2-dimethyloxazolidine) (S3-10); and the (R) isomer thereof (S3-11).
S4) N-acylsulfonamides of the formula (S4) and salts thereof,

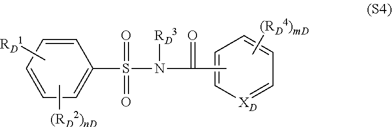

in which the symbols and indices are each defined as follows:
X_{D} is CH or N;
R_{D}¹ is CO—NR_{D}⁵R_{D}⁶ or NHCO—R_{D}⁷;
R_{D}² is halogen, (C₁-C₄)haloalkyl, (C₁-C₄)haloalkoxy, nitro, (C₁-C₄)alkyl, (C₁-C₄)alkoxy, (C₁-C₄)alkylsulfonyl, (C₁-C₄)alkoxycarbonyl or (C₁-C₄)alkylcarbonyl;
R_{D}³ is hydrogen, (C₁-C₄)alkyl, (C₂-C₄)alkenyl or (C₂-C₄)alkynyl;
R_{D}⁴ is halogen, nitro, (C₁-C₄)alkyl, (C₁-C₄)haloalkyl, (C₁-C₄)haloalkoxy, (C₃-C₆)cycloalkyl, phenyl, (C₁-C₄)alkoxy, cyano, (C₁-C₄)alkylthio, (C₁-C₄)alkylsulfinyl, (C₁-C₄)alkylsulfonyl, (C₁-C₄)alkoxycarbonyl or (C₁-C₄)alkylcarbonyl;
R_{D}⁵ is hydrogen, (C₁-C₆)alkyl, (C₃-C₆)cycloalkyl, (C₂-C₆)alkenyl, (C₂-C₆)alkynyl, (C₅-C₆)cycloalkenyl, phenyl or 3- to 6-membered heterocyclyl containing v_{D} heteroatoms from the group of nitrogen, oxygen and sulfur, where the seven latter radicals are substituted by v_{D} substituents from the group of halogen, (C₁-C₆)alkoxy, (C₁-C₆)haloalkoxy, (C₁-C₂)alkylsulfinyl, (C₁-C₂)alkylsulfonyl, (C₃-C₆)cycloalkyl, (C₁-C₄)alkoxycarbonyl, (C₁-C₄)alkylcarbonyl and phenyl and, in the case of cyclic radicals, also (C₁-C₄)alkyl and (C₁-C₄)haloalkyl;
R_{D}⁶ is hydrogen, (C₁-C₆)alkyl, (C₂-C₆)alkenyl or (C₂-C₆)alkynyl, where the three latter radicals are substituted by v_{D} radicals from the group of halogen, hydroxy, (C₁-C₄)alkyl, (C₁-C₄)alkoxy and (C₁-C₄)alkylthio, or
R_{D}⁵ and R_{D}⁶ together with the nitrogen atom which bears them form a pyrrolidinyl or piperidinyl radical;
R_{D}⁷ is hydrogen, (C₁-C₄)alkylamino, di-(C₁-C₄)alkylamino, (C₁-C₆)alkyl, (C₃-C₆)cycloalkyl, where the 2 latter radicals are substituted by v_{D} substituents from the group of halogen, (C₁-C₄)alkoxy, (C₁-C₆)haloalkoxy and (C₁-C₄)alkylthio and, in the case of cyclic radicals, also (C₁-C₄)alkyl and (C₁-C₄)haloalkyl;
n_{D} is 0, 1 or 2;
m_{D} is 1 or 2;
v_{D} is 0, 1, 2 or 3;
   among these, preference is given to compounds of the N-acylsulfonamide type, for example of the formula (S4^{a}) below, which are known, for example, from WO-A-97/45016

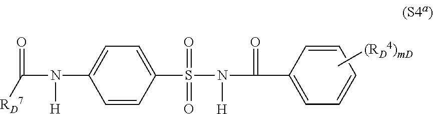

in which
R_{D}⁷ is (C₁-C₆)alkyl, (C₃-C₆)cycloalkyl, where the 2 latter radicals are substituted by v_{D} substituents from the group of halogen, (C₁-C₄)alkoxy, (C₁-C₆)haloalkoxy and (C₁-C₄)alkylthio and, in the case of cyclic radicals, also (C₁-C₄)alkyl and (C₁-C₄)haloalkyl;
R_{D}⁴ is halogen, (C₁-C₄)alkyl, (C₁-C₄)alkoxy, CF₃,
m_{D} is 1 or 2;
V_{D} is 0, 1, 2 or 3;
   and also to acylsulfamoylbenzamides, for example of the formula (S4^{b}) below, which are known, for example, from WO-A-99/16744,

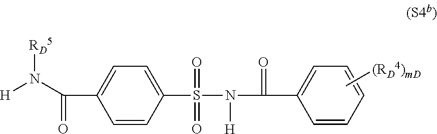

for example those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5)
   and to compounds of the N-acylsulfamoylphenylurea type, of the formula (S4^{c}), which are known, for example, from EP-A-365484,

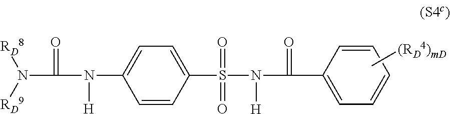

in which
R_{D}⁸ and R_{D}⁹ are each independently hydrogen, (C₁-C₈)alkyl, (C₃-C₈)cycloalkyl, (C₃-C₆)alkenyl, (C₃-C₆)alkynyl,
R_{D}⁴ is halogen, (C₁-C₄)alkyl, (C₁-C₄)alkoxy, CF₃
m_{D} is 1 or 2;
   for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea.
S5) Active ingredients from the class of the hydroxyaromatics and the aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicyclic acid, 2-hydroxycinnamic acid, 2,4-dichlorocinnamic acid, as described in WO-A-2004/084631, WO-A-2005/015994, WO-A-2005/016001.
S6) Active ingredients from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856,

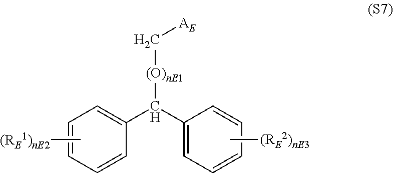

in which the symbols and indices are each defined as follows:
R_{E}¹, R_{E}² are each independently halogen, (C₁-C₄)alkyl, (C₁-C₄)alkoxy, (C₁-C₄)haloalkyl, (C₁-C₄)alkylamino, di-(C₁-C₄)alkylamino, nitro;
A_{E} is COOR_{E}³ or COSR_{E}⁴
R_{E}³, R_{E}⁴ are each independently hydrogen, (C₁-C₄)alkyl, (C₂-C₆)alkenyl, (C₂-C₄)alkynyl, cyanoalkyl, (C₁-C₄)haloalkyl, phenyl, nitrophenyl, benzyl, halobenzyl, pyridinylalkyl and alkylammonium,
n_{E}¹ is 0 or 1
n_{E}², n_{E}³ are each independently 0, 1 or 2,
   preferably diphenylmethoxyacetic acid, ethyl diphenylmethoxyacetate, methyl diphenylmethoxyacetate (CAS reg. no. 41858-19-9) (S7-1).
S8) Compounds of the formula (S8), as described in WO-A-98/27049,

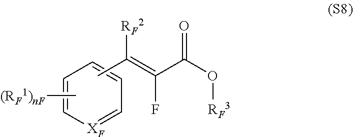

in which
X_{F} is CH or N,
n_{F} in the case that X_{F}═N is an integer from 0 to 4 and in the case that X_{F}═CH is an integer from 0 to 5,
R_{F}¹ is halogen, (C₁-C₄)alkyl, (C₁-C₄)haloalkyl, (C₁-C₄)alkoxy, (C₁-C₄)haloalkoxy, nitro, (C₁-C₄)alkylthio, (C₁-C₄)-alkylsulfonyl, (C₁-C₄)alkoxycarbonyl, optionally substituted phenyl, optionally substituted phenoxy,
R_{F}² is hydrogen or (C₁-C₄)alkyl
R_{F}³ is hydrogen, (C₁-C₆)alkyl, (C₂-C₄)alkenyl, (C₂-C₄)alkynyl, or aryl, where each of the aforementioned carbon-containing radicals is unsubstituted or substituted by one or more, preferably up to three identical or different radicals from the group consisting of halogen and alkoxy; or salts thereof,
   preferably compounds in which
X_{F} is CH,
n_{F} is an integer from 0 to 2,
R_{F}¹ is halogen, (C₁-C₄)alkyl, (C₁-C₄)haloalkyl, (C₁-C₄)alkoxy, (C₁-C₄)haloalkoxy,
R_{F}² is hydrogen or (C₁-C₄)alkyl,
R_{F}³ is hydrogen, (C₁-C₈)alkyl, (C₂-C₄)alkenyl, (C₂-C₄)alkynyl, or aryl, where each of the aforementioned carbon-containing radicals is unsubstituted or substituted by one or more, preferably up to three identical or different radicals from the group consisting of halogen and alkoxy; or salts thereof.
S9) Active ingredients from the class of the 3-(5-tetrazolylcarbonyl)-2-quinolones (S9), e.g.
   1,2-dihydro-4-hydroxy-1-ethyl-3-(5-tetrazolylcarbonyl)-2-quinolone (CAS reg. no.: 219479-18-2), 1,2-dihydro-4-hydroxy-1-methyl-3-(5-tetrazolylcarbonyl)-2-quinolone (CAS reg. no.: 95855-00-8), as described in WO-A-1999/000020.
S10) Compounds of the formulae (S10^{a}) or (S10^{b})
   as described in WO-A-2007/023719 and WO-A-2007/023764
      in which

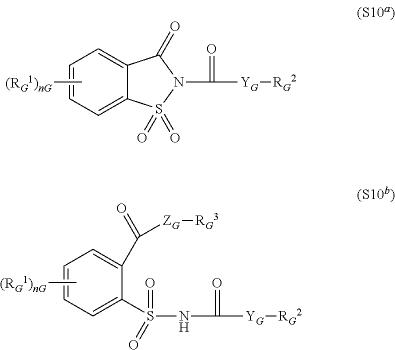

R_{G}¹ is halogen, (C₁-C₄)alkyl, methoxy, nitro, cyano, CF₃, OCF₃ Y_{G}, Z_{G} are each independently O or S,
n_{G} is an integer from 0 to 4,
R_{G}² is (C₁-C₁₆)alkyl, (C₂-C₆)alkenyl, (C₃-C₆)cycloalkyl, aryl; benzyl, halobenzyl,
R_{G}³ is hydrogen or (C₁-C₆)alkyl.
S11) Active ingredients of the oxyimino compound type (S11), which are known as seed-dressing compositions, for example “oxabetrinil” ((Z)-1,3-dioxolan-2-yl-methoxyimino(phenyl)acetonitrile) (S11-1), which is known as a seed-dressing safener for millet/sorghum, against damage by metolachlor, “fluxofenim” (1-(4-chlorophenyl)-2,2,2-trifluoro-1-ethanone O-(1,3-dioxolan-2-ylmethyl) oxime) (S11-2), which is known as a seed-dressing safener for millet/sorghum against damage by metolachlor, and “cyometrinil” or “CGA-43089” ((Z)-cyanomethoxyimino(phenyl)acetonitrile) (S11-3), which is known as a seed-dressing safener for millet/sorghum against damage by metolachlor.
S12) Active ingredients from the class of the isothiochromanones (S12), for example methyl [(3-oxo-1H-2-benzothiopyran-4(3H)-ylidene)methoxy]acetate (CAS reg. no. 205121-04-6) (S12-1) and related compounds from WO-A-1998/13361.
S13) One or more compounds from group (S13): “naphthalic anhydride” (1,8-naphthalenedicarboxylic anhydride) (S13-1), which is known as a seed-dressing safener for corn against damage by thiocarbamate herbicides, “fenclorim” (4,6-dichloro-2-phenylpyrimidine) (S13-2), which is known as a safener for pretilachlor in sown rice, “flurazole” (benzyl 2-chloro-4-trifluoromethyl-1,3-thiazole-5-carboxylate) (S13-3), which is known as a seed-dressing safener for millet/sorghum against damage by alachlor and metolachlor, “CL 304415” (CAS reg. no. 31541-57-8) (4-carboxy-3,4-dihydro-2H-1-benzopyran-4-acetic acid) (S13-4) from American Cyanamid, which is known as a safener for corn against damage by imidazolinones, “MG 191” (CAS reg. no. 96420-72-3) (2-dichloromethyl-2-methyl-1,3-dioxolane) (S13-5) from Nitrokemia, which is known as safener for corn, “MG-838” (CAS reg. no. 133993-74-5) (2-propenyl 1-oxa-4-azaspiro[4.5]decane-4-carbodithioate) (S13-6) from Nitrokemia, “disulfoton” (O,O-diethyl S-2-ethylthioethyl phosphorodithioate) (S13-7), “dietholate” (O,O-diethyl O-phenylphosphorothioate) (S13-8), “mephenate” (4-chlorophenyl methylcarbamate) (S13-9).
S14) Active ingredients which, in addition to herbicidal action against harmful plants, also have safener action on crop plants such as rice, for example “dimepiperate” or “MY-93” (S-1-methyl-1-phenylethylpiperidine-1-carbothioate), which is known as a safener for rice against damage by the herbicide molinate, “daimuron” or “SK 23” (1-(1-methyl-1-phenylethyl)-3-p-tolylurea), which is known as a safener for rice against damage by the herbicide imazosulfuron, “cumyluron”=“JC-940” (3-(2-chlorophenylmethyl)-1-(1-methyl-1-phenylethyl)urea, see JP-A-60087254), which is known as a safener for rice against damage by some herbicides, “methoxyphenone” or “NK 049” (3,3′-dimethyl-4-methoxybenzophenone), which is known as a safener for rice against damage by some herbicides, “CSB” (1-bromo-4-(chloromethylsulfonyl)benzene) from Kumiai, (CAS reg. no. 54091-06-4), which is known as a safener against damage by some herbicides in rice.
S15) Compounds of the formula (S15) or tautomers thereof
   as described in WO-A-2008/131861 and WO-A-2008/131860
      in which

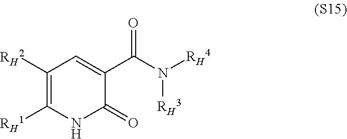

R_{H}¹ is a (C₁-C₆)haloalkyl radical and
R_{H}² is hydrogen or halogen and
R_{H}³, R_{H}⁴ are each independently hydrogen, (C₁-C₁₆)alkyl, (C₂-C₁₆)alkenyl or (C₂-C₁₆)alkynyl, where each of the latter 3 radicals is unsubstituted or substituted by one or more radicals from the group of halogen, hydroxy, cyano, (C₁-C₄)alkoxy, (C₁-C₄)haloalkoxy, (C₁-C₄)alkylthio, (C₁-C₄)alkylamino, di[(C₁-C₄)alkyl]amino, [(C₁-C₄)alkoxy]carbonyl, [(C₁-C₄)haloalkoxy]carbonyl, (C₃-C₆)cycloalkyl which is unsubstituted or substituted, phenyl which is unsubstituted or substituted, and heterocyclyl which is unsubstituted or substituted, or (C₃-C₆)cycloalkyl, (C₄-C₆)cycloalkenyl, (C₃-C₆)cycloalkyl which is fused on one side of the ring to a 4 to 6-membered saturated or unsaturated carbocyclic ring, or (C₄-C₆)cycloalkenyl which is fused on one side of the ring to a 4 to 6-membered saturated or unsaturated carbocyclic ring, where each of the latter 4 radicals is unsubstituted or substituted by one or more radicals from the group of halogen, hydroxy, cyano, (C1-C4)alkyl, (C1-C4)haloalkyl, (C1-C4)alkoxy, (C1-C4)haloalkoxy, (C1-C4)alkylthio, (C1-C4)alkylamino, di[C1-C4)alkyl]amino, [(C1-C4)alkoxy]carbonyl, [(C1-C4)haloalkoxy]-carbonyl, (C3-C6)cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)alkenyloxy, (C₂-C₆)alkynyloxy or (C₂-C₄)haloalkoxy and
R_{H}⁴ is hydrogen or (C₁-C₄)-alkyl or
R_{H}³ and R_{H}⁴ together with the directly bonded nitrogen atom form a four- to eight-membered
   heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)alkyl, (C₁-C₄)haloalkyl, (C₁-C₄)alkoxy, (C₁-C₄)haloalkoxy and (C₁-C₄)alkylthio.
S16) Active ingredients which are used primarily as herbicides but also have safener action on crop plants, for example (2,4-dichlorophenoxy)acetic acid (2,4-D), (4-chlorophenoxy)acetic acid, (R,S)-2-(4-chloro-o-tolyloxy)propionic acid (mecoprop), 4-(2,4-dichlorophenoxy)butyric acid (2,4-DB), (4-chloro-o-tolyloxy)acetic acid (MCPA), 4-(4-chloro-o-tolyloxy)butyric acid, 4-(4-chlorophenoxy)butyric acid, 3,6-dichloro-2-methoxybenzoic acid (dicamba), 1-(ethoxycarbonyl)ethyl 3,6-dichloro-2-methoxybenzoate (lactidichior-ethyl).
   Substances which Influence Plant Maturity:

Combination partners usable for the compounds of the formula (I) in mixture formulations or in tankmixes are, for example, known active ingredients based on inhibition of, for example, 1-aminocyclopropane-1-carboxylate synthase, 1-aminocyclopropane-1-carboxylate oxidase and the ethylene receptors, for example ETR1, ETR2, ERS1, ERS2 or EIN4, as described, for example, in Biotechn. Adv. 2006, 24, 357-367; Bot. Bull. Acad. Sin. 199, 40, 1-7 or Plant Growth Reg. 1993, 13, 41-46 and literature cited therein.

Examples of known substances which influence plant maturity and can be combined with the compounds of the formula (I) include the active ingredients which follow (the compounds are designated by the “common name” according to the International Organization for Standardization (ISO) or by the chemical name or by the code number) and always encompass all use forms, such as acids, salts, esters and isomers, such as stereoisomers and optical isomers. In this list, one or else, in some cases, more than one application form is mentioned:

rhizobitoxine, 2-aminoethoxyvinylglycine (AVG), methoxyvinylglycine (MVG), vinylglycine, aminooxyacetic acid, sinefungin, S-adenosylhomocysteine, 2-keto-4-methyl thiobutyrate, 2-(methoxy)-2-oxoethyl (isopropylidene)aminooxyacetate, 2-(hexyloxy)-2-oxoethyl (isopropylidene)aminooxyacetate, 2-(isopropyloxy)-2-oxoethyl (cyclohexylidene)aminooxyacetate, putrescine, spermidine, spermine, 1,8-diamino-4-aminoethyloctane, L-canaline, daminozide, methyl 1-aminocyclopropyl-1-carboxylate, N-methyl-1-aminocyclopropyl-1-carboxylic acid, 1-aminocyclopropyl-1-carboxamide, substituted 1-aminocyclopropyl-1-carboxylic acid derivatives as described in DE3335514, EP30287, DE2906507 or U.S. Pat. No. 5,123,951, 1-aminocyclopropyl-1-hydroxamic acid, 1-methylcyclopropene, 3-methylcyclopropene, 1-ethylcyclopropene, 1-n-propylcyclopropene, 1-cyclopropenylmethanol, carvone, eugenol

### Herbicides or Plant Growth Regulators:

Combination partners usable for the compounds of the formula (I) in mixture formulations or in tankmixes are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem I, photosystem II, protoporphyrinogen oxidase, as described, for example, in Weed Research 26 (1986) 441-445 or “The Pesticide Manual”, 14th edition, The British Crop Protection Council and the Royal Soc. of Chemistry, 2006 and literature cited therein.

Examples of known herbicides or plant growth regulators which can be combined with compounds of the formula (I) include the active ingredients which follow (the compounds are designated by the “common name” according to the International Organization for Standardization (ISO) or by the chemical name or by the code number) and always encompass all use forms, such as acids, salts, esters and isomers, such as stereoisomers and optical isomers. In this list, one or else, in some cases, more than one application form is mentioned:

acetochlor, acibenzolar, acibenzolar-S-methyl, acifluorfen, acifluorfen-sodium, aclonifen, alachlor, allidochlor, alloxydim, alloxydim-sodium, ametryne, amicarbazone, amidochlor, amidosulfuron, aminocyclopyrachlor, aminopyralid, amitrole, ammonium sulfamate, ancymidol, anilofos, asulam, atrazine, azafenidin, azimsulfuron, aziprotryne, beflubutamid, benazolin, benazolin-ethyl, bencarbazone, benfluralin, benfuresate, bensulide, bensulfuron, bensulfuron-methyl, bentazone, benzfendizone, benzobicyclon, benzofenap, benzofluor, benzoylprop, bicyclopyrone, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbetamide, carfentrazone, carfentrazone-ethyl, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat-chloride, chiornitrofen, chlorophthalim, chlorthal-dimethyl, chlortoluron, chlorsulfuron, cinidon, cinidon-ethyl, cinmethylin, cinosulfuron, clethodim, clodinafop, clodinafop-propargyl, clofencet, clomazone, clomeprop, cloprop, clopyralid, cloransulam, cloransulam-methyl, cumyluron, cyanamide, cyanazine, cyclanilide, cycloate, cyclosulfamuron, cycloxydim, cycluron, cyhalofop, cyhalofop-butyl, cyperquat, cyprazine, cyprazole, 2,4-D, 2,4-DB, daimuron/dymron, dalapon, daminozide, dazomet, n-decanol, desmedipham, desmetryn, detosyl-pyrazolate (DTP), diallate, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dimefuron, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, dipropetryn, diquat, diquat-dibromide, dithiopyr, diuron, DNOC, eglinazine-ethyl, endothal, EPTC, esprocarb, ethalfluralin, ethametsulfuron, ethametsulfuron-methyl, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoprop, fenoxaprop, fenoxaprop-P, fenoxaprop-ethyl, fenoxaprop-P-ethyl, fenoxasulfone, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, fluorochloridone, fluoroxypyr, fluoroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, glufosinate-ammonium, glufosinate-P, glufosinate-P-ammonium, glufosinate-P-sodium, glyphosate, glyphosate-isopropylammonium, H-9201, i.e. O-(2,4-dimethyl-6-nitrophenyl) O-ethyl isopropylphosphoramidothioate, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapyr, imazapyr-isopropylammonium, imazaquin, imazaquin-ammonium, imazethapyr, imazethapyr-ammonium, imazosulfuron, inabenfide, indanofan, indaziflam, indoleacetic acid (IAA), 4-indol-3-ylbutyric acid (IBA), iodosulfuron, iodosulfuron-methyl-sodium, ioxynil, ipfencarbazone, isocarbamid, isopropalin, isoproturon, isouron, isoxaben, isoxachlortole, isoxaflutole, isoxapyrifop, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat-chloride, mesosulfuron, mesosulfuron-methyl, mesotrione, methabenzthiazuron, metam, metamifop, metamitron, metazachlor, metazasulfuron, methazole, methiopyrsulfuron, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monosulfuron ester, monuron, MT-128, i.e. 6-chloro-N-[(2E)-3-chloroprop-2-en-1-yl]-5-methyl-N-phenylpyridazine-3-amine, MT-5950, i.e. N-[3-chloro-4-(1-methylethyl)phenyl]-2-methylpentanamide, NGGC-011, naproanilide, napropamide, naptalam, NC-310, i.e. 4-(2,4-dichlorobenzoyl)-1-methyl-5-benzyloxypyrazole, neburon, nicosulfuron, nipyraclofen, nitralin, nitrofen, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazole, paraquat, paraquat dichloride, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazole, procyazine, prodiamine, prifluraline, profoxydim, prohexadione, prohexadione-calcium, prohydrojasmone, prometon, prometryn, propachlor, propanil, propaquizafop, propazine, propham, propisochior, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, secbumeton, sethoxydim, siduron, simazine, simetryn, SN-106279, i.e. methyl (2R)-2-({7-[2-chloro-4-(trifluoromethyl)phenoxy]-2-naphthyl}oxy)propanoate, sulcotrione, sulfallate (CDEC), sulfentrazone, sulfometuron, sulfometuron-methyl, sulfosate (glyphosate-trimesium), sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, tebutam, tebuthiuron, tecnazene, tefuryltrione, tembotrione, tepraloxydim, terbacil, terbucarb, terbuchlor, terbumeton, terbuthylazine, terbutryne, thenylchlor, thiafluamide, thiazafluoron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, triallate, triasulfuron, triaziflam, triazofenamide, tribenuron, tribenuron-methyl, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0862, i.e. 3,4-dichloro-N-{2-[(4,6-dimethoxypyrimidin-2-yl)oxy]benzyl}aniline, and the following compounds:

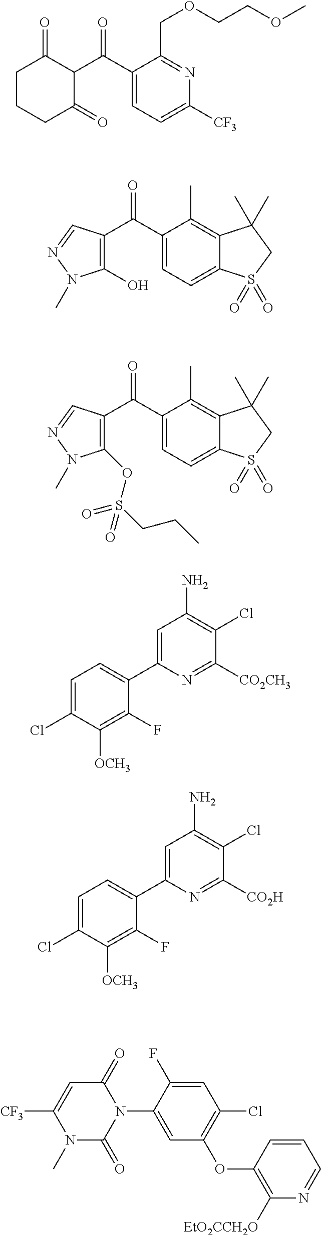

The invention is to be illustrated by the biological examples which follow, but without restricting it thereto.

### Biological Examples:

Seeds of monocotyledonous and dicotyledonous crop plants were placed in sandy loam in wood-fiber pots, covered with soil and cultivated in a greenhouse under good growth conditions. The test plants were treated at the early leaf stage (BBCH10-BBCH13). To ensure uniform water supply before commencement of stress, the potted plants were supplied with the maximum amount of water immediately beforehand by dam irrigation and, after application, transferred in plastic inserts in order to prevent subsequent, excessively rapid drying. The inventive compounds, formulated in the form of wettable powders (WP), wettable granules (WG), suspension concentrates (SC) or emulsion concentrates (EC), were sprayed onto the green parts of the plants as an aqueous suspension at an equivalent water application rate of 600 l/ha with addition of 0.2% wetting agent (agrotin). Substance application was followed immediately by stress treatment of the plants (cold or drought stress). For cold stress treatment, the plants were kept under the following controlled conditions:
“day”: 12 hours with illumination at 8° C.
“night”: 12 hours without illumination at 1° C.

Drought stress was induced by gradual drying out under the following conditions:
“day”: 14 hours with illumination at 26° C.
“night”: 10 hours without illumination at 18° C.

The duration of the respective stress phases was guided mainly by the state of the untreated, stressed control plants and thus varied from crop to crop. It was ended (by re-irrigating or transfer to a greenhouse with good growth conditions) as soon as irreversible damage was observed on the untreated, stressed control plants. In the case of dicotyledonous crops, for example oilseed rape and soybeans, the duration of the drought stress phase varied between 3 and 5 days, in the case of monocotyledonous crops, for example wheat, barley or corn, between 6 and 10 days. The duration of the cold stress phase varied between 12 and 14 days.

The end of the stress phase was followed by an approx. 5-7-day recovery phase, during which the plants were once again kept under good growth conditions in a greenhouse. In order to rule out any influence of the effects observed by any fungicidal action of the test compounds, it was additionally ensured that the tests proceed without fungal infection and without infection pressure.

After the recovery phase had ended, the intensities of damage were rated in visual comparison to untreated, unstressed controls of the same age (in the case of drought stress) or the same growth stage (in the case of cold stress). The intensity of damage was first assessed as a percentage (100%=plants have died, 0%=like control plants). These values were then used to calculate the efficacy of the test compounds (=percentage reduction in the intensity of damage as a result of substance application) by the following formula:

$$EF = \frac{( {DV}_{us} - {DV}_{ts} ) \times 100}{{DV}_{us}}$$

EF: efficacy (%)
DVᵤₛ: damage value of the untreated, stressed control
DVₜₛ: damage value of the plants treated with test compound

The tables below list mean values in each case from three results of the same test.

### Efficacies of Selected Compounds of the Formula (I) Under Cold Stress:

| | | | | EF |
|---|---|---|---|---|
| No. | Substance | Dosage | Unit | (ZEAMX) |
| 1 | I.2-1 | 25 | g/ha | >5 |
| 2 | I.8-1 | 25 | g/ha | >5 |
| 3 | I.10-1 | 25 | g/ha | >5 |
| 4 | I.37-63 | 250 | g/ha | >5 |

### Efficacies of Selected Compounds of the Formula (I) Under Drought Stress:

| No. | Substance | Dosage | Unit | |
|---|---|---|---|---|
| | | | | EF |
| | | | | (BRSNS) |
| 1 | I.1-1 | 25 | g/ha | >5 |
| 2 | I.1-6 | 25 | g/ha | >5 |
| 3 | I.1-39 | 25 | g/ha | >5 |
| 4 | I.2-7 | 250 | g/ha | >5 |
| 5 | I.3-9 | 25 | g/ha | >5 |
| 6 | I.3-26 | 25 | g/ha | >5 |
| 7 | I.3-36 | 25 | g/ha | >5 |
| 8 | I.3-51 | 250 | g/ha | >5 |
| 9 | I.4-39 | 25 | g/ha | >5 |
| 10 | I.4-59 | 25 | g/ha | >5 |
| 11 | I.5-48 | 25 | g/ha | >5 |
| 12 | I.7-6 | 25 | g/ha | >5 |
| 13 | I.8-1 | 25 | g/ha | >5 |
| 14 | I.32-60 | 25 | g/ha | >5 |
| 15 | I.36-1 | 25 | g/ha | >5 |
| 16 | I.37-4 | 25 | g/ha | >5 |
| 17 | I.37-6 | 25 | g/ha | >5 |
| 18 | I.37-7 | 25 | g/ha | >5 |
| 19 | I.37-39 | 25 | g/ha | >5 |
| 20 | I.37-63 | 25 | g/ha | >5 |
| 21 | I.38-1 | 25 | g/ha | >5 |
| 22 | I.43-61 | 25 | g/ha | >5 |
| 23 | I.45-61 | 25 | g/ha | >5 |
| 24 | I.50-60 | 25 | g/ha | >5 |
| 25 | I.66-11 | 25 | g/ha | >5 |
| 26 | I.70-1 | 25 | g/ha | >5 |
| 27 | I.71-1 | 25 | g/ha | >5 |
| | | | | EF |
| | | | | (ZEAMX) |
| 1 | I.1-6 | 25 | g/ha | >5 |
| 2 | I.2-1 | 25 | g/ha | >5 |
| 3 | I.2-7 | 250 | g/ha | >5 |
| 4 | I.3-41 | 25 | g/ha | >5 |
| 5 | I.3-51 | 25 | g/ha | >5 |
| 6 | I.4-1 | 25 | g/ha | >5 |
| 7 | I.4-51 | 25 | g/ha | >5 |
| 8 | I.4-59 | 250 | g/ha | >5 |
| 9 | I.5-48 | 250 | g/ha | >5 |
| 10 | I.6-6 | 250 | g/ha | >5 |
| 11 | I.7-6 | 25 | g/ha | >5 |
| 12 | I.8-1 | 25 | g/ha | >5 |
| 13 | I.10-1 | 25 | g/ha | >5 |
| 14 | I.10-6 | 25 | g/ha | >5 |
| 15 | I.32-60 | 25 | g/ha | >5 |
| 16 | I.36-1 | 25 | g/ha | >5 |
| 17 | I.37-63 | 25 | g/ha | >5 |
| 18 | I.38-1 | 25 | g/ha | >5 |
| 19 | I.43-61 | 25 | g/ha | >5 |
| 20 | I.45-61 | 25 | g/ha | >5 |
| 21 | I.50-60 | 25 | g/ha | >5 |
| 22 | I.61-6 | 250 | g/ha | >5 |
| 23 | I.66-11 | 25 | g/ha | >5 |
| 24 | I.68-11 | 25 | g/ha | >5 |
| 25 | I.71-1 | 25 | g/ha | >5 |
| 26 | I.72-1 | 25 | g/ha | >5 |
| | | | | EF |
| | | | | (TRZAS) |
| 1 | I.1-1 | 25 | g/ha | >5 |
| 2 | I.2-7 | 250 | g/ha | >5 |
| 3 | I.3-36 | 250 | g/ha | >5 |
| 4 | I.3-41 | 25 | g/ha | >5 |
| 5 | I.3-51 | 25 | g/ha | >5 |
| 6 | I.4-1 | 250 | g/ha | >5 |
| 7 | I.4-51 | 25 | g/ha | >5 |
| 8 | I.4-59 | 25 | g/ha | >5 |
| 9 | I.6-6 | 25 | g/ha | >5 |
| 10 | I.7-6 | 250 | g/ha | >5 |
| 11 | I.8-1 | 25 | g/ha | >5 |
| 12 | I.45-61 | 25 | g/ha | >5 |
| 13 | I.70-1 | 25 | g/ha | >5 |

In the above tables:

BRSNS=Brassica napus
HORVS=Hordeum vulgare
TRZAS=Triticum aestivum
ZEAMX=Zea mays

Similar results were also achieved with further compounds of the formula (I), also in the case of application to different plant species.

1. A substituted sulfonamide of formula (I) and/or a salt thereof 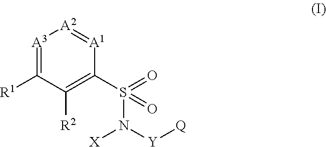
that increases tolerance to abiotic stress in plants,
in which
A¹, A², A³ are the same or different and are each independently N (nitrogen) or the C—W moiety, but there are never more than two adjacent nitrogen atoms, and where W in the C—W moiety in each case has identical or different definitions as defined below,
W in each case is hydrogen, nitro, amino, cyano, thiocyanato, isothiocyanato, halogen, alkyl, cycloalkyl, alkenyl, alkynyl, aryl, arylalkyl, arylalkoxy, heteroaryl, alkoxyalkyl, haloalkyl, halocycloalkyl, alkoxy, haloalkoxy, alkoxycarbonyl, hydroxycarbonyl, aminocarbonyl, alkylaminocarbonyl, cycloalkylaminocarbonyl, alkylamino, alkylthio, haloalkylthio, bisalkylamino, cycloalkylamino, alkylcarbonylamino, cycloalkylcarbonylamino, formylamino, haloalkylcarbonylamino, alkoxycarbonylamino, (alkyl)aminocarbonylamino, alkylsulfonylamino, cycloalkylsulfonylamino, arylsulfonylamino, hetarylsulfonylamino, sulfonylhaloalkylamino, aminoalkylsulfonyl, aminohaloalkylsulfonyl, alkylsulfonyl, cycloalkylsulfonyl, arylsulfonyl, alkylsulfinyl, cycloalkylsulfinyl, arylsulfinyl, N,S-dialkylsulfonimidoyl, S-alkylsulfonimidoyl, alkylsulfonylaminocarbonyl, cycloalkylsulfonylaminocarbonyl, cycloalkylaminosulfonyl,
R¹ is H, nitro, amino, cyano, halogen, alkyl, cycloalkyl, alkenyl, alkynyl, aryl, arylalkyl, heteroaryl, alkoxyalkyl, haloalkyl, halocycloalkyl, alkoxy, haloalkoxy, alkylthio, haloalkylthio, alkoxycarbonyl, hydroxycarbonyl, aminocarbonyl, alkylaminocarbonyl, alkylamino, bisalkylamino, cycloalkylamino, alkylcarbonylamino, cycloalkylcarbonylamino, formylamino, haloalkylcarbonylamino, alkoxycarbonylamino, (alkyl)aminocarbonylamino, sulfonylamino, alkylsulfonylamino, haloalkylsulfonylamino, cycloalkylsulfonylamino, aminoalkylsulfonyl, aminohaloalkylsulfonyl, alkylsulfonyl, cycloalkylsulfonyl, arylsulfonyl, alkylsulfinyl, cycloalkylsulfinyl, arylsulfinyl, N,S-dialkylsulfonimidoyl, S-alkylsulfonimidoyl, alkylsulfonylaminocarbonyl, cycloalkylsulfonylaminocarbonyl, cycloalkylaminosulfonyl,
R² is H, nitro, amino, cyano, halogen, alkyl, cycloalkyl, alkenyl, alkynyl, aryl, arylalkyl, heteroaryl, alkoxyalkyl, haloalkyl, halocycloalkyl, alkoxy, haloalkoxy, alkylthio, haloalkylthio, alkoxycarbonyl, hydroxycarbonyl, aminocarbonyl, alkylaminocarbonyl, alkylamino, bisalkylamino, cycloalkylamino, alkylcarbonylamino, cycloalkylcarbonylamino, formylamino, haloalkylcarbonylamino, alkoxycarbonylamino, (alkyl)aminocarbonylamino, sulfonylamino, alkylsulfonylamino, haloalkylsulfonylamino, cycloalkylsulfonylamino, aminoalkylsulfonyl, aminohaloalkylsulfonyl, alkylsulfonyl, cycloalkylsulfonyl, arylsulfonyl, alkylsulfinyl, cycloalkylsulfinyl, arylsulfinyl, N,S-dialkylsulfonimidoyl, S-alkylsulfonimidoyl, alkylsulfonylaminocarbonyl, cycloalkylsulfonylaminocarbonyl, cycloalkylaminosulfonyl,
R¹ and R² with the atoms to which they are bonded form a fully saturated, partly saturated or fully unsaturated, 5 to 7-membered ring optionally interrupted by heteroatoms and optionally with further substitution,
R¹ and A³, when A³ is a C—W group, with the atoms to which they are bonded form a fully saturated, partly saturated or fully unsaturated, 5 to 7-membered ring optionally interrupted by heteroatoms and optionally with further substitution,
X is H, alkyl, alkylalkenyl, alkynyl, alkylcarbonyl, cycloalkylcarbonyl, arylcarbonyl, heteroarylcarbonyl, alkoxycarbonyl, alkylsulfonyl, cycloalkylsulfonyl, arylsulfonyl, alkoxycarbonylcarbonyl, arylalkoxycarbonylcarbonyl, alkylaminothiocarbonyl, alkylaminocarbonyl, arylalkyl, hetaroarylalkyl, cyanoalkyl, haloalkyl, cycloalkyl, cycloalkylalkyl,
Y is alkanediyl, cycloalkanediyl, alkenediyl, alkynediyl, alkylalkenediyl, arylalkanediyl, alkoxydiyl, alkoxyalkyldiyl, alkylthioalkyldiyl, alkylsulfinylalkyldiyl, alkylsulfonylalkyldiyl or a direct bond between N and Q,
Q is a phenyl ring or a 5-, 6- or 7-membered saturated or unsaturated heterocyclic ring or a 9- or 10-membered fused bicyclic or tricyclic ring system which optionally contains further heteroatoms, where the particular aforementioned ring or the ring system is optionally mono- or polysubstituted, identically or differently, and where the substituents are each independently selected from H, halogen, cyano, nitro, branched or unbranched alkyl, cycloalkyl, alkoxycycloalkyl, arylcycloalkyl, cycloalkylcycloalkyl, cycloalkylalkyl, alkenyl, alkynyl, arylalkyl, alkoxyalkyl, haloalkyl, halocycloalkyl, alkoxy, haloalkoxy, alkoxycarbonyl, hydroxycarbonyl, aminocarbonyl, alkylaminocarbonyl, amino, alkylamino, bisalkylamino, cycloalkylamino, alkylcarbonylamino, alkylcarbonyl, alkylthio, haloalkylthio, haloalkylsulfinyl, haloalkylsulfonyl or where the substituents are each independently selected from phenyl or a 5- or 6-membered heteroaromatic ring, where phenyl or the heteroaromatic ring is optionally mono- or polysubstituted, identically or differently, by alkyl, alkenyl, alkynyl, cycloalkyl, haloalkyl, haloalkenyl, haloalkynyl, halocycloalkyl, halogen, cyano, nitro, alkoxy, haloalkoxy, alkylthio, haloalkylthio.

2. A sulfonamide and/or salt as claimed in claim 1, where, in formula (I),
A¹, A², A³ are the same or different and are each independently N (nitrogen) or the C—W moiety, but there are never more than two adjacent nitrogen atoms, and where W in the C—W moiety in each case may have identical or different definitions as defined below,
W in each case is hydrogen, nitro, amino, cyano, halogen, (C₁-C₆)-alkyl, (C₃-C₈)-cycloalkyl, (C₂-C₈)-alkenyl, (C₂-C₈)-alkynyl, aryl, aryl-(C₁-C₆)-alkyl, heteroaryl, (C₁-C₆)-alkoxy-(C₁-C₆)-alkyl, (C₁-C₆)-haloalkyl, (C₃-C₈)-halocycloalkyl, (C₁-C₆)-alkoxy, aryl-(C₁-C₆)-alkoxy, (C₁-C₆)-haloalkoxy, (C₁-C₆)-alkoxycarbonyl, hydroxycarbonyl, aminocarbonyl, (C₁-C₆)-alkylaminocarbonyl, (C₃-C₈)-cycloalkylaminocarbonyl, (C₁-C₆)-alkylamino, (C₁-C₆)-alkylthio, (C₁-C₆)-haloalkylthio, bis(C₁-C₆)-alkylamino, (C₃-C₈)-cycloalkylamino, (C₁-C₆)-alkylcarbonylamino, (C₃-C₈)-cycloalkylcarbonylamino, formylamino, (C₁-C₆)-haloalkylcarbonylamino, (C₁-C₆)-alkoxycarbonylamino, (C₁-C₆)-(alkyl)aminocarbonylamino, (C₁-C₆)-alkylsulfonylamino, (C₃-C₈)-cycloalkylsulfonylamino, arylsulfonylamino; hetarylsulfonylamino, sulfonyl-(C₁-C₆)-haloalkylamino, amino-(C₁-C₆)-alkylsulfonyl, amino-(C₁-C₆)-haloalkylsulfonyl, (C₁-C₆)-alkylsulfonyl, (C₃-C₈)-cycloalkylsulfonyl, arylsulfonyl, (C₁-C₆)-alkylsulfinyl, (C₃-C₈)-cycloalkylsulfinyl, arylsulfinyl, N,S-di-(C₁-C₆)-alkylsulfonimidoyl, S—(C₁-C₆)-alkylsulfonimidoyl, (C₁-C₆)-alkylsulfonylaminocarbonyl, (C₃-C₈)-cycloalkylsulfonylaminocarbonyl, (C₁-C₆)-cycloalkylaminosulfonyl,
R¹ is H, nitro, amino, cyano, halogen, (C₁-C₆)-alkyl, (C₃-C₈)-cycloalkyl, (C₂-C₈)-alkenyl, (C₂-C₈)-alkynyl, aryl, aryl-(C₁-C₆)-alkyl, heteroaryl, (C₁-C₆)-alkoxy-(C₁-C₆)-alkyl, (C₁-C₆)-haloalkyl, (C₃-C₈)-halocycloalkyl, (C₁-C₆)-alkoxy, (C₁-C₆)-haloalkoxy, (C₁-C₆)-alkylthio, (C₁-C₆)-haloalkylthio, (C₁-C₆)-alkoxycarbonyl, hydroxycarbonyl, aminocarbonyl, (C₁-C₆) alkylaminocarbonyl, (C₃-C₈)-cycloalkylaminocarbonyl, (C₁-C₆)-alkylamino, bis(C₁-C₆)-alkylamino, (C₃-C₈)-cycloalkylamino, (C₁-C₆)-alkylcarbonylamino, (C₃-C₈)-cycloalkylcarbonylamino, formylamino, (C₁-C₆)-haloalkylcarbonylamino, (C₁-C₆)-alkoxycarbonylamino, (C₁-C₆)-(alkyl)aminocarbonylamino, (C₁-C₆)-alkylsulfonylamino, (C₃-C₈)-cycloalkylsulfonylamino, arylsulfonylamino, hetarylsulfonylamino, sulfonyl(C₁-C₆)-haloalkylamino, amino-(C₁-C₆)-alkylsulfonyl, amino-(C₁-C₆)-haloalkylsulfonyl, (C₁-C₆)-alkylsulfonyl, (C₃-C₈)-cycloalkylsulfonyl, arylsulfonyl, (C₁-C₆)-alkylsulfinyl, (C₃-C₈)-cycloalkylsulfinyl, arylsulfinyl, N,S-di-(C₁-C₆)-alkylsulfonimidoyl, S—(C₁-C₆)-alkylsulfonimidoyl, (C₁-C₆)-alkylsulfonylaminocarbonyl, (C₃-C₈)-cycloalkylsulfonylaminocarbonyl, (C₁-C₆)-cycloalkylaminosulfonyl,
R² is H, nitro, amino, cyano, halogen, (C₁-C₆)-alkyl, (C₃-C₈)-cycloalkyl, (C₂-C₈)-alkenyl, (C₂-C₈)-alkynyl, aryl, aryl-(C₁-C₆)-alkyl, heteroaryl, (C₁-C₆)-alkoxy-(C₁-C₆)-alkyl, (C₁-C₆)-haloalkyl, (C₃-C₈)-halocycloalkyl, (C₁-C₆)-alkoxy, (C₁-C₆)-haloalkoxy, (C₁-C₆)-alkylthio, (C₁-C₆)-haloalkylthio, (C₁-C₆)-alkoxycarbonyl, hydroxycarbonyl, aminocarbonyl, (C₁-C₆)-alkylaminocarbonyl, (C₃-C₈)-cycloalkylaminocarbonyl, alkylamino, bis(C₁-C₆)-alkylamino, (C₃-C₈)-cycloalkylamino, (C₁-C₆)-alkylcarbonylamino, (C₃-C₈)-cycloalkylcarbonylamino, formylamino, (C₁-C₆)-haloalkylcarbonylamino, (C₁-C₆)-alkoxycarbonylamino, (C₁-C₆)-(alkyl)aminocarbonylamino, (C₁-C₆)-alkylsulfonylamino, (C₃-C₈)-cycloalkylsulfonylamino, arylsulfonylamino, hetarylsulfonylamino, sulfonyl(C₁-C₆)-haloalkylamino, amino-(C₁-C₆)-alkylsulfonyl, amino-(C₁-C₆)-haloalkylsulfonyl, (C₁-C₆)-alkylsulfonyl, (C₃-C₈)-cycloalkylsulfonyl, arylsulfonyl, (C₁-C₆)-alkylsulfinyl, (C₃-C₈)-cycloalkylsulfinyl, arylsulfinyl, N,S-di-(C₁-C₆)-alkylsulfonimidoyl, S—(C₁-C₆)-alkylsulfonimidoyl, (C₁-C₆)-alkylsulfonylaminocarbonyl, (C₃-C₈)-cycloalkylsulfonylaminocarbonyl, (C₁-C₆)-cycloalkylaminosulfonyl,
R¹ and R² with the atoms to which they are bonded form a fully saturated, partly saturated or fully unsaturated, 5 to 7-membered ring optionally interrupted by heteroatoms and optionally with further substitution,
R¹ and A³, when A³ is a C—W group, with the atoms to which they are bonded form a fully saturated, partly saturated or fully unsaturated, 5 to 7-membered ring optionally interrupted by heteroatoms and optionally with further substitution,
X is H, (C₁-C₆)-alkyl-(C₂-C₈)-alkenyl, (C₁-C₆)-alkylcarbonyl, (C₃-C₈)-cycloalkylcarbonyl, arylcarbonyl, heteroarylcarbonyl, (C₁-C₆)-alkoxycarbonyl, (C₁-C₆)-alkylsulfonyl, (C₃-C₈)-cycloalkylsulfonyl, arylsulfonyl, (C₁-C₆)-alkoxycarbonylcarbonyl, aryl-(C₁-C₆)-alkoxycarbonylcarbonyl, (C₁-C₆)-alkylaminothiocarbonyl, (C₁-C₆)-alkylaminocarbonyl, aryl-(C₁-C₆)-alkyl, heteroaryl-(C₁-C₆)-alkyl, (C₂-C₈)-alkynyl, (C₁-C₆)-cyanoalkyl, (C₁-C₆)-haloalkyl, (C₃-C₈)-cycloalkyl, (C₃-C₇)-cycloalkyl-(C₁-C₆)-alkyl,
Y is (C₁-C₈)-alkanediyl, (C₂-C₈)-alkynyl, (C₃-C₇)-cycloalkanediyl, (C₂-C₇)-alkenediyl, (C₁-C₆)-alkyl-(C₂-C₇)-alkenediyl, aryl-(C₁-C₆)-alkanediyl, C₆)-alkoxydiyl, (C₁-C₆)-alkoxy-(C₁-C₆)-alkyldiyl, (C₁-C₆)-alkoxycarbonyl-(C₁-C₆)-alkyldiyl, (C₁-C₆)-alkylthio-(C₁-C₆)-alkyldiyl, (C₁-C₆)-alkylsulfinyl-(C₁-C₆)-alkyldiyl, (C₁-C₆)-alkylsulfonyl-(C₁-C₆)-alkyldiyl or a direct bond between N and Q,
Q is a phenyl ring or a 5-, 6- or 7-membered saturated or unsaturated heterocyclic ring or a 9- or 10-membered fused bicyclic or tricyclic ring system which may optionally contain further heteroatoms, where the particular aforementioned ring or the ring system is optionally mono- or polysubstituted, identically or differently, and where the substituents are each independently selected from H, halogen, cyano, nitro, branched or unbranched (C₃-C₈)-cycloalkyl, (C₂-C₈)-alkenyl, (C₂-C₈)-alkynyl, aryl-(C₁-C₆)-alkyl, (C₁-C₆)-alkoxy-(C₁-C₆)-alkyl, (C₁-C₆)-haloalkyl, (C₃-C₈)-halocycloalkyl, (C₃-C₈)-alkoxycycloalkyl, (C₃-C₈)-arylcycloalkyl, (C₃-C₈)-cycloalkyl-(C₃-C₈)-cycloalkyl, (C₃-C₈)-cycloalkyl-(C₁-C₈)-alkyl, (C₁-C₆)-alkoxy, (C₁-C₆)-haloalkoxy, (C₁-C₆)-alkoxycarbonyl, hydroxycarbonyl, aminocarbonyl, (C₁-C₆)-alkylaminocarbonyl, amino, (C₁-C₆)-alkylamino, bis(C₁-C₆)-alkylamino, (C₃-C₈)-cycloalkylamino, (C₁-C₆)-alkylcarbonylamino, (C₁-C₆)-alkylcarbonyl, (C₁-C₆)-alkylthio, (C₁-C₆)-haloalkylthio, (C₁-C₆)-haloalkylsulfinyl, (C₁-C₆)-haloalkylsulfonyl or where the substituents are each independently selected from phenyl or a 5- or 6-membered heteroaromatic ring, where phenyl or the heteroaromatic ring is optionally mono- or polysubstituted, identically or differently, by (C₁-C₆)-alkyl, (C₂-C₈)-alkenyl, (C₂-C₈)-alkynyl, (C₃-C₈)-cycloalkyl, (C₁-C₆)-haloalkyl, (C₂-C₈)-haloalkenyl, (C₂-C₈)-haloalkynyl, (C₃-C₈)-halocycloalkyl, halogen, cyano, nitro, (C₁-C₆)-alkoxy, (C₁-C₆)-haloalkoxy, (C₁-C₆)-alkylthio, (C₁-C₆)-haloalkylthio, and Q is additionally one of the following radicals Q-1 to Q-192:

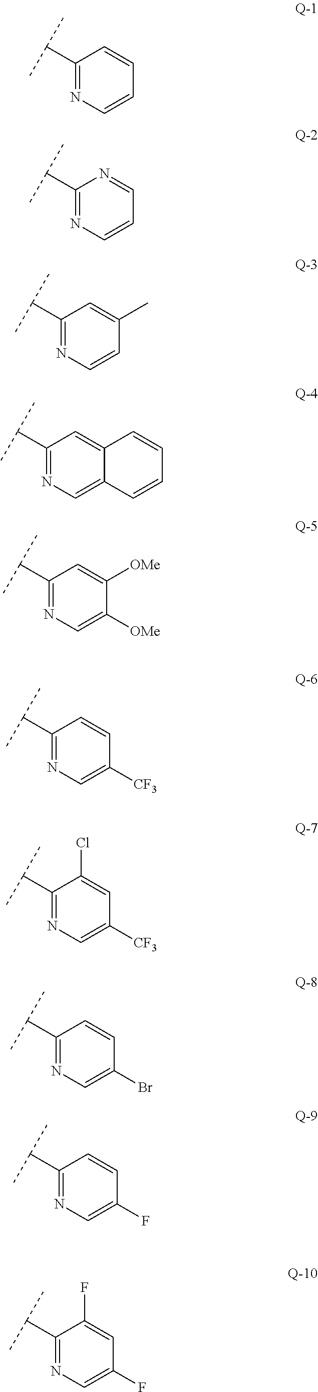

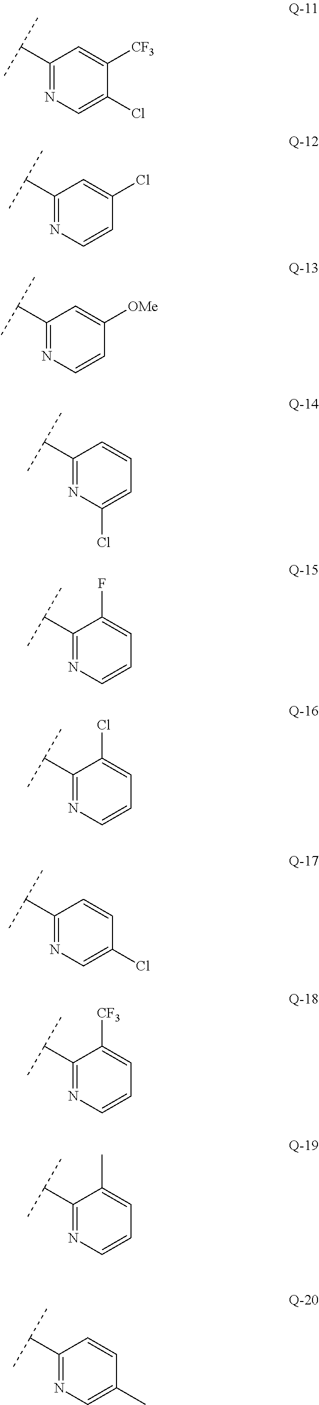

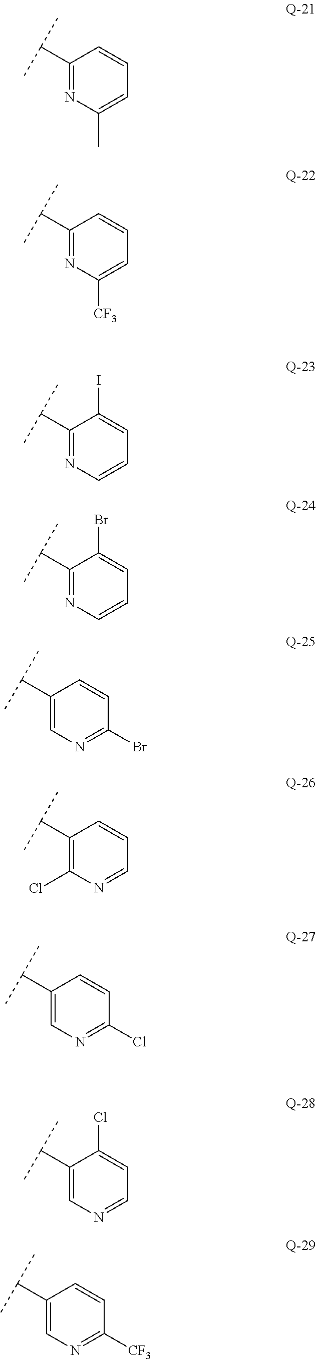

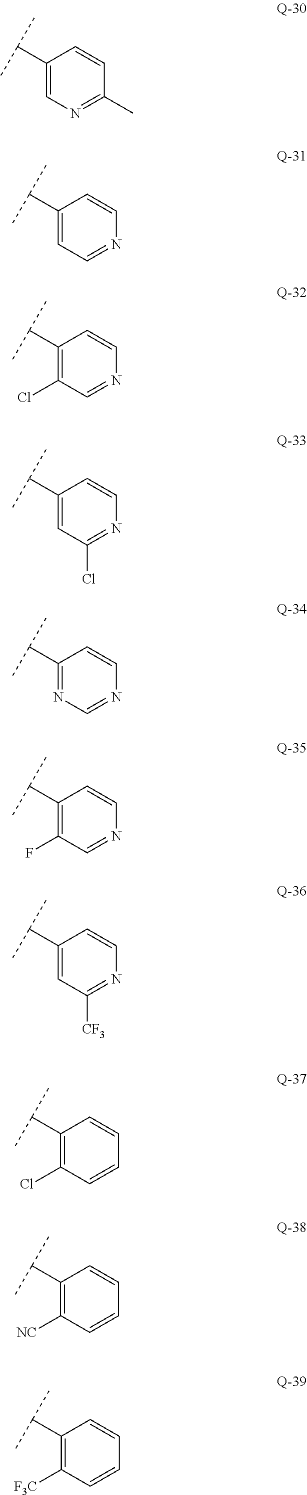

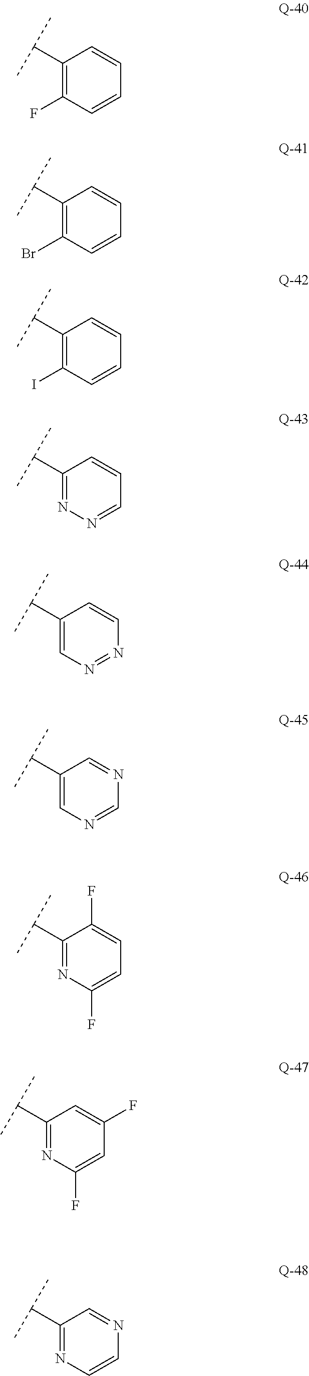

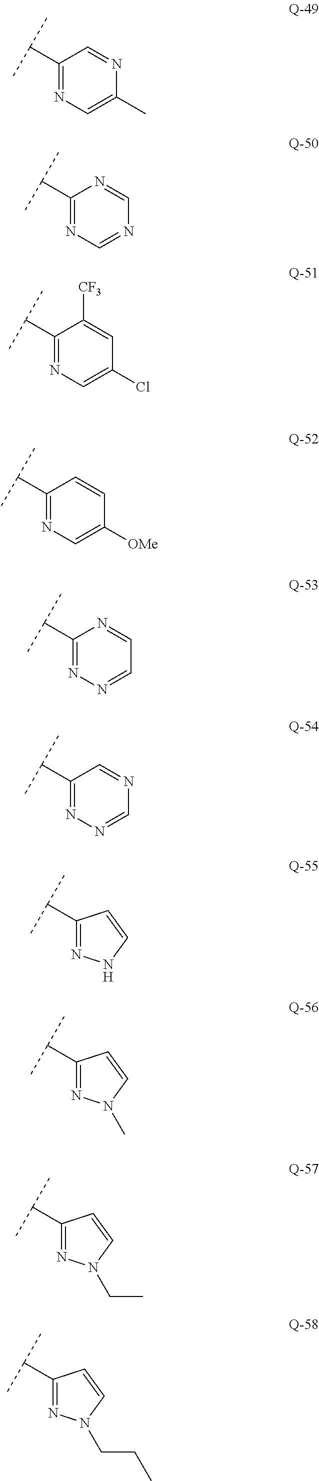

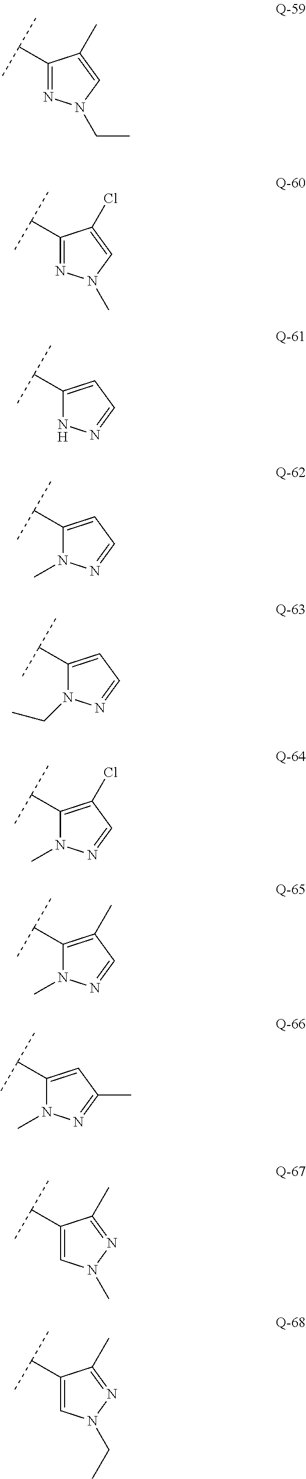

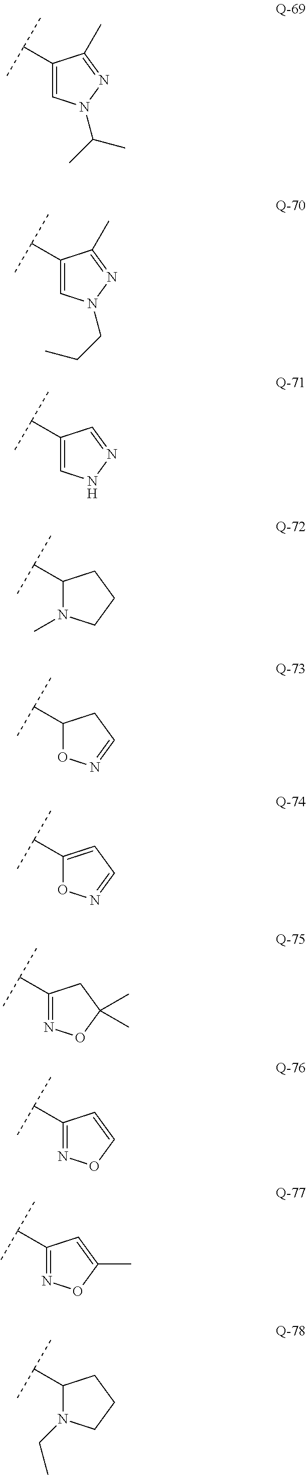

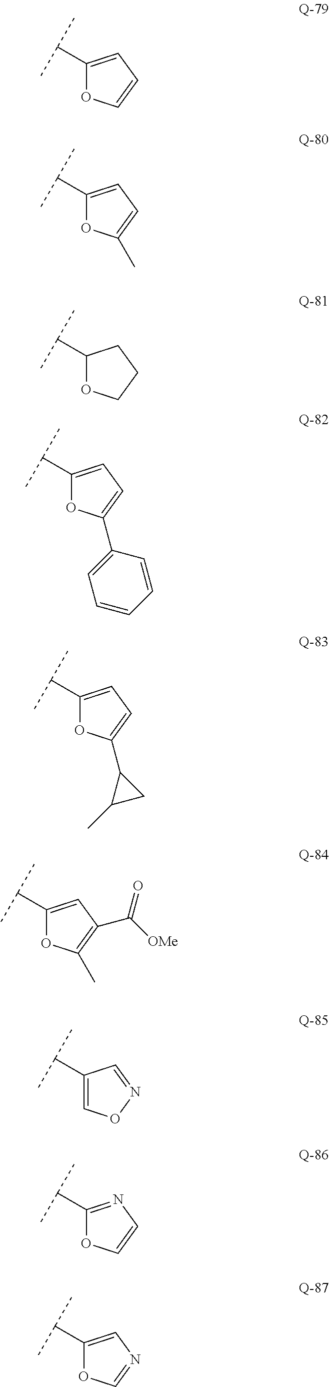

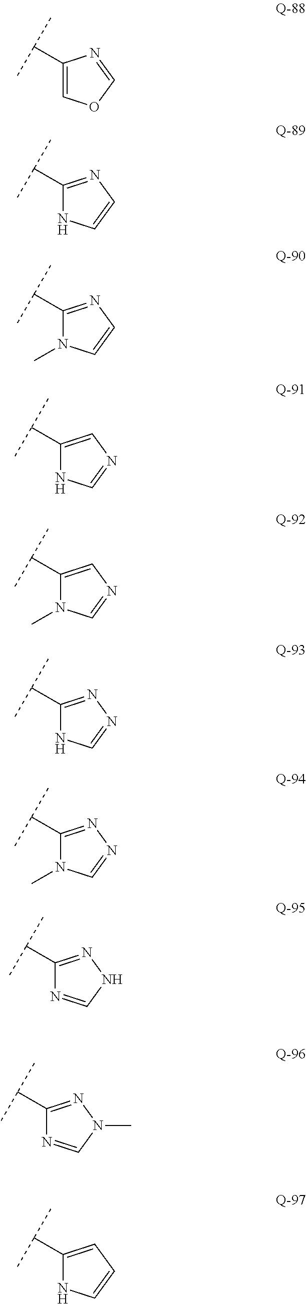

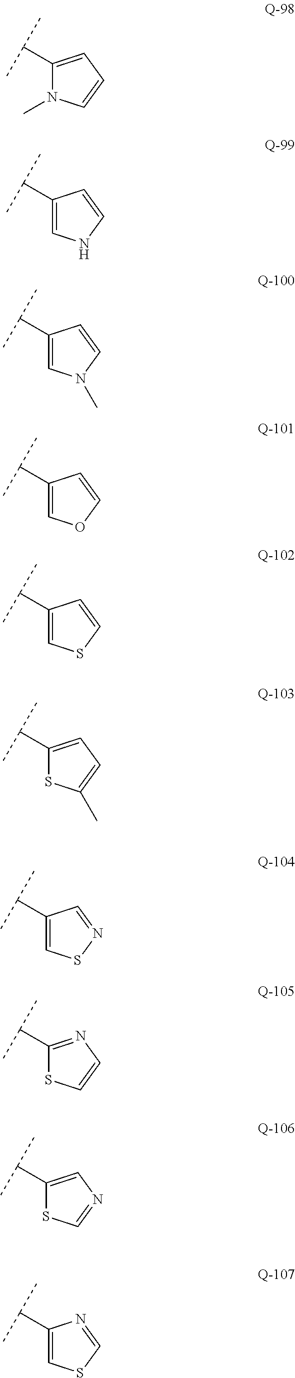

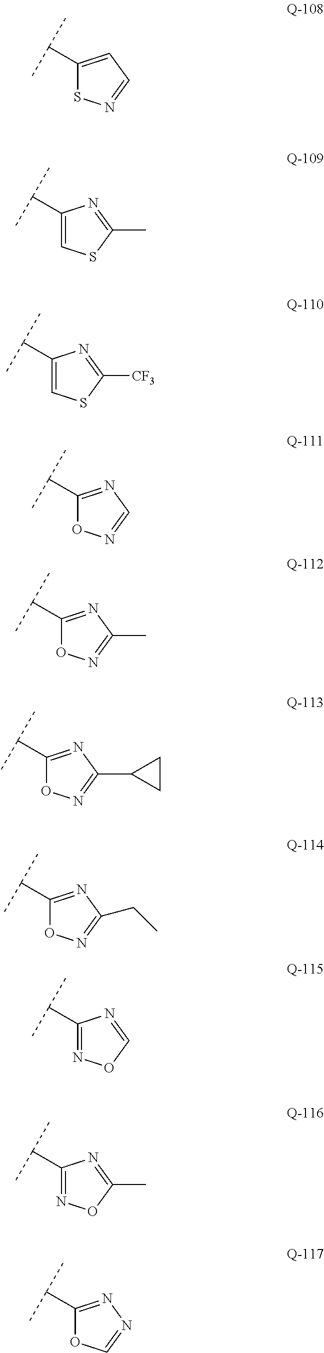

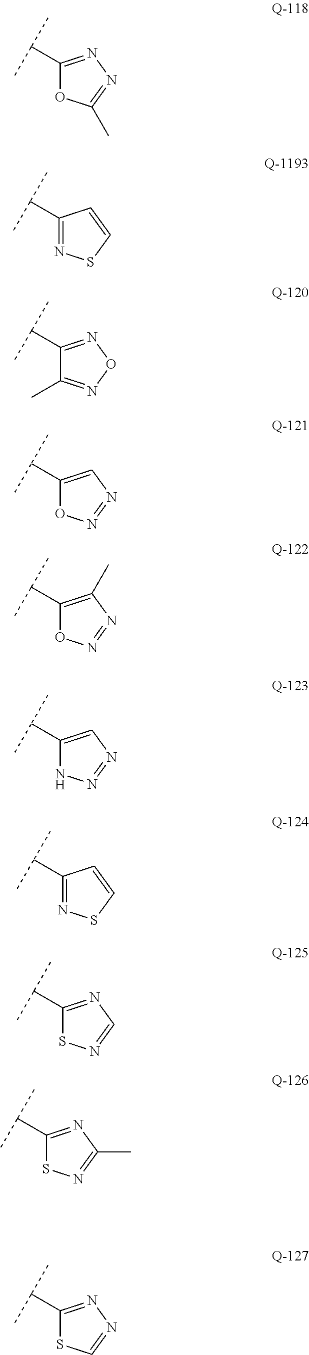

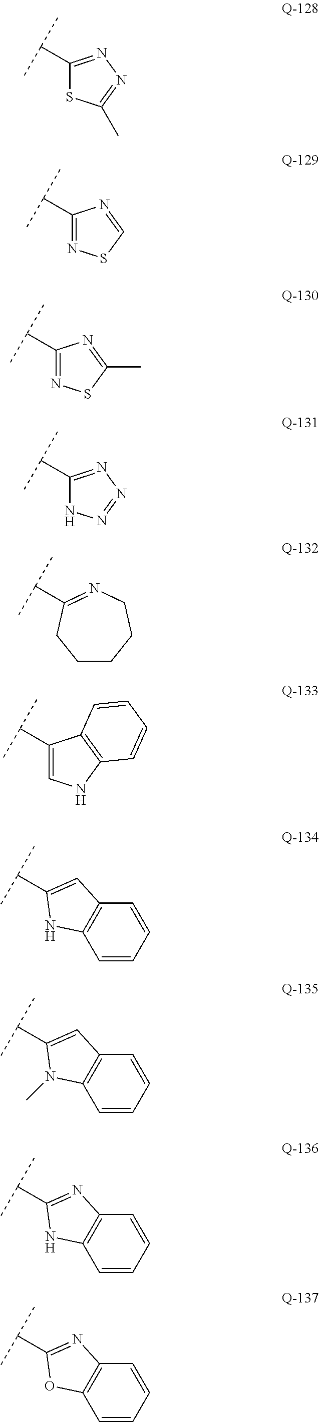

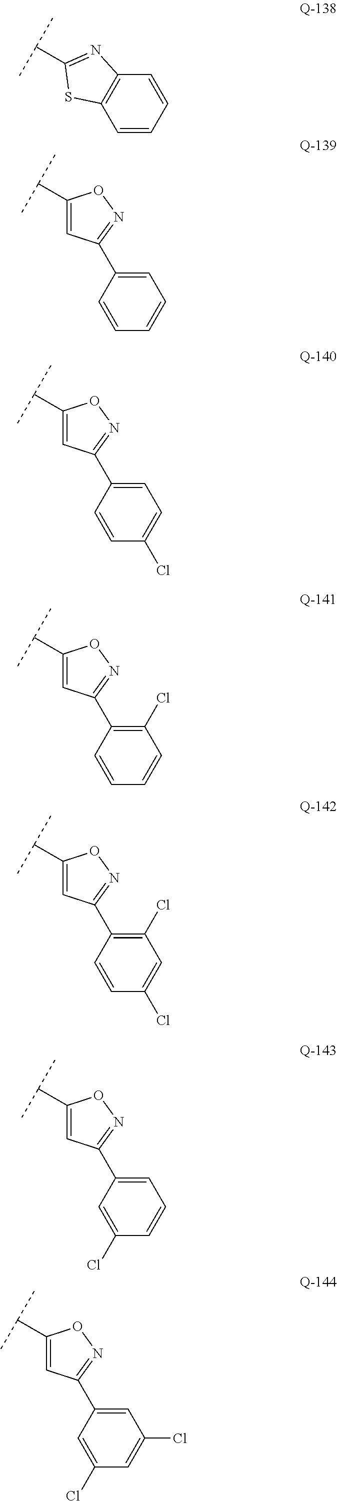

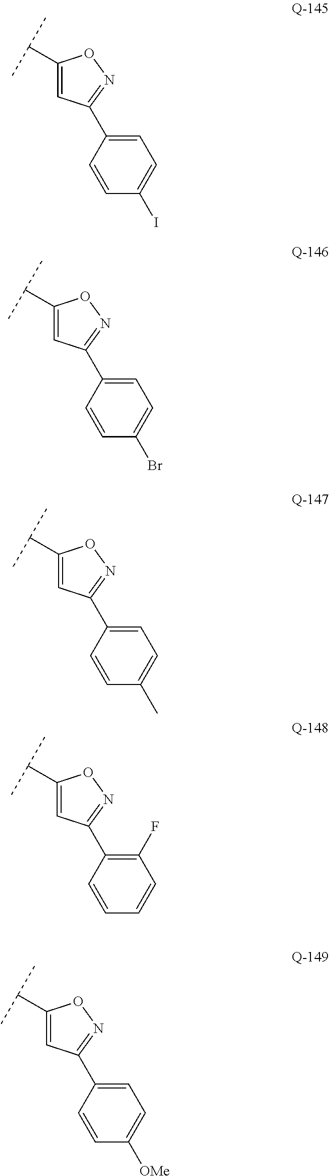

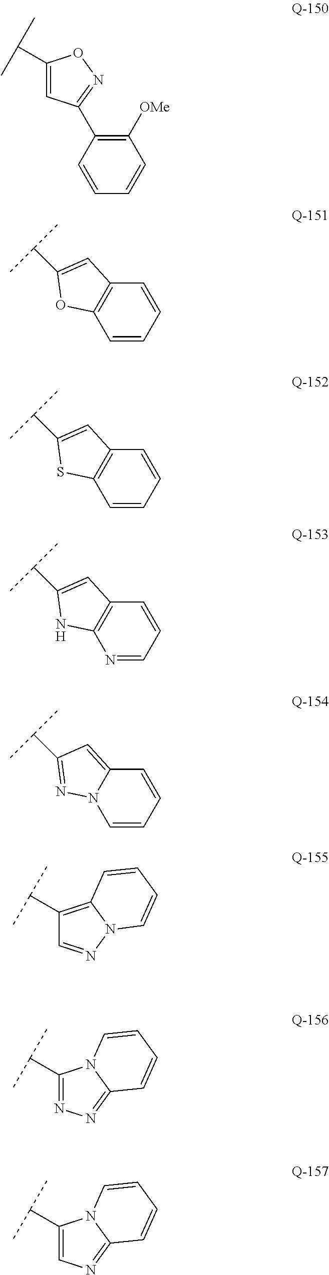

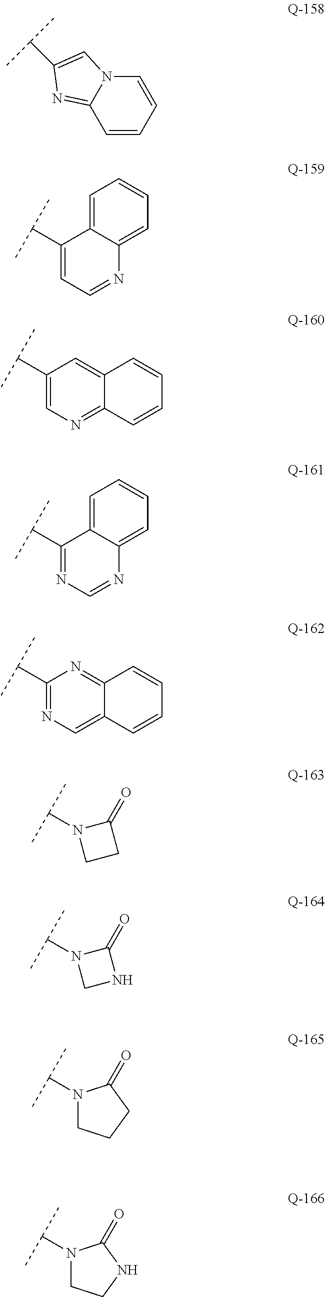

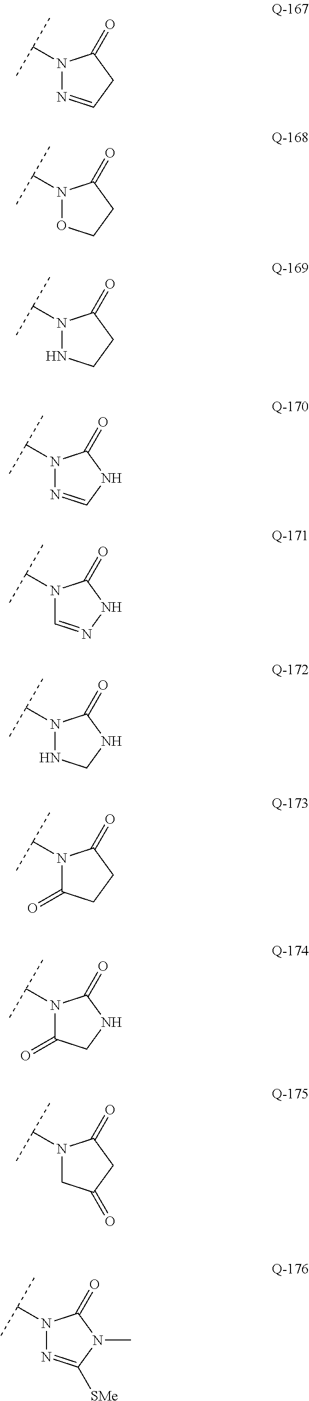

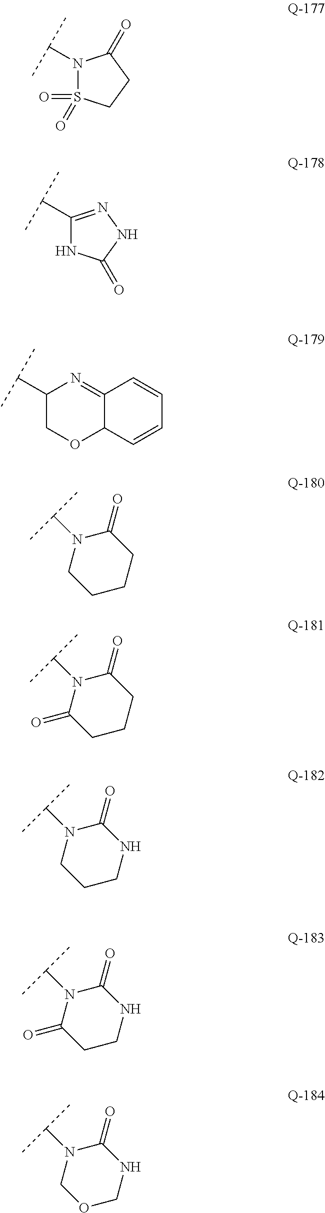

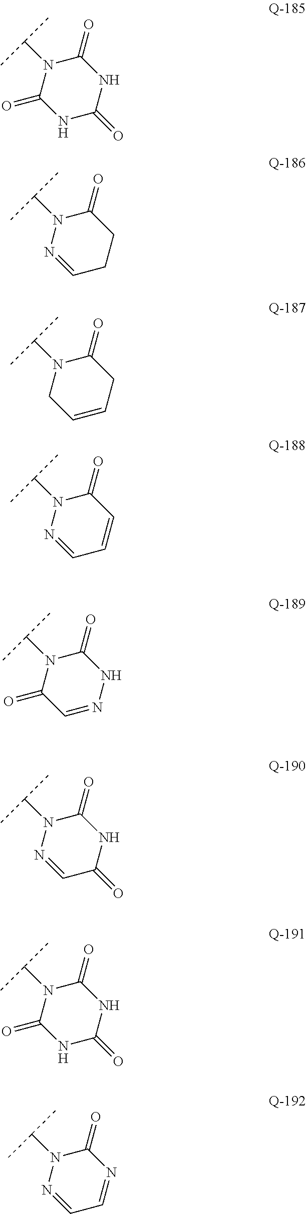

3. A sulfonamide and/or salt as claimed in claim 2, where, in formula (I),
A¹, A², A³ are the same or different and are each independently N (nitrogen) or the C—W moiety, but there are never more than two adjacent nitrogen atoms, and where W in the C—W moiety in each case has identical or different definitions as defined below;
W in each case is hydrogen, nitro, amino, cyano, halogen, (C₁-C₄)-alkyl, (C₃-C₆)-cycloalkyl, (C₂-C₆)-alkenyl, (C₂-C₆)-alkynyl, aryl, aryl-(C₁-C₄)-alkyl, heteroaryl, (C₁-C₄)-alkoxy-(C₁-C₄)-alkyl, (C₁-C₄)-haloalkyl, (C₃-C₆)-halocycloalkyl, (C₁-C₄)-alkoxy, aryl-(C₁-C₄)-alkoxy, (C₁-C₄)-haloalkoxy, (C₁-C₄)-alkoxycarbonyl, hydroxycarbonyl, aminocarbonyl, (C₁-C₄)-alkylaminocarbonyl, (C₃-C₇)-cycloalkylaminocarbonyl, (C₁-C₄)-alkylamino, (C₁-C₄)-alkylthio, (C₁-C₄)-haloalkylthio, bis(C₁-C₄)-alkylamino, (C₃-C₆)-cycloalkylamino, (C₁-C₄)-alkylcarbonylamino, (C₃-C₇)-cycloalkylcarbonylamino, formylamino, (C₁-C₄)-haloalkylcarbonylamino, (C₁-C₄)-alkoxycarbonylamino, (C₁-C₄)-(alkyl)aminocarbonylamino, (C₁-C₄)-alkylsulfonylamino, (C₃-C₆)-cycloalkylsulfonylamino, arylsulfonylamino, hetarylsulfonylamino, sulfonyl-(C₁-C₄)-haloalkylamino, amino-(C₁-C₄)-alkylsulfonyl, amino-(C₁-C₄)-haloalkylsulfonyl, (C₁-C₄)-alkylsulfonyl, (C₃-C₇)-cycloalkylsulfonyl, arylsulfonyl, (C₁-C₄)-alkylsulfinyl, (C₃-C₇)-cycloalkylsulfinyl, arylsulfinyl, N,S-di-(C₁-C₄)-alkylsulfonimidoyl, S—(C₁-C₄)-alkylsulfonimidoyl, (C₁-C₄)-alkylsulfonylaminocarbonyl, (C₃-C₇)-cycloalkylsulfonylaminocarbonyl, (C₁-C₄)-cycloalkylaminosulfonyl,
R¹ is H, nitro, amino, cyano, halogen, (C₁-C₄)-alkyl, (C₃-C₆)-cycloalkyl, (C₂-C₆)-alkenyl, (C₂-C₆)-alkynyl, aryl, aryl-(C₁-C₄)-alkyl, heteroaryl, (C₁-C₄)-alkoxy-(C₁-C₄)-alkyl, (C₁-C₄)-haloalkyl, (C₃-C₆)-halocycloalkyl, (C₁-C₄)-alkoxy, (C₁-C₄)-haloalkoxy, (C₁-C₄)-alkylthio, (C₁-C₄)-haloalkylthio, (C₁-C₄)-alkoxycarbonyl, hydroxycarbonyl, aminocarbonyl, (C₁-C₄)-alkylaminocarbonyl, (C₃-C₇)-cycloalkylaminocarbonyl, (C₁-C₄)-alkylamino, bis(C₁-C₄)-alkylamino, (C₃-C₆)-cycloalkylamino, (C₁-C₄)-alkylcarbonylamino, (C₃-C₇)-cycloalkylcarbonylamino, formylamino, (C₁-C₄)-haloalkylcarbonylamino, (C₁-C₄)-alkoxycarbonylamino, (C₁-C₄)-(alkyl)aminocarbonylamino, (C₁-C₄)-alkylsulfonylamino, (C₃-C₆)-cycloalkylsulfonylamino, arylsulfonylamino, hetarylsulfonylamino, sulfonyl(C₁-C₄)-haloalkylamino, amino-(C₁-C₄)-alkylsulfonyl, amino-(C₁-C₄)-haloalkylsulfonyl, (C₁-C₄)-alkylsulfonyl, (C₃-C₇)-cycloalkylsulfonyl, arylsulfonyl, (C₁-C₄)-alkylsulfinyl, (C₃-C₇)-cycloalkylsulfinyl, arylsulfinyl, N,S-di-(C₁-C₄)-alkylsulfonimidoyl, S—(C₁-C₄)-alkylsulfonimidoyl, (C₁-C₄)-alkylsulfonylaminocarbonyl, (C₃-C₇)-cycloalkylsulfonylaminocarbonyl, (C₁-C₄)-cycloalkylaminosulfonyl,
R² is H, nitro, amino, cyano, halogen, (C₁-C₄)-alkyl, (C₃-C₆)-cycloalkyl, (C₂-C₆)-alkenyl, (C₂-C₆)-alkynyl, aryl, aryl-(C₁-C₄)-alkyl, heteroaryl, (C₁-C₄)-alkoxy-(C₁-C₄)-alkyl, (C₁-C₄)-haloalkyl, (C₃-C₆)-halocycloalkyl, (C₁-C₄)-alkoxy, (C₁-C₄)-haloalkoxy, (C₁-C₄)-alkylthio, (C₁-C₄)-haloalkylthio, (C₁-C₄)-alkoxycarbonyl, hydroxycarbonyl, aminocarbonyl, (C₁-C₄)-alkylaminocarbonyl, (C₃-C₇)-cycloalkylaminocarbonyl, (C₁-C₄)-alkylamino, bis(C₁-C₄)-alkylamino, (C₃-C₆)-cycloalkylamino, (C₁-C₄)-alkylcarbonylamino, (C₃-C₇)-cycloalkylcarbonylamino, formylamino, (C₁-C₄)-haloalkylcarbonylamino, (C₁-C₄)-alkoxycarbonylamino, (C₁-C₄)-(alkyl)aminocarbonylamino, (C₁-C₄)-alkylsulfonylamino, (C₃-C₆)-cycloalkylsulfonylamino, arylsulfonylamino, hetarylsulfonylamino, sulfonyl(C₁-C₄)-haloalkylamino, amino-(C₁-C₄)-alkylsulfonyl, amino-(C₁-C₄)-haloalkylsulfonyl, (C₁-C₄)-alkylsulfonyl, (C₃-C₇)-cycloalkylsulfonyl, arylsulfonyl, (C₁-C₄)-alkylsulfinyl, (C₃-C₇)-cycloalkylsulfinyl, arylsulfinyl, N,S-di-(C₁-C₄)-alkylsulfonimidoyl, S—(C₁-C₄)-alkylsulfonimidoyl, (C₁-C₄)-alkylsulfonylaminocarbonyl, (C₃-C₇)-cycloalkylsulfonylaminocarbonyl, (C₁-C₄)-cycloalkylaminosulfonyl,
R¹ and R² with the atoms to which they are bonded form a fully saturated, partly saturated or fully unsaturated, 5 to 6-membered ring optionally interrupted by heteroatoms and optionally with further substitution,
R¹ and A³, when A³ is a C—W group, with the atoms to which they are bonded form a fully saturated, partly saturated or fully unsaturated, 5 to 6-membered ring optionally interrupted by heteroatoms and optionally with further substitution,
X is H, (C₁-C₄)-alkyl, (C₁-C₄)-alkyl-(C₂-C₆)-alkenyl, (C₁-C₄)-alkylcarbonyl, (C₃-C₆)-cycloalkylcarbonyl, arylcarbonyl, heteroarylcarbonyl, (C₁-C₄)-alkoxycarbonyl, (C₁-C₄)-alkylsulfonyl, (C₃-C₆)-cycloalkylsulfonyl, arylsulfonyl, (C₁-C₄)-alkoxycarbonylcarbonyl, aryl-(C₁-C₄)-alkoxycarbonylcarbonyl, (C₁-C₄)-alkylaminothiocarbonyl, (C₁-C₄)-alkylaminocarbonyl, aryl-(C₁-C₄)-alkyl, hetaroaryl-(C₁-C₄)-alkyl, (C₂-C₆)-alkynyl, (C₁-C₄)-cyanoalkyl, (C₁-C₄)-haloalkyl, (C₃-C₇)-cycloalkyl, (C₃-C₇)-cycloalkyl-(C₁-C₆)-alkyl,
Y is (C₁-C₅)-alkanediyl, (C₃-C₆)-cycloalkanediyl, (C₂-C₆)-alkenediyl, (C₂-C₆)-alkynediyl, (C₁-C₄)-alkyl-(C₂-C₆)-alkenediyl, aryl-(C₁-C₄)-alkanediyl, (C₁-C₄)-alkoxydiyl, (C₁-C₄)-alkoxy-(C₁-C₄)-alkyldiyl, (C₁-C₄)-alkoxycarbonyl-(C₁-C₄)-alkyldiyl, (C₁-C₄)-alkylthio-(C₁-C₄)-alkyldiyl, (C₁-C₄)-alkylsulfinyl-(C₁-C₄)-alkyldiyl, (C₁-C₄)-alkylsulfonyl-(C₁-C₄)-alkyldiyl or a direct bond between N and Q,
Q is a phenyl ring or a 5-, 6- or 7-membered saturated or unsaturated heterocyclic ring or a 9- or 10-membered fused bicyclic or tricyclic ring system which optionally contains further heteroatoms, where the particular aforementioned ring or the ring system is optionally mono- or polysubstituted, identically or differently, and where the substituents are each independently selected from H, halogen, cyano, nitro, branched or unbranched (C₁-C₄)-alkyl, (C₃-C₆)-cycloalkyl, (C₃-C₇)-alkoxycycloalkyl, (C₃-C₇)-arylcycloalkyl, (C₃-C₇)-cycloalkyl-(C₃-C₇)-cycloalkyl, (C₃-C₇)-cycloalkyl-(C₁-C₄)-alkyl, (C₂-C₆)-alkenyl, (C₂-C₆)-alkynyl, aryl-(C₁-C₄)-alkyl, (C₁-C₄)-alkoxy-(C₁-C₄)-alkyl, (C₁-C₄)-haloalkyl, (C₃-C₆)-halocycloalkyl, (C₁-C₄)-alkoxy, (C₁-C₄)-haloalkoxy, (C₁-C₄)-alkoxycarbonyl, hydroxycarbonyl, aminocarbonyl, (C₁-C₄)-alkylaminocarbonyl, amino, (C₁-C₄)-alkylamino, bis(C₁-C₄)-alkylamino, (C₃-C₆)-cycloalkylamino, (C₁-C₄)-alkylcarbonylamino, (C₁-C₄)-alkylcarbonyl, (C₁-C₄)-alkylthio, (C₁-C₄)-haloalkylthio, (C₁-C₄)-haloalkylsulfinyl, (C₁-C₄)-haloalkylsulfonyl, or where the substituents are each independently selected from phenyl or a 5- or 6-membered heteroaromatic ring, where phenyl or the heteroaromatic ring is optionally mono- or polysubstituted, identically or differently, by (C₁-C₄)-alkyl, (C₂-C₆)-alkenyl, (C₂-C₆)-alkynyl, (C₃-C₇)-cycloalkyl, (C₁-C₄)-haloalkyl, (C₂-C₆)-haloalkenyl, (C₂-C₆)-haloalkynyl, (C₃-C₆)-halocycloalkyl, halogen, cyano, nitro, (C₁-C₄)-alkoxy, (C₁-C₄)-haloalkoxy, (C₁-C₄)-alkylthio, (C₁-C₄)-haloalkylthio, and Q is additionally one of the above-described Q-1 to Q-192 radicals.

4. A sulfonamide and/or salt as claimed in claim 2, where, in formula (I),
A¹, A², A³ are the same or different and are each independently N (nitrogen) or the C—W moiety, but there are never more than two adjacent nitrogen atoms, and where W in the C—W moiety in each case has identical or different definitions as defined below;
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene,
Q is an optionally mono- or polysubstituted phenyl ring or an optionally mono- or polysubstituted 5-, 6- or 7-membered saturated or unsaturated heterocyclic ring or a 9- or 10-membered fused heterobicyclic ring system from the above-described group of Q-1 to Q-192, where the substituents are each independently selected from hydrogen, methyl, ethyl, n-propyl, isopropyl, cyclopropyl, methylcyclopropyl, methoxy, ethoxy, trifluoromethyl, fluorine, chlorine, bromine, iodine, cyano, nitro, trifluoromethoxy, methylthio, trifluoromethylthio, OCHF₂, OCClF₂, methoxycarbonyl, phenyl, p-chlorophenyl, m-chlorophenyl, o-chlorophenyl, o,p-dichlorophenyl, m,m-dichlorophenyl, o-iodophenyl, m-iodophenyl, p-iodophenyl, o-bromophenyl, m-bromophenyl, p-bromophenyl, o-methylphenyl, m-methylphenyl, p-methylphenyl, o-fluorophenyl, m-fluorophenyl, p-fluorophenyl, o-methoxyphenyl, m-methoxyphenyl, p-methoxyphenyl, o-trifluoromethylphenyl, m-trifluoromethylphenyl, p-trifluoromethylphenyl, p-trifluoromethoxyphenyl, p-trifluoromethylthiophenyl, o-nitrophenyl, m-nitrophenyl, p-nitrophenyl, o-cyanophenyl, m-cyanophenyl, p-cyanophenyl.

5. The use as claimed in claim 2, where, in formula (I),
A¹, A², A³ are the same or different and are each independently N (nitrogen) or the C—W moiety, but there are never more than two adjacent nitrogen atoms, and where W in the C—W moiety in each case has identical or different definitions as defined below,
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene,
Q is a moiety from the above-described group of Q-2 to Q-192.

6. A sulfonamide and/or salt as claimed in claim 2, where, in formula (I),
A¹, A², A³ are the same or different and are each independently N (nitrogen) or the C—W moiety, but there are never more than two adjacent nitrogen atoms, and where W in the C—W moiety in each case has identical or different definitions as defined below,
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene,
Q is the moieties Q-2, Q-34, Q-43 to Q-45, Q-48 to Q-50 and Q-53 to Q-192 from the above-described groups.

7. A sulfonamide and/or salt as claimed in claim 2, where, in formula (I),
A¹, A², A³ are the same or different and are each independently N (nitrogen) or the C—W moiety, but there are never more than two adjacent nitrogen atoms, and where W in the C—W moiety in each case has identical or different definitions as defined below;
W in each case is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃); spiro-cyclopropylene,
Q is a moiety Q-55 to Q-192 from the above-described group.

8. A sulfonamide and/or salt as claimed in claim 2, where, in formula (I),
A¹, A², A³ are the same or different and are each independently N (nitrogen) or the C—W moiety, but there are never more than two adjacent nitrogen atoms, and where W in the C—W moiety in each case has identical or different definitions as defined below;
W in each case is hydrogen, nitro, amino, cyano, fluorine, chlorine, bromine, iodine, (C₁-C₄-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ is hydrogen, nitro, amino, cyano, fluorine, chlorine, bromine, iodine, (C₁-C₄)-alkoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R² is hydrogen, nitro, amino, cyano, fluorine, chlorine, bromine, iodine, methoxy, ethoxy, isopropoxy, (C₁-C₄)-alkyl, (C₂-C₆)-alkenyl, cyclopropyl, cyclobutyl, methylcarbonylamino, cyclopropylcarbonylamino, cyclobutylcarbonylamino, (C₁-C₄)-alkylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl, methylsulfonylaminocarbonyl, cyclopropylsulfonylaminocarbonyl, cyclopropylaminocarbonyl, cyclopropylaminosulfonyl,
R¹ and R² with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
R¹ and A³, when A³ is a C—W group, with the atoms to which they are bonded form a fully saturated or fully unsaturated 5- to 6-membered carbo- or heterocyclic ring which is optionally further substituted by nitro, amino, cyano, fluorine, chlorine, bromine, iodine, methoxy, ethoxy, isopropoxy, methyl, ethyl, cyclopropyl, methylcarbonylamino, methylamino, trifluoromethyl, trifluoromethylthio, trifluoromethoxy, difluoromethoxy, methoxycarbonyl, ethoxycarbonyl, methoxycarbonylmethyl, dimethylamino, benzyloxy, phenyl,
X is H, methyl, ethyl, n-propyl, cyclopropyl, isopropyl, methylcarbonyl, ethylcarbonyl, 2′-trifluoromethyl-4′-pyridylcarbonyl, cyclopropylcarbonyl, benzyl, CH₂-pyridyl, n-propylcarbonyl, tert-butylcarbonyl, cyclobutylcarbonyl, cyclopentylcarbonyl, cyclohexylcarbonyl, methoxycarbonyl, ethoxycarbonyl, allyl, ethynyl, prop-1-yn-3-yl, but-2-yn-3-yl, cyanomethyl, 2,2-difluoroethyl, 2,2,2-trifluoroethyl, 2,2,3,3,3-pentafluoropropyl, 3,3,2,2-tetrafluoropropyl, 4,4,4-trifluorobutyl, methoxycarbonylmethyl,
Y is CH₂, CH₂CH₂, CH(CH₃), CH(CO₂Me)CH₂, CH(CF₃), spiro-cyclopropylene and
Q is a moiety Q-72, Q-73, Q-75, Q-78, Q-81, Q-132 and Q-163 to Q-192 from the above-described group.

9. A method for treatment of a plant, comprising the application of applying a nontoxic amount, effective for enhancing resistance of said plant to one or more abiotic stress factors, of one or more of the compounds of the formula (I) or salts thereof as claimed in claim 1.

10. The method for treatment as claimed in claim 9, wherein the abiotic stress factors correspond to one or more conditions selected from the group consisting of drought, cold and hot conditions, osmotic stress, waterlogging, elevated soil salinity, elevated exposure to minerals, ozone conditions, strong light conditions, and limited availability of nitrogen nutrients, limited availability of phosphorus nutrients.

11. A sulfonamide and/or salt of claim 1, which is in a form that can be spray applied to plants and/or plant parts, said sulfanomid and/or salt being present in combination with one or more active ingredients selected from the group consisting of insecticides, fungicides and bactericides.

12. A sulfonamide and/or salt of claim 1, which is in a form that can be spray applied to plants and/or plant parts, said sulfanomid and/or salt being present in combination with one or more fertilizers.

13. A sulfonamide and/or salt as claimed in claim 1 for application to genetically modified varieties, the seed thereof, or to cultivated areas on which these varieties grow.

14. A spray solution which comprises one or more of the compounds as claimed in claim 1 for enhancing the resistance of plants to abiotic stress factors.

15. A method for increasing stress tolerance in plants selected from the group of the useful plants, ornamental plants, turfgrass types, or trees, which comprises applied a sufficient, nontoxic amount of one or more of the compounds as claimed in claim 1 to an area where the corresponding effect is desired, and/or to plants, the seed thereof or to an area on which plants grow.

16. The method as claimed in claim 15, wherein the resistance of the plants thus treated to abiotic stress is increased by at least 3% compared to untreated plants under otherwise identical physiological conditions.

17. An aryl- and/or hetarylsulfonamide of formula (I), and/or a salt thereof, 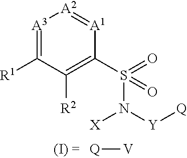
where V is 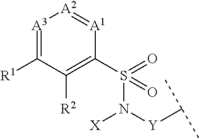
and in which
Q corresponds to the Q-1 radical as claimed in claim 2
and
V is defined as 5-iodonaphthalene-1-sulfonylaminomethyl, 4-fluoronaphthalene-1-sulfonylaminomethyl, 4-methylnaphthalene-1-sulfonylaminomethyl, 4-isopropoxynaphthalene-1-sulfonylaminomethyl, 4-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethyl-benzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 2-chloro-6-methylbenzenesulfonylaminomethyl, 2-chloro-6-methoxybenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 4-trifluoromethylthiobenzenesulfonylaminomethyl, 4-difluoromethoxybenzenesulfonylaminomethyl, 1-(4-bromobenzenesulfonylamino)cyclopropan-1-yl, 1-(4-chlorobenzenesulfonylamino)cyclopropan-1-yl, 1-(4-iodobenzenesulfonylamino)cyclopropan-1-yl, 1-(4-methoxybenzenesulfonylamino)cyclopropan-1-yl, 1-(4-bromonaphthalene-1-sulfonylamino)cyclopropan-1-yl, 1-(4-methoxynaphthalene-1-sulfonylamino)cyclopropan-1-yl, 1-(naphthalene-1-sulfonylamino)cyclopropan-1-yl, 1-(5-iodonaphthalene-1-sulfonylamino)cyclopropan-1-yl, 1-(4-fluoronaphthalene-1-sulfonylamino)cyclopropan-1-yl, 1-(4-methylnaphthalene-1-sulfonylamino)cyclopropan-1-yl, 1-(4-isopropoxynaphthalene-1-sulfonylamino)cyclopropan-1-yl, 1-(5-bromonaphthalene-1-sulfonylamino)cyclopropan-1-yl, 1-(4-bromo-2-fluorobenzenesulfonylamino)cyclopropan-1-yl, 1-(4-bromo-2-trifluoromethylbenzenesulfonylamino)cyclopropan-1-yl, 1-(4-bromo-2-methylbenzenesulfonylamino)cyclopropan-1-yl, 1-(4-trifluoromethoxybenzenesulfonylamino)cyclopropan-1-yl, 1-(4-difluoromethoxybenzenesulfonylamino)cyclopropan-1-yl, 1-(4-trifluoromethylthiobenzenesulfonylamino)cyclopropan-1-yl, 1-(4-methylbenzenesulfonylamino)cyclopropan-1-yl, 1-(4-fluorobenzenesulfonylamino)cyclopropan-1-yl, 1-(4-trifluoromethylbenzenesulfonylamino)cyclopropan-1-yl, 1-(4-bromo-3-methylbenzenesulfonylamino)cyclopropan-1-yl, 1-(2,4-dichlorobenzenesulfonylamino)cyclopropan-1-yl, or
Q corresponds to the Q-2 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-methylthiobenzenesulfonylaminomethyl, 4-difluoromethoxybenzenesulfonylaminomethyl, or
Q corresponds to the Q-11 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-6 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-7 radical as claimed in claim 2
and
V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, or
Q corresponds to the Q-9 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-10 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-12 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-13 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, dimethylaminonaphthalene-1-sulfonylaminomethyl, methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 3,4-dichlorobenzenesulfonylaminomethyl, 2,4-dichlorobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-14 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-17 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-26 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-27 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-29 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-31 radical as claimed in claim 2
and
V is defined as 5-iodonaphthalene-1-sulfonylaminomethyl, 4-fluoronaphthalene-1-sulfonylaminomethyl, 4-N-acetylaminonaphthalene-1-sulfonylaminomethyl, 4-methylnaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, 5-bromonaphthalene-1-sulfonylaminomethyl, 4-bromo-2-fluorobenzenesulfonylaminomethyl, 4-trifluoromethylthiobenzenesulfonylaminomethyl, 4-difluoromethoxybenzenesulfonylaminomethyl, or
Q corresponds to the Q-32 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-51 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-52 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-57 radical as claimed in claim 2
and
V is defined as 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-iodobenzenesulfonylaminomethyl, 4-bromo-3-methylbenzenesulfonylaminomethyl, 4-trifluoromethoxybenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl; 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-58 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-60 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-64 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-77 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 3,4-dichlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 2,4-difluorobenzenesulfonylaminomethyl, 2,5-dichlorobenzenesulfonylaminomethyl, 2-chloro-6-fluorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-78 radical as claimed in claim 2
and
V is defined as 4-bromonaphthalene-1-sulfonylaminomethyl, 5-iodonaphthalene-1-sulfonylaminomethyl, 4-fluoronaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-trifluoromethoxybenzenesulfonylaminomethyl, 2-chloro-6-methoxybenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-difluoromethoxybenzenesulfonylaminomethyl, or
Q corresponds to the Q-84 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-109 radical as claimed in claim 2
and
V is defined as 4-bromonaphthalene-1-sulfonylaminomethyl, 5-iodonaphthalene-1-sulfonylaminomethyl, 4-fluoronaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-trifluoromethoxybenzenesulfonylaminomethyl, 2-chloro-6-methoxybenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, or
Q corresponds to the Q-110 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-113 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl,
or
Q corresponds to the Q-114 radical as claimed in claim 2
and
V is defined as 4-bromobenzenesulfonylaminomethyl, naphthalene-1-sulfonylaminomethyl, 4-bromonaphthalene-1-sulfonylaminomethyl, quinoline-8-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-fluorobenzenesulfonylaminomethyl, 3,4-dichlorobenzenesulfonylaminomethyl, 2,4-dichlorobenzenesulfonylaminomethyl, 4-chlorobenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-methoxybenzenesulfonylaminomethyl, 4-methylbenzenesulfonylaminomethyl, 4-trifluoromethoxybenzenesulfonylaminomethyl, 2,4-difluorobenzenesulfonylaminomethyl, 3-chlorobenzenesulfonylaminomethyl, 2,5-dichlorobenzenesulfonylaminomethyl, 2-chlorobenzenesulfonylaminomethyl, 2-chloro-6-methylbenzenesulfonylaminomethyl, 2-chloro-6-fluorobenzenesulfonylaminomethyl, 2,6-dichlorobenzenesulfonylaminomethyl, 2-chloro-6-methoxybenzenesulfonylaminomethyl, 2,5-dimethoxybenzenesulfonylaminomethyl, 2,4,5-trichlorobenzenesulfonylaminomethyl, 3-bromobenzenesulfonylaminomethyl, 2,3-dichlorobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, pyridine-3-sulfonylaminomethyl, quinoline-3-sulfonylaminomethyl, pyridine-2-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 2-methoxybenzenesulfonylaminomethyl, 4-N-acetylaminobenzenesulfonylaminomethyl, 4-trifluoromethylthiobenzenesulfonylaminomethyl, 4-methylthiobenzenesulfonylaminomethyl, 4-difluoromethoxybenzenesulfonylaminomethyl, or
Q corresponds to the Q-139 radical as claimed in claim 2
and
V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 3-bromobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, or
Q corresponds to the Q-140 radical as claimed in claim 2
and
V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 3-bromobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, or
Q corresponds to the Q-141 radical as claimed in claim 2
and
V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 3-bromobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl, or
Q corresponds to the Q-149 radical as claimed in claim 2
and
V is defined as 4-bromobenzenesulfonylaminomethyl, 4-bromonaphthalene-1-sulfonylaminomethyl, 5-iodonaphthalene-1-sulfonylaminomethyl, 4-fluoronaphthalene-1-sulfonylaminomethyl, 4-methoxynaphthalene-1-sulfonylaminomethyl, 4-N-acetylaminonaphthalene-1-sulfonylaminomethyl, 4-methylnaphthalene-1-sulfonylaminomethyl, 4-isopropoxynaphthalene-1-sulfonylaminomethyl, 5-aminonaphthalene-1-sulfonylaminomethyl, 5-dimethylaminonaphthalene-1-sulfonylaminomethyl, 5-methylaminonaphthalene-1-sulfonylaminomethyl, 5-N-acetylaminonaphthalene-1-sulfonylaminomethyl, 7-ethoxynaphthalene-1-sulfonylaminomethyl, N-methyl-1H-indole-7-sulfonylaminomethyl, 5-bromonaphthalene-1-sulfonylaminomethyl, 2,2,4,6,7-pentamethyl-2,3-dihydro-1-benzofuran-5-sulfonylaminomethyl, 4-methoxy-2,5,6-trimethylbenzenesulfonylaminomethyl, 8-chloroquinoline-5-sulfonylaminomethyl, 5,6,7,8-tetrahydronaphthalene-1-sulfonylaminomethyl, 4-bromo-2-fluorobenzenesulfonylaminomethyl, 4-bromo-2-trifluoromethylbenzenesulfonylaminomethyl, N-methyl-1H-indole-4-sulfonylaminomethyl, 4-bromo-3-trifluoromethylbenzenesulfonylaminomethyl, 4-bromo-2-methylbenzenesulfonylaminomethyl, 4-iodobenzenesulfonylaminomethyl, 4-bromo-3-methylbenzenesulfonylaminomethyl, 4-trifluoromethylbenzenesulfonylaminomethyl, 4-trifluoromethoxybenzenesulfonylaminomethyl, 3-bromobenzenesulfonylaminomethyl, 2-fluoropyridine-3-sulfonylaminomethyl, 2-trifluoromethylpyridine-5-sulfonylaminomethyl, 3-bromopyridine-5-sulfonylaminomethyl, quinoline-3-sulfonylaminomethyl, 2,3-dichloropyridine-5-sulfonylaminomethyl, 3-bromo-2-chloropyridine-5-sulfonylaminomethyl, 2-bromobenzenesulfonylaminomethyl, 4-trifluoromethylthiobenzenesulfonylaminomethyl, 4-difluoromethoxybenzenesulfonylaminomethyl.

18. A spray solution for treatment of plants, comprising an amount, effective for enhancing the resistance of plants to abiotic stress factors, of one or more of the compounds as claimed in claim 17.
